(19) United States
(12) Patent Application Publication
(10) Pub. No.: US 2026/0322520 A1
(43) Pub. Date: Oct. 15, 2026

(54) SYSTEMS, DEVICES, AND METHODS FOR WIRELESS COMMUNICATION BETWEEN DEVICES IN AN ANALYTE MONITORING ENVIRONMENT

(71) Applicant: ABBOTT DIABETES CARE INC., Alameda, CA (US)

(72) Inventors: Theodore J. Kunich, Pleasanton, CA (US); Xuandong Hua, Mountain View, CA (US); Jean-Pierre J. Cole, Tracy, CA (US)

(21) Appl. No.: 19/630,766

(22) Filed: Mar. 27, 2026

Related U.S. Application Data
(60) Provisional application No. 63/779,120, filed on Mar. 27, 2025.
(60) Provisional application No. 63/889,745, filed on Sep. 29, 2025.

(51) Int. Cl. H04J 3/06; A61B 5/145; H04W 12/06; H04W 56/00; H04W 76/10

(52) U.S. Cl. CPC H04J 3/0685; A61B 5/14503; A61B 5/1451; H04W 12/06; H04W 56/0015; H04W 76/10

(57) ABSTRACT

Systems, devices, and methods for power management and/or for synchronous advertising-based communication in an analyte monitoring environment are set forth. Information pertaining to clock accuracy is exchanged between a receiver device and an on-body device. The system is configured to make power management decisions based on the clock accuracy information. Avoidance of extended listening windows can provide power savings through prevention or minimization of communication with receiver devices having relatively low clock accuracies. Wireless transmission and reception between devices in an analyte monitoring system in accordance with a synchronous advertising-based protocol are set forth. Advertisements are made by an on-body device and responses are transmitted back from receiver devices. The protocol is highly adaptable for power management. Supplementary information request techniques using synchronous advertising-based protocols and/or secondary channels are set forth. Many variations are set forth.

### CROSS-REFERENCE TO RELATED APPLICATIONS

This application claims the benefit of and priority U.S. Provisional Application Ser. No. 63/889,745, filed Sep. 29, 2025, and to U.S. Provisional Application Ser. No. 63/779,120, filed Mar. 27, 2025, both of which are incorporated by reference herein in their entireties for all purposes.

### FIELD

The present subject matter relates to wireless communication between two or more devices in an analyte monitoring environment.

### BACKGROUND

The human body is a complex biological system in which many chemical substances are involved in a myriad of biological processes. An understanding of the concentration level of these substances and how they vary can provide beneficial information about a person's body. Systems have been developed to conveniently monitor concentration levels of target substances, or analytes, and provide these levels over time for consideration, analysis, and/or further action. These systems typically include an electronic on-body device that is placed on the person's body and is operable in conjunction with a sensor for measuring the concentration levels of the analyte while that analyte is within the body. These systems often operate with a receiver device that can receive analyte concentration levels from the on-body device and display them to the user in a convenient, aesthetic, and informative manner. The sensor component of these analyte monitoring systems can have various configurations that, while operating in conjunction with the on-body device, can be positioned wholly in vivo (within the human body), partially in vivo, or wholly ex vivo to measure the analyte while within the body. These on-body analyte monitoring systems are advantageous over devices used for discrete testing with in vitro techniques such as those that rely on removing a fluid sample from the body (e.g., by a fingerstick) and testing for analyte concentration outside of the body, such as with a test strip and meter. In vitro systems can be inconvenient and painful, and only provide one measurement when the test is performed which leaves large gaps in time for which no measurement readings are available.

A single on-body analyte monitoring system can measure concentration levels for one, two, three, or more different analytes. These analytes can be selected from a range of numerous different analyte types having beneficial relevance for a variety of states pertaining to the human body. These states include medical conditions impacting physical and/or mental health, states of well-being, response to physical exertion, states of fitness, a state of impairment, response to diet, response to physical or mental stress, and/or response to environment generally, to name a few.

On-body analyte monitoring systems are often used by individuals that have or may be susceptible to the medical condition diabetes mellitus (often referred to as diabetes), which is a serious disease where the body does not produce or properly utilize insulin. Insulin is a hormone produced by the pancreas that regulates the substance blood glucose (also referred to as blood sugar). In particular, when blood glucose levels rise (e.g., after a meal), insulin lowers the blood glucose levels by facilitating glucose to move from the blood into the body cells. When the pancreas does not produce sufficient insulin (a condition known as Type I diabetes) or body cells do not properly utilize insulin (a condition known as Type II diabetes), the glucose remains in the blood, resulting in hyperglycemia (high glucose). The inability to control glucose levels can also lead to hypoglycemia (low glucose). These are urgent problems and can even be life-threatening.

The vast and uncontrolled fluctuations in glucose levels in people suffering from diabetes can cause long-term serious health complications. For example, complications may include blindness, kidney failure, and nerve damage. Additionally, it is known that diabetes is a factor in accelerating cardiovascular diseases such as atherosclerosis (hardening of the arteries), leading to stroke, coronary heart disease, and other diseases. It is therefore important to monitor and control glucose levels to manage diabetes.

The body's ketone levels may be dysregulated in people suffering from diabetes. Diabetic ketoacidosis (DKA) is a condition where lack of insulin can lead to build up of harmful ketones in the blood. The monitoring of a ketone as an analyte can thus help manage diabetes; however, ketone monitoring is beneficial for other reasons unrelated to diabetes. For example, individuals with non-diabetic medical conditions that can lead to ketoacidosis, such as eating disorders, gastrointestinal and digestive disorders such as those involving vomiting and/or diarrhea for an extended period, or alcohol use disorders, can benefit from ketone monitoring. Ketone monitoring is also useful for individuals on ketogenic diets, carb-cycling diets, and other diets low in carbohydrates, as well as individuals that are pregnant or performing rigorous exercise routines.

Another analyte that is beneficial to monitor is lactate (also called lactic acid). Lactate monitoring can provide insight for individuals that desire to analyze exercise performance by examining lactate thresholds, peak tolerance, and/or fatigue. Lactate monitoring can also be used to assess medical conditions such as heart failure, liver disease, and/or others.

Ethanol (ethyl alcohol) is yet another analyte that is beneficial to monitor. Ethanol is the primary alcohol found in adult alcoholic beverages. On-body analyte monitoring systems capable of measuring in vivo ethanol levels can be useful in determining cognitive impairment, which in turn can assist in avoiding operation of motor vehicles or other machinery while impaired. Such systems can additionally or alternatively be used in the context of assisting individuals to achieve sobriety as well as in a legal enforcement context.

The on-body device of the system is typically attached to the subject's skin, often in a location on the arm, abdomen, or upper buttock. The amount of time the on-body device has been active on the body is referred to as the wear duration. The full completed extent of time the on-body device was active on the body, from assumption of an activated state on the body to termination of the on-body device's functional measurement capability on the body (e.g., such as by expiration, fault, or removal) is referred to as the completed wear duration. In an effort to ensure reliable measurements, some systems with partially implanted sensors may have a maximum duration the on-body device and sensor are permitted to operate together while on the subject's body, and this is referred to as the maximum wear duration. The maximum wear duration can be enforced by the software of a system component, such as the on-body device, the sensor itself, and/or a receiver device, so that new analyte level measurements can no longer be taken and/or displayed after the maximum wear duration has been reached. The maximum wear duration varies based on the implementation of the sensor and on-body device but is in the range of 7 to 15 days for conventional systems. This range may include an initial warm-up period, usually in the range of one hour to one day, during which the on-body device is active on the body, but the system is not permitted to output new analyte measurements to the subject. Some but not all systems require a warm-up period, for example, to allow the body's biological response to the sensor's presence to subside and/or to allow the inputting of in vitro measurements to calibrate the sensor.

System developers design the on-body device and sensor to remain on the body while generating reliably accurate analyte measurements for as long of a wear duration as is practical and commercially viable. Conditions that the on-body device and sensor are subjected to will vary from one subject to the next due to biological conditions in and/or on the subject's body and/or environmental conditions the system is exposed to. This variance leads to unpredictability in determining the likely completed wear duration the on-body device and sensor will achieve before requiring replacement. Nevertheless, system developers can estimate, with a high degree of confidence derived from analysis and testing, a wear duration during which the on-body device and sensor will remain operable if used within a range of operating conditions specified by the developer. This estimate is referred to herein as the designed wear duration.

The designed wear duration of the on-body device is often limited by the power supply life, which is the amount of time the power supply can provide the requisite power for operation of the on-body device (and also the sensor if the on-body device is responsible for powering the sensor). The power supply life is dependent on the power capacity of the power supply and the power consumption by the on-body device electronics, as well as the power consumption of the sensor in cases where the sensor does not have its own power supply. The designed wear duration of the on-body device can additionally or alternatively be limited by the attachment life, which is the amount of time the attachment structure holds the on-body device to the body. This attachment structure can be an adhesive patch, a non-adhesive wrap or bandage, a combination of both or otherwise. In the case of on-body devices adhesively attached to the skin, a significant factor in determining the attachment life is the amount of time sufficient adhesive coupling with the skin can be maintained. For on-body devices having a partially implanted sensor or wholly ex vivo sensor, the designed wear duration can additionally or alternatively be limited by the sensor life, which is the length of time the sensor can produce a reliably accurate measurement signal, which in turn is often limited by either or both of the performance of the electrochemical or other primary technique by which the sensor generates the measurement signal and, for partially and wholly implanted sensors, the body's biological response to the presence of the sensor which can degrade the measurement signal and/or lead to irritation and/or infection.

It is desirable to extend the designed wear duration of on-body devices to improve convenience for the user by increasing the time between replacements. Replacing an on-body device can be inconvenient since it requires the user to be available and have a replacement on-body device on hand at the required time. It is therefore desirable to reduce the frequency at which this is necessary. Providing a longer designed wear duration may also reduce costs for the user since fewer on-body devices would be required over a given period of time. This can also reduce waste as well as energy and resource consumption involved in production for a given user. On-body devices with relatively longer designed wear durations can also exemplify higher quality and/or sophistication in the marketplace, which can lead to greater revenue for the system developer.

Thus, a need exists for on-body devices for analyte monitoring having relatively longer designed wear durations.

### SUMMARY

Systems, devices, and methods for power management in an analyte monitoring environment are described by way of example embodiments set forth throughout this document. The system includes an on-body device that is configured for placement on the skin of a subject. The on-body device includes device electronics located within a housing. The device electronics include sensor interface electronics for interfacing with a sensor configured to measure the analyte level, which is a glucose level in many embodiments. The sensor can be wholly implanted within the subject's body, partially implanted within the subject's body, or wholly ex vivo as described herein. The device electronics include processing electronics for processing data and executing instructions pertaining to the operation of the on-body device. The device electronics include wireless communication electronics for sending and receiving wireless communications with one or more receiver devices. The wireless communications include analyte measurements obtained by use of the sensor and other information related to the operation of the system.

The one or more receiver devices take various forms across the embodiments. In one form, the receiver device is a multi-purpose mobile receiver device, such as a smart phone or a smart wearable device. In another form, the receiver device is a dedicated receiver device. In a further form, the receiver device is a drug delivery device. Receiver devices of two or more of these forms may be used together in the system. The receiver devices execute analyte monitoring software that reads the received analyte measurements and initiates one or more actions to process the analyte measurements, such as to assist in a drug delivery function, and further to display the analyte measurements on a display of the receiver device, and/or to wirelessly relay the analyte measurements to another receiver device for display thereon. The receiver devices include wireless communications circuitry that sends and receives wireless communications to and from the on-body device as well as any other receiver devices in the system.

As mentioned, several significant factors that can impact an on-body device's wear duration are power supply life, sensor life, and/or attachment structure life. The embodiments described herein relate to improvements to the power supply life component of wear duration by reducing wireless communication inefficiencies due to clock accuracy issues.

A receiver device includes one or more clocks. At least one of the clocks generates a timing reference for the sending and receiving of wireless communications to and from the on-body device. Receiver devices can have wireless communication clocks with clock accuracies that vary, sometime substantially. Receiver devices that utilize a wireless communication clock with a clock accuracy that is relatively low, as compared with clocks of other receiver devices available to the user, can send transmissions to the on-body device at incorrect times due to the low accuracy clock. The on-body device must consume power in the operation of its wireless receiver to monitor for incoming transmissions from the receiver devices and when the incoming transmission is substantially delayed from the time when it should have been received, the result is inefficient power use by the on-body device.

The example embodiments set forth herein allow the on-body device to receive information or data indicative of the clock accuracy of the receiver device. In some embodiments herein this data or information takes the form of a clock accuracy parameter. The receiver device can send this clock accuracy parameter to the on-body device. The on-body device can receive it and assess the clock accuracy parameter. The on-body device can then progress one or more power management actions based on the assessed clock accuracy parameter. The power management actions assist the user and/or the on-body device in managing its power to extend power supply life and potentially last a longer wear duration and/or to mitigate the potential loss of wear duration due to excessive power supply drainage.

In other embodiments the receiver device can alternatively or additionally send other data or information indicative of the clock accuracy such as type information indicating the type of receiver device, e.g., a dedicated receiver device or a drug delivery device. The type information can indicate a model and/or manufacturer, such as a smart phone of a specific generation and/or made by a specific manufacturer. This type information can be known to the on-body device as correlating to a clock accuracy that is or is not acceptable for use with the on-body device.

As explained in more detail herein, the on-body device can monitor its elapsed wear duration and/or the remaining wear duration before reaching a target wear duration for itself. The target wear duration can be a time value that is representative of the amount of time the on-body device is expected to operate in an analyte monitoring capacity while on the body. The target wear duration can be the same as, or representative of, the designed wear duration, though such is not required. The target wear duration can be the same as or less than the maximum wear duration (if applicable). The target wear duration can be adjusted by the on-body device to reflect power or energy usage.

The power management action takes various forms across the embodiments. The power management action is (a) one or more determinations of one or more power or energy consequences to the on-body device that can result from communication with a receiver device having the assessed clock accuracy; (b) one or more adjustments to an operating aspect of the on-body device; (c) one or more determinations of a target wear duration consequence to the on-body device that can result from communication with a receiver device having the assessed clock accuracy; (d) one or more issuances of a notification to a user based on the assessed clock accuracy; (e) one or more combinations of the aforementioned, including combinations of: (a) with (b); (a) with (c); (a) with (d); (b) with (c); (b) with (d); (c) with (d); (a) with (b) and (c); (a) with (c) and (d); (a) with (b) and (d); (b) with (c) and (d); and (a) with (b), (c) and (d).

The on-body device can progress the power management action by initiating one or more of them. For example, the on-body device can instruct the receiver device of the unacceptability of the clock accuracy, which can result in the receiver device issuing the notification to the user on the display of the receiver device. The on-body device can progress the power management action by furthering one or more actions already initiated by itself or another device.

The system can perform power management actions at entities other than the on-body device. The system can be configured so that entities other than the on-body device perform the tasks to assist the on-body device in managing power. Power management actions that do not directly involve the on-body device can be performed by other entities within the system, such as: by one or more of the receiver devices, including by an app such as a glucose and/or other analyte monitoring app being executed by a receiver device; by a computing device other than a receiver device in direct communication with the on-body device; and/or by a trusted computer system such as a remote server.

The system can be configured such that the on-body device communicates glucose and/or other analyte measurements to multiple receiver devices throughout the target wear duration as part of a multi-device connectivity (MDC) environment or setting. The on-body device and receiver devices of the system are arrangeable in various MDC wireless communication topologies including one-to-many, mesh, and partial mesh topologies. In an MDC environment, information indicative of the respective clock accuracy of the receiver devices is communicated between entities (such as different on-body and receiver devices or between layers of a single receiver device protocol stack) and the information indicative of the clock accuracy is assessed. Subsequently, either or multiple ones of the on-body device and the receiver devices can take one or more power management actions based on the assessment. The power management actions can be those described above and/or others.

In some embodiments, the system can change the manner of communication with one or more of the multiple receiver devices based on the assessed clock accuracy of one of the respective receiver devices. One of the receiver devices having relatively higher clock accuracy is designatable as a central point of communication through which communications to it and other receiver devices are routed. This can avoid unnecessary communications with a receiver device having a relatively lower clock accuracy. The system can thus change the communication topology in response to the clock accuracy assessments. Other changes to the manner of communication can additionally or alternatively be made, such as the mode of communication being changed between a connection-oriented mode of communication such as a paired or a direct-connection communication session, and a connection-less mode of communication, such as communication by way of advertising transmissions and responses. A rate of transmission, or an interval between transmissions, can additionally or alternatively be a manner of communication that is changed based on the assessed clock accuracy. A change in the manner of communication can affect one or more of the receiver devices within the MDC environment.

In some embodiments, the system can adjust a clock accuracy parameter (e.g., a first clock accuracy parameter) received from a transmitting device to a different clock accuracy parameter (e.g., a second clock accuracy parameter) that more accurately reflects the actual clock accuracy of that device. The substitution of one clock accuracy parameter for another mitigates the risk that a reported clock accuracy parameter significantly underestimates the actual clock accuracy of the transmitting device, and mitigates the associated risk of excessive power consumption by activating receive circuitry of wireless communication electronics of the receiving device too early to account for clock drift that is beyond the range of actual clock drift likely to occur. The receiving device can use the second clock accuracy parameter (as well as clock accuracy information of the receiving device itself) to then determine the amount of clock drift likely to occur before the next incoming transmission is scheduled to be received, and can determine the time at which the wireless communication electronics of the receiving device should be utilized (e.g., activated, monitor for receipt, transmit, change between power states, and the like).

The identification and/or determination of the second clock accuracy parameter can be performed by the receiving device itself, or another device in the system and then relayed back to the receiving device. The received first clock accuracy parameter can be evaluated according to a substitution condition that guides whether the first clock accuracy parameter should be substituted with a second clock accuracy parameter. The substitution condition can take various forms as described in greater detail herein, and the technique for choosing the value of the second clock accuracy parameter can likewise take various forms as also described herein.

The system can also be configured to dynamically adjust the clock accuracy parameter, e.g., tune the parameter, to arrive at one that more accurately reflects the actual clock accuracy of the transmitting device. In some embodiments, the receiving device can detect if a timing error occurred indicative of use of a clock accuracy parameter that overestimated the clock accuracy of the transmitting device, in which case the receiving device can adjust the clock accuracy parameter to a new value representing a more inaccurate clock. The clock accuracy parameter can also be adjusted to a new value representing a more accurate clock if successful communication is consistently exhibited. In this manner, with one or more adjustments, the receiving device can iteratively tune the clock accuracy parameter to a more accurate parameter than that originally reported and that first substituted. Methods of using the system to perform these functions are also described.

The embodiments herein can utilize one or more of numerous wireless communication protocols for communication between the on-body device and the one or more receiver devices. The embodiments herein can additionally utilize one or more of numerous wireless communication protocols for communication between different receiver devices, including receiver devices of all types. These communication protocols include, but are not limited to, one or two or more of any of those in the “Statement of Wireless Communication Protocols for the Present Embodiments” recited herein.

Embodiments are described herein pertaining to on-body analyte monitoring systems and methods that communicate between devices using a synchronous advertising-based wireless communication protocol. In this synchronous protocol, information (e.g., data) is sent at scheduled times where the transmitting and receiving devices can determine those times with accuracy because their respective timing references (e.g., clocks) are aligned. The devices can be considered as operating with a shared timing reference. These embodiments can improve designed wear duration by allowing more efficient wireless communication where wireless communication electronics of on-body devices and receiver devices can be kept in a relatively lower power state, such as a deactivated state, for longer periods of time given the benefits of synchronous operation. Other non-synchronous protocols, such as asynchronous and isochronous protocols, require the wireless communication electronics to be on for longer periods of time to monitor for potential transmissions. Even certain asynchronous protocols operate in near synchronous fashion, but inject random timing perturbations (e.g., short variable delays) to mitigate risk of data collisions, and thus do not provide the same level of benefit as the synchronous protocols of the present embodiments.

In these embodiments where the synchronous advertising-based transport protocol is used to conduct a communication session, the on-body device synchronously transmits or sends an advertisement every transmission interval and one or more receiver devices receive the advertisement and synchronously transmit a response. The advertisement can include data indicative of the level of an analyte of the wearer of the on-body device. Each receiver device that transmits a response does so in an assigned response time slot. The response can indicate whether or not the receiver device validly received the advertisement. Valid reception can include verifying that advertisement was transmitted by an authenticated on-body device and/or by error checking the received data.

If all receiver devices validly confirm reception, then the on-body device can cease transmitting, or alternatively transmit a confirmatory advertisement that the responses were received. If a receiver device does not confirm valid reception, sometimes referred to as an non-confirming or unresponsive receiver device, then the on-body device can retransmit the data indicative of the level of the analyte in a second advertisement. That advertisement can be sent to all receiver devices or can be addressed or directed to only the non-confirming or unresponsive receiver device. The process of resending in the second advertisement can occur in a subsequent transmission interval. Also, or alternatively, the second advertisement can be sent in a transmission subinterval of the first transmission interval. Each transmission interval can be divided into transmission subintervals, which each have the same temporal length and contain response time slots for receiver device responses. A transmission interval can have two, three, four, or more subintervals, including dozens of subintervals, based on the transmission interval and subinterval lengths. The process of resending in the second advertisement can occur repeatedly, in third, fourth, or fifth advertisements, and so forth, until all receiver devices confirm valid receipt. After each time the on-body device sends an advertisement, it can optionally transition its wireless communication electronics to a lower power state (e.g., deactivate it) until the next time the on-body device needs to monitor for a response. Once a receiver device validly confirms receipt, the on-body device can cease monitoring the response time slot for that receiver device to minimize power consumption. Once a receiver device receives a response in a response time slot it can cease monitoring that response time slot even if the time slot has not yet expired. Many additional examples and embodiments of scenarios of communication in the synchronous advertising-based communication session are described further herein.

The devices can take actions to increase the likelihood that the advertisement will be validly received, as well as the response thereto. These can include adjusting transmit power, response slot assignments, and/or transmitting responses in multiple response time slots. If repeatedly unsuccessful, the on-body device can cease attempting to confirm receipt with a particular receiver device after a duration of time, threshold number of attempts, or a combination thereof. A number of subsequent actions that can be taken in this regard are described herein.

All of the embodiments of synchronous advertising-based transport protocols can be performed in accordance with a Bluetooth protocol, such as the Bluetooth Low Energy protocol. One example of a suitable advertising-based protocol is periodic advertising with response (PAwR). All of the embodiments of synchronous advertising-based transport protocols can be performed in accordance with other compatible protocols, including non-Bluetooth protocols, a number of examples of which can be found in the “Statement of Wireless Communication Protocols for the Present Embodiments” section.

The on-body device and one or more receiver devices can negotiate or exchange parameters to setup a communication session in accordance with the synchronous advertising-based protocol. This can be done as part of a separate communication session, e.g., a non-synchronous session. Examples of communication sessions that can be used to negotiate setup information include a 1:1 pairing between the on-body device and the receiver device seeking to participate, an advertising-based protocol that can be non-synchronous, and/or an induced wireless communication session such as one performed with Near Field Communication (NFC) or Radio Frequency Identification (RFID). Setup information can also be exchanged via an intermediary, such as an internet connection through a router, and/or a remote server. The types of setup information are described in further detail herein.

The on-body device can be configured to manage response slot assignments to maintain a predetermined degree of power efficiency, which may provide the highest degree of power efficiency as receiver devices leave and/or join the synchronous communication session. The on-body device can reassign response slots as desired or needed to avoid gaps where wireless communication electronics would otherwise remain active without receiving transmissions. Examples of response slot management are described further herein.

The system can be configured to take numerous different actions to improve power efficiency. Examples of such actions can include, but are not limited to: (a) managing slot assignments; (b) adjusting transmit power; (c) adjusting physical format of transmissions; (d) adjusting transmission interval durations; (e) adjusting transmission subinterval durations; (f) adjusting response time slot durations; (g) performing selective response monitoring; (h) changing transport protocols (e.g., from synchronous to non-synchronous, or from non-synchronous to synchronous); (i) performing multiple communication sessions in parallel using different transport protocols; (j) consolidating multiple communication sessions into less or one communication session, and/or the like. Embodiments performing these actions are described in further detail herein. These actions can be performed individually or in combination with one or more other actions. For example, action (a) can be performed with any one or more of actions (b), (c), (d), (e), (f), (g), (h), (i), and/or (j). Action (b) can be performed with any one or more of actions (a), (c), (d), (e), (f), (g), (h), (i), and/or (j). Action (c) can be performed with any one or more of actions (a), (b), (d), (e), (f), (g), (h), (i), and/or (j). Action (d) can be performed with any one or more of actions (a), (b), (c), (e), (f), (g), (h), (i), and/or (j). Action (e) can be performed with any one or more of actions (a), (b), (c), (d), (f), (g), (h), (i), and/or (j). Action (f) can be performed with any one or more of actions (a), (b), (c), (d), (e), (g), (h), (i), and/or (j). Action (g) can be performed with any one or more of actions (a), (b), (c), (d), (e), (f), (h), (i), and/or (j). Action (h) can be performed with any one or more of actions (a), (b), (c), (d), (e), (f), (g), (i), and/or (j). Action (i) can be performed with any one or more of actions (a), (b), (c), (d), (e), (f), (g), (h), and/or (j). Action (j) can be performed with any one or more of actions (a), (b), (c), (d), (e), (f), (g), (h), and/or (i).

Described herein are example embodiments of response monitoring by other observers. For example, while the on-body device can monitor for a response transmitted by a receiver device, other receiver devices can monitor for the response as well. The system can be configured such that all receiver devices can monitor the responses of all other receiver devices. The receiver devices can interpret the responses and take action based on the information contained in the response. This permits the system an additional degree of freedom through which to exchange information. This can allow time sensitive information, such as alarm state information, to be exchanged between devices in a more rapid fashion than going through an intermediary, such as the on-body device or a remote server. Many different types of information can be used with response monitoring, and examples are described further herein, along with additional embodiments of the same.

The on-body device can act as the advertiser and the one or more receiver devices can act as observers. The embodiments herein can be performed with the roles of the devices reversed, where the actions taken by the on-body device with respect to the synchronous advertising-based communication session can performed instead by the receiver device. In such a configuration, the receiver device can use advertisements to solicit data indicative of a level of an analyte of the wearer, and the on-body device can send the analyte data in the response to the advertisement. Thus a receiver device can act as an advertiser, and the on-body device and/or other non-advertising receiver devices, can act as observers. Receiver devices acting as observers can utilize the receiver response monitoring embodiments described herein to receive, read, and/or store analyte data that may be transmitted by the on-body device in a response to an advertisement transmitted by another receiver device.

Described herein are example embodiments of processing supplementary information requests. The requests can be transmitted and responded to within a synchronous advertising-based communication session. The requests can also, or alternatively, be transmitted and responded to within a non-synchronous extended advertising-based session, utilizing secondary channels. The supplementary information being requested can be any type of information that a device within the on-body analyte monitoring system would benefit from obtaining from another device within the same system or operating with the same wearer. Many examples of the information types are described herein along with example embodiments. The supplementary information request can be particularly useful for analyte data that a receiver device may have missed receipt of previously, or is otherwise without access to, as provision of the missing analyte data to the receiver device will allow presentation of the analyte data to a user without gaps in time. Supplementary information requests of analyte data are sometimes referred to as backfilling requests. The receiver device can issue a supplementary information request to the on-body device, which processes the request and responds with the requested supplementary information. The on-body device can alternatively issue a supplementary information request to the receiver device, which processes the request and responds with the requested supplementary information. A first receiver device can issue a supplementary information request to a second receiver device if desired. The various embodiments described herein can be performed in each of these different device configurations by switching roles of the devices within the system.

All of the embodiments described with respect to synchronous advertising-based communication sessions and supplementary information requests can be performed in combination with all of the embodiments pertaining to the assessment of clock accuracy parameter.

Other systems, devices, methods, features and advantages of the subject matter described herein will be or will become apparent to one with skill in the art upon examination of the following figures and detailed description. It is intended that all such additional systems, devices, methods, features and advantages be included within this description, be within the scope of the subject matter described herein and be protected by the accompanying claims. In no way should the features of the example embodiments be construed as limiting the appended claims, absent express recitation of those features in the claims.

### BRIEF DESCRIPTION OF FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter, unless otherwise stated and/or evident. Moreover, all illustrations are intended to convey concepts, where relative sizes, shapes and other detailed attributes may be illustrated schematically rather than literally or precisely, unless otherwise stated and/or evident.

FIGS. 1A, 1B, 1C, and 1D are block diagrams depicting example embodiments of an on-body analyte monitoring system.

FIG. 1E is a partial cross-sectional view depicting an example embodiment of an on-body device on the body of a subject.

FIG. 1F is a partial cross-sectional view depicting an example embodiment of an on-body device on the body of a subject in communication with a wholly implanted sensor.

FIGS. 1G, 1H, and 1I are perspective views depicting an example embodiment of an inserter for an on-body device having a partially implantable sensor at various stages of use.

FIGS. 1J, 1K, and 1L are perspective views depicting another example embodiment of an inserter for an on-body device having a partially implantable sensor at various stages of use.

FIGS. 1M, 1N, and 1O are perspective views depicting an example embodiment of an inserter for a wholly implantable sensor at various stages of use.

FIGS. 1P and 1Q are perspective views depicting an example embodiment of an applicator at various stages of application of an on-body device to the skin for use in conjunction with a wholly implantable sensor.

FIGS. 1R and 1S are perspective views depicting an example embodiment of an applicator at various stages of application of an on-body device having a wholly ex vivo sensor to the skin.

FIGS. 2A and 2B are perspective views of example embodiments of a partially implantable sensor.

FIG. 2C is a cross-sectional view of another example embodiment of a partially implantable sensor.

FIG. 2D is a front view depicting another example embodiment of a partially implantable sensor.

FIG. 2E is a perspective view depicting another example embodiment of a partially implantable sensor.

FIG. 2F is a perspective view depicting an example embodiment of a partially implantable sensor.

FIG. 2G is a perspective view depicting another example embodiment of a partially implantable sensor.

FIGS. 3A and 3B are block diagrams depicting example embodiments of electronics of on-body device.

FIG. 3C is a block diagram depicting an example embodiment of electronics of a receiver device.

FIGS. 4A and 4B are timing diagrams depicting examples of situations of wireless communication between an on-body device and a receiver device.

FIG. 5A is a flow diagram depicting an example embodiment of a method of power management in an on-body analyte monitoring system.

FIG. 5B is a flow diagram depicting an example embodiment of a method of power management in an on-body analyte monitoring system.

FIG. 5C is a front view depicting an example embodiment of a display and input interface of a receiver device.

FIG. 5D is a flow diagram depicting an example embodiment of a method of power management in an on-body analyte monitoring system.

FIGS. 6A-6C are block diagrams depicting example embodiments of wireless communication topologies in an on-body analyte monitoring system.

FIG. 7A is a flow diagram depicting an example embodiment of a method of power management in an on-body analyte monitoring system.

FIG. 7B is a block diagram depicting an example embodiment of a change of state in a wireless communication topology of an on-body analyte monitoring system.

FIG. 7C is a flow diagram depicting an example embodiment of a method of power management in an on-body analyte monitoring system.

FIG. 7D is a block diagram depicting an example embodiment of a change of state in a wireless communication topology of an on-body analyte monitoring system.

FIGS. 7E-7F are flow diagrams depicting example embodiments of methods of using an on-body analyte monitoring system.

FIG. 8A is a sequence diagram depicting an example embodiment of a sequence of transmissions in an on-body analyte monitoring system.

FIG. 8B is a timing diagram depicting an example embodiment of a synchronous advertising-based communication session with an on-body analyte monitoring system.

FIG. 8C is a flow diagram depicting an example embodiment of a method of wireless communication in a synchronous advertising-based communication session with an on-body analyte monitoring system.

FIG. 8D is a flow diagram depicting another example embodiment of a method of wireless communication in a synchronous advertising-based communication session with an on-body analyte monitoring system.

FIG. 8E is a sequence diagram depicting another example embodiment of a sequence of transmissions in an on-body analyte monitoring system.

FIG. 8F is a timing diagram depicting another example embodiment of a synchronous advertising-based communication session with an on-body analyte monitoring system.

FIG. 8G is a flow diagram depicting another example embodiment of a method of wireless communication in a synchronous advertising-based communication session with an on-body analyte monitoring system.

FIG. 9A is a sequence diagram depicting another example embodiment of a synchronous advertising-based communication session with an on-body analyte monitoring system.

FIG. 9B is a timing diagram depicting another example embodiment of a synchronous advertising-based communication session with an on-body analyte monitoring system.

FIG. 9C is a timing diagram depicting another example embodiment of a synchronous advertising-based communication session with an on-body analyte monitoring system.

FIG. 9D is a timing diagram depicting another example embodiment of a synchronous advertising-based communication session with an on-body analyte monitoring system.

FIG. 9E is a timing diagram depicting another example embodiment of a synchronous advertising-based communication session with an on-body analyte monitoring system.

FIG. 9F is a sequence diagram depicting another example embodiment of a synchronous advertising-based communication session with an on-body analyte monitoring system.

FIG. 9G is a timing diagram depicting another example embodiment of a synchronous advertising-based communication session with an on-body analyte monitoring system.

FIG. 10A is a sequence diagram depicting another example embodiment of a synchronous advertising-based communication session with an on-body analyte monitoring system.

FIG. 10B is a timing diagram depicting another example embodiment of a synchronous advertising-based communication session with an on-body analyte monitoring system.

FIG. 11 is a flow diagram depicting another example embodiment of a method of wireless communication in a synchronous advertising-based communication session with an on-body analyte monitoring system.

FIGS. 12A, 12B, and 12C are timing diagrams depicting additional example embodiments of synchronous advertising-based communication sessions with an on-body analyte monitoring system.

FIG. 13A is a flow diagram depicting another example embodiment of a method of wireless communication in a synchronous advertising-based communication session with an on-body analyte monitoring system.

FIG. 13B is a timing diagram depicting another example embodiment of a synchronous advertising-based communication session with an on-body analyte monitoring system.

FIG. 13C is a timing diagram depicting another example embodiment of a synchronous advertising-based communication session with an on-body analyte monitoring system.

FIG. 13D is a timing diagram depicting another example embodiment of a synchronous advertising-based communication session with an on-body analyte monitoring system.

FIG. 13E is a timing diagram depicting another example embodiment of a synchronous advertising-based communication session with an on-body analyte monitoring system.

FIG. 13F-1 is a timing diagram depicting another example embodiment of a synchronous advertising-based communication session with an on-body analyte monitoring system.

FIG. 13F-2 is a timing diagram depicting another example embodiment of a synchronous advertising-based communication session with an on-body analyte monitoring system.

FIG. 13F-3 is a timing diagram depicting another example embodiment of a synchronous advertising-based communication session with an on-body analyte monitoring system.

FIG. 13F-4 is a timing diagram depicting another example embodiment of a synchronous advertising-based communication session with an on-body analyte monitoring system.

FIG. 13F-5 is a timing diagram depicting another example embodiment of a synchronous advertising-based communication session with an on-body analyte monitoring system.

FIGS. 14A, 14B, 14C, and 14D are timing diagrams depicting additional example embodiments of wireless communication with an on-body analyte monitoring system.

FIG. 14E is a flow diagram depicting an example embodiment of a method of wireless communication that includes switching between transport protocols.

FIG. 14F is a flow diagram depicting an example embodiment of a method of evaluating whether to add a receiver device to a synchronous communication session.

FIG. 15A is a flow diagram depicting an example embodiment of a method of wireless communication by monitoring responses in a synchronous advertising-based communication session with an on-body analyte monitoring system.

FIG. 15B is a timing diagram depicting another example embodiment of a synchronous advertising-based communication session with an on-body analyte monitoring system.

FIG. 15C is a flow diagram depicting an example embodiment of a method of wireless communication by monitoring responses in a synchronous advertising-based communication session with an on-body analyte monitoring system.

FIG. 16A is a timing diagram depicting an example embodiment of a wirelessly communicating missing analyte data with an on-body analyte monitoring system.

FIG. 16B is a flow diagram depicting an example embodiment of a method of wirelessly communicating missing analyte data in a synchronous advertising-based communication session with an on-body analyte monitoring system.

FIG. 16C is a timing diagram depicting another example embodiment of a wirelessly communicating missing analyte data with an on-body analyte monitoring system.

FIG. 16D is a flow diagram depicting an example embodiment of a method of wirelessly communicating missing analyte data using extended advertising with an on-body analyte monitoring system.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

### On-Body Analyte Monitoring Systems

The subject matter described herein generally relates to assessing the accuracy of a clock and then progressing an action in response to that assessment within an on-body analyte monitoring system (or on-body monitoring system). As explained herein, the clock can be responsible for acting as a reference for the sending of wireless transmission. Examples of actions that can be taken vary considerably and are described in more detail herein (e.g., a power management action). As used herein the term “analyte” refers to a target substance for detection according to a chemical technique, an electrical technique, an electro-chemical technique, a photonic technique based on non-ionizing radiation, or a combination of two or more thereof.

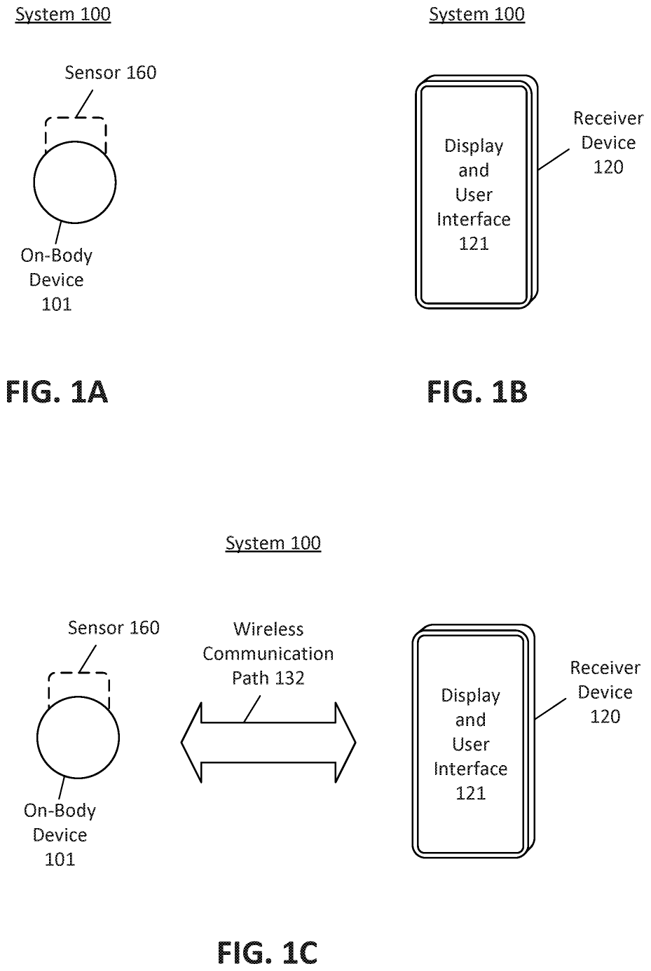

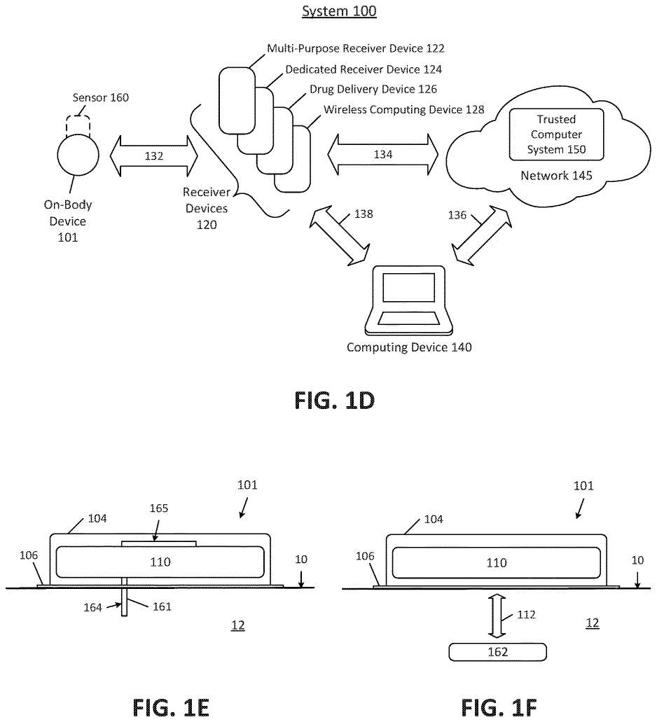

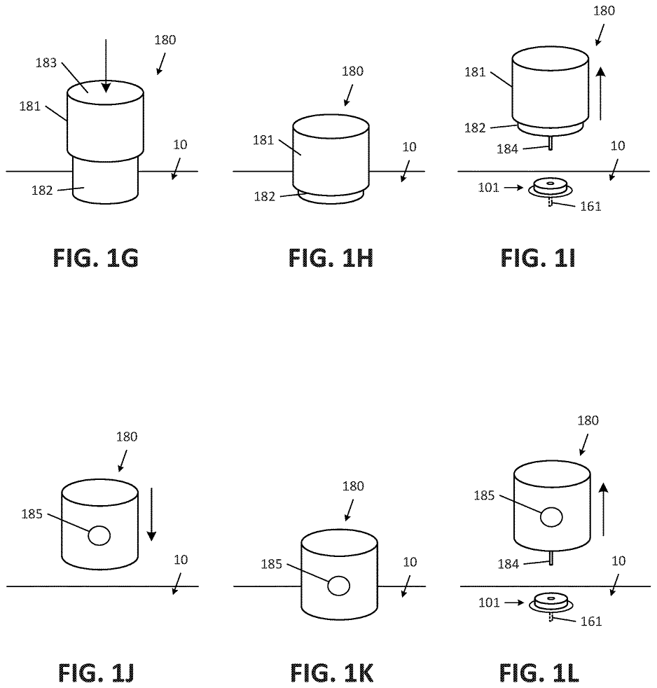

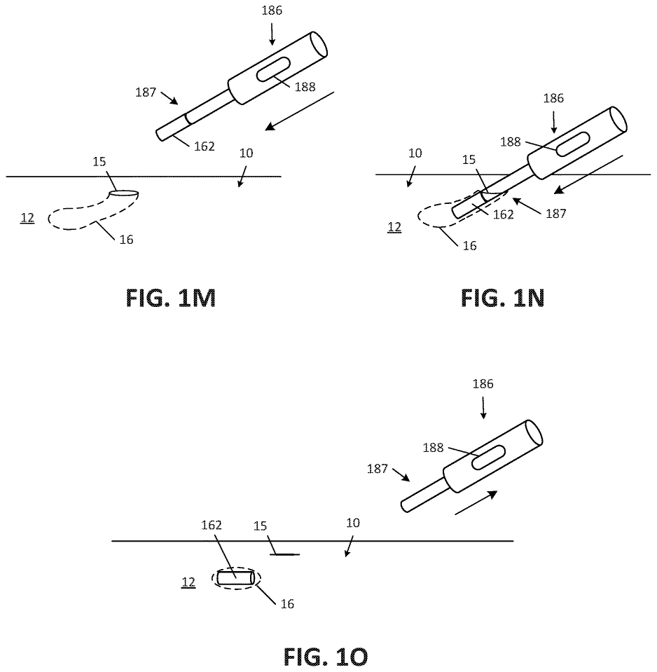

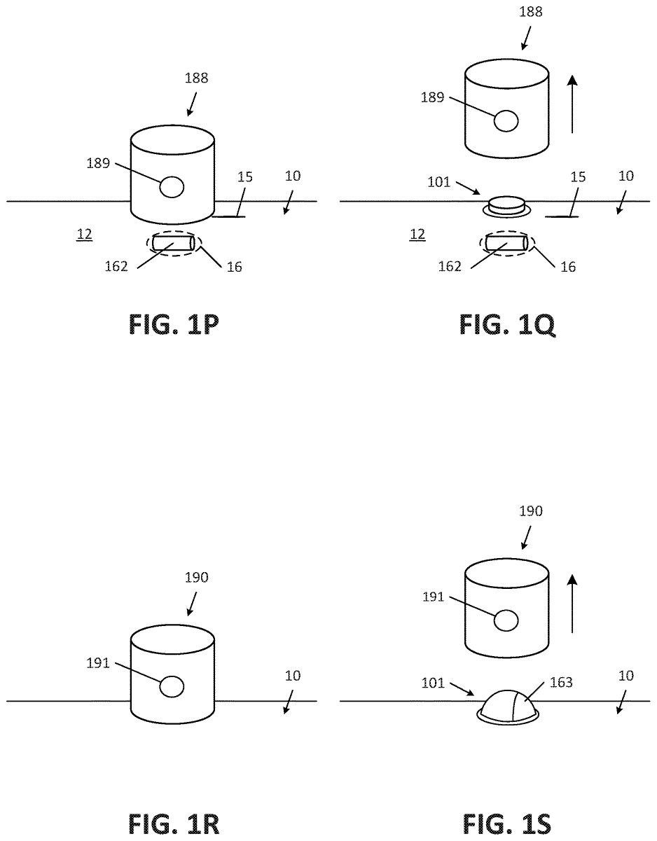

Embodiments of this subject matter can be performed on, by, or with only one device within the on-body analyte monitoring system, or on, by, or with two, three, four or more devices of the system. FIGS. 1A and 1B are block diagrams depicting example embodiments of an on-body analyte monitoring system 100 having a single device that embodies the subject matter described herein. FIG. 1A depicts an example embodiment of system 100 having an on-body device (OBD) 101 configured to operate in conjunction with an analyte sensor 160. In this embodiment, OBD 101 alone is configured to assess the clock accuracy and progress an action in response to the assessment. The clock accuracy can pertain to a different device and be previously reported to OBD 101. Alternatively the clock accuracy can be that of the OBD 101 itself. For example, the OBD 101 may be configured to report the clock accuracy from one protocol layer to a higher layer within the electronics architecture of OBD 101. FIG. 1B depicts an example embodiment of system 100 having a receiver device 120 configured as a multi-purpose mobile receiver device (e.g., a smart phone) with a display that also acts as an input interface 121 (e.g., touchscreen and/or some other input interface). In this embodiment, receiver device 120 alone is configured to assess the clock accuracy and progress an action in response to the assessment. The clock accuracy can pertain to a different device and be previously reported to the receiver device 120. Alternatively, the clock accuracy can be a clock accuracy of the receiver device 120 itself. For example, the receiver device 120 may be configured to report the clock accuracy from one layer to a higher layer within the electronics architecture of receiver device 120.

FIG. 1C is a block diagram depicting an example embodiment of an on-body analyte monitoring system 100 having two devices embodying the subject matter described herein. Here, system 100 has an on-body device (OBD) 101, configured to operate in conjunction with an analyte sensor 160, and a receiver device 120. OBD 101 and receiver device 120 are configured to wirelessly communicate using one or more wireless protocols over path 132. Receiver device 120 can report its clock accuracy to OBD 101 over path 132. OBD 101 can assess the clock accuracy of the receiver device 120, and then take an action in response to that assessment, if needed.

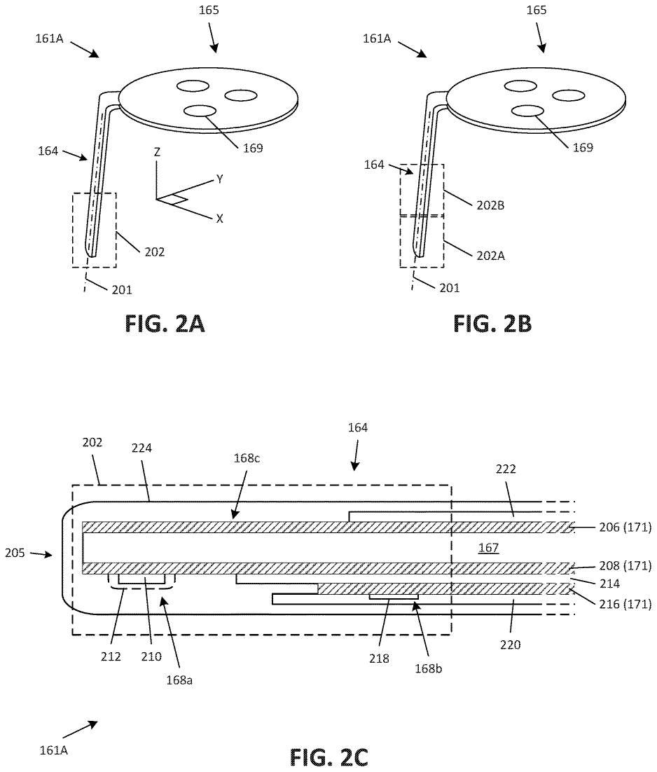

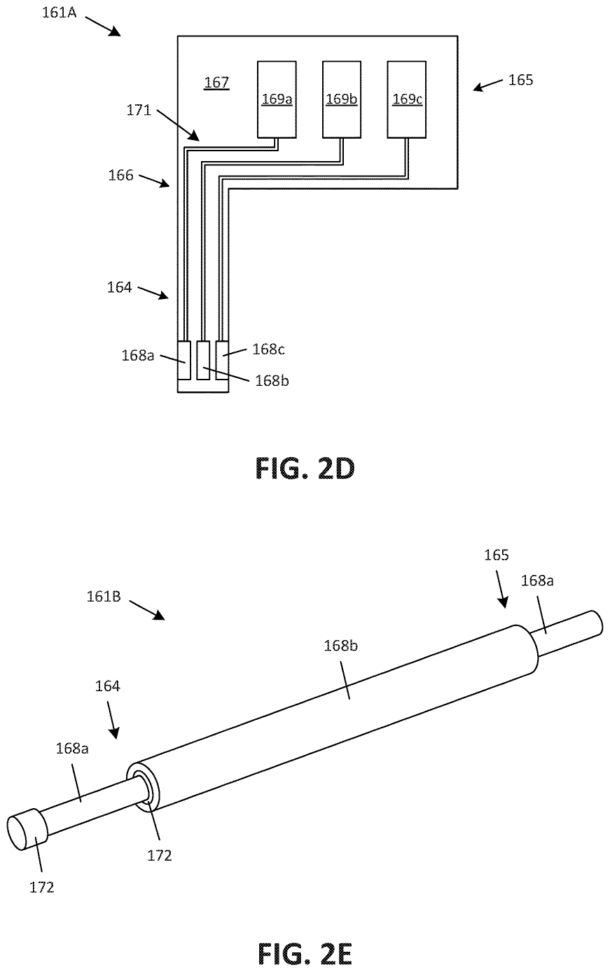

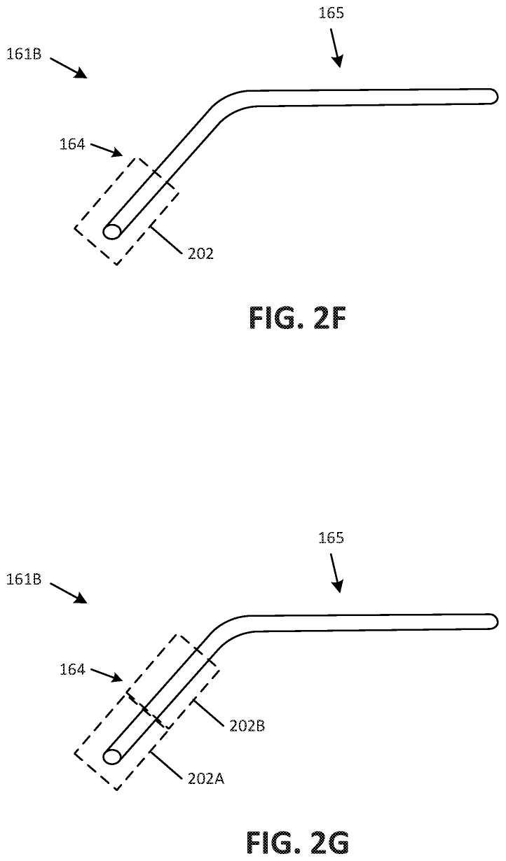

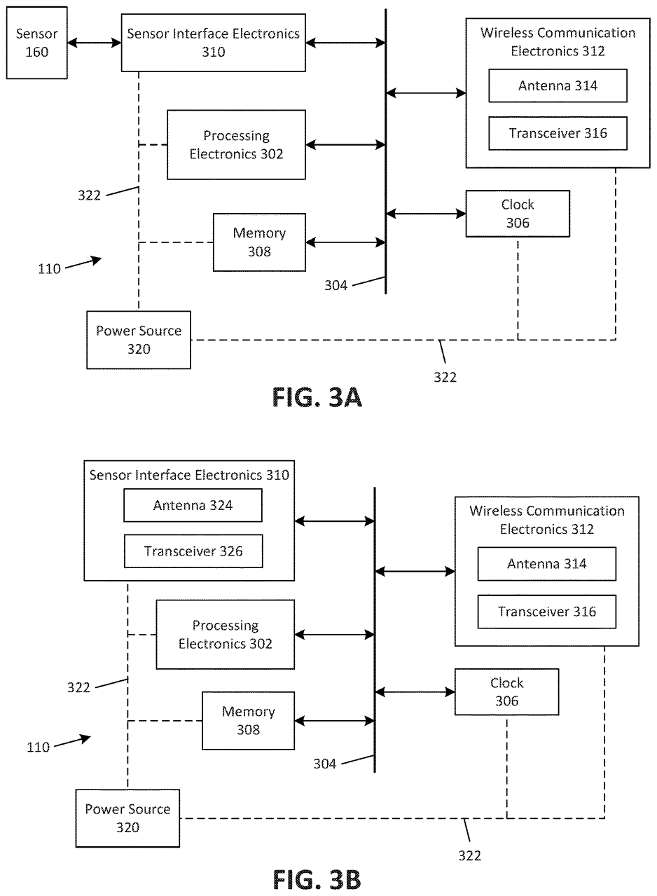

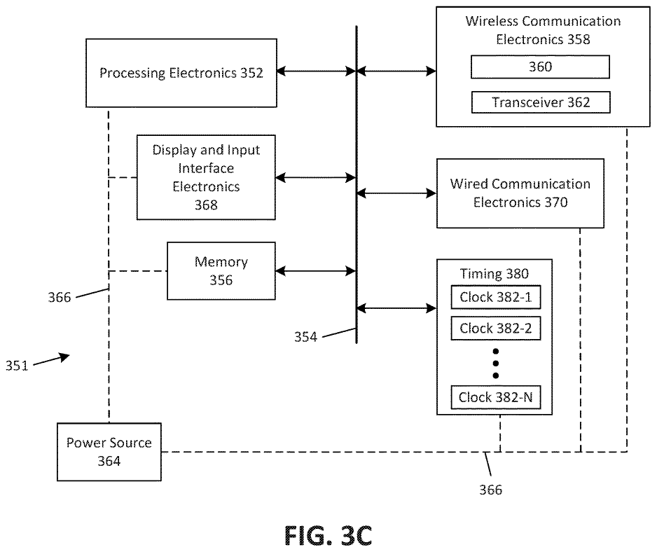

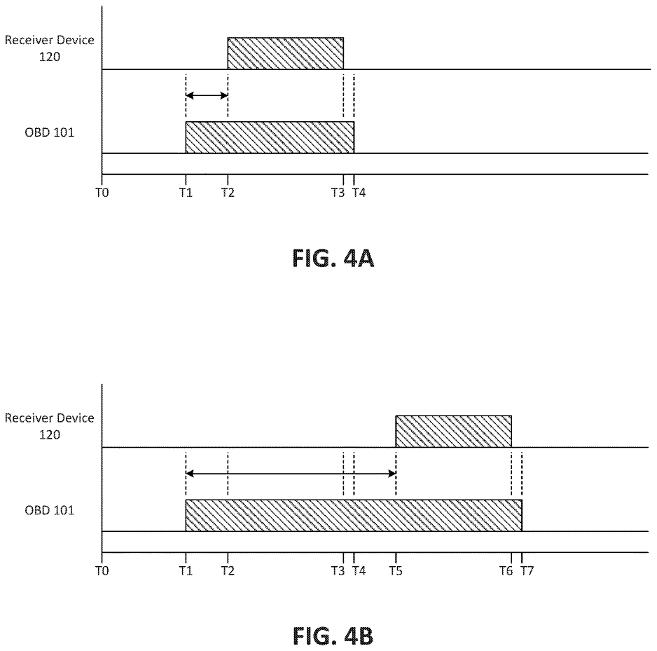

The embodiments of FIGS. 1A, 1B, and 1C are brief examples showing the various manners in which the assessment of clock accuracy and an action in response can be progressed with an on-body analyte monitoring system 100. These and other examples are explained and expanded on in greater detail herein. Before doing so, an example of system 100 in a highly customizable and configurable form with additional optional devices is described (e.g., FIG. 1D) along with its operation, as are examples of the use of OBD 101 (e.g., FIGS. 1E and 1F), examples of sensors (e.g., FIGS. 2A-2G), examples of OBD electronics (e.g., FIGS. 3A and 3B), and an example of receiver device electronics (FIG. 3C). Embodiments utilizing the subject matter pertaining to clock accuracy are further detailed herein and with respect to FIGS. 4A through 7D.

FIG. 1D depicts an operating environment of an on-body analyte monitoring system 100 capable of embodying the subject matter described herein. Reference is made to system 100 herein for the purpose of facilitating the description of the present subject matter including all embodiments, and not for the purpose of limitation of the same. System 100 can include devices and/or components configured to measure and communicate information pertaining to one or more analytes of a human subject. For example, system 100 can be configured to measure an analyte level that is a glucose level of the subject. System 100 can alternatively be configured to measure one or more analyte levels that are not a glucose level of the subject (e.g., a ketone level and/or a lactate level). System 100 can be configured to monitor both a glucose level and one or more of the analyte levels that are not a glucose level of the subject. In each instance, system 100 can be additionally configured to process, communicate, and output—for example, display—the measured analyte level (e.g., concentration of the analyte), as well as other system relevant information, to the user. The user can be the person whose analyte level is being sensed, sometimes referred to as the subject or recipient. The user can be a caregiver or guardian for the subject. The user can be a medical professional for the subject.

System 100 can be configured to take additional action upon the subject beyond analyte measurement, such as delivery of a drug or other medicant from a drug delivery device into the body of the subject. That drug or medicant then accomplishes a therapeutic result on the subject. That drug can be insulin or insulin-alternative drug. The therapeutic result can be a change to the body chemistry of the sensor recipient, such as the lowering of a glucose level or other modification to an analyte level.

System 100 includes an on-body device (OBD) 101 configured to operate in conjunction with an analyte sensor 160 to measure the analyte level as explained further herein. Sensor 160 is shown with broken lines to indicate the various forms it can take as described herein. OBD 101 is configured to wirelessly communicate analyte data, non-analyte data, and/or operation information to one or more receiver devices 120 over one or more wireless communication paths 132. A reference herein to a, or the, receiver device 120 is a reference to any one or more of the receiver devices 120 disclosed herein. The analyte data includes analyte measurement data indicative of an analyte level of the sensor recipient. The analyte data can additionally, or alternatively, include other information related to the analyte, such as a trend in the analyte level including, e.g., either or both of a rate of change of the analyte at a point in time and a profile of analyte levels over time, an analysis of analyte levels, and/or an important notification about the analyte level, such as an alarm or alert. The non-analyte data collected by OBD 101 can include a temperature measurement of the sensor recipient's body and/or a temperature measurement of the ambient environment. Operation information can include instructions or commands to receiver device 120, or a request to receiver device 120, that pertains to the operation of system 100, such as the manner in which communications are sent and received, including indications of schedules, modes, and/or data types, information about system operation or status of OBD 101 including sensor 160, and/or notifications to the user.

Receiver device 120 is configured to act as a user interface to the analyte measurement data collected by OBD 101. If receiver device 120 has a display, then the receiver device 120 can display the analyte level to a user. Alternatively, or additionally, receiver device 120 can further communicate analyte data, the other data, and/or information to, and receive analyte data, the other data, and/or information from, a remote server 150 by way of communication path 134, to a network 145 such as a cloud-based network or internet.

Receiver device 120 can have multiple forms and functions as part of system 100. For example, a receiver device 120 can be a multi-purpose mobile receiver device 122, configured to accomplish a range of generic functions using wireless communications, where those functions extend beyond analyte monitoring alone. A multi-purpose mobile receiver device 122 is typically developed by a manufacturer that does not specialize in analyte monitoring and analyte monitoring sensors. Examples of a multi-purpose mobile receiver device include a smart-phone, a wearable device such as a smart-watch or smart-glasses, smart-ring, and a tablet. Receiver device 120 can be a dedicated receiver device 124 designed for the primary purpose of operation in analyte monitoring within system 100. A dedicated receiver device 124 can be described as built for the purpose (purpose-built) of operating as part of an analyte monitoring system. The dedicated receiver device will, in certain cases, be manufactured by or on behalf of the same entity that manufactures OBD 101. The dedicated receiver device 124 can also be described, in such cases, as having a design controlled by the same manufacturer as that of the OBD and sensor. Receiver device 120 can be a drug delivery device 126 such as an infusion pump or infusion pen. Drug delivery device 126 can be configured like a dedicated receiver device 124 but with drug delivery components and capability. Receiver device 120 can be another wireless computing device 128, such as a laptop, desktop computer, or a processing unit within a vehicle. In instances of system 100 with multiple receiver devices 120, each device 120 can be operated by the same user or a collection of two or more different users, such as the sensor recipient, a caregiver, and/or a medical professional. The group of receiver devices 122, 124, 126, 128 and all other variants are collectively referred to herein as a receiver device 120 or receiver devices 120. In the embodiment of FIG. 1D, there are four receiver devices 122, 124, 126, and 128. In other embodiments, the system 100 includes an OBD 101 and a single receiver device 120, provided by one of the receiver devices 122, 124, 126, 128 and any variant thereof. In further embodiments, the system 100 includes an OBD 101 and two receiver devices 120, each or both provided by one of the receiver devices 122, 124, 126, 128 and/or any variant thereof respectively. In still further embodiments, the system 100 includes an OBD 101 and three receiver devices 120, each or all provided by any one of the receiver devices 122, 124, 126, 128 and/or any variant thereof respectively. The description herein of the system 100 and its operation correspondingly applies to each of these embodiments.

One or more other computing devices 140 having a display and capable of communicating with remote server 150 can be used to access and view the analyte data by communication with server 150 over communication path 136 and network 145. Computing device 140 can be the same configuration as multi-purpose mobile receiver device 122 described herein. Computing devices 140 can be laptop computers, desktop computers, smart wearable devices (e.g., watch or glasses), drug delivery devices, vehicles, and the like. Such computing devices 140 can be located in geographically disperse locations (e.g., a medical professional's office, a school, workplace, residence, or hotel) and can provide convenient access to the subject's analyte data history via an internet browser or other software tool. The one or more computing device 140 can be further configured to wirelessly communicate with receiver device 120 over communication path 138. In the embodiment of FIG. 1D, computing device 140 is a laptop computer.

In this embodiment, remote server 150 is a secure system for storing confidential user data with security capabilities for authenticating users seeking access and subsequently communicating confidential user data to the authenticated users in encrypted form. Remote server 150 in such instances can be, or include, a trusted server or trusted computer system.

Although certain embodiments of system 100 may be described as including only one of each of sensor 160, OBD 101, receiver device 120 (or any one of its forms 122, 124, 126, 128), and computing device 140, this disclosure recognizes that system 100 can incorporate multiples of each component interacting with other devices throughout system 100. For example, the embodiments disclosed herein include multiple sensors 160 that can be associated with multiple users which are in communication with remote server 150. Additionally, remote server 150 is illustrated as a single entity 150, however it can encompass multiple networked servers that can be geographically distributed to reduce latency and introduce deliberate redundancy to avoid monitoring system downtime.

FIGS. 1E and 1F are partial cross-sectional views depicting example embodiments of OBD 101 in place on a skin surface 10 of the body 12 of a human subject. OBD 101 includes a housing 104. OBD 101 can have a size conducive with wearing on the body of the subject, in a location such as the upper arm, abdomen, lower back and/or upper buttock. While not limited to such, in any and all embodiments herein, housing 104 can have a size that is: (a) 0.5 to 10.0 centimeters (cm) in width, 0.5 to 10.0 cm in length, and 0.1 to 3.0 cm in height (distance above the skin); (b) 0.5 to 5.0 cm in width, 0.5 to 5.0 cm in length, and 0.1 to 2.0 cm in height; (c) 0.5 to 3.0 cm in width, 0.5 to 3.0 cm in length, and 0.1 to 0.8 cm in height; (d) 0.5 to 2.5 cm in width, 0.5 to 2.5 cm in length, and 0.1 to 0.7 cm in height; and/or (e) 0.5 to 1.3 cm in width, 0.5 to 1.3 cm in length, and 0.1 to 0.5 cm in height. OBD 101 can be a single-use or reusable device. OBD 101 can have a sealed interior to housing 104. This may improve ease of cleaning and/or reduce risk of tampering and/or reduce risk of moisture damage. Housing 104 can include one or more discrete parts. Housing 104 can be formed as a single component (e.g., over molded electronics). Housing 104 can alternatively be fabricated from multiple pre-formed housing sections. Housing 104 has an interior (with or without free space) that holds sensor interface electronics configured for interfacing with an analyte sensor 160. OBD 101 can be coupled to the skin surface 10 directly by adhesive placed between housing 104 and the skin surface 10. Alternatively, as shown here, OBD 101 can be coupled by way of a flexible pad 106 placed between housing 104 and the skin surface 10. The pad 106 can be adhesively coupled to the underside of housing 104 and can include adhesive on the skin-facing side to couple with skin surface 10. The pad 106 can also be referred to as a patch. In other embodiments, OBD 101 can be held to the body 12 by a non-adhesive band or wrap alone, or in combination with pad 106. An adhesive overpatch can be used to add further securement for longer durations of wear, and in some examples may be used instead of a pad 106. In some embodiments, OBD 101 can include a user interface that provides a visual, audible, tactile and/or haptic notification to a user relating to the analyte data. Housing 104 can have any shape as viewed from the top, side, and/or bottom. When viewed from the top or bottom pad 106 can have any shape, which can be the same shape as housing 104 or a different shape from housing 104.

### Analyte Sensor

Sensors 160 can have various shapes, layouts, and principles of operation in the present embodiments. Described herein are just a few examples of sensors 160. Sensor 160 is configured to generate a signal indicative of an analyte level of the subject. Sensor 160 may be provided in any of the wireless communication devices 101 and systems 100 described herein.

Sensor 160 can be a glucose sensor configured to generate a signal indicative of the glucose level of the subject. Sensor 160 can be used to monitor other analytes in addition to, or instead of, glucose. The other monitored analyte may be, for example, ammonium, asparagine, aspartate, cholesterol, choline, creatinine, one or more diaphorase inhibitors, ethanol, glutamate, glycerol, one or more ketones, lactate, lactose, oxygen, phosphate, potassium, pyruvate, and/or uric acid.

Two or more analytes may be monitored by a given sensor 160 or combination of sensors 160 operating in conjunction with a single OBD 101. For example, OBD 101 can be configured to monitor glucose and one or more ketones. In another example, OBD 101 is configured to monitor glucose and lactate. In another example, OBD 101 is configured to monitor glucose, ketones, and lactate. In another example, the monitored analytes may be one or more ketones and lactate.

The term “sensor 160” collectively refers to partially implantable sensors 161 (also referred to as partially in vivo sensors or partially insertable sensors), wholly implantable sensors 162 (also referred to as wholly in vivo sensors), the wholly ex vivo sensors (can also be referred to as wholly non-implantable sensors) described herein, and otherwise within the scope of the present description. Example embodiments of partially implantable sensors 161 are described with respect to FIGS. 1E, 2A, 2B, 2C, 2D, 2E, 2F and 2G. Partially implantable sensors 161 collectively refers to both planar substrate variants 161A and wire variant 161B. Partially implantable sensors 161 have a first portion that is configured for implantation into the body for contacting bodily fluid for the purpose of measuring one or more analytes, and a second portion that is configured to remain outside of the body (not inserted) for the purpose of connecting the first portion to the electronics of OBD 101. Example embodiments of wholly implantable (also referred to as wholly insertable) sensors 162 are described with respect to FIG. 1F. A wholly implantable sensor 162 is configured such that its entirety is implantable within the body with no portion extending outside of the body, nor any physical (wired) connection existing between the wholly implantable sensor 162 and OBD 101 during operation. Examples of wholly ex vivo (also referred to as non-invasive or non-insertable) sensors are also described. Wholly ex vivo sensors are configured such that no portion of the sensor is implanted in the body, with the entirety of the sensor remaining outside of the body while operating in conjunction with the on-body device.

In the embodiment of FIG. 1E, partially implantable sensor 161 has a first end portion 164 that is inserted into the subject's body 12 into contact with bodily fluid (e.g., interstitial fluid, dermal fluid, and/or blood) to sense the analyte level while placed in body 12. An opposite end portion 165 of sensor 161 is held by OBD 101 and electrically in contact with the OBD electronics 110 contained within housing 104. Example embodiments of such partially implantable sensors 161 are shown and described herein with respect to FIGS. 2A-2G, and example embodiments of electronics for interfacing with such sensors 161 are described with respect to FIG. 3A. As described in greater detail herein, partially implantable sensors 161 can be placed in the subject's body using an inserter that may be provided (e.g., sold) with OBD 101. The subject can perform this procedure in any suitable location when convenient without the aid of a medical professional. OBD 101 with a partially implantable sensor 161 is designed to be worn on the body with sensor 161 in the body through the entire wear duration.

Sensor 161 can be configured to be inserted in the body of the subject such that a sensing region of sensor 161 is under the skin, in a subcutaneous location. More specifically, a region of sensor 161 can be placed under the skin (e.g., the sensing region that is configured to measure the analyte level). Thus, sensor 161 can be configured to extend through the skin and, in such embodiments, sensor 161 may be referred to as a transcutaneous sensor 161. Sensor 161 is configured to be in contact with bodily fluid. Sensor 161 can be configured to be in contact with interstitial fluid. In other examples, the bodily fluid may be blood from a capillary in addition to fluid from other sources. In other examples, sensor 161 can be configured to be inserted into the skin such that the sensing region resides in the epidermis and the bodily fluid is fluid from the epidermis, or inserted into the skin such that the sensing part resides in the dermis (the dermal layer) and the bodily fluid is fluid from the dermis. When configured for sensing epidermal or dermal fluid, portion 164 can be configured to be inserted into but not entirely through the skin. Sensor 161 can be of a type referred to as minimally invasive. Sensor 161 can be a microneedle or an array of microneedles. Sensor 161 can be inserted, and OBD 101 applied, by the subject or another user to the subject, for example by using an inserter or applicator.

Sensor 161 is configured to sample the analyte level of the subject and output a signal (e.g., a current or voltage) for receipt by electronics 110. Sensor 161 can be configured to sample the analyte level continuously, at random intervals, at periodic intervals (where each sampling is a discrete single sampling or a sampling for a longer duration), and/or in response to a solicitation.

FIG. 2A is a perspective view of an example embodiment of a partially implantable sensor 161A in a transverse configuration with first end portion 164 extending along an axis 201 transverse to an X-Y plane occupied by second end portion 165. First end portion 164 of sensor 161 can be referred to as an insertion portion or a distal portion and is configured to be positioned in the body of the subject. Portion 164 is configured to be positioned into the skin of the subject and can, in some embodiments, extend through the skin into a subcutaneous space. Portion 164 includes a sensing region 202 that has detection capability for an analyte measured by system 100. Sensing region 202 is configured to be positioned in contact with analyte-bearing bodily fluid of the subject, such as interstitial fluid, dermal fluid, epidermal fluid, or blood.

In these embodiments, second portion 165 of sensor 161A includes contacts 169 and can be referred to as a contact portion or a proximal portion. Portion 165 is configured to be positioned outside of the body of the subject. Portion 165 is configured to be electrically coupled to electronics 110. Portion 165 may also be configured to be physically coupled to electronics 110 (not shown).

In this manner, sensor 161A is configured to be partially implantable into the body of the subject. For example, part of sensor 161A is positioned in the body (e.g., the first portion 164) and part of sensor 161 is positioned out of the body (e.g., the second portion 165).

Sensor 161A can be fabricated in this transverse configuration of FIG. 2A or can be fabricated in a continuous planar configuration and then bent to form the transverse configuration.

FIG. 2B is a perspective view of another example embodiment of a partially implantable sensor 161A in a transverse configuration. Here, first end portion 164 includes two sensing regions 202A and 202B. Sensing region 202A is configured to detect a first analyte (such as any of those disclosed herein) and sensing region 202B is configured to detect a second analyte (such as any of those disclosed herein) that is different from the first analyte. Here the sensing regions 202A and 202B are adjacent to each other along a length of portion 164, but in other embodiments the sensing regions 202A and 202B can be overlaid so that the working electrodes are each at or near the terminus of portion 164 in the subject's body.

FIG. 2C is a cross-sectional view depicting an example embodiment of a first end portion 164 of sensor 161A having a sensing region 202 with electrodes on different sides of a base substrate 167. The base substrate 167 may be configured to be insulative and optionally bendable. The electrodes are configured in a stacked configuration. In this embodiment, sensor 161A is an electrochemical sensor, configured to measure the level of an analyte via electrochemical oxidation/reduction reactions. Sensor 161A includes electrodes 168. In particular, sensor 161A includes at least one working electrode 168a and at least one further electrode. In this embodiment, sensor 161 includes three electrodes 168 labeled as 168a, 168b, and 168c. In this embodiment, the electrodes 168 include a working electrode 168a, a reference electrode 168b, and a counter electrode 168c. The working electrode 168a is the electrode at which the analyte (or a product which depends on the analyte level) is electrolyzed (e.g., electro-oxidized or electro-reduced). The counter electrode 168c provides an electrode paired with the working electrode, through which passes a current equal in magnitude and opposite in polarity to the current passing through the working electrode. The reference electrode 168b has a stable and known potential. In other examples, the reference electrode 168b and the counter electrode 168c are provided by the same electrode (a counter/reference electrode). Thus, there may be only two electrodes 168.

In some examples, the counter and/or the reference electrode may be omitted. For example, sensor 161A can be self-biasing (e.g., self-powered) and there may be no need for a reference electrode. In some examples, a plurality of working electrodes may be provided, such as when measuring two different analytes with the same sensor 161A.

Electrodes 168 may be made from conductive material. For example, one or more of electrodes 168 (e.g., the working electrode, the counter electrode, and/or the reference electrode) may be made from carbon, graphite, a conductive polymer, platinum, gold, palladium, iridium, graphite, and/or an alloy (e.g., platinum-iridium). In some examples, reference electrode and/or the counter electrode may be made from silver/silver chloride. In some examples, the working electrode and the counter electrode may be made from carbon, and the reference electrode may be made from silver/silver chloride.

Substrate 167 may be a dielectric (e.g., an electrical insulator). For example, substrate 167 may be a polymer or a plastic, such as polycarbonates, polyesters (e.g., Mylar™ and polyethylene terephthalate (PET)), polyvinyl chloride (PVC), polyurethanes, polyethers, polyamides, polyimides, or copolymers of these thermoplastics, such as PETG (glycol-modified polyethylene terephthalate). In other examples, substrate 167 may be a ceramic material, such as aluminum oxide or silicon dioxide. Substrate 167 provides a structure for sensor 161A, and also allows the various components attached to the substrate 167 to be electrically insulated from each other if desired.

Substrate 167 has a first conductive layer 206 on its first side and a second conductive layer 208 on its second side. A first insulative layer 222 covers a portion of conductive layer 206. A second insulative layer 214 covers a portion of conductive layer 208. While depicted as extending to the terminus 205 of substrate 167, one or both of conductive layers 206 and 208 may terminate short of terminus 205. Conductive layer 208 includes a working electrode 168a having a sensing component 210 disposed on at least a portion of conductive layer 208. While a single sensing component 210 is shown, in other embodiments a plurality of spatially separated sensing components or layers may be utilized (e.g., a series of dots) on layer 208.

A conductive layer 216 is located on insulative layer 214. Conductive layer 216 is configured to include a reference electrode 168b which includes a secondary layer of conductive material 218, e.g., Ag/AgCl, disposed on a distal portion of conductive layer 216. An insulative layer 220 is located on a first surface of conductive layer 216 including reference electrode 168b. At least the ends of secondary conductive material 218 which extend along the side edges of sensor 161A are not covered by insulative layer 220 and, as such, are exposed to the environment for operation of the reference electrode 168b when in operative use. First insulative layer 222 does not extend to the terminus of conductive layer 206 leaving an exposed region of layer 206 that is configured to act as a counter electrode 168c.

As described in greater detail herein, one or more membranes may be provided about sensor 161A, e.g., as one or more of the outermost layer(s). Here, working electrode 168a has a first membrane layer 212 over sensing component 210 to modulate the rate of diffusion or flux of the analyte to the sensing component. A second membrane layer 224 is coated over the remaining surface area of end portion 164. The second membrane layer 224 may be provided to serve as a biocompatible conformal coating. The second membrane layer 224 may additionally or alternatively provide smooth edges over the entirety of the sensor. Additional membranes to accomplish other functions described herein can be included. In other embodiments, a single, homogenous membrane performing the functionality of both membranes 212 and 224 (and other functions described herein) may be coated over the entire end portion 164.

Conductive layer 208 can extend between working electrode 168a and a first contact 169a on end portion 165 and form an electrical connection 171 therebetween. Conductive layer 216 can extend between reference electrode 168b and a second contact 169b on end portion 165 and form an electrical connection 171 therebetween. Conductive layer 216 can extend between counter electrode 168c and a third contact 169c on end portion 165 and form an electrical connection 171 therebetween.

FIG. 2D is a front view depicting an example embodiment of partially implantable sensor 161, specifically sensor 161A. Sensor 161A is generally planar and can be operated in this configuration with OBD 101. In other embodiments, such as that shown in FIGS. 1E and 2A, sensor 161 can be folded or bent at or near intermediate portion 166 to form a transverse configuration with portion 164 at an angle with respect to portion 165. The second portion 165 can be coupled to electronics 110 (not shown in FIG. 2D), with the first portion 164 lying in a plane generally transverse, optionally perpendicular to the sensor interface electronics (e.g., perpendicular to a printed circuit board (PCB) holding some or all the sensor interface electronics).

In this embodiment, electrodes 168 are arranged on substrate 167. In particular, electrodes 168 are arranged on the surface of substrate 167. In other examples, electrodes 168 may be arranged on different sides of substrate 167. Electrodes 168 may be electrically separated from each other by substrate 167 and/or an additional dielectric material or electrical insulator. For example, electrodes 168 may be arranged in layers (e.g., planar layers) separated by dielectric material.

Electrodes 168 are positioned on portion 164. In particular, electrodes 168 are configured to be inserted into the body when portion 164 is inserted into the body. For example, electrodes 168 may be located towards the tip of portion 164.

In this embodiment, sensor 161A includes sensor contacts 169 labeled as 169a, 169b, and 169c. In this embodiment, sensor 161A includes three sensor contacts 169. In particular, each of the sensor contacts 169a, 169b, 169c corresponds to a particular electrode 168a, 168b, 168c. In this embodiment, sensor contacts 169 are positioned on portion 165. Contacts 169 are electrically conductive and configured to allow an electrical connection between sensor 161A and the sensor interface electronics. For example, sensor contacts 169 may be configured to be in electrical contact with the electrical contacts of the sensor interface electronics.

In the embodiments of sensor 161A, sensor 161A includes connections 171 that may be configured as traces. Connections 171 are electrically conductive and each connection 171 forms a respective electrical connection from one of the electrodes 168 to a corresponding contact 169. In this manner, an electrical signal generated at electrodes 168 can be passed along connections 171 to the sensor contacts 169. The signal can then be passed to the sensor interface electronics. This signal corresponds to the measured analyte level. Connections 171 can be arranged in the substrate 167, such as by embedding them in the substrate 167, or the connections 171 can be positioned on a surface of the substrate 167. In some examples, the electrodes 168, the sensor contacts 169, and/or the connections 171 may be fabricated from a conductive material, such as carbon, graphite, a conductive polymer, platinum, gold, palladium, iridium, graphite, and/or an alloy (e.g., platinum-iridium). Each of the connected electrodes 168, connections 171, and sensor contacts 169 may be made from the same material.

FIG. 2F is a perspective view depicting another example embodiment of analyte sensor 161, referred to as sensor 161B, which includes the same features as sensor 161A of FIGS. 2A-2D, except as explained below. Various optional features and alternatives described in relation to the sensor 161A may be applied to sensor 161B in a corresponding manner.

In this embodiment, sensor 161B is a wire sensor with a sensing region 202. In particular, sensor 161B does not have a planar substrate (e.g., the substrate 167 of sensor 161A), but instead has a generally cylindrical shape. Sensor 161B has a transverse configuration with end portion 164 at an angle with respect to end portion 165. Sensing region 202 is located on end portion 164.

FIG. 2E is a perspective view depicting an example embodiment of sensor 161B in greater detail. Here, sensor 161B includes a working electrode 168a that can have a similar function to the working electrode 168 of sensor 161A. Working electrode 168a may be made from conductive material, for example, carbon.

In this embodiment, working electrode 168a is a central conductive wire. Electrode 168a can be generally cylindrical with a circular or elliptical cross-section. The other layers of sensor 161B are placed circumferentially around electrode 168a. This means that a substrate is not required. Thus, sensor 161B has a conductive core. In other examples, a central substrate (e.g., a non-conductive wire) may be provided and the working electrode 168a may be arranged around the central substrate.

In this embodiment, sensor 161B includes a dielectric 172, which can be a layer (e.g., cylindrical) around working electrode 168a (e.g., arranged concentrically). Dielectric 172 is an electrical insulator. For example, dielectric 172 can be a polymer or a plastic, such as polyester, polyamide, or polyurethane. In other examples, dielectric 172 can be a ceramic material, such as aluminum oxide or silicon dioxide.

In this embodiment, sensor 161B includes a counter/reference electrode 168b. In particular, there is one electrode that performs the function of both the counter and reference electrodes. The counter/reference electrode 168b is a layer (e.g., cylindrical) around dielectric 172. Thus, electrodes 168 can be in concentric layers (separated by dielectric 172). The counter/reference electrode 168b can be made from conductive material, such as silver/silver chloride or carbon.

In this embodiment, sensor 161B includes one or more regions in which the counter/reference electrode 168b and the dielectric 172 is removed. In this manner, the analyte sensor 161B includes a region at which the working electrode 168a is exposed at the proximal end. For example, dielectric 172 can be stripped or skived to expose the working electrode 168a. The exposed section may act as part of the working electrode 168a, along with any additional sensing componentry and membranes. This forms the sensing region 202 of sensor 161B. This is at the portion 164 is configured to be inserted into the body.

At the tip of sensor 161B (e.g., at portion 164), dielectric 172 is arranged over working electrode 168a. For example, this may protect electrode 168a and may provide a more biocompatible surface for insertion into the human body.

The working electrode 168a may similarly be exposed at the first portion 165 for electrically coupling to the sensor interface electronics. For example, one electrical contact of the sensor interface electronics may be in contact with the working electrode 168a, and another electrical contact may be in contact with the counter/reference electrode 168b. Sensor 161B may thus not have separate connections and sensor contacts (such as the connections 171 and sensor contacts 169 of sensor 161A). The portion 165 may be maintained out of the body 12, as described herein.

Although the portion 164 and the portion 165 are shown along one axis in FIG. 2E, sensor 161B can be bent or folded such that the portion 164 is at an angle, such as perpendicular, to the portion 165, similar to sensor 161A.

Sensor 161 (e.g., 161A and 161B) can include one or more membranes and one or more sensing components. The sensing component may be referred to as a catalyst layer or region or an enzyme layer or region. The sensing component may include a catalyst to catalyze the reaction of the analyte. For example, the catalyst may be an enzyme. Some analytes, such as oxygen, can be directly electro-oxidized or electro-reduced at the working electrode. Other analytes, such as glucose, may use a catalyst to facilitate the electro-oxidation or electro-reduction of the analyte. In this embodiment, where the analyte is glucose, the enzyme may be glucose oxidase. Glucose oxidase reacts with glucose and oxygen to produce hydrogen peroxide, which reacts at the working electrode to produce an electrical signal proportional to the glucose concentration. In other examples, other enzymes may be provided (either for monitoring glucose or for other analytes). For example, for monitoring lactate, lactate oxidase may be used as an enzyme.

The sensing component can include an electron transfer agent to transfer electrons between the analyte and working electrode. An electron transfer agent is a compound that carries electrons between the analyte and working electrode. For example, the electron transfer agent may be a redox mediator. In some examples, the electrolysis can be performed without an electron transfer agent.

In some examples, sensor 161 can include an interferent layer. The interferent layer may prevent interferents or contaminants from interfering with the sensing components (e.g., electrodes 148) of sensors 161. For example, interferents may include acetaminophen (paracetamol), urate (uric acid), and/or ascorbate (ascorbic acid or vitamin C). Interferents may otherwise react at the electrodes, leading to a false signal.

In some examples, sensor 161 can include a diffusion limiting layer. This may otherwise be referred to as a mass transport limiting layer, flux limiting layer, or a resistance layer. This may limit diffusion of the analyte (e.g., glucose and/or oxygen) to maintain a linear signal response and improve signal stability. In some examples, one or more membrane layers or regions (e.g., an outer layer) is biocompatible. The biocompatibility allows sensor 161 to be safely positioned in the body of the subject and may reduce the impact of a physiological response on operation of sensor 161.

FIG. 2G is a perspective view depicting another example embodiment of analyte sensor 161B. Here, first end portion 164 includes two sensing regions 202A and 202B. Sensing region 202A is configured to detect a first analyte (such as any of those disclosed herein) and sensing region 202B is configured to detect a second analyte (such as any of those disclosed herein) that is different from the first analyte. Here the sensing regions 202A and 202B are adjacent to each other along a length of the wire sensor, but in other embodiments the sensing regions 202A and 202B can be overlaid so that the working electrodes are each at or near the terminus of portion 164 of the wire sensor in the subject's body.

In this and other embodiments of sensors 161A and 161B having multiple sensing regions 202A and 202B, each sensing region can have its own discrete electrodes such that none are shared. Alternatively, one or more electrodes can be shared between the sensing regions 202A and 202B, and one or more electrodes can be specialized for the analyte particular to that sensing region 202 (and not shared). For example, a single reference electrode can be shared between sensing regions 202A and 202B, and sensing region 202A can have its own working electrode, while sensing region 202B can have its own separate working electrode. In embodiments with a discrete counter electrode, the single counter electrode can be shared between sensing regions 202A and 202B or each region 202A and 202B can have its own counter electrode.

Turning now to embodiments with a wholly (or fully) implantable sensor 162, FIG. 1F is a partial cross-sectional view depicting an example embodiment with wholly (or fully) implantable sensor 162 in wireless communication with OBD 101 over communication path 112. OBD 101 and sensor 162 can communicate using one or more wireless communication protocols. The one or more wireless communication protocols may be the same as or different from those used for OBD 101 to communicate with receiver device(s) 120. Sensor 162 is wholly implanted within body 12 of the subject such that no physical connection exists with OBD 101. Sensor 162 can have its own power source within it or can rely on power harvesting from the wireless signals transmitted to it by OBD 101, or both. The subject can visit a medical professional and undergo an implantation procedure (e.g., inpatient or outpatient operation or surgery) where an incision is created in the subject's body 12 and sensor 162 is placed at a location wholly within the subject's body. As described further herein, the operation can be performed manually or with a specialized implantation tool. This implant location can be, in whole or in part, within the epidermis, within the dermis, within the subcutaneous layer, or elsewhere provided sensor 162 has access to the analyte being measured and can communicate wirelessly with OBD 101. The incision is then closed (e.g., with a suture, staple, or adhesive) so that it may heal.

In this embodiment, wholly implanted sensor 162 wirelessly communicates the glucose level, or other analyte level, to OBD 101 which remains outside of the body on the skin in a position within wireless communication range of sensor 162. Sensor 162 is thus not maintained in physical and electrical contact with OBD 101 while monitoring the analyte level. An example embodiment of sensor interface electronics for interfacing with a wholly implantable sensor 162 is described with respect to FIG. 3B. OBD 101, configured to operate with a wholly implantable sensor 162, can be a single-use (e.g., non-rechargeable) or reusable (e.g., rechargeable) device. OBD 101 may be configured with a designed wear duration (e.g., one day, 14 days, 20 days, 30 days, 60 days, etc.). The sensor life of wholly implanted sensor 162 can be considerably longer than that of sensor 161.

Such a sensor 162 can operate using the electrochemical sensing configurations, techniques, and variations described herein with respect to sensor 161. Sensor 162 can alternatively sense a glucose and/or other analyte level through a photonic-based sensing technique and data indicative of the glucose and/or other analyte level can be wirelessly communicated from sensor 162 through the body 12 to OBD 101. Various photonic-based sensing techniques can be used. For example, an indicator chemical in sensor 162 can bond to or react in the presence of glucose and/or another analyte and then fluoresce in proportion to the glucose and/or the other analyte level in response to received light. The amount of fluorescence can be measured with a detector and the level can be determined based on the measured fluorescence. Sensor 162 can have multiple sensing regions for sensing different analytes. These sensing regions can each utilize their own discrete sensing componentry. Alternatively, as with sensors 161A and 161B, the multiple sensing regions of sensor 162 can share sensing componentry, such as electrodes or optical indicators, and also have discrete different sensing componentry for sensing the different analytes.

In still other embodiments, sensor 160 may sense the glucose level and/or other analyte level while remaining wholly ex vivo, without implantation (neither partial nor full implantation). In these embodiments the sensor 160 and OBD 101 are physically and electrically coupled together, by the manufacturer or a user, and can be placed on the skin surface 10 but remain outside of the subject's body 12, e.g., the ex vivo sensor can be held adjacent to the body by OBD 101, either in contact with the skin surface or spaced apart from the skin but adjacent thereto. The wholly ex vivo sensor is five (5) millimeters or less from the skin in certain embodiments. The wholly ex vivo sensor is ten (10) millimeters or less from the skin surface in certain embodiments. OBD 101 with wholly ex vivo sensors can have a target wear duration (e.g., one day, 10 days, 14 days, 20 days, 30 days, or longer, etc.).

Such ex vivo sensors operate in the embodiments herein by utilizing a sensing architecture (technique and hardware). Such ex vivo architectures include, but are not limited to: electrochemical architectures such as Reverse Iontophoresis (RI) Technology; optical architectures such as Fluorescence Methods, Near-Infrared (NIR) and Mid-Infrared (MIR) Spectroscopy, Raman Spectroscopy, Optical Coherence Tomography (OCT), or Optical Polarimetry (OP); and microwave architectures utilizing parameters such as the reflection coefficient, resonance frequency, bandwidth, and quality factor (Q). Wholly ex vivo sensors can also be configured to detect and measure the concentration levels of two or more different analytes, such as any of those described herein.

Embodiments of OBD 101 operating in conjunction with an ex vivo sensor 160 are different from, and do not include, in vitro devices that rely on penetration of the subject's body 12 (e.g., with a sharp or conduit) to remove bodily fluid from body 12 and test the bodily fluid while outside of the subject's body to determine the analyte level. One example of an in vitro device is a blood glucose meter where the subject performs a fingerstick to place blood on a test strip that is then inserted into the meter for glucose level measurement.

In embodiments of system 100 including a partially implantable sensor 161, OBD 101 operates in conjunction with that sensor 161 through a physical connection and electrical contact between the partially implantable sensor 161 and the OBD electronics 110. End portion 165 of the sensor 161 can be contained within housing 104. When connected together OBD 101 can be considered to include sensor 161. In embodiments of system 100 including a wholly implantable sensor 162, OBD 101 operates in conjunction with that sensor 162 through communication over the wireless communication path 112 between OBD 101 and sensor 162. Sensor 162 is included within system 100 but is not included within OBD 101 as the two are spaced apart in operation and not connected physically nor via direct electrical contact. In embodiments of system 100 including a wholly ex vivo sensor, OBD 101 operates in conjunction with that sensor through a physical connection and one or more signal connections (e.g., electrical, photonic) between the wholly ex vivo sensor and the OBD electronics 110. A portion of the wholly ex vivo sensor can be contained within housing 104. When connected together OBD 101 can be considered to include wholly ex vivo sensor.

### Insertion and Application Devices

The on-body analyte monitoring systems 100 described herein can include a tool for insertion of the sensor (if applicable) into, and application of OBD 101 to, the subject's body by a user (who can be the subject or a different person). In systems 100 utilizing partially implantable sensors 161, an inserter 180, also referred to as an insertion device, can be included. Inserter 180 holds the sensor 161 and inserts it into the body of the subject with the aid of a sharp while concurrently placing OBD 101 on the body. FIGS. 1G, 1H, and 1I are perspective views depicting a first example embodiment of an inserter 180 at stages during an insertion and application procedure on the recipient's skin 10. In this embodiment the inserter 180 has a first part 181 that slides with respect to a second part 182. Application of a manual push force to a surface 183 (e.g., a backside) of the first part 181 telescopes the first part (either into or over) the second part 182 while part 182 is pressed against the skin 10. The range of sliding motion of the first part from the initial position of FIG. 1G to the advanced position of FIG. 1H advances a sharp 184 (see FIG. 1I) within the inserter 180 into the skin 10 to create an opening in which the sensor 161 can be inserted. Motion of the first part 181 to the advanced position also concurrently advances the sensor 161 and OBD 101 into position on the recipient's body, with the sensor 161 positioned within the opening created by the sharp 184 and OBD 101 attached to the skin 10 (FIG. 1H). The inserter 181 can then be removed from the skin 10 leaving behind the sensor 161 and OBD 101 adhered to the skin 10 as shown in FIG. 1I. While the sharp 184 is shown here extending from within inserter 180, in other embodiments the inserter 180 can be configured to automatically retract the sharp 184 into a position within the inserter 180 (such as by release of a spring) to lessen the risk of accidental stick by the sharp 184.

FIGS. 1J, 1K and 1L are perspective views depicting a second example embodiment of an inserter 180 at stages during an insertion and application procedure. In FIG. 1J the inserter 180 is brought towards the skin 10 with the on-body device 101 loaded therein. This embodiment of inserter 180 functions similarly to the preceding embodiment but here, after the inserter is brought into contact with skin 10, the inserter 180 is actuated by an actuator 185 (e.g., a depressible button) which causes internal advancement of the sharp 184 into the skin (e.g., such as by release of a spring), as well as advancement of the sensor 161 into the opening created by the sharp 184 and advancement of OBD 101 to an attached position on the skin 10 (FIG. 1K). Actuator 185 can be located on the side or top of the housing of inserter 180. The actuator 185 can be seamlessly integrated with the housing of the inserter 180. Thus, the continuous manual push force applied to a surface that drives the sharp into the skin, as described with respect to FIGS. 1G and 1H, is not required for operation of this second embodiment, as the advancement of the internal components is performed automatically upon triggering of the actuator 185. The inserter 180 can then be removed from the skin as shown in FIG. 1L. While the sharp 184 is shown here as extending from within inserter 180, in other embodiments the inserter 180 can be configured to automatically retract the sharp 184 into a position within the inserter 180 (such as by release of a spring) to lessen the risk of accidental stick by the sharp 184.

With respect to inserter 180 for use with systems having a partially implanted sensor 161, the inserter 180, sensor 161, and OBD 101 can be provided to the end-user in various states of assembly. Each component 180, 161, and 101 is subjected to a sterilization process and placed in a shipment state (e.g., sealed packaging or container) for the transfer from the manufacturer's possession to another party downstream, such as a distributor, doctor's office, or end user. The sterilization process for sensor 161, OBD 101, and inserter 180 can be a Spaulding critical level sterilization process, such as by ionizing (gamma) radiation or sterilizing agent (e.g., ethylene oxide (EtO)). The sterilization processes can be performed independently on each component, or they can be sterilized together. Each component remains sterile while in their shipment states.

The inserter 180, sensor 161, and OBD 101 can be placed in their shipment state while not connected or combined. The end user can assemble the components prior to using the inserter 180 to perform the insertion and application procedure. The user can couple the sensor 161 with OBD 101 into a connected assembly, and then load the connected assembly into the inserter 180, and then perform insertion and application procedure.

OBD 101 and sensor 161 can be coupled together by the manufacturer into a connected assembly. The connected assembly can be placed in its shipment state while not loaded within inserter 180. The end user, after receipt, can load the connected assembly into the inserter 180, and then perform insertion and application procedure.

OBD 101 can be loaded into the inserter 180 by the manufacturer to form a pre-loaded assembly and the pre-loaded assembly can be placed in its shipment state. The sensor 161 can be placed in its shipment state separate from the pre-loaded assembly (OBD 101 and inserter 180). The end user, after receipt, can load the sensor 161 into OBD 101 while within the inserter 180, and then perform the insertion and application procedure.

The sensor 161 can be loaded into the inserter 180 by the manufacturer to form a pre-loaded assembly and the pre-loaded assembly can be placed in its shipment state. OBD 101 can be placed in its shipment state separate from the pre-loaded assembly (sensor 161 and inserter 180). The end user, after receipt, can load OBD 101 into the inserter 180. OBD 101 can be loaded into the inserter 180 such that the sensor 161 and OBD 101 are placed in their connected state, or the two can be connected by the inserter 180 during the insertion process. The user can then perform the insertion and application procedure.

The sensor 161 and OBD 101 can be loaded into the inserter 180 by the manufacturer to form a pre-loaded assembly and the pre-loaded assembly can be placed in its shipment state. The end user, after receipt, does not need to load any component into the inserter 180 as the shipment state is a ready-to-use state. The user can then perform the insertion and application procedure.

Thus, for the partially implantable sensor embodiments described herein, the components 180, 161, and 101 can be assume one of a variety of pre-loaded configurations when placed in the shipment state, which is then ultimately received by the end user (e.g., the recipient or other person).

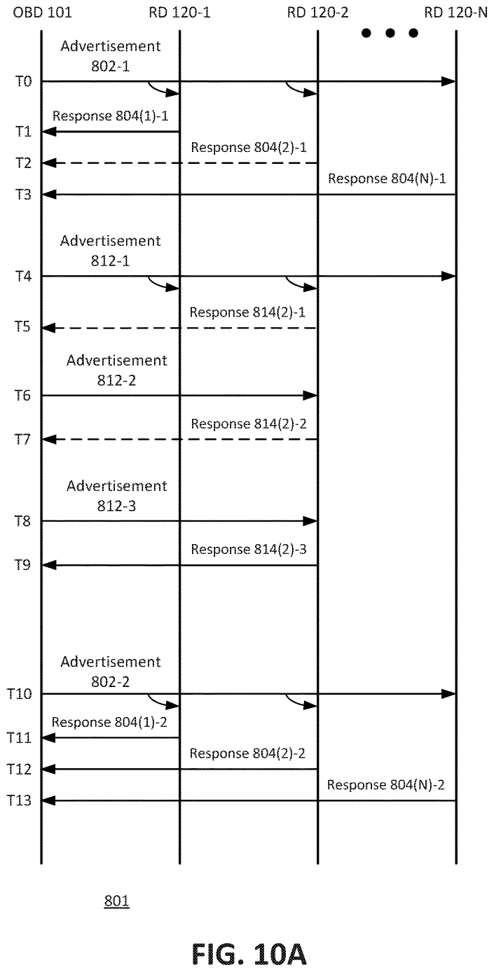

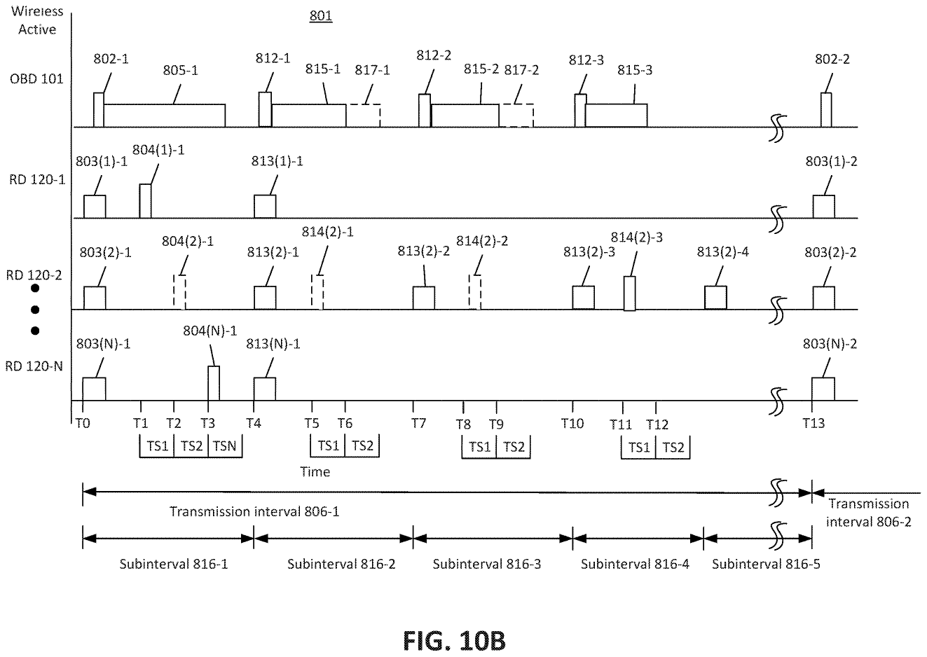

FIGS. 1M, 1N, and 1O are perspective views depicting an example embodiment of an inserter 186 for insertion of a wholly implantable sensor 162 through the skin 10 and into the subject's body. In FIG. 1M, a medical professional has created an incision 15 in the skin 10 and then a pocket 16 within the recipient's body under the skin 10. Inserter 186 has an elongate portion 187 having sensor 162 detachably mounted thereon. Elongate portion 187 and sensor 162 are inserted through incision 15 and sensor 162 is positioned within pocket 16 as shown in FIG. 1N. Sensor 162 is then released from portion 187 by actuation of an actuator 188 (e.g., a depressible button or slidable release mechanism) on inserter 186 and portion 187 is withdrawn from within body 12 as shown in FIG. 10. Incision 15 is then closed by suture or other means.

OBD 101 can then be placed on the skin 10 over sensor 162. Wireless communication electronics in OBD 101 and sensor 162 can be used to sense when OBD 101 is optimally located over sensor 162, and this positioning feedback can be output to the user by OBD 101 (e.g., visually, audibly, and/or in tactile manner) or can be relayed to a receiver device (not shown) for outputting to the user therefrom (e.g., visually, audibly, and/or in tactile manner). Once the optimal position is determined, OBD 101 can be attached to the skin 10. This can be performed by the user manually holding OBD 101 and directly pressing it against the skin 10 to adhere OBD 101 to the skin 10.

In an alternative embodiment, an applicator 188 can have OBD 101 held therein and can be used to apply OBD 101 to the skin 10 in the appropriate location. FIGS. 1P and 1Q are perspective views depicting an example embodiment of an applicator 188 during and after application of OBD 101 over the implant location of sensor 162, respectively. This applicator 188 can be configured like that of inserter 180 with either the automatic release configuration of FIGS. 1J-1L or the manual advancement configuration of FIGS. 1G, 1H, and 1I, but in both embodiments without the sharp 184 and sensor 161 as such are not needed. Here applicator 188 has an actuator 189 for automatic deployment of OBD 101 to the skin 10.

With respect to inserter 186 for use with systems having a wholly implantable sensor 162, the inserter 186, sensor 162, OBD 101, and applicator 188 (if utilized) can be provided to the end-user in various states of assembly. Each component 186, 162, 101, and 188 is subjected to a sterilization process and placed in a shipment state (e.g., sealed packaging or container) for the transfer from the manufacturer's possession to another party downstream, such as a distributor, doctor's office, or end user. The sterilization process for sensor 162, OBD 101, inserter 186, and applicator 188 can be a Spaulding critical level sterilization process, such as by ionizing (gamma) radiation or sterilizing agent (e.g., ethylene oxide (EtO)). The sterilization processes can be performed independently on each component, or they can be sterilized together. Each component remains sterile while in their shipment states.

The inserter 186, sensor 162, OBD 101, and applicator 188 (if utilized) can be placed in their shipment state while not connected or combined. The user can couple the sensor 162 with the inserter 186 into a connected assembly and then perform insertion procedure. The user can load OBD 101 into the applicator 188 (if used) and then perform the application procedure.

Alternatively, the sensor 162 can be coupled with the inserter 186 by the manufacturer to form a pre-connected assembly and the pre-connected assembly can be placed in its shipment state for transfer from the manufacturer's possession. The end user, after receipt, can then perform the insertion procedure without need to connect the sensor 162 to the inserter 186 beforehand. OBD 101 and applicator 188 can be placed in their shipment states independently while unloaded, and the end user can load OBD 101 into the applicator 188 and then perform the application procedure. In an alternative embodiment, OBD 101 can be loaded into applicator 188 by the manufacturer and then placed in the shipment state as a pre-loaded assembly. The end user can perform the application procedure without the need to first load OBD 101 into the applicator 188.

An OBD 101 having a wholly ex vivo sensor can be applied manually by direct placement by a user's hand or by use of an applicator. FIGS. 1R and 1S are perspective views depicting the application of an OBD 101 having a wholly ex vivo sensor 163 with an applicator 190 during and after application, respectively. Like applicator 188, applicator 190 can have OBD 101 held therein and can be used to apply OBD 101 to the skin 10 in a desired location, though wireless positioning assistance is not needed. Applicator 190 can be configured like that of inserter 180 with either the automatic release configuration of FIGS. 1J-1L or the manual advancement configuration of FIGS. 1G, 1H, and 1I, but in both embodiments without the sharp 184 and sensor 161 as such are not needed. Here applicator 190 has an actuator 191 for automatic deployment of OBD 101 to the skin 10.

The sensor 163, OBD 101, and applicator 190 (if utilized) can be provided to the end-user in various states of assembly. Each component 163, 101, and 190 is subjected to a sterilization process and placed in a shipment state (e.g., sealed packaging or container) for the transfer from the manufacturer's possession to another party downstream, such as a distributor, doctor's office, or end user. The sterilization process for sensor 163, OBD 101, and applicator 190 can be a Spaulding critical level sterilization process (though not required), such as by ionizing (gamma) radiation or sterilizing agent (e.g., ethylene oxide (EtO)). The sterilization processes can be performed independently on each component, or they can be sterilized together. Each component remains sterile while in their shipment states.

The sensor 163, OBD 101, and applicator 190 (if utilized) can be placed in their shipment state while not connected or combined. The user can couple the sensor 163 with OBD 101 into a connected assembly and then load the connected assembly into the applicator 190 and perform the application procedure.

The sensor 163 and OBD 101 can be connected by the manufacturer and placed in their shipment state while connected. The applicator 190 can be separately placed in its shipment state. After receipt, the user can load the connected assembly of sensor 163 and OBD 101 into the applicator 190, and then perform the application procedure.

The sensor 163 and OBD 101 can be connected by the manufacturer and placed in their shipment state while connected. The manufacturer can load the connected assembly of sensor 163 and OBD 101 into the applicator 190, and then place the entire connected and loaded assembly into its shipment state. After receipt, the user can perform the application procedure without the need to connect sensor 163 and OBD 101, and without the need to first load the applicator 190.

### System Electronics

For purpose of illustration and not limitation, reference is made to the example embodiments of OBD 101 shown in FIGS. 3A and 3B as for use with the disclosed subject matter. OBD 101 can be designed to be power-efficient. OBD 101 is also preferably low-cost, and possibly disposable. FIG. 3A is a block diagram depicting an example embodiment of an OBD 101 configured for operation with sensor 160, which may be a partially implantable sensor 161 or an ex vivo sensor. FIG. 3B is a block diagram depicting an example embodiment of an OBD 101 configured for operation with a wholly implantable sensor 162. In these embodiments, like components are referred to with like reference numerals.

OBD 101 includes electronics 110 that interface with sensor 160, perform functions of the system, process measurement data, and perform wireless communication tasks. Operation of OBD 101 can include state management of OBD 101 (e.g., control of operating states such as awake, partially awake, and/or sleep states), control of sensor 160, power source life assessment (e.g., measuring power source voltage and/or prediction of power source life), alarm or alert generation and/or management, and/or authentication of receiver devices 120 (e.g., by verification of a unique ID associated with sensor 160). Wireless communication tasks can include controlling a state of a transceiver, wireless communication according to a custom wireless protocol or a standardized protocol (e.g., open or proprietary), encryption and decryption of data, encoding and decoding of data, and/or error correction.

The componentry of electronics 110 can include integrated circuitry (IC) hardware such as one or more semiconductor chips. The semiconductor chip or chips of electronics 110 can be embodied as or with a system-on-chip (SoC) topology, a microcontroller unit (MCU), programmable gate array or field-programmable gate array (PGA, FPGA), and/or an application-specific integrated circuit (ASIC). Electronics 110 can include peripherals, input/output (I/O) ports and/or the like. Electronics 110 can include discrete active and/or passive components of various forms distributed on one or more printed circuit boards (PCBs) with the IC hardware.

As shown in FIGS. 3A and 3B, the componentry of OBD electronics 110 can include processing electronics 302. Processing electronics 302 can include a general-purpose processing unit or processor (e.g., a central processing unit according to a Complex Instruction Set Computer (CISC) or Reduced Instruction Set Computer (RISC) architecture), a PGA or FPGA, a controller, a microcontroller, and/or a custom processing architecture. Processing electronics 302 are centralized or distributed within electronics 110. For example, processing electronics 302 can be centralized as a module within a single chip, distributed throughout a single chip, or distributed in one or more modules within a first ASIC chip and a second wireless communication electronics chip. The processing capability of processor electronics 302 can be implemented in the various IC forms described herein using hardware, firmware, or software, or a suitable combination of hardware, firmware, and/or software.

As shown in FIGS. 3A and 3B, electronics 110 include memory 308 in communication with processing electronics over communication interface 304. Memory 308 may include non-transitory memory such as volatile memory and/or non-volatile memory for storing instructions, information, and/or collected data. Instructions are stored in memory 308 in the form of software and/or firmware that is executable by processing electronics 302 in its locations throughout electronics 110. Instructions having different functions can be performed in the locations within electronics 110 most conducive to those functions. These instructions can be embedded, installed, or both embedded and installed in the electronics. Many types of memory can be used in OBD 101 including, but not limited to one or two or more of random access memory (RAM) (e.g., Dynamic RAM (DRAM), Static RAM (SRAM), Synchronous Dynamic RAM (SDRAM), Double Data Rate SDRAM (DDR SDRAM), Magnetoresistive RAM (MRAM), Resistive RAM (RRAM)), read only memory (ROM) (e.g., flash memory, Programmable ROM (PROM), Erasable PROM (EPROM), Electrically Erasable PROM (EEPROM)), variants thereof (e.g., virtual memory, register memory, cache memory), and/or others. Memory 308 can be in the form of one or more discrete memory chips, can be distributed throughout the semiconductor chips of OBD 101, such as within a processor chip and within a wireless communications chip, or can be both in the form of one or more discrete chips and distributed throughout one or more chips. Utilization of multiple types of different memories and integration of the memories with the various processing circuitry segments allows relatively greater power conservation as memory can be chosen and allocated to different segments according to the manner in which those segments use the memory (e.g., read only, and/or read and write) and thus power consumption can be minimized by avoiding higher power memories where they are not needed and placing memory closer to those segments to minimize losses between them.

As shown in FIGS. 3A and 3B, electronics 110 include wireless communication electronics 312 in communication with processing electronics 302 and memory 308 over communication interface 304. Wireless communication electronics 312 include hardware and software for performing communications with the various types of receiver devices 120. Processing electronics 302, in whole or in part, can be integrated within wireless communication electronics 312 for efficient handling of communications tasks. In all of the embodiments herein, wireless communication electronics 312 are configured to communicate with receiver devices according to a Bluetooth Low Energy protocol. However, wireless communication electronics 312 can have the hardware and software to perform wireless communications with receiver devices in accordance with the communication protocols with which OBD 101 is configured to operate. These communication protocols include, but are not limited to, one or two or more of any of those in the following “Statement of Wireless Communication Protocols for the Present Embodiments” that is referenced throughout this document: Bluetooth; Bluetooth Low-Energy (“BLE”); Near-Field Communication (“NFC”); ultra-wideband (UWB); mobile protocols (e.g., Fifth Generation (5G) technology, Long Term Evolution (LTE), Global System for Mobile Communications (GSM), Code Division Multiple Access (CDMA)); Zigbee; Z-Wave; a personal area network according to Institute of Electrical and Electronics Engineers (IEEE) 802.15 protocols, Adaptive Network Topology (ANT), or IPv6 over Low-Power Wireless Personal Area Networks (6LoWPAN); IEEE 802.11 protocols (e.g., 802.11a, 802.11b, 802.11g, 802.11n (aka Wi-Fi 4), 802.11ac (aka Wi-Fi 5), 802.11ah (Wi-Fi HaLow), 802.11ax (aka Wi-Fi 6)); infrared communications according to the Infrared Data Association standards (IrDA); Light Fidelity (Li-Fi); low-power wide-area network (LPWAN) protocols such as LoRa (Long Range); and wireless communication protocols that are proprietary, customized, and not subject to standardization by an international, national, nor regional standard setting body.

Communication electronics 312 can include communication circuitry for each protocol with which OBD 101 operates. Shown here are an antenna 314 communicatively coupled with a transceiver 316. Transceiver 316 has both transmit and receive hardware and capability. Transceiver 316 can further include transmit and receiver amplification circuitry. The communication circuitry has the capability to support all the requisite communication protocols for the implementation of system 100 and can include more than one antenna and transceiver if desired. Communication electronics 312 can control for transmission and reception according to various different physical formats and data rates. For brevity, only a single set of the communication circuitry is illustrated in this embodiment. The transceiver 316 can be implemented as a single component or as a separate transmitter and a separate receiver, collectively referred to herein as the transceiver. The one or more transmit amplifiers can be selectively connected between an output of the transmitter of the transceiver 316 and the antenna 314. The one or more receive amplifiers are configured to be selectively connected between the antenna 314 and an input of the receiver of the transceiver 316.

As shown in FIGS. 3A and 3B, OBD 101 includes sensor interface electronics 310 in communication with processing electronics 302, memory 308, and communication electronics 312 over interface 304. Sensor interface electronics 310 interface with sensor 160 in its various configurations. In the embodiment of FIG. 3A, sensor interface electronics 310 interface with partially implantable sensor 161 and include contacts for electrically contacting contacts 169 of sensor 161. In a similar embodiment, sensor interface electronics 310 interface with an ex vivo sensor.

Sensor interface electronics 310 can include analog front end (AFE) circuitry 260 for generating and applying a bias current or voltage (e.g., a poise voltage) to sensor 161. Sensor 161 can generate an analog voltage or current signal corresponding to the level of analyte (e.g., glucose) in the subject's bodily fluid. AFE circuitry 260 can receive the generated analog signal indicative of the sensed analyte level, optionally precondition the signal, convert the analog signal to a digital data form indicative of the sensed analyte level using an analog-to-digital converter, and output it to processing electronics 302 for further processing and buffering (e.g., conversion from a raw form into a form more readily representative of the concentration value of the analyte (e.g., in units of mg/dL or mmol/L)). For example, the digital analyte data can be algorithmically adjusted to account for known or estimated offsets resulting from use of sensor 160 (e.g., calibration processing, sensor shelf duration compensation, sensor wear duration compensation, and/or other sensor attenuation compensation). Processing electronics 302 can be partially integrated within sensor interface electronics 310 for these and other purposes. The raw or processed analyte data can be output to communication electronics 204 to be transmitted to a receiver device 120. In other examples, the algorithmic adjustment is not performed by OBD 101 and may instead be performed by receiver device 120. In an embodiment the sensor interface electronics 310 are present on a first ASIC semiconductor chip and the wireless communication electronics 312 are present on a second semiconductor chip, with the processing electronics 302 and memory 308 present on either chip or distributed therebetween.

In the embodiment of FIG. 3B, sensor interface electronics 310 interface with a wholly implantable sensor 162 and are configured with wireless communication electronics similar to electronics 312. Sensor interface electronics 310 receive analyte measurement data wirelessly from wholly implantable sensor 162. Sensor interface electronics 310 can also transmit wirelessly to wholly implantable sensor 162. Sensor interface electronics 310 receive analyte measurement data wirelessly from wholly implantable sensor 162. Sensor interface electronics 310 can also transmit wirelessly to wholly implantable sensor 162, for example with a command to respond with analyte measurement data or report other sensor information such as a unique identifier, the existence of a fault, power supply status, and the like.

Sensor interface electronics 310 can include a transceiver 326 and antenna 324 configured to operate according to the communication protocol implemented by sensor 162. Sensor interface electronics 310 in this embodiment can be implemented discretely from wireless communications electronics 312 as shown here or can be part of wireless communication electronics 312 such that electronics 312 is configured to wirelessly communicate with both wholly implanted sensor 162 and one or more receiver devices 120 using the same or different communication protocols (e.g., communication with sensor 162 using NFC and communication with one or more receiver devices using Bluetooth or Bluetooth Low Energy).

Sensor interface electronics 310 in FIG. 3B can perform wireless communications in accordance with the communication protocols with which sensor 162 is configured to communicate, which include, but are not limited to, one or two or more of any of those in the “Statement of Wireless Communication Protocols for the Present Embodiments” recited herein.

OBD 101 includes a power source 320. Power source 320 can be a battery. Power source 320 supplies electrical voltage and current to all components of electronics 110 by way of a power connection 322 that can include a power bus and PCB power and ground planes. Power source 320 can be accompanied by power management circuitry for managing the connection and disconnection of the power source from the electronics 110 and/or for boosting voltage (e.g., with a charge pump). In some embodiments power source 320 is or includes a capacitive energy storage element that can be wirelessly charged with NFC transmissions (or other inductive coupling). Also, OBD 101 can be configured without wide-area network communication capability.

OBD 101 includes one or more clocks 306. Clocks 306 can include circuitry to produce one or more clock signals to act as timing references for electronics 110. Clocks 306 can be communicatively coupled with processor electronics 302, sensor interface electronics 310, wireless communication electronics 312, and/or other components via interface 304. Clocks 306 can be centralized in OBD 101 or distributed in various locations to serve the circuitry that requires each timing reference. The clock signals can have different frequencies from each other. The clock signals can be used, among other things, to wake OBD 101 from a sleep state and/or to synchronize the timing of advertising or connection events and/or for entering a sleep state between the advertising or connection events. The clock signals can determine when OBD 101 should wake up next after processing the advertising or connection events before going to sleep. OBD 101 can then set an event to wake up in time for the next advertising or connection events. Clocks 306 can be configured similar to the clocks 382 of receiver device 120, described in more detail herein.

Communication interface 304 can be electrical traces or connections that communicatively couple two or more entities together. Interface 304 is distributed throughout OBD 101 to permit communication between all components requiring interaction. Interface 304 can include a main communication bus and one or more input and/or output (I/O) ports. Interface 304 can be within a chip, external to a chip (e.g., on a PCB), or both.

Additionally, processing electronics 302 can execute a startup module stored in memory 308. The startup module can include program instructions that, when executed, are utilized to control circuitry within OBD 101, such as memory 308, sensor interface electronics 310, wireless communication electronics 312, and the like. Processing electronics 302 can execute an operating system module stored in memory 308. The operating system module supports the applications or other individual functions that run within OBD 101. Processing electronics 302 and wireless communication electronics 312 can be configured to execute operations according to a protocol stack, which can include a controller and a host, each containing various communication layers. The protocol stack can include or embody the operations for OBD 101 to communicate with other devices using one or more communication protocols.

As explained herein, while the communication electronics 312 is initialized, the transmitter of transceiver 316 can transmit advertisements (e.g., communication packets including information to facilitate the initiation of a communication session with another device, information relevant to conditions of transmission of a subsequent communication from OBD 101, and/or information relevant to analyte monitoring or the analyte monitoring system) arranged in complexes, followed by relatively lower power (e.g., sleep) states in accordance with an advertisement interval. The receiver of the transceiver 316 can monitor for communications pertaining to formation of a connection (e.g., a Bluetooth or BLE scan request or scan response), during a receive window. Connection solicitations can be sent in response to advertisement notices or independently by other devices. The connection monitoring operation, during an individual receive window, can be performed during the same period of time as transmission of the advertisement notices over corresponding advertisement channels. Optionally, the receive window and connection monitoring operation can continue after completion of transmission of the advertisement notices. Hence, the connection monitoring operation and receive window can temporarily align with the complex of advertisement notices or extend beyond the complex of advertisement notices. The transmission and/or receipt of advertisements by communication electronics 312 can occur in synchronized or aligned fashion with the sensing of an analyte level by sensor 160 and sensor interface electronics 310 such that most or all of the electronics of OBD 101 are in a relatively high power (e.g., active) state at the same time. Alternatively, advertisement communications can occur in a non-synchronized fashion such that communication electronics 312 and sensor interface electronics 310 can be in different states at the same time, e.g., with one being in a relatively higher power state (e.g., an active state) and the other being in a relatively lower power state (e.g. a sleep state). Advertisement communications can take place more frequently than analyte sensing, at the same rate as analyte sensing, and/or at a lower rate than analyte sensing.

As embodied herein a first layer of security for communications between OBD 101 and other devices (120, 162) can be established based on security protocols specified by and integrated in the communication protocols used for the communication (e.g., BLE security protocols, and/or Wi-Fi security protocols). An additional, or alternative, layer of security can be based on communication protocols that necessitate very close positioning (within 6 inches or 15 cm) of communicating devices as with NFC, as such require the communicating devices to be very close to each other which makes it practically difficult to breach data security. Furthermore, certain packets and/or certain data included within packets can be encrypted while other packets and/or data within packets is otherwise encrypted or not encrypted. As an example, connection data and/or connection packets devoted to establishing communication connections between devices can be largely unencrypted (sensitive analyte data can be encrypted, even if included in a connection packet) in order to facilitate discovery by other devices.

Additionally, or alternatively, another layer of security can be based on use, by the OBD 101 and other devices in communication, of application layer encryption using one or more block ciphers to establish mutual authentication and encryption of other devices in system 100. The use of an encryption design implemented in the application layer has several benefits. One benefit of this approach is that in certain embodiments the user can complete the pairing of OBD 101 and another device with minimal interaction, e.g., using only a passive or active NFC data transfer operation (sometimes referred to as an NFC scan) and without requiring additional input, such as entering a security pin or confirming pairing.

Referring still to FIG. 3A, OBD 101 can be configured to dynamically generate authentication and/or encryption keys using the data retained within memory 308. Memory 308 can also have a set of valid authentication and/or encryption keys stored thereon to use with particular classes of devices. OBD 101 can be further configured to perform authentication procedures with other devices (e.g., handshake, and/or mutual authentication, etc.) using received data and apply the generated key to sensitive data prior to transmitting the sensitive data, such as sending the sensitive data to the remote server 150 via communication electronics 312. The generated key can be unique to OBD 101 or sensor 160, unique to a pair of devices (e.g., unique to a particular pairing of OBD 101 and a dedicated data receiving device 120), unique to a communication session between OBD 101 and other device, unique to a message sent during a communication session, or unique to a block of data contained within a message.

OBD 101 can be implemented with or without user attachable or detachable pieces. For example, OBD 101 can be implemented in a monolithic manner from the wearer's perspective, e.g., as a single-piece OBD, where no portion of OBD 101 (except, e.g., the sensor 161 or 163) is required to be connected to another portion in order to achieve functionality on the body. Alternatively, OBD 101 can be implemented in a multi-piece (e.g., two piece) manner from the user's perspective, where two or more portions of OBD 101 are connected together by the user to achieve functionality. For example, OBD 101 can be implemented with first piece including a receptacle and adhesive patch that is first applied to the wearer's body with a partially implantable sensor 161, and then a second piece is attached to the receptacle where the second piece includes OBD electronics 110 and a housing, and upon attachment the sensor 161 is connected to the electronics 110. Alternatively, the first piece can have sensor 161 and sensor interface electronics 310 and the second piece can have the remaining electronics 110 that are connected to the sensor 161 and sensor interface electronics.

OBD 101 can, in some embodiments, be configured to include drug delivery capability. OBD 101 can be configured as an on-body injector (e.g., a pump) attachable to the subject's body and capable of automatically delivering an injection or infusion of a fixed or user-selected dose of a drug over a controlled or selected period of time. In such embodiments, OBD 101 can include a primary container for storing a drug or medicament, a drive mechanism configured to drive or permit the release of a plunger to discharge the drug from the primary container, a trocar (e.g., a solid core needle), a flexible cannula disposed around the trocar, an insertion mechanism configured to insert the trocar and/or flexible cannula into the subject and optionally retract the trocar leaving the flexible cannula in the subject, a fluid pathway connector configured to establish fluid communication between the primary container and the flexible cannula upon device activation, and an actuator (e.g., a user displaceable button) configured to activate the device. As embodied herein, the on-body injector can be pre-filled and/or pre-loaded. OBD 101 can receive instructions from a receiver device 120 that informs as to the amount of drug to be injected or infused and when. Alternatively, OBD 101 can include a flexible cannula for drug infusion connected by tubing to a receiver device 120 configured as a drug delivery device 126.

The embodiments described herein can be applied to devices that do not reside on the body. For example, the sensor communication functionality of OBD 101 can be incorporated into a pen drug delivery mechanism that drives a needle and a plunger of a syringe in order to deliver a drug into the body of the subject. The syringe can be pre-filled with the drug and can operate in response to a triggering event. For example, the mechanism can drive the needle into the subject and advance the plunger to deliver the drug subcutaneously via the needle. A component of the pen, either the pen body or the pen cap that attaches to the pen body to cover the sharp, can be configured to wirelessly communicate with an OBD 101 while on the body (used in conjunction with any of sensors 161, 162, and 163 described herein) to read analyte data and/or otherwise communicate utilizing the clock accuracy embodiments described herein. Similarly, the component of the pen can wirelessly communicate with receiver device 120 utilizing the clock accuracy embodiments described herein in a manner similar to that of the embodiments of communications between OBD 101 and the receiver devices 120 described herein.

Each major functional component of electronics 110 (e.g., processing electronics 302, wireless communication electronics 312, sensor interface electronics 310, and/or memory 308) can be independently configured as circuitry type that is an IC, ASIC, and/or SoC, can be the same or different circuit type from the others, and/or can be integrated together on a single semiconductor chip.

FIG. 3C is a block diagram depicting an example embodiment of receiver device electronics 351 of a receiver device 120. Receiver device 120, as depicted here, can have components and functionality germane to many variants of receiver device 120 described herein (e.g., multi-purpose mobile receiver device 122, dedicated receiver device 124, drug delivery device 126, and/or wireless-enabled computing device 128). Receiver device 120 can optionally not be as memory- and/or processing-power constrained as OBD 101, and as embodied herein, receiver device 120 can include sufficient memory for operational software storage and data storage, and sufficient RAM for software execution to communicate with OBD 101 as described herein.

Like OBD 101, the componentry of receiver device 120 can include integrated circuitry (IC) and discrete active and passive components of various forms to accomplish its tasks within system 100. Such integrated circuitry can be or include a system-on-chip (SoC), a processor (e.g., a central processing unit), a controller, a microcontroller, a programmable gate array (PGA, FPGA), an Application-Specific Integrated Circuit (ASIC), peripherals, input/output (I/O) ports and/or the like. The IC can be in the form of one or more chips or chipsets and can include (either integrated therein or as a discrete chip) non-transitory memory such as volatile and non-volatile memory for storing data and instructions for execution by the integrated circuitry (e.g., by a processing module). Many types of memory can be used in OBD 101 including, but not limited to one or two or more of random access memory (e.g., DRAM, SRAM, SDRAM, DDR SDRAM, MRAM, RRAM), read only memory (e.g., flash memory, PROM, EPROM, EEPROM), variants thereof (e.g., virtual memory, register memory, cache memory), and/or the like.

In FIG. 3C, receiver device electronics 351 include processing electronics 352. Processing electronics 352 can be similar to electronics 302 of OBD 101 or can be configured to include additional computing power and/or functionality. Processing electronics 352 can include a general-purpose processing unit or processor (e.g., a central processing unit according to a Complex Instruction Set Computer (CISC) or Reduced Instruction Set Computer (RISC) architecture), a PGA or FPGA, a controller, a microcontroller, and/or a custom processing architecture. Processing electronics 352 are centralized or distributed within electronics 351. For example, processing electronics 352 can be centralized as a module within a single chip, distributed throughout a single chip, or distributed in one or more modules within a first ASIC chip and a second wireless communication electronics chip. The processing capability of processor electronics 352 can be implemented in the various IC forms described herein using hardware, firmware, and/or software, or a suitable combination of hardware, firmware, and/or software. Processor electronics 352 can include a graphics processor unit (GPU) as a discrete component or integrated into a larger more comprehensive processor.

As shown in FIG. 3C, electronics 351 include memory 356 in communication with processing electronics 352 over communication interface 354. Memory 356 may include non-transitory memory such as volatile memory and/or non-volatile memory for storing instructions, information, and/or collected data. Instructions are stored in memory 356 in the form of software and/or firmware that is executable by processing electronics 352 in its locations throughout electronics 351. Instructions having different functions can be performed in the locations within electronics 351 most conducive to those functions. These instructions can be embedded, installed, or both embedded and installed in the electronics. Many types of memory can be used in receiver device 120 including, but not limited to one or two or more of random access memory (e.g., DRAM, SRAM, SDRAM, DDR SDRAM, MRAM, RRAM), read only memory (e.g., flash memory, PROM, EPROM, EEPROM), variants thereof (e.g., virtual memory, register memory, cache memory), and/or others. Memory 356 can be in the form of one or more discrete memory chips, can be distributed throughout the semiconductor chips of receiver device 120, such as within a processor chip and within a wireless communications chip, or can be both in the form of one or more discrete chips and distributed throughout one or more chips.

Also, like OBD 101, receiver device 120 includes wireless communication electronics 358 communicatively coupled with processor electronics 352 and memory 356 over interface 354. Wireless communication electronics 358 can include hardware and software for performing communications with OBD 101, the various types of receiver devices 120, computing devices 140, and/or remote server 150 via network 145. Processing electronics 352, in whole or in part, can be integrated within wireless communication electronics 358 for efficient handling of communications tasks. Wireless communication electronics 358 can perform these communications, as appropriate depending on the configuration of OBD 101 and other devices 120 being communicated with, and these communication protocols include, but are not limited to, one or two or more of any of those in the “Statement of Wireless Communication Protocols for the Present Embodiments” recited herein. In instances where receiver device 120 is a multi-purpose mobile receiver device 122 such as a smart-phone, communication capability according to mobile protocols is included. Communication electronics 358 can transmit and receive data and commands via interaction with similarly-capable communication electronics of OBD 101, other receiver devices 120, computing devices 140, and/or remote server 150 via network 145.

Communication electronics 358 can include communication circuitry for each protocol with which receiver device 120 operates. Shown here are an antenna 360 communicatively coupled with a transceiver 362. Transceiver 362 has both transmit and receive hardware and capability. Transceiver 362 can further include transmit and receiver amplification circuitry. The communication circuitry has the capability to support all the requisite communication protocols for the implementation of system 100 and can include more than one antenna and transceiver if desired. For brevity, only a single set of the communication circuitry is illustrated in this embodiment. The transceiver 362 can be implemented as a single component or as a separate transmitter and a separate receiver. The one or more transmit amplifiers can be selectively connected between an output of the transmitter of the transceiver 362 and the antenna 360. The one or more receive amplifiers are configured to be selectively connected between the antenna 360 and an input of the receiver of the transceiver 362.

Receiver device 120 can also include wired communication electronics 370 for communicating according to a wired protocol with computing devices 140 and remote server 150 via network 145. Examples of such wired protocols include one or two or more of Universal Serial Bus (USB), Universal Asynchronous Receiver Transmitter (UART), Universal Synchronous Asynchronous Receiver Transmitter (USART), RS-232, RS-485, and/or ethernet. Wired communication electronics 370 includes the hardware and software and physical interface (e.g., port) for effecting communication according to these or other standard or non-standard protocols. With electronics 370, receiver device 120 can, for example, receive software or firmware updates, receive bulk data, and/or upload data to remote server 150 via computing device 140. USB connections can be authenticated on each plug event. Authentication can use, for example, a two-, three-, four-, or five-pass design with different keys. The USB system can support a variety of different sets of keys for encryption and authentication. Keys can be aligned with differential roles (clinical, manufacturer, and/or user, etc.). Sensitive commands that can leak security information can trigger authenticated encryption using an authenticated additional keyset.

Power for all components of receiver device 120 can be delivered by a power source 364. As embodied herein, the power source 364 can include a rechargeable battery, allowing for sustained operations and continued use. Power is supplied from source 364 to the various electronics components by power connection 366.

Receiver device 120 can include timing circuitry 380 having one or more clocks 382 configured to provide a timing reference for tasks such as processing and/or communications. As shown in FIG. 3C, timing circuitry 382 includes N clocks 382-1 through 382-N, where N is an integer ranging from one to ten or more. Typically N is two to six. A clock 382 generates an output signal that acts as a timing reference by oscillating from a low value to a high value and back and repeating this continuously. A single clock period can start at a transition of the clock signal from high-to-low, include a brief low duration then a transition from low-to-high followed by remaining high for another brief duration, with the period ending when the clock signal next transitions from high-to-low. The period can alternatively start and end on a low-to-high transition. The clock continuously repeats this period at a rate determined by the clock's frequency. The clock frequency can be set as needed, for example, ranging from a few kilohertz (kHz) to multiple Megahertz (MHz).

Ideally the clock's output signal reliably maintains the correct time. But this can be difficult in practice. The clock output signal can deviate or drift from the correct time in a direction that is either fast or slow, or the clock may shift between being both fast and slow. The temperature that clock 382 operates at can impact this deviation, as can other factors such as the quality factor (Q value), or variations in the crystal or other components that serve as a reference for the clock. Each clock 382 has an associated clock accuracy that is indicative of the amount of deviation or drift that may be exhibited in a clock's output timing as compared to correct timing. The clock accuracy can be expressed as a quantifiable value (e.g., a number). Clock accuracy is often expressed in the units of parts per million (ppm), but can be expressed in other units as well (e.g., parts per thousand, parts per billion). A clock with an accuracy of +/−50 ppm will generate an output signal that is accurate to within 50 clock periods (either fast or slow) of the correct value when that correct value reaches one million periods. Put differently, a clock accuracy of +/−50 ppm will generate a time value that is between 50 seconds fast and 50 seconds slow of the correct time after one million seconds have passed. The clock accuracy of a relatively less accurate clock will have a higher value (e.g., +/−500 ppm) than the clock accuracy of a relatively more accurate clock (e.g., +/−5 ppm).

Each clock 382 includes hardware components and can be implemented in various designs for the embodiments herein. For example, the clock 382 can be based upon a crystal oscillator circuit, an RC oscillator circuit, or an LC oscillator circuit, to name a few. A crystal oscillator circuit will generally have a higher accuracy than an RC oscillator circuit or an LC oscillator circuit, but a crystal oscillator circuit can be more complex and costly. Clock 382 can be implemented as an on-chip clock or as an external clock. Multiple clocks 382 within receiver device 120 can each have different primary functions. For example, a first clock 382-1 can be an active clock for timing operations while all or a portion of receiver device 120 is in an awake state, while a second clock 382-2 can be a sleep clock for providing timing while all or a portion of receiver device 120 is in a sleep state. Clock 382-2 can control the timing of sending wireless communications to OBD 101 in instances where receiver device 120 is coming out of a sleep state to send a wireless communication, for example, as part of a connection-oriented mode of communication. This description of clock types is not exhaustive, and other types of clock circuits can be used with the embodiments herein as well. Clocks 382 can be communicatively coupled with processor electronics 352, wireless communication electronics 358, wired communication electronics 370, display and input interface electronics 370, and/or other components via interface 304. Clocks 306 can be centralized in OBD 101 or distributed in various locations to serve the circuitry that requires each timing reference.

Receiver device 120 can further include display and input interface electronics 368 for facilitating review of analyte data received from OBD 101 or other device (e.g., user device 140 or remote server 150). The display can be a power-efficient display with a relatively low screen refresh rate to conserve energy use and further reduce the cost of receiver device 120. The display can be a non-interactive screen or a low-cost touch screen that acts as an input interface to receive user input. Although not illustrated, receiver device 120 can include separate input interface components (e.g., buttons, physical keys, toggle switches, light sensors, and/or microphones, etc.) in addition to or as an alternative to a touchscreen. Display and input interface electronics 368 can be communicatively coupled to processing electronics 352, and other components, over communication interface 354.

Communication interface 354 can be electrical traces or connections that communicatively couple two or more entities together. Interface 354 is distributed throughout receiver device 120 to permit communication between all components requiring interaction. Interface 354 can include a main communication bus and one or more I/O ports. Interface 354 can be within a chip, external to a chip (e.g., on a PCB), or both.

As embodied herein, memory 356 of receiver device 120 can be capable of storing analyte data received from OBD 101 over an extended period of time. Further, receiver device 120 or computing device 140 as embodied herein can be configured to communicate with a remote server 150 via a network 145. OBD 101 can provide sensitive data to receiver device 120, which can display that data and/or transmit the data to the computing device 140 and/or remote server 150 for processing, formatting, and/or analysis. In communicating with remote server 150, receiver device 120 or computing device 140 can generate unique user tokens according to authentication credentials entered by a user and stored at the respective device. The authentication credentials can be used to establish a secure connection to remote server 150 and can optionally be further used to encrypt any sensitive data provided to remote server 150 as appropriate. As embodied herein receiver device 120 and computing device 140 can optionally not be as restricted in their use of processing power, and therefore, standard data encryption and/or transmission techniques can be used in transmitted to remote server 150.

Receiver devices such as dedicated receiver device 124 and drug delivery device 126 can further include in vitro blood glucose meter device capability for measuring the level of glucose on a blood glucose test strip. The presence of in vitro blood glucose meter device capability is optional and not a requirement of any embodiment herein.

Receiver devices 120 can include a software or firmware library or application incorporated (e.g., embedded) therein that is configured to manage or control communication with OBD 101. Multi-purpose mobile receiver device 122 can be a suitably configured hardware device that incorporates the software or firmware library or is configured to execute the application. Dedicated receiver device 124 can be a hardware device with embedded software specifically developed and implemented for communicating with OBD 101 within system 100.

### Power Management Based on Clock Accuracy

In many embodiments, OBD 101 is designed for operation as a portable device that is worn on the subject's body to monitor glucose and/or other analyte levels for the designed wear duration. The designed wear duration can extend for not only a matter of days, but one or more weeks (e.g., 10 days, 14 days, 21 days), a month (e.g., 30 days) or longer. The designed wear duration can be limited by a maximum wear duration. OBD 101 may be subject to a regulatory review process that approves use of a single OBD 101 for no longer than the maximum wear duration. Alternatively, a provider (e.g., a manufacturer) of OBD 101 may independently recommend a maximum wear duration that does not exceed the limit set by a regulatory authority. In systems 100 having a maximum wear duration, that maximum wear duration may be the same as the designed wear duration, though the designed wear duration may be more or less. For example, OBD 101 may have a designed wear duration of 14 days and a maximum wear duration of 15 days. Alternatively, OBD 101 may have a designed wear duration of 16 days and a maximum wear duration of 15 days so as to maximize the likelihood the OBD 101 will last as long as permitted. The designed wear duration can be used by the system provider for advertising purposes with consumers. It can also be determined in accordance with a regulatory approval process. If a significant degree of operating condition variance is expected, OBD 101 and sensor may have a designed wear duration that is a range of time, for example, from 7 to 10 days or from 10 to 14 days. In embodiments where system 100 includes either a sensor physically connected to OBD 101, such as with a partially implanted sensor 161 or a wholly ex vivo sensor 163, the designed wear duration of the sensor and OBD 101 will likely be the same. In embodiments where system 100 includes a wholly implanted sensor 162 and wholly separate OBD 101, both the sensor 162 and OBD 101 can each have their own individual designed wear durations, which may differ by days, weeks, or months.

Design and implementation of OBD 101 balances competing concerns of maintaining a form factor that is lightweight, practical, and convenient for the user to maintain on his or her body, while at the same time carrying enough electrical energy in its power source to permit OBD 101 to be worn continuously on the subject's body and operated as needed for the entire designed wear duration.

In many embodiments power source 320 of OBD 101 is a battery having an energy storage capacity (e.g., in milliamp hours (mAh)) sufficient to supply electrical current for the entire designed wear duration of OBD 101. The battery may be relatively small (e.g., less than an inch, or 2.5 cm, in its longest dimension), such as a button cell or coin cell battery, with a generally cylindrical shape having a width (or diameter) measured in an x-y plane that is typically greater than its height measured along the z axis. The battery may alternatively be a printed battery, where the electrodes and electrolytes are printed onto a substrate (e.g., plastic, PCB, foil, paper) using a printing technique that often results in a planar, laminated, flexible structure. Other types of batteries can be used as well. The battery can have one cell or multiple cells (connected in series and/or parallel). The battery can utilize any suitable cell chemistry including, but not limited to, lithium-ion, alkaline, zinc-air, zing-manganese dioxide, zinc-silver oxide, and silver oxide.

In most embodiments, the battery is a non-rechargeable battery. The embodiments herein can be used with a rechargeable (or secondary) battery, but integration of a rechargeable battery can require additional electronics to manage the recharging process, as well as the presence of an externally accessible outer contact or internally disposed wireless charging interface circuitry, both of which add cost and size. In addition, a rechargeable battery requires the user to wait during the recharging process. For a partially implantable sensor 161, the recharging process would take place while OBD 101 is inconveniently on the subject's body. This could also be true for a wholly implantable sensor 162, or the OBD 101 interfacing with the wholly implantable sensor 162 (or a removable-battery containing portion thereof) is removed from the body and recharged, thereby requiring the user to reattach the OBD 101 or the relevant portion, which is inconvenient and causes an interruption in glucose and/or other analyte monitoring. Recharging adds risk in that it requires the user to remember to maintain substantial charge in OBD 101 else, if the battery dies, the analyte monitoring functionality may shut down while the user is relying on it.

A non-rechargeable battery therefore offers benefits, but a relatively large non-rechargeable battery with higher energy storage capacity (as compared to other batteries of the same chemistry type) adds cost, size and weight to OBD 101. The additional weight, in turn, makes it more difficult for OBD 101 to remain adhered to the subject's body as the weight will act as a force that can pull against, and counter to, the adhesive binding force holding OBD 101 to the subject's body. A larger battery requires housing 104 of OBD 101 to increase in size to accommodate that battery. The larger housing 104 adds additional weight of its own and its increased surface area provides greater risk that housing 104 will come into contact with the subject's clothes or other objects or portions of the subject's body. Contact with other entities can result in torque, lateral forces, and/or pulling forces applied to housing 104 and/or pad 106, all of which can cause housing 104 to pull away from pad 106 or can cause pad 106 to peel away from the subject's skin.

Furthermore, a larger battery and housing 104 can require a larger pad 106 and more adhesive for reliable anchoring on the skin, and this larger pad 106 covers more surface area of the skin. This may be less comfortable for the subject and may detrimentally impact the wear duration of OBD 101, as larger pads 106 can result in significant moisture accumulation between the housing and skin. The larger OBD 101 requires greater material costs both in manufacturing OBD 101 itself and in manufacturing a larger applicator or inserter that applies OBD 101 to the subject's body. These increased costs resulting from a larger size are multiplied downstream when the additional costs of handling, shipping, and distributing these larger components are factored in.

Yet, on the other hand, a small battery with relatively low energy storage capacity may not have enough stored energy to permit OBD 101 to operate for the entirety of the designed wear duration. A relatively longer designed wear duration for OBD 101 can be quite beneficial. For example, the user does not need to purchase new on-body devices as frequently, and thus the annual cost for the user is reduced as is any inconvenience of maintaining a regular supply. The user need not perform the OBD application procedure as often, which is more convenient as the application procedure requires piercing the skin with a sharp and some users view that as undesirable. There can also be gaps in monitoring after a OBD 101 expires, as the user must have a new OBD 101 on hand and ready for use, and the user must be in a location and situation conducive to immediately reapplying the new OBD 101. This may not be the case if a user is active or at work or school. As described herein, some conventional OBDs require a warm-up period immediately after activation or their application.

Each OBD 101 can have a target wear duration that is representative of an amount of time the on-body device is expected to operate in an analyte monitoring capacity while on the body. This can include a warm-up period, if any. The target wear duration can be the same as, or representative of, the designed wear duration, though such is not required (such as, in cases where there is no designed wear duration, or the designed wear duration is unclear). For example, if a designed wear duration is 21 days, the target wear duration can also be 21 days. Or if a designed wear duration is a range of days, then the target wear duration can be a duration within that range. The target wear duration can be the same as or less than the maximum wear duration (if applicable). For example, the target wear duration can be 21 days, and the maximum wear duration can be 22 days. Or the target wear duration and maximum wear duration can both be 21 days.

The target wear duration is or is indicative of a time value. The target wear duration can be stored in memory 308 of OBD 101, can be represented in software instructions executable by the processing electronics 302 of OBD 101, and/or or can be implemented in hardware of OBD 101. Elapsed and/or remaining wear duration can be monitored in software and/or hardware. The target wear duration can be a value of actual time (in a familiar sense), such as seconds, minutes, hours, days, and/or weeks. The target wear duration can be a value representative of time conducive for use by a device, such as a count, a timer, a clock of various frequency, or otherwise.

The target wear duration can be fixed and non-adjustable during operation of OBD 101. The target wear duration can alternatively be adjustable by OBD 101 during its operation. The adjustment can be based on power source life. The target wear duration can be adjusted by the on-body device to reflect power or energy usage. OBD 101 can monitor its elapsed wear duration and/or the remaining wear duration before reaching its target wear duration.

The target wear duration can be measured in a manner suitable to the particular implementation of system 100. For example, the target wear duration can be measured from OBD 101's assumption of an active operation state (on or off the body) to termination of the active operation state due to, e.g., reaching the end of power source life or reaching a maximum wear duration if applicable. The target wear duration can be a continuous period of time that excludes periods of temporary inactivity, such as for power saving or intervals between measurements. The target wear duration can be measured from OBD 101's placement on the body with an implanted sensor 161 to termination of its active operation state (e.g., by removal, expiration, and/or power source drainage). The target wear duration can be measured from a time of initiation (such as the examples described) to a time where replacement or removal is recommended (e.g., upon reaching a power source remaining life threshold).

In some embodiments, system 100 and OBD 101 can implement the target wear duration as a power source life that is indicative of a time before the power source reaches a voltage, current, energy, and/or power level that is beneath the minimum required or recommended for continued operation of OBD 101.

Returning to the considerations of OBD development, the designer of OBDs 101 must carefully balance the need to maintain the size and weight of OBD 101 as small as is practical, with the need to have a power source with storage capacity substantial enough to provide the benefits of an extended designed wear duration.

For these and other reasons, example embodiments of system 100 are described herein that permit OBD 101 to communicate wirelessly with less current consumption from its power source 320. This reduction in current consumption, in turn, permits OBD 101 to consume less power over a given duration of use, reducing the storage capacity requirement for the power source (e.g., battery) over the target wear duration of OBD 101. A significant amount of the power budget for OBD 101 is needed to operate wireless communication electronics 312, for example, to operate transceiver 316 for making outgoing wireless transmissions and for monitoring for receipt of incoming transmissions.

In order to conserve power, OBD 101 can be configured to operate the receiver of transceiver 316 only during a period of time in which OBD 101 expects to receive a transmission from receiver device 120. This period of time can be referred to as a listening window. FIG. 4A is a timing diagram depicting an example of wireless communication between OBD 101 and receiver device 120. Transceiver activity of the devices is shown on the Y axis and time is shown on the X axis. At time T0 both OBD 101 and reader device 120 are inactive with neither device sending transmissions nor enabling their receivers to receive transmissions. At time T1 OBD 101 activates its listening window and supplies power to the receiver to monitor for a wireless transmission from receiver device 120. The receiver turns on at time T1 because both devices 101 and 120 are synchronized and have agreed to schedule transmissions according to a common clock. Each device operates its own clock to track the time in relation to the agreed upon schedule. At time T2 receiver device 120 begins transmitting a message to OBD 101 and continues this transmission until time T3. OBD 101 detects the receipt of this transmission and continues with its receiver in the active state until it detects the end of the transmission at time T3, at which point OBD 101 deactivates its receiver at time T4. Here, due to inaccuracy of clock 382 of receiver device 120, the initiation of the transmission at time T2 was delayed from the time T1 at which OBD 101 activated its receiver. The receiver was thus operating during this time interval T1-T2 without receiving any transmission and the power required to do so was consumed unnecessarily and thus wasted. Design of the communication scheme will incorporate extra time into the listening window, for example by starting the window early, so as not to miss the initiation of a transmission by receiver device 120. However too long of a delay between the start of the listening window and the receipt of the transmission at time T2 is an inefficient use of energy from power source 320 of OBD 101.

This problem is exacerbated when the delay between the initiation of the listening window and the start of transmission by receiver device 120 becomes so long that the listening window must be extended beyond its normal planned duration. This situation is depicted in FIG. 4B. Here, OBD 101 activates the receiver and commences the listening window at time T1. Time T4 is reached without receipt of the transmission from receiver device 120 and OBD 101 determines to keep the receiver active and extends the listening window beyond time T4. At time T5 receiver device 120 starts the transmission which continues until time T6. The cessation of the transmission is detected by OBD 101, and the receiver is deactivated, and the listening window is terminated at time T7. In this example, the duration of time that the listening window is active from T1 to T5 does not accomplish the goal of receiving a transmission and thus the power to maintain the receiver in an active state is wasted.

Transmission time deviations like those described with respect to FIGS. 4A and 4B can be the result of the incorporation of clocks 382 in receiver devices 120 that do not have the quality to prevent deviations of the magnitude that impacts system performance. If the clock accuracy of a clock 382 is not good enough, then the timing of clock 382 will drift significantly from the actual or real time, and receiver device 120 transmits at a time that is correct as far as receiver device 120 can tell, but in actuality is significantly incorrect.

In the example embodiments described herein, system 100 can be configured such that the clock accuracy of the clock 382 that is used to generate the timing reference signal for triggering the sending of wireless transmissions from the receiver device 120 to OBD 101 is made known or available to other operating entities within system 100, such as OBD 101 and/or an analyte monitoring application (e.g., an app) being executed by receiver device 120. The clock accuracy is represented by a clock accuracy parameter. The clock accuracy parameter is known by the provider or manufacturer of receiver device 120. The clock accuracy can be determined by testing the clock 382 of each receiver device 120 during manufacturing or post-manufacturing testing. The clock accuracy can be determined by characterizing a representative sample of receiver devices 120 that share the same or similar clock design. The clock accuracy can be provided to the provider or manufacturer of receiver device 120 by a supplier of components to the provider or manufacturer, based on the testing or characterization performed by the supplier. In embodiments using certain features of Bluetooth Low Energy as protocols governing at least some wireless transmissions between devices, such as advertisements, the clock accuracies are referred to as the main clock accuracy (MCA) which is associated with the synchronizing device, e.g., the OBD 101 or receiver device 120 depending on the desired system communication configuration, and the sleep clock accuracy (SCA) which is associated with the synchronizing follower device, e.g., the other one of the OBD 101 and receiver device 120 depending on the system communication configuration.

The clock accuracy can be known with precision, e.g., a discrete value at a given set of operating conditions. However as the amount of drift in clock timing is often dependent on environmental factors like temperature, clock accuracy is appropriate to be described as within a clock accuracy range, or satisfying a clock accuracy limit, where the range considers variations in environmental factors, manufacturing, and/or component quality. A clock 382 may have a clock accuracy that is qualified to be within the range of, or no worse than a limit of, for example, +/−5 ppm, +/−10 ppm, +/−15 ppm, +/−20 ppm, +/−40 ppm, +/−50 ppm, +/−100 ppm, +/−200 ppm, +/−300 ppm, +/−400 ppm, +/−500 ppm and so forth. In such cases while the clock accuracy is not known to be a single discrete value, it is known that the clock accuracy can deviate during normal operating conditions to any accuracy value within the specified clock accuracy range (e.g., from −500 ppm to +500 ppm, or within a limit of +/−500 ppm from the real value) and a determination can be made as to the relative quality of the clock as compared to other clocks since a higher quality clock will have a lower value clock accuracy limit.

The clock accuracy can be represented by data that is indicative of the clock accuracy. For example, the clock accuracy can be coded such that a defined code corresponds to a particular clock accuracy. In this way, system 100 need not convey the clock accuracy in the same way that is presented here for human understanding, but rather can read and interpret clock accuracy as is suitable for machine operation. Table 1 below gives examples of clock accuracies and coded format that may be used in an example embodiment of system 100.

TABLE 1

| Code | Clock Accuracy |
|---|---|
| 000 | +/−0 to 20 ppm |
| 001 | +/−21 to 50 ppm |
| 010 | +/−51 to 100 ppm |
| 011 | +/−101 to 150 ppm |
| 100 | +/−151 to 200 ppm |
| 101 | +/−201 to 250 ppm |
| 110 | +/−251 to 500 ppm |
| 111 | greater than +/− 500 ppm |
| other | Invalid parameter |

The clock accuracy in the above examples has a value that corresponds to a quantitative representation of the clock accuracy. The clock accuracies can be partitioned so as to be mutually exclusive, e.g., a clock accuracy limit of +/−225 ppm would fall within the range of +/−201 to 250 ppm of the example embodiment of Table 1 and be classified as code 101. The clock accuracy can be coded in this manner by the manufacturer or provider of receiver device 120 when stored in the memory 356 of receiver device 120. Alternatively, processor electronics 352 of receiver device 120 can make this determination and classification when the clock accuracy parameter is requested as described below.

The clock accuracy can additionally, or alternatively, have a value that corresponds to a qualitative representation of the clock accuracy. For example, a clock 382 can have a clock accuracy parameter that equates to a suitable clock accuracy for use in system 100, or a clock accuracy parameter that equates to an unsuitable clock accuracy for use in system 100, where the suitability of the clock accuracy value is based on the impact that the clock accuracy would have on the power budget of OBD 101. The qualitative representation may be more granular, for example, values for the clock accuracy can correspond to a suitable clock accuracy with no impact on power budget, a suitable clock accuracy with minor impact on power budget, an unsuitable clock accuracy that may result in failure to meet the target wear duration of OBD 101, or an unsuitable clock accuracy that will result in failure to meet the target wear duration of OBD 101.

The clock accuracy can be stored in memory 356 of receiver device 120. For example, the manufacturer of receiver device 120 may store the clock accuracy parameter during installation of the software or firmware of reader device 120 or the individual chips therein, or through the manufacturing of ROM in receiver device 120, or by installation or downloading of device parameters into the memory of receiver device 120 during a post-manufacturing process, prior to sale or other provision to the end user.

Receiver device 120 can retrieve the clock accuracy parameter from memory 356 in response to a request and then provide it to the requesting entity. Alternatively, the receiver device 120 can retrieve its clock accuracy parameter by communication exchange with a remote cloud server. In some example embodiments the clock accuracy parameter is available to a lower layer of a protocol stack architecture implemented in receiver device 120. Retrieving the clock accuracy parameter may require a request to be sent from a first protocol layer to a second protocol layer for the clock accuracy, at which time the clock accuracy is retrieved by the second layer and made available or passed to the first layer. The two layers may be adjacent or non-adjacent. For example, OBD 101 can request the clock accuracy parameter from reader device 120, where the transmitted request is received by wireless communication electronics 358 and parsed, with the request then interpreted and/or processed by an application being executed within an application layer of a protocol stack implemented in the electronics 351 of receiver device 120. The clock accuracy parameter can be stored in or accessible by a lower layer, such as a link layer adjacent or in proximity with a physical layer, such that the request is passed between intervening layers and interfaces (e.g., a host-controller interface), processed by the responsible lower layer, and the clock accuracy parameter is then passed back through the intervening layers and interfaces to the higher application layer. The application layer can then optionally code the clock accuracy parameter such as described with respect to Table 1, format the clock accuracy parameter into the appropriate data format and message location, and then pass the message to the wireless communication electronics 358 for transmission back to OBD 101.

Alternatively, the application being executed on the receiver device 120 can request the clock accuracy parameter as part of a programmed routine, which may be executed upon installation of the application on the receiver device 120 or may be executed upon or after establishing communication with OBD 101. The clock accuracy parameter can then be transmitted by the receiver device 120 to OBD 101 automatically without an independent request from OBD 101.

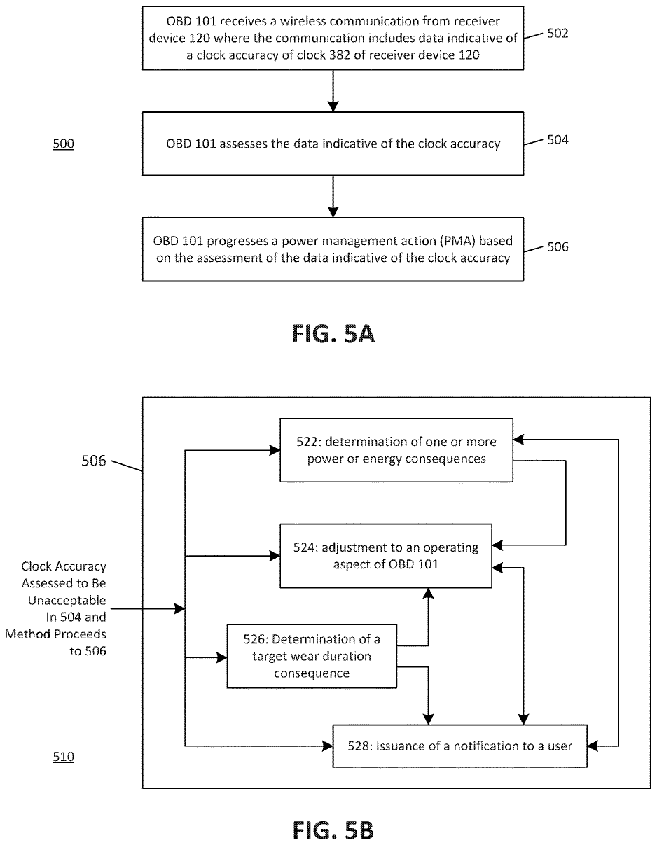

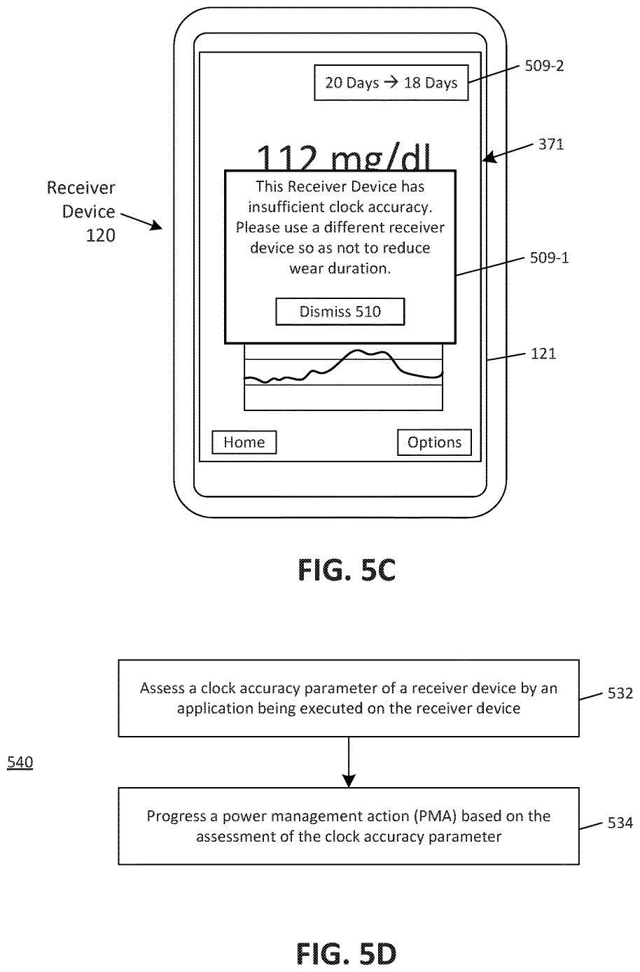

FIG. 5A is a flow chart depicting an example embodiment of a method 500 of managing power for OBD 101 in system 100 based on the assessment of clock accuracy of receiver device 120. At 502, OBD 101 receives a wireless communication from receiver device 120 where the communication includes data indicative of a clock accuracy of a clock 382 of receiver device 120. The data indicative of the clock accuracy is a clock accuracy parameter in this embodiment. As mentioned herein the clock accuracy parameter can be a value that is indicative of a quantitative assessment of the clock accuracy or a qualitative assessment of the clock accuracy. As mentioned herein, the clock accuracy parameter can be an actual value of the clock accuracy, a range or limit of the clock accuracy, a code that is representative of the actual value, range, or limit of the clock accuracy, or otherwise.

In other embodiments, the data indicative of the clock accuracy can be an indication of a type of the receiver device. The type of the receiver device can be a manufacturer and/or a model of the receiver device 120, where the type is known to OBD 101 as being associated with a particular clock accuracy that is acceptable or not acceptable. For example, the receiver device 120 can be designated as being of a type having an adequate or acceptable clock accuracy, based on an indication of the manufacturer of that device 122, and or the model of that device 122, and pre-existing knowledge that the manufacturer and/or model are of a trusted acceptable quality level.

Indication that the receiver device is a dedicated receiver device 124 or a drug delivery device 126 can be indicative of an acceptable clock accuracy as those devices can be manufactured with the glucose monitoring environment as a primary use case. Identifying information of the type can be reported by device 122 to OBD 101 as the data indicative of the clock accuracy. System 100 can be configured such that the data indicative of the clock accuracy includes the clock accuracy parameter in addition to type, manufacturer, and/or model information.

At 504, OBD 101 assesses the data indicative of the clock accuracy. In this embodiment the assessment of the data indicative of the clock accuracy is an assessment of the clock accuracy parameter as explained below. In other embodiments the assessment is of the indication of the type of the receiver device 120, which involves a determination as to whether the type of the receiver device 120 is associated with (or corresponds to) a receiver device 120 having a clock accuracy that is or is not acceptable based on type data stored within OBD 101. For example, data indicating receiver device 120 corresponds to a first type (e.g., a first manufacturer and/or a first model, or a dedicated receiver or drug delivery device) can be associated within OBD 101 (e.g., within memory or by software) with an acceptable clock accuracy, while data indicating receiver device 120 corresponds to a second type (e.g., a second manufacturer and/or a second model, or not a dedicated receiver nor drug delivery device) can be associated within OBD 101 (e.g., within memory or by software) with an unacceptable clock accuracy.

The assessment of the clock accuracy parameter can be a determination as to whether the clock accuracy parameter is acceptable for timing transmissions from receiver device 120 to OBD 101. In this embodiment, step 504 is performed by software instructions executed by processing electronics 302 of OBD 101. In an alternative embodiment, the assessment is performed by hardware. The assessment can produce at least one of two outputs, namely, that the clock accuracy is acceptable or is not acceptable. This assessment can be made by comparing the clock accuracy to a threshold accuracy value, where a clock accuracy that deviates by less than the threshold is acceptable and a clock accuracy that deviates by more than the threshold is unacceptable.

Similarly, the assessment can be made by comparing the received clock accuracy parameter to a data structure (or identifying the received clock parameter in the data structure), such as a list, array, table or look up table, register, or database that contains values of potential clock accuracy parameters and associated indications of whether or not those clock accuracy parameters are acceptable. The data structure can be maintained in memory 308 of OBD 101. OBD 101 can thus determine whether the clock accuracy parameter is acceptable or unacceptable based upon the information contained in that data structure. If reference is made to information about the receiver device type, model, manufacturer, and/or version for determination of an associated clock accuracy parameter, then this information and the corresponding clock accuracy parameter can be maintained in the data structure. The data structure can be uploaded to OBD 101 during manufacturing. Additionally, or alternatively, system 100 can be configured such that receiver device information will be delivered to the OBD, e.g., during activation or validation steps in establishing communication between OBD 101 and a particular receiver device 120.

The assessment can produce one or more of a range of three or more outputs, such as, the clock accuracy is acceptable, the clock accuracy is not acceptable, and if the clock accuracy is not acceptable then a measure of the degree by which the clock accuracy differs from an acceptable value. For example, if an acceptable clock accuracy parameter is a threshold of ±50 ppm or less, and the received clock accuracy parameter is ±200 ppm, then the assessment can output an indication that the clock accuracy parameter is unacceptable as well as an indication that the clock accuracy is 150 ppm greater than the acceptable threshold.

The clock accuracy threshold can be a desired clock accuracy or a required clock accuracy that leads to confidence that OBD 101 will have enough power source capacity to continue operation over the target wear duration. The threshold and/or methods for determining the threshold are based upon testing, modeling, calculations, or a combination of two or more of the aforementioned.

In some embodiments, there is one clock accuracy threshold that is used for acceptability assessments during the target wear duration of OBD 101. In other embodiments, multiple clock accuracy thresholds can be used during the target wear duration, with each clock accuracy threshold corresponding to a particular amount or range of time that is left in the target wear duration of OBD 101. In an example, a OBD 101 has a target wear duration of 30 days, with a different clock accuracy threshold for every day of the 30 days, where the threshold on the first day is the relatively most stringent threshold (the threshold requiring the relatively most accurate clock), each subsequent day has a threshold that is relatively less stringent than the prior, and the threshold for the 29th day is the relatively least stringent threshold (the threshold requiring the relatively least accurate clock compared to the other thresholds), given that the amount of time that OBD 101 may have to operate with the given clock 382, and thus the potential power budget impact, decreases as OBD 101 progresses through the target wear duration. In other embodiments, clock accuracy thresholds do not change daily, but rather by a different amount of time that is less than a day (e.g., second, minute, hourly, or 12-hour period), or more than a day (e.g., a threshold assigned to each group of two days, three days, a week, or more).

Alternatively, OBD 101 can determine an appropriate threshold by processing two or more conditions according to an algorithm, formula, or set of rules. The conditions can include a time condition and a remaining power or energy condition. The time condition can be indicative of the remaining amount of time left in the target wear duration of OBD 101, e.g., a percentage of the target wear duration that has passed, or a quantity of time (e.g., seconds, minutes, hours, half-days, etc.), that remains in the target wear duration of OBD 101. The remaining power condition can be indicative of the amount of energy or available power capacity that remains in the power source 320 of OBD 101, for example, a present state of charge in the battery. OBD 101 can then determine the threshold based on the time and remaining power conditions. An example formula for threshold determination is given by (1) below, where P_{R} is the remaining power capacity (e.g., in milliwatt hours (mWh)), P_{C} is the average power consumption (e.g., mW) for a baseline clock accuracy (e.g., 5 ppm, 20 ppm, 40 ppm, etc.), K is a factor representing the average amount of power consumed per unit of clock accuracy deviation from the baseline (e.g., mW/ppm), D_{A} is the acceptable deviation from the baseline (e.g., ppm), and T_{R} is the time remaining in the target wear duration (e.g., in hours).

P_{R}>(P_{C}+(K·D_{A}))T_{R} (1)

OBD 101 can utilize (1) to solve for D_{A}, which can be added to the baseline threshold to determine a new threshold clock accuracy that accounts for the remaining power capacity in OBD 101 and the remaining time in the target wear duration of OBD 101. The results of the algorithm can be predetermined for many possible combinations of the time condition and remaining power condition. These results can be logged in a data structure, e.g., a table, look-up table, or arrays, and/or can be coded directly into software instructions to permit OBD 101 to directly look up a new clock threshold given the present time condition and remaining power conditions. This alleviates the need to algorithmically calculate the new clock accuracy threshold, as particularly complex algorithms can require significant power in their performance.

Referring to FIG. 5A, at 506, OBD 101 can progress a power management action (PMA) based on the assessment of the data indicative of the clock accuracy, which in this embodiment is a clock accuracy parameter. Different PMAs can be taken based on whether the assessment of the clock accuracy parameter indicates that continued operation with the receiver device 120 will or will not significantly impact the power budget of OBD 101, such as whether the clock accuracy parameter is expected to cause the target wear duration of OBD 101 to be reduced and not meet the target wear duration. This can involve initiation of a PMA or furtherance of a PMA already in effect. To name a few examples, the PMA can be an act that is taken to maintain operation of OBD 101 within its power budget or target wear duration, to seek intervention of the user to assist in operating OBD 101 in a fashion that will allow it to remain within its power budget or target wear duration, and/or to notify the user of the impact that continued operation of OBD 101 with the receiver device will have on OBD 101's power budget or target wear duration. The PMA can be taken by OBD 101 and/or another entity in system 100, such as receiver device 120. For example, the action can be initiated by OBD 101 and transmitted to the receiver device 120 which can further the action or complete the action (such as presentation of a notification). Embodiments of method 500 are configured to facilitate the use or operation of OBD 101 in a manner that does not reduce its wear duration beyond the target, whether the target is or is not self-enforced by the software of OBD 101.

In the instance that the clock accuracy is acceptable, then no PMA need to be taken, though a notification of the suitability, adequacy, or quality of the receiver device 120 or its clock 382 can be issued. A host of different actions can qualify as the PMA in a circumstance where the clock accuracy parameter is assessed to be unacceptable. FIG. 5B is a flow diagram depicting examples of PMAs that can be taken in step 506 of an example embodiment of method 500 represented here as method 510. The assessment of clock accuracy (e.g., 504 of FIG. 5A) is shown at left and a determination is made that the clock accuracy is not acceptable and warrants a PMA. Four PMAs, or groups of PMAs, 522-528 are shown and any and/or each can be taken by OBD 101 and/or another component of system 100, such as receiver device 120, computing device 140, and/or remote network 150, or a combination of components of system 100. PMAs 522-528 are each optional and can be taken independently of all other actions, or in combination with one, two, or three other actions 522-528.

Although not exhaustive of all possible PMA actions, the four independent PMA actions that can be taken in response to assessment of the clock accuracy as being unacceptable are shown in FIG. 5B as: determination of one or more power or energy consequences 522; adjustment to an operating aspect of OBD 101 in action 524; determination of a target wear duration consequence 526; and issuance of a notification to a user 528. Determination of one or more power or energy consequences 522 can be the only PMA taken, or it can be a first PMA that leads to a further PMA of one, two, or all of adjustment to an operating aspect of OBD 101, determination of a target wear duration consequence 526 and/or issuance of a notification to a user 528. Adjustment to an operating aspect of OBD 101 can be the only PMA taken, or it can be a first PMA that leads to a further PMA of issuance of a notification to a user 528. Determination of a target wear duration consequence 526 can be the only PMA taken, or it can be a first PMA that leads to a further PMA of either or both of adjustment to an operating aspect of OBD 101 and/or issuance of a notification to a user 528. Issuance of a notification to a user 528 can be the only PMA taken, or it can be a first PMA (such as in the case where the notification is a prompt or query to the user) that leads to a further PMA of one, two, or all of determination of one or more power or energy consequences 522, adjustment to an operating aspect 524 of OBD 101, and/or determination of a target wear duration consequence 526, each of which can lead back to another instance of issuance of a (second or subsequent) notification to a user 528.

Regarding a determination of one or more power consequences 522, the PMA can be or include a determination of a new rate of power consumption (P_{NR}), a power budget (e.g., surplus, break even, or deficit conditions), an adjustment to rate of power consumption (A_{PNR}) for OBD 101, or a combination of two or all of these.

The new clock accuracy can be used to determine a new rate at which OBD 101 will, or is expected to, consume power P_{NR}. An example formula for a new rate determination is given by (2) below, where P_{NR} is the new rate power of power consumption (e.g., in milliwatt hours (mWh)), P_{BR} is the baseline rate of power consumption (e.g., mWh) based on a baseline acceptable clock accuracy (e.g., 5 ppm, 20 ppm, 40 ppm, etc.), X is a factor representing the rate of power consumption per unit of clock accuracy (e.g., mWh/ppm), D_{B} is the deviation of the receiver device's clock accuracy from the baseline (e.g., ppm).

P_{NR}=P_{BR}+(X·D_{B}) (2)

OBD 101 can utilize (2) to solve for P_{NR}, and thus have a quantified new rate of power consumption based on the receiver's clock accuracy. P_{NR} can be multiplied by the remaining time condition to determine a cumulative amount of power that OBD 101 will consume based on the remaining target wear duration of OBD 101. A power budget can be determined by comparing the new cumulative amount of power with the remaining power available in power source 320 of OBD 101. A power deficit exists if the new cumulative amount exceeds the remaining power and OBD 101 will be unable to operate until the end of the target wear duration without remedial action. A power surplus exists if the remaining power exceeds the new cumulative amount, and a break-even condition exists if they are equal.

If there is a deficit, then OBD 101 may need to adjust the new rate of power consumption to maintain operation through the end of the target wear duration or change and lower the target wear duration to accommodate the greater use of power, or a combination of both. To determine the adjustment (A_{PNR}) to the new rate P_{NR}, OBD 101 can determine a maximum rate of power consumption that is necessary to continue operation until the end of the target wear duration (e.g., by dividing the available power by the remaining time condition), and the difference between this maximum rate and P_{NR} can be a quantification of the adjustment (A_{PNR}) to the new rate of consumption (P_{NR}) that is required to operate for the remainder of the target wear duration.

As with determinations of the clock accuracy threshold according to equation (1), determinations of power consequences 522 (including a new rate of power consumption (P_{NR}), a power budget, and/or an adjustment to the new rate of power consumption (A_{PNR}) for OBD 101) can be preset or precalculated (e.g., set or calculated by the manufacturer or provider prior to provision to the user) for many possible combinations of the clock accuracy deviation (DCA), the baseline rate of power consumption (P_{BR}), remaining time condition, and/or remaining power condition. These preset results can be logged in a data structure, e.g., a table, look-up table, arrays, register, database or can be coded directly into a software function, in order to permit OBD 101 to directly look up (and thus determine) a new rate of power consumption, a power deficit, and/or an adjustment to rate of power consumption given the relevant conditions, such as clock accuracy deviation (DCA), the baseline rate of power consumption (P_{BR}), present remaining time condition and/or present remaining power condition. Reliance on a preset data structure alleviates the need to algorithmically calculate these new values, as particularly complex algorithms can require significant power in their performance.

In some embodiments, OBD 101 may be managed in terms of energy consumption instead of, or in addition to, power consumption. In those instances, the PMA can be or include the determination of consequence 522 in terms of energy (determination of an energy consequence 522). The energy consequence can be or include a new rate of energy consumption (E_{NR}), an energy budget (e.g., surplus or deficit), or an adjustment to the new rate of energy consumption for OBD 101 (A_{ENR}), or a combination of two or all of these. These values can be arrived at in similar fashion by substituting corresponding energy values for the power values described above and with reference to (2).

In the embodiments herein, assessment of the clock accuracy parameter and/or progressing a PMA (and substeps thereof), including use in formulas (1) and (2), can be performed utilizing only the clock accuracy parameter of a given receiving device 120 and no other device. In these embodiments, if desired the assessment of the clock accuracy parameter and/or progressing a PMA (and substeps thereof), including use in formulas (1) and (2), can be performed utilizing the clock accuracy parameter of multiple receiving devices 120, or alternatively the receiving device 120 having the relative lowest clock accuracy. Still further, in these embodiments, if desired the assessment of the clock accuracy parameter and/or progressing a PMA (and substeps thereof), including use in formulas (1) and (2), can be performed utilizing the clock accuracy parameter of one or all receiving devices 120 in addition to the clock accuracy of the OBD 101 itself, which can be known to OBD 101 by virtue or storage of that clock accuracy parameter in memory or inclusion in software instructions. The clock accuracy parameter for OBD 101 can be a minimum threshold value that is the same for all OBDs 101 of a particular model or class or can be measured independently for each OBD 101 or subset of OBDs 101 and written to memory during manufacturing or testing.

Referring now to the determination of a target wear duration consequence 526, the consequence can be or include a new target wear duration, or an adjustment to the present target wear duration of OBD 101, given the additional power consumption required by operation with the reduced clock accuracy. To determine the new target wear duration OBD 101 can divide the remaining available power by P_{NR} to arrive at a duration of operation that can be expected for OBD 101. If this duration is more than the remainder of the target wear duration, then no adjustment to the target wear duration is required. If this duration is less than the remainder of the target wear duration, then a new reduced target wear duration results, unless further action is taken to mitigate the increased power consumption. The user can be notified of the modification to the target wear duration, as described below regarding step 528.

Referring now to adjustment to an operating aspect 524 of OBD 101, the PMA can be the implementation of a new power or energy management scheme in OBD 101 to conserve power in the case where the clock accuracy is unacceptable. For example, OBD 101 can reduce its new rate of power or energy consumption by the adjusted amount (A_{PNR} or A_{ENR}) to maintain operation through the entirety of the target wear duration. To do this, OBD 101 can alter its schedule of operation to increase the amount of time that OBD 101 is in a first lower power consuming state as compared to a second higher power consuming state. For example, OBD 101 can alter its schedule of operation to increase the amount of time OBD 101 is in a sleep state relative to a partially awake state or fully awake state (or just awake state), where a sleep state corresponds to a period that consumes relatively less power than the partially awake state which, in turn, corresponds to a period that consumes relatively less power than the fully awake state. Additionally or alternatively, OBD 101 can alter its schedule of operation to increase the amount of time that OBD 101 is in a partially awake state relative to an awake or fully awake state.

The different states of operation can involve the consumption of power to processor electronics 302, such as a sleep state having less power consumed by processor electronics 302 than a partially awake state or a fully awake state. This can result from a portion of processor electronics 302 not being fully powered, or a portion of processor electronics 302 operating at a lower clock rate, and/or processor electronics 302 not being fully initialized. The different states of operation can involve different amounts of power source to wireless communication electronics 302, such as a sleep state having less power consumed by a greater interval between the sending of communications as compared to a partially awake state or a fully awake state. For example, OBD 101 can increase an interval between transmissions from one minute to two minutes, e.g., reduce the rate of transmissions, and thereby reduce the power consumption due to wireless transmission by 50%.

Further, OBD 101 need not operate according to different formal states such as sleep, partially awake, and/or fully awake (or just awake) in order to accomplish the relative reductions in power consumption. For example, OBD 101 can alter its schedule of communication with receiver device 120 and switch from a first rate of transmitting glucose and/or other analyte level measurements to a second rate of transmitting glucose and/or other analyte level measurements that is slower than the first rate. By way of example, the rate reduction can be a one minute, two minute, three minute, or four minute reduction, and so forth. By way of additional examples, the rate reduction can be from every one minute to every two minutes, from every one minute to every 3 minutes, from every two minutes to every 3 minutes, from every 3 minutes to every 4 minutes, from every 3 minutes to every 5 minutes, from every 4 minutes to every 5 minutes, and so forth. The rate reduction can occur without affecting an alarm generation and transmission routine that originates at OBD 101. In another embodiment, if analyte alarms (e.g., low analyte, high analyte, high rate of change of analyte, etc.) are generated by OBD 101, then the adjustment to an operating aspect of OBD 101 can be transitioning to a mode of communication where analyte measurements are not automatically transmitted to receiver device 120 without prompting and only alarms are transmitted to receiver device 120 whenever such an alarm is generated by OBD 101 in the course of taking analyte measurements from the subject.

The adjustment to an operating aspect 524 of OBD 101 can additionally, or alternatively, include an adjustment to a mode of wireless communication between OBD 101 and the receiver device 120. For example, OBD 101 can transition from a first mode of communication to a second mode of communication wherein the second mode of communication consumes less power. The first mode of communication can be a connection-oriented mode of communication such as a paired communication session, and second mode of communication can be a connection-less mode of communication, such as communication by way of advertising transmissions and responses. Alternatively, the first mode of communication can be a connection-less mode of communication and the second mode of communication can be a connection-oriented mode of communication. Determination of which mode of communication is more power efficient is dependent upon the design and implementation of OBD 101. In some embodiments, the determination of which mode is more power efficient is dependent upon the rate of transmissions that are sent in that mode. For example, the sending of glucose and/or other analyte level measurements by OBD 101 at a rate of every three minutes or less can result in a connection-oriented mode of communication being more power efficient than a connection-less mode, while the sending of glucose and/or other analyte level measurements by OBD 101 at a rate of more than every 3 minutes can result in a connection-less mode of communication being more power efficient than a connection-oriented mode. Thus, a determination whether to switch from a first mode to a second mode can be based in part on the rate of transmission of glucose and/or other analyte level measurements. The determination of whether to transmit in a connection-less mode or connection-oriented mode can be based on the type of information being transmitted, in addition or in alternative to the power consumption factor of the mode. n an example embodiment a determination to use a connection-less mode can be based on the relative power consumption of the mode and/or a data type that corresponds to routine data being transmitted, while a determination to use a connection-oriented mode can be based on the relative power consumption of the mode and/or a data type that corresponds to critical data being transmitted such as an alarm or analyte data that is outside of a threshold or trending towards violating a threshold. In some embodiments, a first PMA can be a modification to the rate of transmissions and a second PMA can be changing the mode of communication.

The adjustment to an operating aspect 524 of OBD 101 can also or alternatively be an adjustment to a temporal length of a receive window operated by OBD 101 to listen or monitor for a communication from a receiver device 120. For example, a receive window for a transmission from a receiver device 120 having a relatively high clock accuracy (e.g., a clock accuracy that satisfies a threshold) can be set at a minimum length, or a length that is relatively shorter than a receive window for a transmission from a receiver device 120 having a relatively low clock accuracy (e.g., a clock accuracy that fails to satisfy a threshold). The receive window length can be set based on a linear scale in proportion to the degree of clock accuracy for the receiver device 120.

The adjustment to an operating aspect 524 of OBD 101 can be dependent on user behavior. The frequency at which a user accesses analyte measurements on receiver device 120 can be monitored and a time period of relatively infrequent accessing of the measurements can be identified (e.g., a user asleep time period, a user at work time period, and/or a user at rest time period) such as by historical analysis of the user's behavior. Alternatively, a user can be prompted (in step 528) to enter one or more time periods of relative inactivity. System 100 can adjust the operating aspect 524 of OBD 101 during this time period of relative inactivity. The above embodiments pertaining to adjustments to an operating aspect 524 by implementation of a new power or energy management scheme in OBD 101 to conserve power in the case where the clock accuracy is unacceptable by, e.g., alter a schedule of operation to increase the amount of time that OBD 101 is in a first lower power consuming state as compared to a second higher power consuming state, alter a schedule of communication with receiver device 120, alter a first rate of transmitting glucose and/or other analyte level measurements to a second rate of transmitting glucose and/or other analyte level measurements that is slower than the first rate, transitioning to a mode where only alarms are transmitted and analyte measurements are not, and/or altering a mode of wireless communication between OBD 101 and the receiver device 120, can each be applied during this identified time period of relative inactivity to reduce power consumption.

The adjustment to an operating aspect 524 of OBD 101 can be dependent on an environmental factor such as temperature. Temperature of OBD 101, particularly power source 320 and the remaining electronics 110, can impact the rate of power consumption and available power in source 320. For example, electronics 110 will tend to consume relatively more power to perform relatively the same operation at higher temperatures than at lower temperatures. Conversely, power source 320 can experience relatively worse efficiency resulting in greater power use at colder temperatures than at higher temperatures. A component of system 100, such as a temperature sensor of OBD 101, can be used to monitor temperature of OBD 101 or the ambient environment surrounding OBD 101. Alternatively, a receiver device 120 can monitor the temperature. Electronics 110 can identify and/or detect if a temperature threshold has been violated where the threshold can be for an unacceptably high temperature or an unacceptably low temperature, or there can be multiple thresholds for both. System 100 can adjust the operating aspect 524 of OBD 101 during a time period where the detected temperature threshold violation is in effect. The above embodiments pertaining to adjustments to an operating aspect 524 by implementation of a new power or energy management scheme in OBD 101 to conserve power in the case where the clock accuracy is unacceptable by, e.g., alter a schedule of operation to increase the amount of time that OBD 101 is in a first lower power consuming state as compared to a second higher power consuming state, alter a schedule of communication with receiver device 120, alter a first rate of transmitting glucose and/or other analyte level measurements to a second rate of transmitting glucose and/or other analyte level measurements that is slower than the first rate, transitioning to a mode where only alarms are transmitted and analyte measurements are not, and/or altering a mode of wireless communication between OBD 101 and the receiver device 120, can each be applied during this time period of temperature violation to reduce power consumption at those times when it would result in relatively greater inefficiencies of operation.

In some instances, the adjustment to the rate of power or energy consumption (A_{PNR} Or A_{ENR}) may be so large that OBD 101 cannot scale back operation enough to satisfy the adjustment. If a lesser partial adjustment can be made, one that reduces the rate of power (or energy) consumption but not the full amount required to meet the target wear duration (A_{PNR} or A_{ENR}), then OBD 101 can divide the remaining available power by the P_{NR} modified by this lesser partial adjustment to determine a new revised target wear duration. In this manner, OBD 101 can adjust both the target wear duration (step 526) and the rate of power consumption (step 524).

Turning now to the issuance of a notification to a user 528, the notification can be generated by OBD 101 first as an instruction that is sent from OBD 101 to receiver device 120 to which receiver device 120 can respond by presenting a notification to the user on the display of receiver device 120. OBD 101 can thus instruct receiver device 120 to notify the user and receiver device 120 can issue that notification. In embodiments where OBD 101 has user notification capability, such as visual, audible, haptic, and/or tactile indicators, OBD 101 can issue the notification to the user in addition to instructing receiver device 120 to do so, or instead of receiver device 120.

The notification itself can take various forms. The notification can be the presentation of a graphical user interface element in the form of a window, screen, and/or banner on a display (see, e.g., FIG. 5C). The window can be a pop-up window that is overlaid over background graphics. The screen can be a full screen element that takes up the entirety of the display. The banner can be similar to a pop-up window and can appear at an edge of the display (e.g., top, bottom, left side, right side). The notification can be presented audibly, such as by an automated voice reading the notification from receiver device 120 or OBD 101, and/or by non-voice audible indications (e.g., a tone or beep). The notification can additionally or alternatively include a haptic vibration or other motion of receiver device 120 or OBD 101.

One or more notifications can be presented, including any combination of two or more of the following notifications. The notification can inform the user of the result of trying to utilize system 100 with the receiver device 120 having the unacceptable clock accuracy. The notification can recommend that the user take an action such as ceasing use of the receiver device 120 having the low clock accuracy and switching to a different receiver device 120 having a relatively higher quality clock accuracy. The notification can state that receiver device 120 has an insufficient quality level, e.g., a poor quality or a low quality. The notification can state that the receiver device 120 has a clock with insufficient quality. The notification can state that the receiver device 120 has a clock with insufficient clock accuracy.

The notification can be a revision or update to a displayed target wear duration of OBD 101. For example, if the remaining target wear duration of OBD 101 is displayed or displayable on receiver device 120, then the notification can be a revision or update to this displayed remaining target wear duration, such as for example by reducing a remaining target wear duration from 10 days to 8 days or otherwise. This revision or update can be accompanied by an additional notification to the user that the remaining target wear duration has been adjusted.

If OBD 101 has implemented a change of state operation or a change of communication schedule as described earlier, then the notification can inform the user of that change. For example, the notification can inform the user that glucose and/or other analyte level measurements are being reported to receiver device 120 less frequently.

In some embodiments, OBD 101 may not have the capability to adjust its power consumption without user intervention. In such embodiments, the only PMA that may be taken is the presentation of one or more of the above notifications to the user. As these notifications may result in the user switching to a receiver device 120 having a higher clock accuracy, which would benefit the power budget of OBD 101, then presentation of any of these notifications are power management actions. In some embodiments, the power management action can be an affirmative action taken by OBD 101 and/or receiver device 120 that is not the issuance of a notification.

In some embodiments, the notification is a prompt or query to the user, a response to which indicates whether an additional PMA will be taken. For example, receiver device 120 can issue a query to the user as to whether the user will proceed with the receiver device 120 having unacceptable clock accuracy and, if yes, the relevant component of system 100 can proceed with one or more of actions 522, 524, or 526.

FIG. 5C is a block diagram depicting an example embodiment of a graphical user interface depiction 371 on a touch screen display 121 of receiver device 120, which is configured as a smart phone receiver device 122. In this embodiment, a first notification 509-1 is presented in the form of a pop-up window that informs the user that the receiver device 122 has a clock accuracy that is relatively poor quality and requests the user to switch to a new receiver device 120. The user can dismiss the notification by way of button 510. A second notification 509-2 is present at the upper right of display 121 where a quantification of the remaining target wear duration of OBD 101 is presented. The second notification 509-2 can be a modification to a visual element bearing wear duration information presented on a graphical user interface shown on display 121, such as an indicator. Examples of the modification include an added emphasis to the remaining target wear duration, such as a changed color and/or the addition of bold, and/or a change in font size, and/or a change in display location, etc., indicating that this remaining target wear duration has recently changed. Here, the target wear duration is shown for example as being reduced from 20 to 18 days (20 days→18 days) as a result of the lesser clock accuracy of receiver 120.

System 100 can perform power management actions at entities other than OBD 101. For example, it can be advantageous to offload processing tasks from OBD 101 to other entities within system 100 that do not have comparable power constraints. Each of the aforementioned PMAs 522-528 can be performed by OBD 101 itself (e.g., by processor electronics 302), or by other entities within system 100, such as: by one or more receiver devices 120, including by an app such as a glucose and/or other analyte monitoring app being executed by a receiver device 120 such as a smart phone; by computing device 140; and/or by remote server 150. Each of entities 120, 140, and 150 are in direct or indirect communication with OBD 101, as described with respect to FIG. 1D, and are also in communication with each other. The clock accuracy parameter of a given receiver device 120 can be output to these entities 120, 140, and 150 for performance of steps 502, 504, and 506 of FIG. 5A. In the case of PMA 524, where an adjustment is made to an operating aspect of OBD 101, any of entities 120, 140, and 150 can determine the adjustment to be made, and then output an instruction to make the adjustment to OBD 101 through the various communication paths, such that OBD 101 can then make the requested adjustment. In the case of PMA 528, where a notification is issued to a user, any of entities 120, 140, and 150 can determine whether a notification should be issued, and can also determine what the notification should be, and output an instruction to the notifying device, which can be OBD 101 itself in the examples where OBD 101 has a user interface, and can also be one or more of receiver devices 120 in communication with OBD 101, or computing device 140.

For example, receiver device 120 can output the clock accuracy parameter to the remote server 150 via the network 145. The remote server 150 can perform one or more PMAs 522-528, and can forward the PMA results or determinations (e.g., power consequence, energy consequence, and/or target wear duration), or instructions based on the results or determinations, to the receiver device 120, which can take action (such as issuance of a notification) and/or forward the results, determinations and/or instructions to OBD 101. This can be beneficial as remote server 150 can have access to the most accurate data and information (e.g., K and X factors) for making power, energy, and/or target wear duration determinations.

In the case of the computing device 140, the receiver device 120 can output the clock accuracy parameter to the computing device 140 directly, or indirectly by communication to network 145 and remote server 150, which then passes the clock accuracy parameter to computing device 140, which can perform one or more PMAs 522-528, and can forward the results or determinations (e.g., power consequence, energy consequence, and/or target wear duration), and/or instructions based on the results or determinations, to the receiver device 120, which can take action (such as issuance of a notification) and/or forward the results, determinations and/or instructions to OBD 101. This can be beneficial as computing device 140 can have access to the most accurate data and information (e.g., K and X factors) for making power, energy, and/or target wear duration determinations.

FIG. 5D is a flow diagram depicting an example embodiment of a method 540 of managing power for an OBD 101 in system 100 based on the assessment of clock accuracy of receiver device 120. In this embodiment, receiver device 120 is a smart phone and the clock accuracy parameter pertains to a clock 382 of the smart phone. At 532, a software application (e.g., an app) executable on receiver device 120 assesses the clock accuracy parameter of the clock 382 of receiver device 120. The app can assess the clock accuracy parameter in any of the manners described herein, such as with respect to step 504 of FIG. 5A.

Prior to assessing the clock accuracy parameter, the app can optionally first act to obtain the clock accuracy parameter of the receiver device 120. The app can issue a request from its layer of operation, which is a first layer such as an application layer, to a different protocol layer within the receiver device, which is a second layer such as a link layer relatively lower in the layer hierarchy (i.e., relatively closer to the lowest layer, which is typically the physical layer). The second layer can output the clock accuracy parameter to the first layer directly or by way of one or more intervening layers and interfaces, such as a host-controller interface.

At 534, the app can progress a power management action (PMA) based on the assessment of the clock accuracy data. The PMA can be any one or more of the PMAs 522-528 described herein. Receiver device 120 can make the determination of one or more power or energy consequences 522 as described herein. Receiver device 120 can communicate the one or more power or energy consequences to OBD 101 for further processing and/or implementation of an adjustment by OBD 101. Receiver device 120 can determine an adjustment to an operating aspect 524 of OBD 101 and instruct OBD 101 to implement the adjustment by a wireless communication. Receiver device 120 can make the determination of a target wear duration consequence 526 to OBD 101. Receiver device 120 can communicate the one or more target wear duration consequences to OBD 101 for further processing and/or implementation of an adjustment by OBD 101. Receiver device 120 can determine a notification to be issued to a user and perform the issuance of a notification to the user 528.

The receiver device 120 can progress the PMA by initiating a series of actions that, when concluded by receiver device 120 or OBD 101 as appropriate, brings the PMA to conclusion. The receiver device 120 can additionally, or alternatively, progress the PMA by taking the entire PMA series of actions itself, such as determining that a notification to the user should be issued and then issuing that notification by way of the electronics and display of the receiver device 120.

### Power Management Based on Clock Accuracy in an MDC Environment

System 100 can be configured such that OBD 101 communicates glucose and/or other analyte measurements to multiple receiver devices 120 throughout the target wear duration as part of a multi-device connectivity (MDC) environment or setting. In the MDC environment, these multiple receiver devices 120 are authorized by the user to communicate with OBD 101, such as by designating each receiver device 120 as being authorized through software being executed on the receiver devices, e.g., a glucose and/or other analyte monitoring software on a dedicated receiver device 124 or drug delivery device 126, or a glucose and/or other analyte monitoring app being executed by a smart phone or wearable receiver device or other multi-purpose mobile receiver device 122.

The authorization performed by the user can be an affirmative action taken by the user to indicate that communication is permitted, beyond an attempt to merely connect to the OBD 101, which might be performed by unauthorized users. This authorization step can include, for example, entering a code into receiver device 120 that enables communication with OBD 101. The code can be a unique identifier associated with OBD 101, for example, a code including or calculated from a serial number of sensor 160. The code can be a personal code such as a pin number or passcode. Authorization based on biometrics can additionally or alternatively be used, such as fingerprint recognition, face recognition, and/or voice recognition, where the subject or the subject's guardian or caregiver is the source of the recognized biometric.

A receiver device 120 can be authenticated in various ways in system 100, several examples of which are listed here. For example, the receiver device 120 can be authenticated by OBD 101 collecting authentication information about the receiver device 120 (or user operating the receiver device), such as a user ID and/or passcode, and comparing it to a list of authentic IDs and/or passcodes. In another example, the receiver device 120 can be authenticated by first providing authentication information, such as an identifier unique to OBD 101 (or sensor 160), to the receiver device 120 (e.g., a serial number), and having receiver device 120 transmit the identifier back to the OBD 101, which can compare the received identifier to the true identifier stored in memory. The unique identifier can be transmitted by OBD 101 to receiver device 120 (as part of the initial process of establishing a connection) or can be input by a user into receiver device 120 (such as by manual entry by the user or by the user scanning a printed code (e.g., barcode or QR code) supplied with the OBD 101). Receiver device 120 can additionally, or alternatively, transmit a modified version of the unique identifier to OBD 101, where OBD 101 checks the modification to see if valid. The modification can be input of the unique identifier to an algorithm, such as a hash algorithm, which generates a modified version of the unique identifier that the OBD 101 can validate, such as by performing a reverse hash algorithm to determine if the output is the original unique identifier, or such as by comparing the modified result to a list of one or more valid modified results. The authentication information can be the unique identifier itself, or a random number, or a combination of both, as a few examples.

By way of another example, the receiver device can be authenticated by using a private key and public key data exchange. In yet another example, the receiver device 120 can be authenticated by performing an inductive coupling-based communication with OBD 101. NFC is an example of a communication protocol based on inductive coupling. In embodiments implementing inductive-coupling based authentication, OBD 101 and receiver device 120 both have capabilities to communicate wirelessly by way of inductive-coupling and at least one other (second) wireless protocol. The inductive coupling communication can include an exchange of authentication information. The exchange can include receiver device 120 sending a request to OBD 101 for an authentication code and the OBD 101 responding with the code. OBD 101 and the receiver device 120 can subsequently conduct wireless communications using the exchanged authentication code according to a second protocol having a longer range than inductive coupling. The second protocol can be Bluetooth Low Energy or any of the other protocols described herein.

As described herein, the use of BLE can optionally not rely on standard BLE implementation of security but can instead use application layer encryption using one or more block ciphers to establish mutual authentication and encryption. The use of a non-standard encryption design implemented in the application layer has several benefits. One benefit of this approach is that the user can complete the pairing of OBD 101 and receiver device 120 with only an NFC data transfer operation and without involving the user providing additional input, such as entering a security pin or confirming BLE connection-oriented pairing between receiver device 120 and OBD 101. Another benefit is that this approach mitigates the potential to allow devices that are not in the immediate proximity of OBD 101 to inadvertently or intentionally pair, at least in part because the information used to support the pairing process is shared via the secondary short-range communication link (e.g., NFC) over a short range instead of over the longer-range BLE channel. Furthermore, as BLE pairing and bonding schemes are not involved, pairing of OBD 101 can avoid implementation issues by chip vendors or vulnerabilities in the BLE specification.

Concurrent authentications allow multiple receiver devices 120 to be eligible at the same time to receive and read glucose and/or other analyte measurement data from OBD 101, as opposed to an environment where only one receiver device 120 is authorized to receive glucose and/or other analyte data directly from OBD 101 and no second receiver device 120 can receive glucose and/or other analyte data directly from OBD 101 without first deauthorizing the first receiver device. Concurrent authentications are possible regardless of the specific mode of communication being employed, such as whether the receiver device 120 is communicating with OBD 101 in a connection-oriented mode or a connection-less mode. The communications between OBD 101 and each receiver device 120 can be simultaneous, such as by way of an advertising communication broadcast to all receiver devices 120 at the same time. The communications with OBD 101 can alternatively be at different times, such as by way of a connection-oriented communication mode, for example a paired or directed connection mode, where during a first time range, data (e.g., glucose and/or other analyte measurement data), information, and/or instructions are communicated between OBD 101 and a particular one of the receiver devices 120-1. Then during a second time range, data (e.g., glucose and/or other analyte measurement data), information, and/or instructions are communicated between OBD 101 and a different one of the receiver devices 120-2. During a third time range, data (e.g., glucose and/or other analyte measurement data), information, and/or instructions can be communicated between OBD 101 and a third receiver device 120-3, and so forth for as many receiver devices as are authorized and in range of OBD 101. The cycle can repeat intermittently such that all receiver devices 120 authorized by the user can communicate with the OBD 101. Of note, these receiver devices 120 can change from time-to-time during the wear duration of OBD 101, with the user authorizing one or more new receiver devices 120 and de-authorizing (or simply no longer connecting or using) other receiver devices 120.

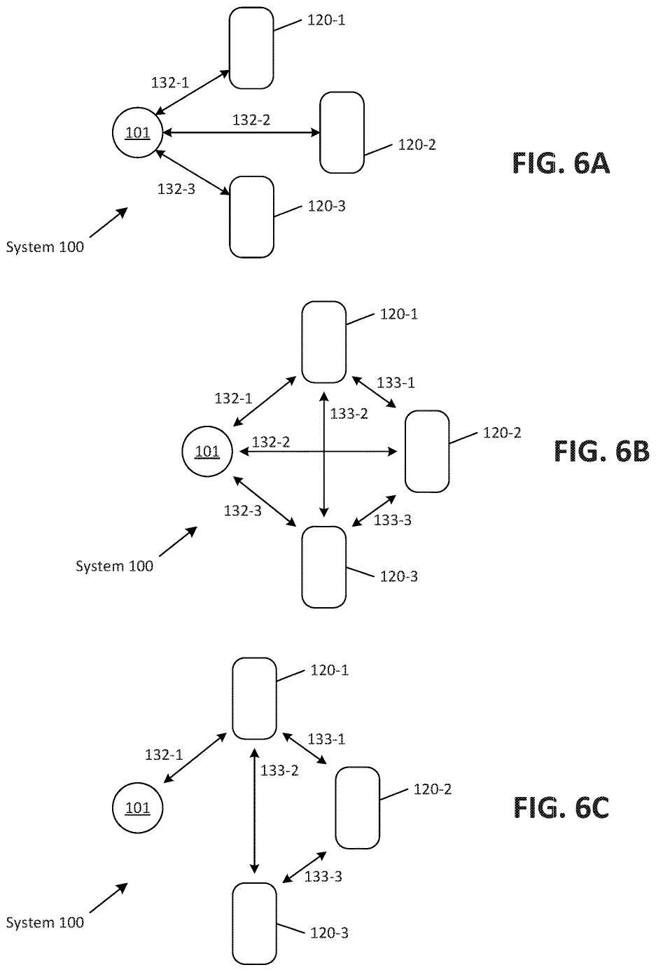

Examples are described with respect to FIGS. 6A-6C. FIG. 6A is a block diagram depicting an example embodiment of system 100 where OBD 101 wirelessly communicates with three receiver devices 120-1, 120-2, and 120-3 over wireless communication paths 132-1, 132-2, and 132-3, respectively, according to a one-to-many topology of a wireless communication protocol. Communications over paths 132-1, 132-2, and 132-3 are bidirectional in this embodiment, such that the power consumption of OBD 101 can be affected by lengthy listening windows incurred by unacceptable clock accuracies of the receiver devices 120.

FIG. 6B is a block diagram depicting an example embodiment of system 100 where OBD 101 wirelessly communicates bidirectionally with three receiver devices 120-1, 120-2, and 120-3 over wireless communication paths 132-1, 132-2, and 132-3, respectively. Further, each receiver device can communicate with each other receiver device over communication paths 133-1 through 133-3, such that the devices 101 and 120 of system 100 are in a mesh topology of a wireless communication protocol. Communications over paths 132-1, 132-2, 132-3, 133-1, 133-2, and 133-3 are preferably bidirectional, but can be unidirectional in some cases where data is transmitted from a receiver device 120 to another receiver device or other receiver devices 120 in a connection-less mode.

FIG. 6C is a block diagram depicting an example embodiment of system 100 where OBD 101 wirelessly communicates bidirectionally with a first receiver device 120-1 over wireless communication path 132-1. Receiver device 120-1 communicates with the remaining receiver devices 120-2 and 120-3 over wireless communication paths 133-1 and 133-2, and receiver devices 120-2 and 120-3 can each communicate with each other over path 133-3, such that the devices 101 and 120 of system 100 are in a partial mesh topology of a wireless communication protocol. Communications over paths 132-1, 133-1, 133-2, and 133-3 are preferably bidirectional, but can be unidirectional in some cases where data is transmitted from a receiver device to another receiver device or other receiver devices in a connection-less mode.

The wireless communication protocol of FIGS. 6A-6C can be any protocol that demonstrates one or more aspects of the subject matter described herein, including, but not limited to, one or two or more of any of those wireless communication protocols in the “Statement of Wireless Communication Protocols for the Present Embodiments” recited herein.

In a multi-device connectivity (MDC) environment, OBD 101 (or other entity of system 100) can take the PMAs 522-528 described herein. In an MDC environment, additional PMAs can be taken that relate specifically to the presence of more than one authorized and in range receiver device 120 eligible to receive glucose and/or other analyte measurement data from OBD 101. When more than one receiver device 120 is present, there is a possibility of a difference in clock accuracy between two or more of the receiver devices 120. PMAs such as adjustment to an operating aspect 524 of OBD 101 can thus be: an adjustment to a mode of communication with one or more receiver devices 120 based on assessed clock accuracies of those receiver devices 120; an adjustment to a schedule of communication with one or more receiver devices 120 based on assessed clock accuracies of those receiver devices 120; and/or an adjustment to a receiver device 120 that is the primary recipient of communications from OBD 101 based on assessed clock accuracies of those receiver devices 120, such as by designating a receiver device 120 having the highest (or a relatively higher) clock accuracy or other distinguishing aspect as a preferential central point of communication (CPOC) for the receipt of communications from OBD 101, where the remaining receiver device or devices 120 either receive glucose and/or other analyte measurement data from the CPOC receiver device 120 directly (and not from OBD 101) or receive glucose and/or other analyte measurement data at a less frequent rate from OBD 101 than the CPOC device 120. The CPOC receiver device 120 acts as a waystation through which all communications having glucose and/or other analyte measurements sent by OBD 101 and destined for receiver devices 120 can be routed or relayed.

A PMA such as an adjustment to an operating aspect 524 of OBD 101 can also or alternatively be an adjustment to a temporal length of each receive window operated by OBD 101 to listen for a communication from each respective receiver device 120. For example, a receive window for a transmission from a receiver device 120 having a relatively high clock accuracy can be set relatively shorter than a receive window for a transmission from a receiver device 120 having a relatively low clock accuracy. The receive window length can be scaled linearly in proportion to the degree of clock accuracy for each receiver device 120.

In an MDC environment, PMAs can be taken that relate specifically to the issuance of notifications to a user 528 in the presence of more than one authorized and in range receiver device 120 eligible to receive glucose and/or other analyte measurement data from OBD 101. These PMAs can be in addition to those PMAs 528 already described herein, including all types of notifications and their eligibility to be performed in whole or in part, as the case may be, for each of entities 120, 140, and 150 within system 100. One or more notifications can be presented, including any combination of two or more of the following notifications, on any one, two, or more of the receiver devices 120 concurrently in system 100.

The notification can inform the user of the result of trying to utilize system 100 with the receiver device 120-1 having the unacceptable clock accuracy. The notification can recommend that the user take an action such as ceasing use of the receiver device 120-1 having the lower clock accuracy and switching to a different one of the authorized receiver devices 120-2, etc., having a relatively higher quality clock accuracy. The notification can inform the user that one of the receiver devices 120 having a relatively high clock accuracy will be designated as a CPOC for receipt of glucose and/or other analyte measurement data, and any implications of that change. Alternatively, the notification can ask for the user's permission to treat a receiver device 120 having a relatively high clock accuracy as a CPOC. The notification can further inform the user of a change in the remaining target wear duration of OBD 101 if the user continues with use of the receiver device 120 having the low clock accuracy. The notification can inform the user that a mode of communication with the receiver device 120 having the low clock accuracy is going to be changed, optionally including a notification of how that change will affect the user, such as a lower frequency or rate of glucose and/or other analyte measurement data transmission (e.g., lengthening the interval between reports of glucose and/or other analyte measurements) to the receiver device 120 having the low clock accuracy. The notification can inform the user that switching to a receiver device 120 having a higher clock accuracy well result in an unreduced target wear duration of OBD 101, or alternatively a target wear duration that is not reduced by as much as would be the case with the lower accuracy receiver device. The notification can be an alarm or alert.

In each instance, the notification can include a prompt or query to the user, a response to which indicates whether the user acknowledges the notification and/or whether an additional PMA will be taken. For example, receiver device 120 can issue a query to the user as to whether the user will proceed with the receiver device 120 having unacceptable clock accuracy and, if yes, the relevant component of system 100 can proceed with one or more of actions 522, 524, or 526.

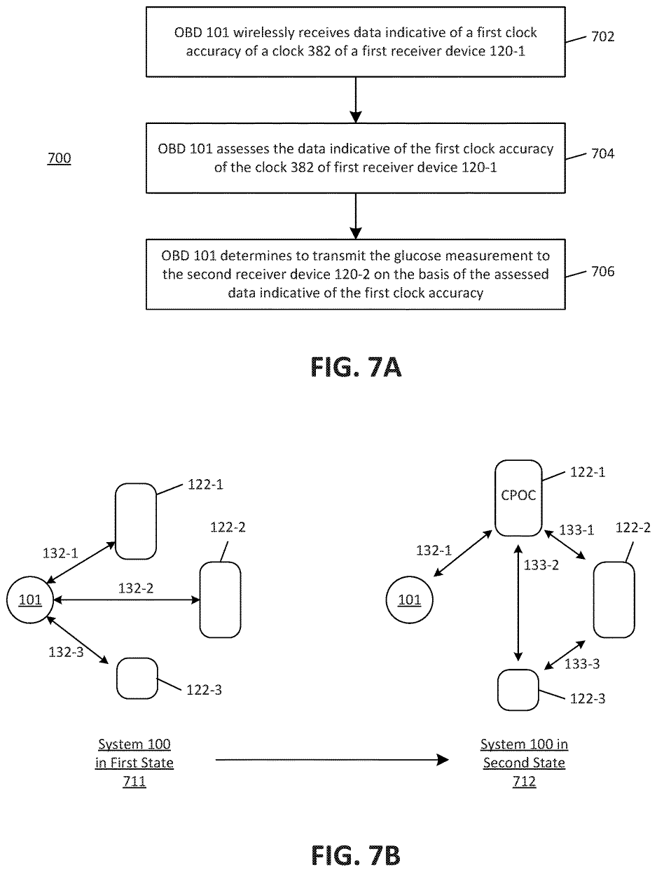

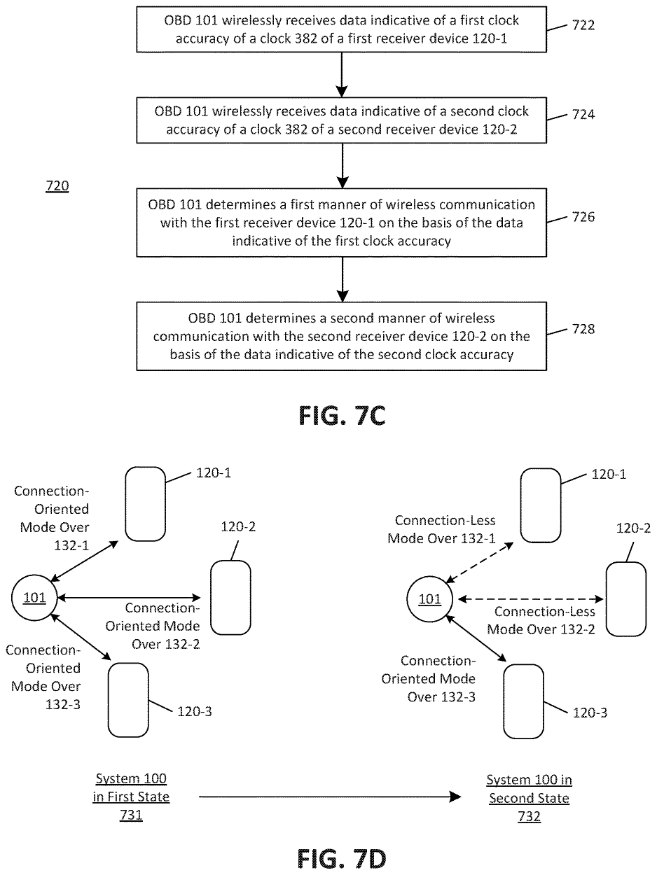

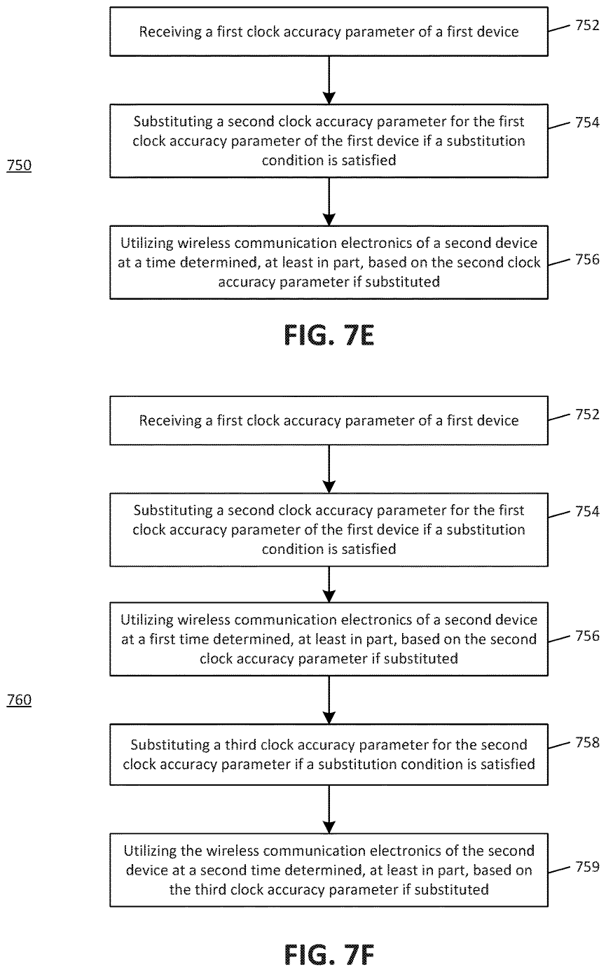

FIG. 7A is a block diagram depicting an example method 700 of power management in a system 100 having two or more receiver devices 120 authorized for communication with OBD 101 (e.g., receipt of glucose and/or other analyte measurement data from OBD 101). At 702, OBD 101 wirelessly receives data indicative of a first clock accuracy of a clock 382 of a first receiver device 120-1. This can be performed in accordance with the various embodiments described herein. The data indicative of the first clock accuracy can be a first clock accuracy parameter. The data indicative of the first clock accuracy can be an indication of a type of the first receiver device 120-1.

At 704, OBD 101 assesses the data indicative of the first clock accuracy of the clock 382 of first receiver device 120-1. This can be performed in accordance with the various embodiments described herein. For example, OBD 101 examines whether a first clock accuracy parameter satisfies a threshold clock accuracy, where the threshold can be determined as described herein. The assessment can alternatively be a comparison of the first clock accuracy parameter with a clock accuracy parameter of a second receiver device 120-2 to determine which clock accuracy parameter is relatively higher.

A direct comparison of clock accuracy parameters need not be made. For example, if a second receiver device 120-2 is of a type known to have an acceptable clock quality, such as a dedicated receiver device 124 or a drug delivery device 126, then the first clock accuracy parameter can be assessed against a threshold and, if the threshold is not met, then OBD 101 can assume the second receiver device 120-2 has a higher clock accuracy without having received a clock accuracy parameter from that second receiver device 120-2. As described herein, in some embodiments multi-purpose mobile receiver devices 122 can be designated as being of a type having an adequate or acceptable clock accuracy, based on an indication of the manufacturer of that device 122, and/or the model of that device 122, and pre-existing knowledge that the manufacturer and/or model are of a trusted acceptable quality level. Identifying information of which can be reported by device 122 to OBD 101.

At 706, OBD 101 can determine to transmit the glucose and/or other analyte measurement to the second receiver device 120-2 on the basis of the assessed data indicative of the first clock accuracy. This determination can be made because OBD 101 has assessed that receiver device 120-2 has, or is likely to have, a higher quality clock accuracy than receiver device 120-1, either by direct comparison of the clock accuracy parameters of both devices 120-1 and 120-2, or by an understanding of the type of receiver device 120-1 and/or 120-2, or both. The determination can include a determination to not transmit glucose and/or other analyte measurement data to the first receiver device 120-1 having the lower clock accuracy. Thus, system 100 can select a receiver device 120 to transmit to from a group of authorized receiver devices 120 on the basis of known or expected clock accuracy.

The selected receiver device 120 can then be designated as a CPOC by OBD 101, or by the selected receiver device 120 within system 100, and this designation can be communicated to the other devices 120 within system 100. The remaining receiver devices 120 can maintain wireless communication with the CPOC device 120 in order to receive the glucose measurement data directly from the CPOC device 120 as opposed to OBD 101.

Such a configuration is depicted in FIG. 7B, which is a block diagram depicting an example embodiment of system 100 in a first, one-to-many state 711 and subsequently reconfiguring into a second, CPOC state 712 which is a partial mesh topology. In this example, system 100 includes an OBD 101 and three receiver devices configured as multi-purpose mobile receiver devices 122-1 (e.g., a first smart phone), 122-2 (e.g., a second smart-phone), and 122-3 (e.g., a watch), although each receiver device could alternatively be a dedicated receiver 124, drug delivery device 126, or wireless-enabled computing device 128. Computing device 140 and remote server 150 can optionally be present but are not shown.

OBD 101 can initially be in the one-to-many state 711 where OBD 101 has established a connection-oriented mode of communication with each receiver device 122. OBD 101 can then receive a clock accuracy parameter from each receiver device 122 and assess the clock accuracy parameters of all three. OBD 101 can then select the receiver device 122 having the highest clock accuracy which is the most efficient for wireless communication without consuming unnecessary power or energy. Here, OBD 101 selects receiver device 122-1 as the CPOC or relay device. OBD 101 can inform all receiver devices 122-1, 122-2, and 122-3 of this selection, or can only inform receiver device 122-1 which will in turn communicate that designation to receiver devices 122-2 and 122-3 such that receiver devices 122-2 and 122-3 can expect glucose and/or other analyte measurements to be received from receiver device 122-1. Receiver device 122-1 can then relay glucose and/or other analyte measurement data from OBD 101 to receiver devices 122-2 and 122-3 over wireless communication paths 133-1 and 133-2. If a viable communication path 133 between a non-CPOC receiver device 122-2 or 122-3 and CPOC device 122-1 is lost, then any or all receiver devices having lost the communication path can notify the user of the same, and corrective action can be taken. The corrective action can include reestablishment of communication between the non-CPOC and CPOC receiver devices 120, or reestablishment of communication between the non-CPOC receiver device 120 and OBD 101.

FIG. 7C is a flow diagram depicting another example embodiment of a method 720 of power management in a system 100 having two or more receiver devices 120 authorized for communication with OBD 101 (e.g., receipt of glucose measurement data from OBD 101). At 722, OBD 101 wirelessly receives data indicative of a first clock accuracy of a clock 382 of a first receiver device 120-1. At 724, OBD 101 wirelessly receives data indicative of a second clock accuracy of a clock 382 of a second receiver device 120-2. As with other embodiments herein, the data indicative of the clock accuracy can be a clock accuracy parameter for the respective clock, and/or an indication of a type of the respective receiver device 120-1 or 120-2.

At 726, OBD 101 can determine a first manner of wireless communication with the first receiver device 120-1 on the basis of the data indicative of the first clock accuracy. At 728, OBD 101 can determine a second manner of wireless communication with the second receiver device 120-2 on the basis of the data indicative of the second clock accuracy. The first manner of communication can be the same as the second manner of communication. Alternatively, the first manner of communication can be different than the second manner of communication.

As with the embodiment of FIG. 7A, a direct comparison of clock accuracy parameters need not be made. In alternative embodiments, OBD 101 can receive an indication that the first receiver device 120-1, the second receiver device 120-2, or both receiver devices 120-1 and 120-2 is/are of a type that is known to have acceptable clock accuracy, such as being a dedicated receiver device 124 or being a drug delivery device 126. The type can also be indicative of a multi-purpose mobile receiver device 122 or wireless-enabled computing device 128 that is provided by an entity (e.g., a manufacturer) or that is a particular model, that is known to OBD 101 as having a clock accuracy that is acceptable, as described herein. The determinations of 726 and 728 can be on the basis of the received type information instead of received clock accuracy parameters.

The determination of the manner of wireless communication can be based on the clock accuracy in a manner that results in use of a manner of wireless communication that potentially consumes more power (or energy) listening for transmissions from receiver devices 120 that have a relatively higher clock accuracy or are of a type indicative of an acceptable clock accuracy. In this manner, the risk of potential power loss from operating the transceiver of OBD 101 without receipt of transmission from a receiver device 120 (e.g., extended listening windows), is lessened or mitigated by virtue of the receiver device 120 being verified to have a relatively high or acceptable clock accuracy that transmits on time. Conversely, if a receiver device 120 has an unacceptable clock accuracy or a relatively lower clock accuracy, then OBD 101 can determine to use a manner of wireless communication that will likely consume less power (or energy) listening for transmissions from that receiver device. The risk of potential power loss from operating the transceiver of OBD 101 without receipt of transmission from a receiver device 120 (e.g., extended listening windows) is lessened or mitigated by virtue of the selected manner of communication having fewer instances where OBD 101 is required to operate its transceiver to listen for potentially delayed communications from the receiver device 120.

The manner of communication that is selected can include a mode of communication. The selected mode can be a connection-oriented mode of communication such as a paired or a direct-connection communication session, or a connection-less mode of communication, such as communication by way of advertising transmissions and responses. Determination of which mode of communication is more power efficient is dependent upon the design and implementation of OBD 101. In many embodiments, a connection-oriented mode with repeat listening windows synchronized to a common clock shared by OBD 101 and the receiver device 120 will be generally more prone to consume power than a connection-less mode with listening windows that are not as frequent or that are timed from another event such as transmission by the OBD 101 to the receiver device 120.

As described herein, in some embodiments, the determination of which mode has a power consumption more suitable for use with low accuracy or unacceptable clock can be dependent upon a connection interval, e.g., the rate of transmissions that are sent and received, in that mode. For example, the sending of glucose and/or other analyte level measurements by OBD 101 at a rate of every three minutes or less can result in a connection-oriented mode of communication being more power efficient than a connection-less mode, while the sending of glucose and/or other analyte level measurements by OBD 101 at a rate of more than every 3 minutes can result in a connection-less mode of communication being more power efficient than a connection-oriented mode. Thus, a determination in accordance with steps 726 and 728 as to what mode to select can be based in part on the rate of transmission of glucose and/or other analyte level measurements in each mode.

In some embodiments, the determination of a manner of communication can include a determination of the rate of transmissions between OBD 101 and receiver device 120 to be utilized when communicating.

FIG. 7D is a block diagram depicting an example embodiment of system 100 which implements method 720. Here, system 100 is in a first, one-to-many state 731 where each receiver device 120-1, 120-2, and 120-3 operates in a connection-oriented wireless communication mode over communication paths 132-1, 132-2, and 132-3 respectively. In this mode, each receiver device 120 transmits data indicative of the clock accuracy of the receiver device type to OBD 101, as described herein. OBD 101 can assess the data indicative of the clock accuracy of the receiver devices 120-1, 120-2, and 120-3. In this example, OBD 101 determines that receiver devices 120-1 and 120-2 have clock accuracies that are unacceptable (e.g., below a threshold) and receiver device 120-3 has a clock accuracy that is acceptable. OBD 101 determines to reconfigure the connection modes, and transitions to a new one-to-many state 732 where communications with receiver devices 120-1 and 120-2 transition to connection-less wireless communication mode. In an alternative embodiment, OBD 101 can transition such that communications with receiver devices 120-1 and 120-2 remain in a connection-oriented mode but have a renegotiated lower transmission interval or rate to save power. Receiver device 120-3 can remain in the connection-oriented mode with the same transmission interval or rate in state 732.

Manufacturers or other providers of receiver devices 120 set the clock accuracy parameter within the receiver device 120 such that it is retrievable and can be communicated to OBD 101 (or to a higher layer app being executed on the receiver device 120, or other device or trusted computer system 150 in system 100). The set clock accuracy parameter can be stored in memory 356 of receiver device 120. In some cases, a manufacturer or other provider sets the clock accuracy parameter to a value that accurately represents the actual clock accuracy of the relevant clock 382 of that specific receiver device, e.g., where that value was determined through careful testing. In other cases, the manufacturer or other provider sets the clock accuracy parameter to a value that less accurately represents the actual clock accuracy of the relevant clock 382, e.g., a value that may be a generic minimum clock accuracy for receiver devices of a particular model-type or class as a whole. These generic values can be undesirable if they do not accurately represent the actual clock accuracy of the receiver device's relevant clock 382 as OBD 101 uses that clock accuracy parameter to determine the time at which the OBD's wireless communication electronics 312 should be activated to receive an incoming transmission from receiver device 120. If the clock accuracy parameter reported by receiver device 120 represents the clock accuracy as being worse than it actually is (e.g., 500 ppm when the accuracy is actually 250 ppm), then this can result in OBD 101 activating wireless communication electronics 312 too early and consuming excess power, in turn shortening the power source life. If the clock accuracy parameter reported by receiver device 120 represents the clock accuracy as being better than it actually is (e.g., 250 ppm when the accuracy is actually 500 ppm), then this can result in OBD 101 activating wireless communication electronics 312 too late and potentially missing incoming transmissions from receiver device 120, resulting in a dropped connection or other data integrity issue, which in turn can require more power for reconnection attempts or to regain lost data again potentially shortening the power source life.

FIG. 7E is a flow diagram depicting an example embodiment of a method 750 of using an analyte monitoring system 100. At 752 a first clock accuracy parameter of a first device (e.g., a receiver device 120) is received (e.g., by a second device OBD 101 or by an app operating on the first device). The information received can be a clock accuracy parameter and/or any data indicative of a clock accuracy of a clock of the first device. Although not limited to such, for ease of description the first device is described in this embodiment as receiver device 120 and the first clock accuracy parameter is described as being received by a second device that, by example, is OBD 101.

At 754 a second clock accuracy parameter is substituted for the first clock accuracy parameter of the first device if a substitution condition is satisfied. While not limited to any particular purpose, step 754 can be performed because the second clock accuracy parameter is considered to more accurately reflect the actual clock accuracy of the relevant clock (e.g., clock 382) of the first device than the first clock accuracy parameter. The second device can be configured (e.g., via software instructions executed on processing electronics 302) to detect whether the first clock accuracy parameter satisfies the substitution condition, satisfaction of which indicates that a substitution operation for the first clock accuracy parameter is to be performed.

The substitution condition can take many forms in various embodiments. The substitution condition can be a software check that compares the received first clock accuracy parameter to a substitution condition in the form of a threshold (e.g., a quantitative threshold), and if the first clock accuracy parameter satisfies the threshold then it will not be used and a substitution will be made. For example, the first clock accuracy parameter may be, e.g., 500 ppm, and the threshold is greater than 300 ppm, and thus the first clock accuracy parameter satisfies the threshold and will be substituted. While the substitution condition is described as a positive condition satisfaction of which leads to substitution (e.g., a threshold of greater than 300 ppm), the substitution condition can likewise be a negative condition such as, e.g., a threshold of less than or equal to 300 ppm, in which case failure to satisfy the substitution condition leads to substitution. The substitution condition can alternatively, or also, include a software comparison to one or more discrete values. For example, if the received first clock accuracy parameter is present in a data structure (e.g., an array, list, and/or table) including one or more discrete values of clock accuracy parameters that should not be used, and if the received first clock accuracy parameter is present in the data structure, then it is not to be used, similar to a blacklist. Alternatively, if the first clock accuracy parameter is in the data structure then that signifies that it should be used, similar to a white list. The substitution condition can include one or more thresholds, one or more software comparisons to discrete values, or a combination of both. Other forms of substitution conditions can also be used.

If OBD 101 determines to substitute a second clock accuracy parameter for the first clock accuracy parameter, then OBD 101 can be configured to identify and/or determine the appropriate second clock accuracy parameter to be used. The second clock accuracy parameter can be identified from software instructions, a data structure, and/or an algorithm. In some embodiments, if the substitution condition involves a threshold of a value X (e.g., 200 ppm, 250 ppm, 300 ppm, 400 ppm, 500 ppm), and the first clock accuracy parameter is less accurate than that value X (e.g., has a ppm value that exceeds X), then OBD 101 can be configured to perform the substitution with a second clock accuracy parameter of the value X, such that all clock accuracies falling below the threshold are then changed to equal that threshold. For example, TABLE 2 below is an example of a human readable table that can be implemented in software instructions and/or as a data structure stored in memory 308 of OBD 101 having various substitution condition thresholds and corresponding second clock accuracy parameters (CAP2) to be used if a received first clock accuracy parameter (CAP1) falls within a given threshold.

TABLE 2

| | Second Clock Accuracy |
|---|---|
| Substitution Condition (ppm) | Parameter (CAP2)(ppm) |
| 250 < CAP1 ≤ 300 | 250 |
| 300 < CAP1 ≤ 350 | 300 |
| 350 < CAP1 ≤ 400 | 350 |
| 400 < CAP1 ≤ 450 | 400 |
| 450 < CAP1 ≤ 500 | 450 |
| 500 < CAP1 | 500 |

Alternatively, the second clock accuracy parameter can be a value different than X, that is either greater than or lower than X, depending on the desired adjustment to be made. In other embodiments, if the substitution condition involves comparison of the received first clock accuracy parameter to one or more discrete values, e.g., that may be stored in a data structure or directly in software instructions, then the one or more discrete values can each be associated with a second clock accuracy parameter to use for substitution or as a replacement. For example, TABLE 3 below is an example of a human readable table that can be implemented as a data structure stored as data or software instructions in memory 308 of OBD 101 having discrete values of first clock accuracy parameters with corresponding second clock accuracy parameters.

TABLE 3

| First Clock Accuracy | Second Clock Accuracy |
|---|---|
| Parameter (ppm) | Parameter (ppm) |
| 250 | 200 |
| 300 | 200 |
| 350 | 250 |
| 400 | 250 |
| 450 | 300 |
| 500 | 300 |

In still other embodiments, the second clock accuracy parameter can be determined algorithmically, such as by improving the first clock accuracy parameter by a percentage (e.g., 5% or more, 10% or more, 20% or more, and so forth), by improving it by a set amount (e.g., 50 ppm or more, 100 ppm or more, 150 ppm or more, and so forth), a combination of the two, or otherwise. The second device can, if desired, overwrite the first clock accuracy parameter in memory with the second clock accuracy parameter, which can then permit the second device's software stack to proceed normally according to similar software instructions without having to take further action to account for the substitution. All of these actions associated with step 756 can be accomplished in software (e.g., by an application program interface (API) that can be called by the software stack to execute these actions), hardware, and/or a combination of both software and hardware.

In embodiments, method 750 of FIG. 7E can end after step 754 (or continue to other actions different than step 756). In other embodiments, method 750 can continue to step 756, where wireless communication electronics 312 of the second device (e.g., OBD 101) are utilized at a time determined, at least in part, based on the second clock accuracy parameter. Use of the more accurate second clock accuracy parameter results in utilization (of the wireless communication electronics 312 at the time that also more accurately reflects the proper time for scheduled or synchronous operations. Utilization of the wireless communication electronics 312 can, in some embodiments, be activation of some or all of the wireless communication electronics 312 (e.g., a transmitter portion, a receiver portion, a processing portion, and/or a memory portion) to place electronics 312 in a state for transmitting and/or receiving; can, in embodiments, be changing of the power consumption state of some or all of the wireless communication electronics 312, e.g., wake-up from one power state to a relatively higher power state or entering sleep from a relatively higher power state to a relatively lower power state; and/or can, in embodiments, be changing of the power consumption state of some or all of the wireless communication electronics 312, e.g., wake-up from one power state to a relatively higher power state or entering sleep from a relatively higher power state to a relatively lower power state; and/or can, in embodiments, be initiation and/or operation of a software routine by processing circuitry of the wireless communication electronics 312. Use of the more accurate second clock accuracy parameter may result, for example, in activation of the wireless communication electronics 312 at the time that also more accurately reflects the earliest possible time an incoming transmission may be received from receiver device 120, which in most cases is later in time as compared to when activation would occur if the first clock accuracy parameter had been used. Algorithms that use clock accuracy to determine a utilization time are known to those of ordinary skill in the art and need not be repeated here. These algorithms can account for both the clock accuracy of the first device (e.g., receiver device 120) (as reflected in the first and/or second clock accuracy parameter) as well as the clock accuracy of the second device (e.g., OBD 101) which also has a bearing on determining the appropriate wake up time. The algorithm can determine a temporal offset from a scheduled time of next receipt of the incoming transmission and deduct this offset from that scheduled time to determine the actual wake up time for the second device.

System 100 can be further configured to dynamically adjust or tune the clock accuracy parameter to improve the accuracy of the clock accuracy parameter. FIG. 7F is a flow diagram depicting an example embodiment of a method 760 of using an analyte monitoring system 100. Method 760 includes steps 752, 754, and 756 (and all variants thereto) described with respect to method 750 and which are not described again herein. After step 756, method 760 proceeds to step 758, where a third clock accuracy parameter is substituted for the second clock accuracy parameter if a substitution condition is satisfied, which can be the same as or different from the substitution condition of 754. Method 760 then proceeds to step 759 where the wireless communication electronics of the second device are utilized at a second time determined, at least in part, based on the third clock accuracy parameter.

The step 758 of substituting the third clock accuracy parameter for the second clock accuracy parameter if a substitution condition is satisfied can be prompted by the detection, by the second device, of a failure to receive an incoming transmission from the first device (or other timing related failure) after step 756. For example, the second clock accuracy parameter may have overestimated the actual clock accuracy of the first device, resulting in a failure of the second device to activate the wireless communication electronics in time to receive the incoming transmission. The third clock accuracy parameter can be, e.g., a value, between that of the first and second clock accuracy parameters so as not to revert fully to the original first clock accuracy parameter. The process of detecting a failure to receive an incoming transmission and then substituting the current clock accuracy parameter with an adjusted clock accuracy parameter can be continued iteratively, (e.g., substituting fourth, fifth, sixth, and additional clock accuracy parameters) until no failure to receive a transmission occurs for a predetermined time period (e.g., an hour, six hours, 12 hours, one day, and so forth), or for a predetermined number of successful wireless communication electronics utilization instances (e.g., one, two, three, five, ten, and so forth), selected as desired to be representative of having arrived at a substantially accurate clock accuracy parameter.

In this embodiment, the third (and/or subsequent) clock accuracy parameter can be identified or determined similarly to the manners described with respect to the second clock accuracy parameter, including by reference to a data structure or predetermined parameter values in software instructions, and/or by use of an algorithm for selection of the clock accuracy parameter.

The dynamic adjustment can revise the current clock accuracy parameter (e.g., first, second, third, fourth, etc.) downward (increase) to a less accurate value (e.g., from 250 ppm to 300 ppm) when a failure to receive a transmission from the first device, or other timing error, occurs or is detected. The dynamic adjustment can also revise the current clock accuracy parameter upward (decrease) to a more accurate value (e.g., from 350 ppm to 300 ppm) when failures are not occurring and there is a probability that power savings can occur by revising the clock accuracy parameter further as it may be underestimating the quality and accuracy of the clock.

The clock accuracy parameter that is being utilized at any one time by the second device (e.g., OBD 101), whether it is a first, second, third, fourth, or other clock accuracy parameter, can be used with the other embodiments described herein, such as those involving processes pertaining to assessing clock accuracy parameters (e.g., as described with respect to step 504 of method 500) and progressing one or more PMAs based on the assessment (e.g., as described with respect to step 506 of method 500 and the steps 522-528 described with respect to step 506 of FIG. 5B and elsewhere herein) so that those other processes are not interrupted. Alternatively, those other processes can be temporarily paused until the clock accuracy parameter stabilizes at one value, or a narrow range of values.

A host of various wireless communication modes can be used with all of the MDC embodiments described herein. The modes can be modes of operation in accordance with wireless communication protocols described herein which include, but are not limited to, one or two or more of any of those wireless communication protocols in the “Statement of Wireless Communication Protocols for the Present Embodiments” recited herein.

In all of the embodiments herein, a connection-oriented communication mode can involve a negotiation procedure where OBD 101 and receiver device 120 each transmit to and receive from the other device to negotiate parameters at the outset of a formal connected session that govern how that session between the devices will be performed. The connection-oriented mode can be a paired mode between two devices, i.e., a one-to-one pairing. The connection-oriented mode can be a bonded mode. The negotiation can be an initial step in the session. An example parameter can be the communication interval between times where one of devices 101 or 120 sends or asks for a communication containing, e.g., glucose and/or other analyte measurement data. Another example parameter can be a latency parameter that allows OBD 101 to refrain from sending data at the expiration of a communication interval, where the latency parameter can specify a maximum number of times to successively refrain from sending data, or a maximum duration of time during which OBD 101 can refrain from sending data. Another example parameter can be a timeout parameter that sets a maximum number amount of time that can pass without receiving a communication before one of the devices can terminate the existence of a formal connection session under the connection-oriented mode of communication. The negotiation procedure can involve the sending of an advertisement for a connection from a first device followed by sending a response from a second device back to the first device. The advertisement can include the parameters desired for establishment of the formal connection-oriented session, and the response can include an affirmation or acceptance of those parameters, which can then lead to establishment of the formal connection-oriented session, or a rejection of those parameters which can deny the establishment of the formal connection-oriented session. During the established connection-oriented session, data that is transmitted from a first device, after being received by a second device, can be acknowledged by sending an acknowledgement from the second device back to the first device.

In all of the embodiments herein, a connection-less communication mode can involve communications that take place between devices 101 and 120 without first negotiating parameters that define a formal connected session. A single communication from a first device, such as OBD 101, can be received by one or more other devices, such as receiver devices 120. An example of a connection-less mode of communication is a broadcast mode or a multicast mode. An example of a connection-less communication mode can be an advertising mode, where a first device sends an advertisement that can be received by one or more other devices. In some embodiments, the advertising device can send the advertisement with one or more parameters informing the receiving devices how the connection-less communications will be conducted by the advertising device in unilateral fashion, such that the receiving devices cannot accept or decline.

In systems 100 utilizing a wholly implantable sensor 162, the sensor 162 can have power source capacity and transmission range that is great enough to communicate directly with one or more receiver devices 120 without first communicating to an OBD 101. In such embodiments, the sensor 162 has receiver device communication and data processing functionality of the OBD 101 integrated therein. Thus, references to OBD 101 in all of the embodiments described herein likewise refers to the alternative embodiment where sensor 162 incorporates the wireless communication capability of OBD 101 to communicate with the receiver device(s) 120. As such, all clock accuracy embodiments described herein apply to the embodiments where the wholly implanted sensor 162 acts as a sensor integrated with the requisite limited OBD 101 functionality to communicate directly with the receiver device 120. In those embodiments, the system 100 can include just the sensor 162 by itself, alternatively the sensor 162 in combination with a receiver device 120, or if the sensor 162 can transmit to both OBD 101 and directly to the receiver device 120 (such as in the case of OBD 101 failure or for high priority transmissions containing alarms), the system 100 can include any of the following: just the sensor 162; the sensor 162 and the OBD 101; the sensor 162 and one or more receiver devices 120; or the sensor 162, the OBD 101, and one or more receiver devices 120.

Wireless Communication with Synchronous Advertising

Asynchronous wireless communication protocols provide flexibility for transmitting on an as needed basis, e.g., when data is ready for transmission, but can suffer from increased power usage due to the uncertainty of when communications will arrive, requiring wireless receivers to be active to monitor for incoming transmissions and thus consuming more power. Even some wireless communication protocols that appear at first glance to be synchronous can introduce asynchronous or random variability (or perturbation) into transmission timing, e.g., variable delays, to mitigate the likelihood of collisions between device transmissions. This again requires wireless receivers to be active for longer periods of time. Asynchronous protocols suffer for lack of regimented response capability to accommodate on-body analyte monitoring systems in multiple receiver device 102 environments. The use of a connection-oriented, direct device-to-device (or one-to-one (1:1)) pairing can permit a power efficient connection to be established for some data transmission rates but not others, and furthermore become less power efficient as multiple receiver devices 102 are added as each independent 1:1 pairing requires a power-consuming negotiation process to establish the pairing, and further requires multiple transmissions by the OBD 101 of the same data to communicate to each receiver device 102 independently. Some mesh networking protocols require intensive scanning for transmissions, in some cases requiring a duty cycle of close to 100%, which is not practical for energy constrained devices like OBD 101. For these and other reasons needs exist for synchronous wireless communication protocols in on-body analyte monitoring systems 100.

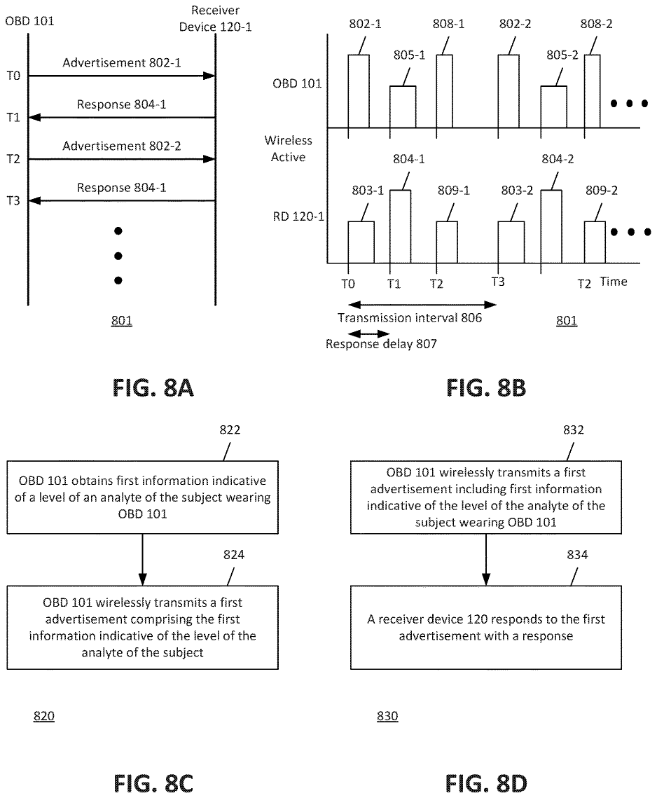

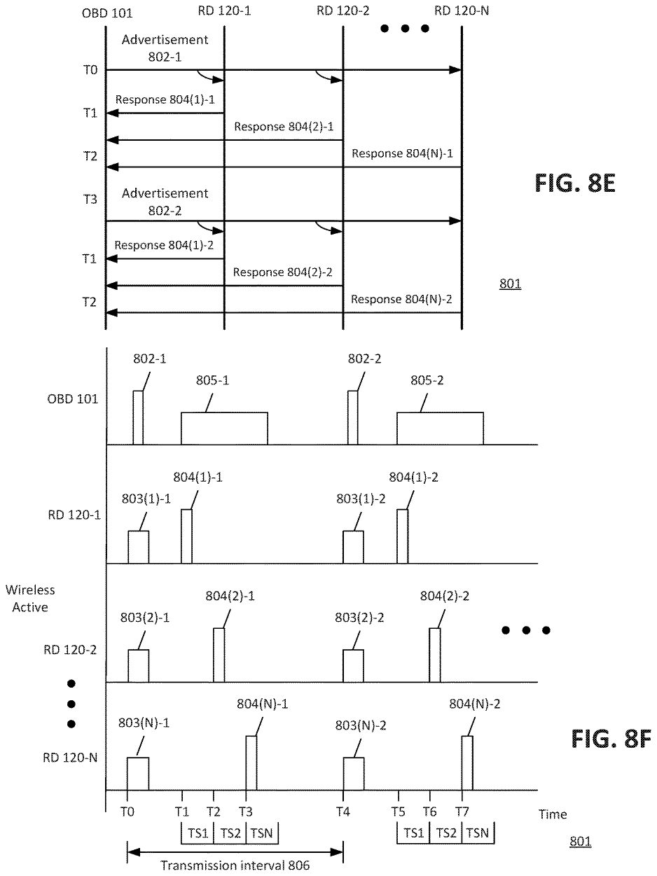

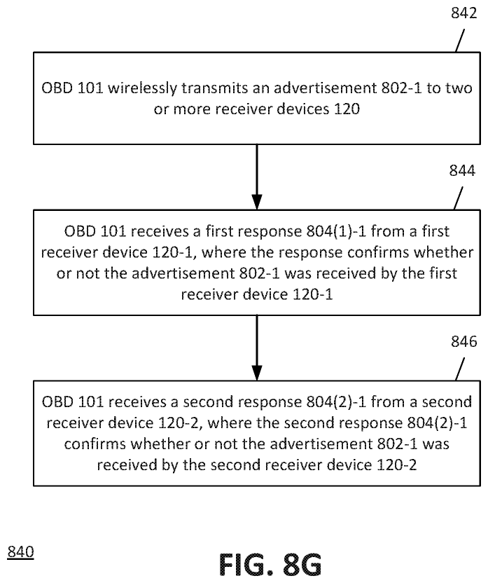

System 100 can be configured to communicate between devices using a synchronous advertising-based wireless communication protocol. Numerous example embodiments of such are described herein with reference to FIGS. 8A through 16B. In this synchronous advertising-based wireless communication protocol, information (e.g., data) is sent at scheduled times where the transmitting and receiving devices (101 and 120) can determine those times with accuracy because their respective timing references (e.g., clocks) are aligned. The devices 101 and 120 can be considered as operating with a shared timing reference.

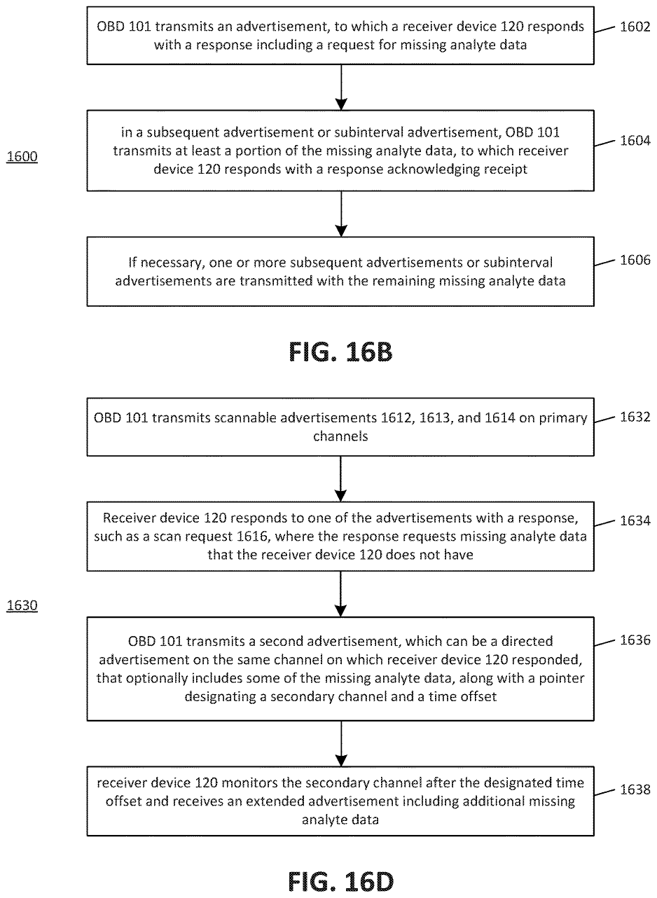

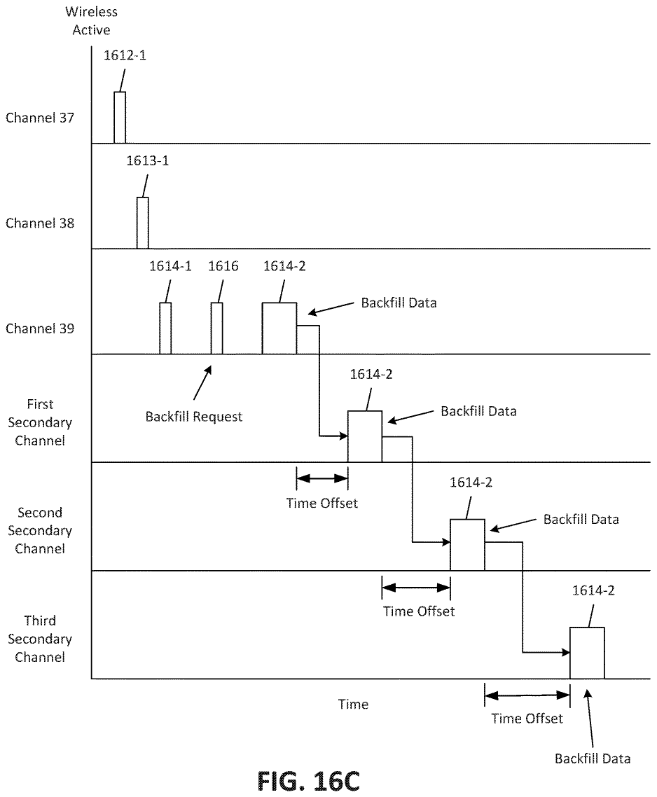

Each of the embodiments of system 100 described with respect to FIGS. 8A through 16B, as well as FIGS. 16C and 16D, can be configured to also apply the embodiments pertaining to clock accuracy described herein, including all of those embodiments of system 100 described with respect to FIGS. 5A, 5B, 5C, 5D, 6A, 6B, 6C, 7A, 7B, 7C, and 7D. These embodiments can operate with defined times for transmission of advertisements and responses with no random variability or other delay inserted to adjust the transmission times for the purpose of collision avoidance.

FIG. 8A is a sequence diagram depicting an example embodiment of a sequence of transmissions in a system 100 having an OBD 101 and at least one receiver device 120 communicating with a synchronous advertising-based wireless protocol in a synchronous communication session 801. FIG. 8B is a timing diagram depicting this embodiment of session 801 with the times at which the wireless communication electronics of OBD 101 and receiver device 120 can be active (e.g., powered on with the desired power for transmission and/or reception of a wireless signal). The description proceeds here with reference to both FIGS. 8A and 8B.

Here, OBD 101 has activated its wireless communication electronics and transmits a first advertisement 802-1 to receiver device (RD) 120 at time T0. Starting at time T0, receiver device 120 has also activated its wireless communication electronics and is monitoring for advertisement 802-1 and receives it during a receive window 803-1. In this and all embodiments herein, both OBD 101 and receiver device 120 track time using their respective clock 306 and timing circuitry 380 (not shown) and thus the clock accuracy of both devices can be significant as described in further detail herein. Advertisement 802-1 includes first information indicative of an analyte level of a wearer of OBD 101, e.g., first analyte data. The following embodiments will refer to this first information as first analyte data for ease of discussion. Receiver device 120 can validate the received advertisement by, for example, verifying that advertisement 802-1 was transmitted by an authenticated OBD 101 and/or by error checking received data (e.g., such as by a cyclic redundancy check) in advertisement 802-1. Receiver device 120 can decrypt the received data in advertisement 802-1 if it was encrypted by OBD 101.

In all embodiments validation can be performed for all transmissions sent by OBD 101 to receiver device 120 and sent by receiver device 120 to OBD 101, but validation is not required. In all of the embodiments, in addition to or as an alternative to authentication and/or error checking, validation can also, or alternatively, be accomplished by: performing a check that OBD 101 from which advertisement 802-1 was received is the expected OBD 101 from which the advertisement 802-1 was expected; performing a check that OBD 101 from which advertisement 802-1 was received is an OBD 101 known to receiver device 120; and/or performing a check that OBD 101 from which advertisement 802-1 was received is an OBD 101 authorized to communicate with receiver device 120.

At time T1, receiver device 120 responds with a first response 804-1. Also at T1, OBD 101 has also activated its wireless communication electronics (either by leaving it activated or deactivating it after advertisement 802-1 is transmitted and reactivating it at time T1) and is monitoring for response 804-1 and receives it during a receive window 805-1. Response 804-1 includes an acknowledgement or indication of whether or not advertisement 802-1 was received. OBD 101 can validate the received response 804-1 by, for example, verifying that response 804-1 was transmitted by an authenticated receiver device 120 and/or by error checking received data (e.g., such as by a cyclic redundancy check) in response 804-1. OBD 101 can decrypt the received data in response 804-1 if it was encrypted by receiver device 120. OBD 101 can then interpret the response 804-1 to determine if advertisement 802-1 was received (e.g., validly received) by receiver device 120. OBD 101 can interpret the response 804-1 by reading a header and/or payload of the response. In this embodiment, response 804-1 is a valid acknowledgement response indicating receipt, and OBD 101 need take no further action and deactivates its wireless communication circuitry until time T2. Optionally, OBD 101 can transmit a confirmation 808-1 to receiver device 120-1 indicating that response 804-1 was received as a valid acknowledgement response (e.g., a bit or bits indicating acknowledgement of receipt (ACK)), which can be received during receive window 809-1. In other embodiments, if response 804-1 indicates advertisement 802-1 was not received (e.g., a first bit or bits indicating a non-acknowledgement for non-receipt (NACK1)), or was received but invalid (e.g., a second bit or bits indicating a non-acknowledgement for invalidity (NACK2)), then OBD 101 can take further action such as indicating receipt of response 804-1 in confirmation 808-1 and then retransmission of the analyte data in that or a subsequent advertisement as described in more detail herein. For ease of illustration, many embodiments will be described herein and shown in the accompanying figures without the presence of a return confirmation transmissions 808 and receive windows 809 after the sending of a response 804 or group of adjacent responses 804; however, although not shown all embodiments can incorporate that functionality at the option of the implementation.

OBD 101 waits until time T2 and reactivates its wireless communication electronics and can repeat the process of transmitting a second advertisement 802-2 to receiver device 120, which monitors for it and receives it during receive window 803-2. In this embodiment, while OBD 101 was deactivated before time T2, OBD 101 has obtained second information indicating the analyte level of the OBD wearer. This can be the result of a second, more recent measurement made by sensor 160. Second advertisement 802-2 contains this second information. Receiver device 120 receives and reads second advertisement 802-2 in similar fashion to receipt of first advertisement 802-1 during receive window 803-2, and responds at time T3 with second response 804-2, which OBD 101 receives during receive window 805-2 and reads similar to first response 804-1. This process can repeat regularly for as long as the OBD 101 is active and communicating updated analyte data to receiver device 120 (e.g., for the entire wear duration of the OBD 101).

Here, both OBD 101 and receiver device 120 know the amount of time (e.g., time duration or delay) between time T0 and T2, which can be referred to as the OBD transmission interval 806, or primary transmission interval. Both OBD 101 and receiver device 120 know the amount of time between time T0 and T1, which can be referred to as the response delay 807. In this manner, both OBD 101 and receiver device 120 know with substantial precision when to activate their respective wireless communication electronics for receipt of incoming transmissions. Thus, OBD 101 can regularly transmit updated analyte level information to receiver device 120 and OBD 101 can receive immediate indications of receipt from receiver device 120 in synchronous fashion without requiring lengthy periods where wireless communication electronics is activated and consuming power but not making or receiving transmissions.

In the response 804, receiver device 120 can either acknowledge receipt of advertisement 802 or can indicate advertisement 802 was not received (or was received but not validated (such as by failing an authentication and/or failing to pass an error check, possibly due to transmission error)) by responding with a non-acknowledgement response. If in this embodiment response 804-1 instead indicates that the preceding advertisement 802-1 was not received by receiver device 120, or was received but was not validated, then after receiving and reading response 804-1, OBD 101 can determine to include the first analyte data again in the next advertisement 802-2, optionally along with the second analyte data, and transmit advertisement 802-2 with a longer payload having both the first and second analyte data. Receiver device 120 will thus have a second opportunity to receive the missed first analyte data. This respond and resend process can be repeated as desired whenever receiver device 120 indicates failure to receive a valid advertisement. The resend process can likewise occur if OBD 101 fails to receive response 804-1 altogether, or receives response 804-1 but is not able to validate it.

In the embodiment of FIGS. 8A and 8B and other embodiments herein, in the intervals between transmissions and receive windows, the wireless communication electronics of OBD 101 can transition from the activated state, consuming the requisite power for transmission and/or reception, to a lower power state that consumes less power. In this manner OBD 101 can reduce and, in some embodiments, minimize power consumption (and thus extend power source life). In the embodiments herein, the length of OBD 101 transmissions is preferably minimized to likewise minimize the length of the activated state. Similarly, the time at which incoming transmissions from receiver devices 120 is preferably known with substantial accuracy by OBD 101 (e.g., within margin of error accounting for expected jitter and other timing variance) to allow activation of the circuitry to be synchronized with the anticipated times of receipt and thus to minimize the amount of time the OBD wireless communication electronics is activated.

FIG. 8C is a flow diagram depicting an example embodiment of a method 820 of wireless communication in a synchronous advertising-based communication session 801 with system 100. At 822, OBD 101 obtains first information indicative of a level of an analyte of the subject wearing OBD 101. At 824, OBD 101 wirelessly transmits a first advertisement including the first information indicative of the level of the analyte of the subject.

FIG. 8D is a flow diagram depicting an example embodiment of a method 830 of wireless communication in a synchronous advertising-based communication session 801 with system 100. At 832, OBD 101 wirelessly transmits a first advertisement including first information indicative of the level of the analyte of the subject wearing OBD 101. At 834, a receiver device 120 responds to the first advertisement with a response.

FIG. 8E is another sequence diagram depicting an example embodiment of synchronous communication session 801 performed by a system 100 having an OBD 101 and two or more receiver devices 120-1 through 120-N communicating with a synchronous advertising-based wireless protocol. FIG. 8F is a timing diagram depicting this embodiment of session 801 with the times at which the wireless communication electronics of OBD 101 and receiver devices 120-1 through 120-N are active (e.g., powered on with the desired power for transmission and/or reception of a wireless signal). The description proceeds here with reference to both FIGS. 8E and 8F. Because OBD 101 and receiver devices 120 operate here in similar fashion to the embodiment described with respect to FIGS. 8A and 8B, the same and similar features will not be discussed, with emphasis instead being placed on differences in operation between the embodiments.

In this example there are N receiver devices 120, where N is an integer of two or greater, typically between 2 and 10 inclusive, though system 100 can operate with more than 10 receiver devices 120. Operation of three receiver devices 120-1, 120-2, and 120-N will be described, with the understanding that the timing and operation described in common between the receiver devices 120 can be duplicated and extended to the desired number N of receiver devices 120 included in a particular implementation. Thus the operation of all receiver devices 120 up to and including the Nth receiver device 120 need not be described. In all embodiments of synchronous communication sessions 801 herein, the number of receiver devices 120 may vary from one to three or more receiver devices 120, but in all embodiments sessions 801 can be conducted with only one receiver device 120, two receiver devices 120, three receiver devices 120, or more unless expressly noted otherwise.

Here, OBD 101 has activated its wireless communication electronics and transmits a first advertisement 802-1 to receiver devices 120-1, 120-2, and 120-N at time T0. Starting at time T0, receiver devices 120-1, 120-2, and 120-N have also activated their wireless communication electronics and are monitoring for advertisement 802-1 and receive it during the respective receive windows 803(1)-1, 803(2)-1, and 803(N)-1 (where the numeral in parentheses, e.g., (1), (2), or (N), designates the iteration of receiver device 120-1, 120-2, or 120-N, and the number after the dash, e.g., -1, -2, indicates the position in sequence of receive windows 803 for the respective iteration of receiver device 120). Advertisement 802-1 includes first information indicative of an analyte level of a wearer of OBD 101, e.g., first analyte data. Receiver devices 120-1, 120-2, and 120-N can validate, decrypt (if the advertisement is encrypted), and read the received advertisement 802-1 in accordance with the implementation.

At time T1, receiver device 120-1 responds with a first response 804(1)-1. Also at T1, OBD 101 has also activated its wireless communication electronics and is monitoring for response 804(1)-1 and receives it during a receive window 805-1. Specifically, OBD 101 receives response 804(2)-1 during synchronous time slot TS1. Response 804(1)-1 includes an indication of whether or not advertisement 802-1 was received by receiver device 120-1. OBD 101 can validate and decrypt (if the response is encrypted) the received data in response 804(1)-1 in accordance with the implementation. OBD 101 can then read response 804(1)-1 to determine if advertisement 802-1 was received (e.g., validly received) by receiver device 120-1. In this embodiment, response 804(1)-1 indicates receipt. In other embodiments, if response 804(1)-1 indicates advertisement 802-1 was not received, or was received but invalid, then OBD 101 can take further action such as retransmission as described in more detail herein.

At time T2, receiver device 120-2 responds with a first response 804(2)-1. OBD 101 keeps its wireless communication electronics activated and is monitoring for response 804(2)-1 and receives it during a receive window 805-1. Specifically, OBD 101 receives response 804(2)-1 during synchronous time slot TS2. Response 804(2)-1 includes an indication of whether or not advertisement 802-1 was received by receiver device 120-2. OBD 101 can validate and decrypt (if the response is encrypted) the received data in response 804(2)-1 in accordance with the implementation. OBD 101 can then read response 804(2)-1 to determine if advertisement 802-1 was received (e.g., validly received) by receiver device 120-2. In this embodiment, response 804(2)-1 indicates receipt. In other embodiments, if response 804(2)-1 indicates advertisement 802-1 was not received, or was received but invalid, then OBD 101 can take further action such as retransmission as described in more detail herein.

At time T3, receiver device 120-N responds with a first response 804(N)-1. OBD 101 keeps its wireless communication electronics activated and is monitoring for response 804(N)-1 and receives it during receive window 805-1. Specifically, OBD 101 receives response 804(N)-1 during synchronous time slot TSN. Response 804(N)-1 includes an indication of whether or not advertisement 802-1 was received by receiver device 120-N. OBD 101 can validate and decrypt (if the response is encrypted) the received data in response 804(N)-1 in accordance with the implementation. OBD 101 can then read response 804(N)-1 to determine if advertisement 802-1 was received (e.g., validly received) by receiver device 120-N. In this embodiment, response 804(N)-1 indicates receipt. In other embodiments, if response 804(N)-1 indicates advertisement 802-1 was not received, or was received but invalid, then OBD 101 can take further action such as retransmission as described in more detail herein. OBD 101 can then deactivate its wireless communication electronics.

OBD 101 waits until time T4 and reactivates its wireless communication electronics and can repeat the process of transmitting a second advertisement 802-2 to receiver devices 120-1 through 120-N, which monitor for it and receive it during the respective receive windows 803(1)-2, 803(2)-2, and 803(N)-2. In this embodiment, while OBD 101 was deactivated before time T2, OBD 101 has obtained second information indicating the analyte level of the OBD wearer. This can be the result of a second, more recent measurement made by sensor 160 (not shown). Second advertisement 802-2 contains this second information. Receiver devices 120-1, 120-2, and 120-N read second advertisement 802-2 in similar fashion to first advertisement 802-1 and, if validly received, respond with acknowledgements in response time slots TS1, TS2, and TSN at times T5, T6, and T7, respectively. This process can repeat regularly for as long as the OBD 101 is active and communicating updated analyte data to receiver device 120 (e.g., for the entire wear duration of the OBD 101).

Here, both OBD 101 and receiver devices 120-1 through 120-N know the OBD transmission interval between consecutive advertisements 802 as well as the time at which time slots TS1 through TSN occur (e.g., the time from the start of interval 806 (or the start of the first subinterval 816 (not shown)) to the time of the start of the first response slot, the length of time for each response slot, and optionally the number of response slots or length of the subinterval 816), and are thus capable of regularly communicating analyte data and confirming whether or not each receiver device 120 validly received such data. This can occur in synchronous fashion without lengthy periods where wireless communication electronics is activated and consuming power but not making or receiving transmissions.

Each time slot can have a set duration known by all devices 101 and 120. The set duration of each time slot can be the same. Each receiver device 120 can be assigned to one of the time slots such that each time slot is associated with only one receiver device 120. This can avoid response collisions. OBD 101 can be configured to manage time slot assignments, inform receiver devices 120 of their time slot assignment, and track which time slot is assigned to which receiver device 120. Alternatively, each receiver device 120 can be assigned a time slot via system 100 through trusted server 140 or can be pre-programmed with a fixed time slot. A receiver device 120 can assign the time slots to itself and other receiver devices 120 if the system 100 is configured to permit time slot management by receiver devices 120 directly. Receiver devices 120 and OBD 101 can identify the start and stop times of the time slots by knowing the response interval between the start of the advertisement transmission and the start of the first time slot, and then by knowing the length of each subsequent time slot, which can be iteratively summed to locate the start times of each subsequent slot. Each response transmission 804 is synchronized to occur with a particular time slot without drifting into an adjacent time slot. As such, the time slot durations should be set to be slightly longer than the expected response transmission duration.

In the embodiments herein, session 801 can be implemented according to a Bluetooth protocol, but such is not required. Session 80 can be a connection-less session, which does not require the formation and maintenance of a formal link between OBD 101 and the one or more receiver devices 120 (as opposed to a connection-oriented session that does require the formation and maintenance of a formal link between OBD 101 and a receiver device 120).

In embodiments where valid receipt of analyte data has not occurred, OBD 101 can proceed with the sending of updated second analyte data in advertisement 802-2 but also with the inclusion of the first analyte data a second time. The receiver devices 120-1 through 120-N can read both the first and second analyte data and those devices that already received the first analyte data can ignore that first analyte data, and any receiver devices (e.g., 120-2) that did not validly receive the first analyte data now has a second opportunity to receive such and validate it. This approach has the benefit of ease of implementation but requires enough available room in the payload of advertisement 802 to include both the first and second analyte data, and also requires lengthening the transmission time of advertisement 802. Further, if a particular receiver device 120 fails to validly receive multiple consecutive analyte data transmissions, then advertisement 802 may quickly exceed available payload capacity through inclusion of multiple successive sets of analyte data. This approach also results in the receiver device 120-2 not having access to the first analyte data in the time between transmission intervals which, in some embodiments, may be lengthy to conserve transmit power at OBD 101.

FIG. 8G is a flow diagram depicting an example embodiment of a method 840 of wireless communication in a synchronous advertising-based communication session 801 with system 100. At 842, OBD 101 wirelessly transmits an advertisement 802-1 to two or more receiver devices 120. At 844, OBD 101 receives a first response 804(1)-1 from a first receiver device 120-1, where the response confirms whether or not the advertisement 802-1 was received by the first receiver device 120-1. At 846, OBD 101 receives a second response 804(2)-1 from a second receiver device 120-2, where the second response 804(2)-1 confirms whether or not the advertisement 802-1 was received by the second receiver device 120-2. This process can be extended to a third receiver device or more, and can be continued repeatedly for as many advertisement and response cycles as desired.

The embodiments and variations thereto described with respect to FIGS. 8A-8G can be implemented along with all of the clock accuracy embodiments described herein, including each one of those embodiments described with respect to FIGS. 5A, 5B, 5C, 5D, 6A, 6B, 6C, 7A, 7B, 7C, and 7D. For example, the clock accuracy parameter of receiver devices 120-1 through 120-N can be supplied to OBD 101 by the sending of a response 804 or can be supplied to OBD 101 as part of a preceding connection establishment negotiation. OBD 101 can elect to take a power management action in response to the clock accuracy parameter, such as determining whether or not to communicate with an iteration of receiver device 120 using the synchronous advertising-based wireless protocol described with respect to FIGS. 8A-8G, or determining the duration of receive windows 805 based on the clock accuracy parameter, and/or another one of the power management actions described herein.

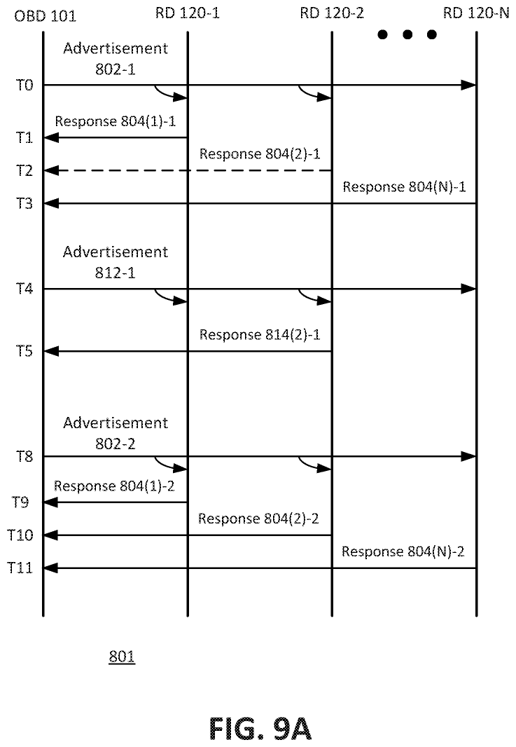

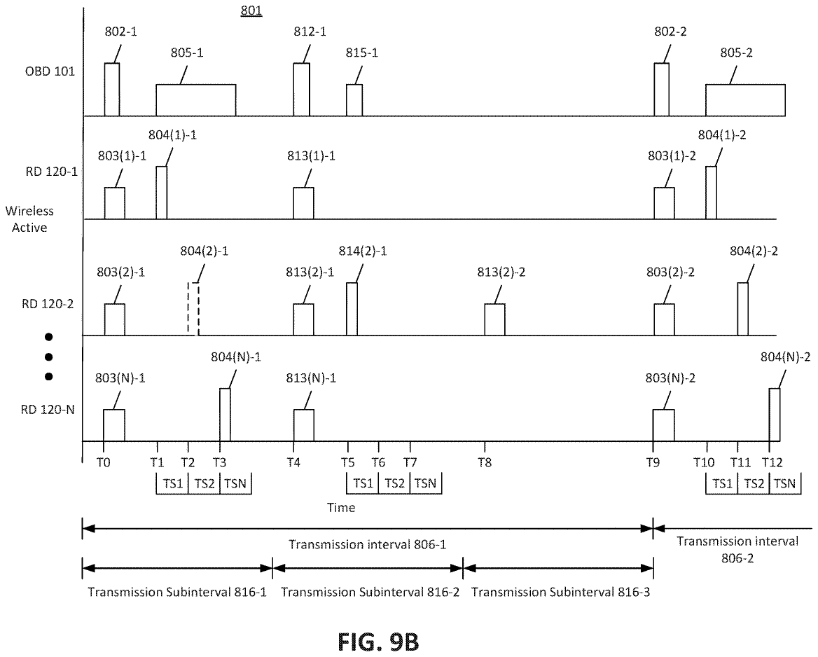

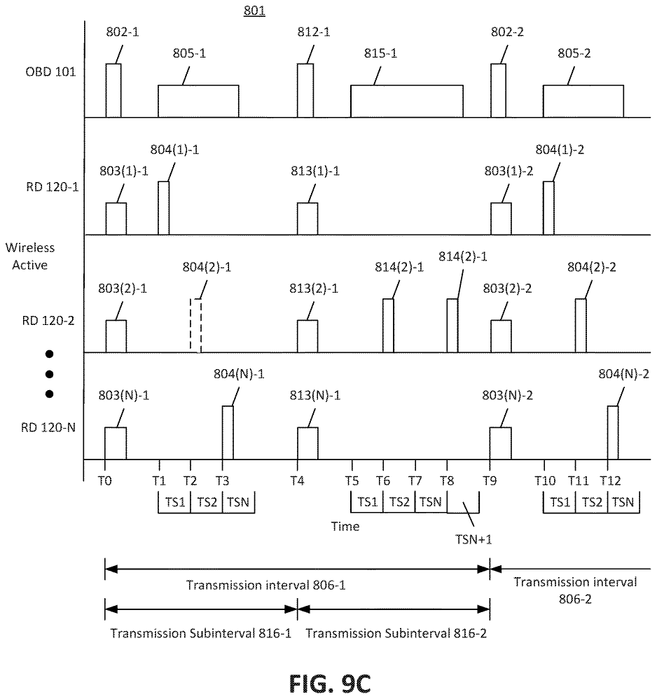

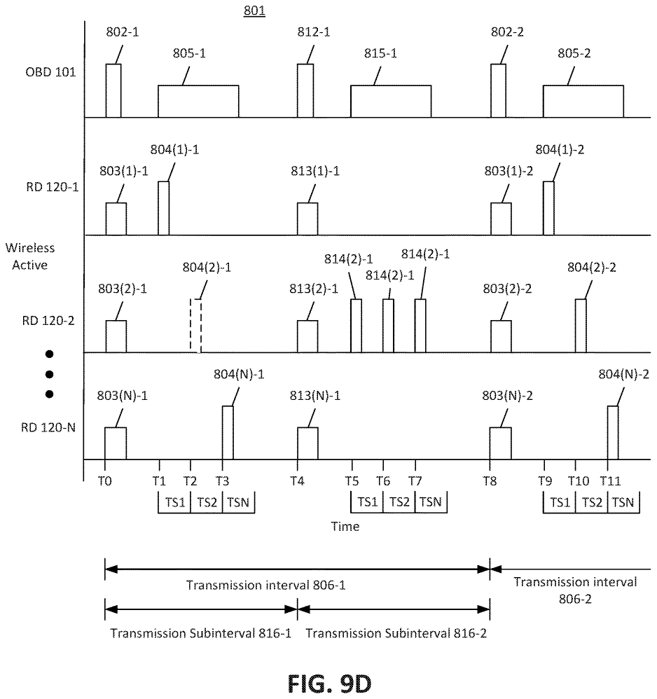

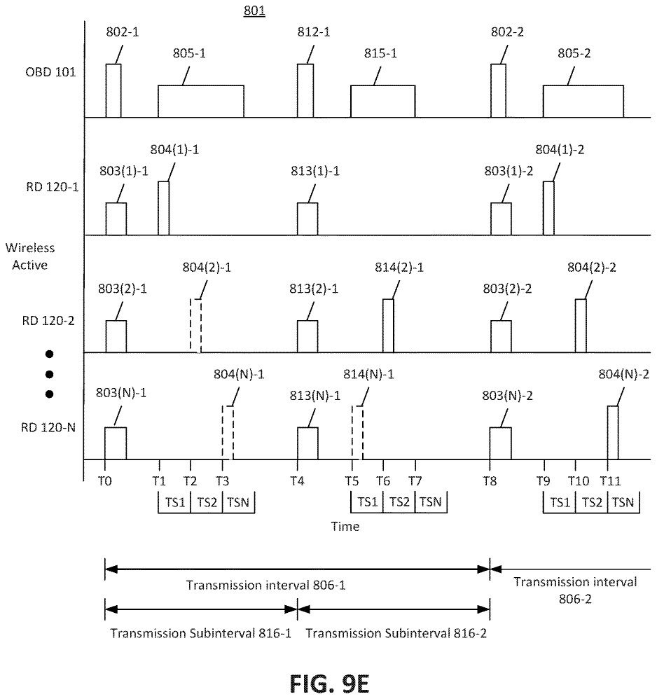

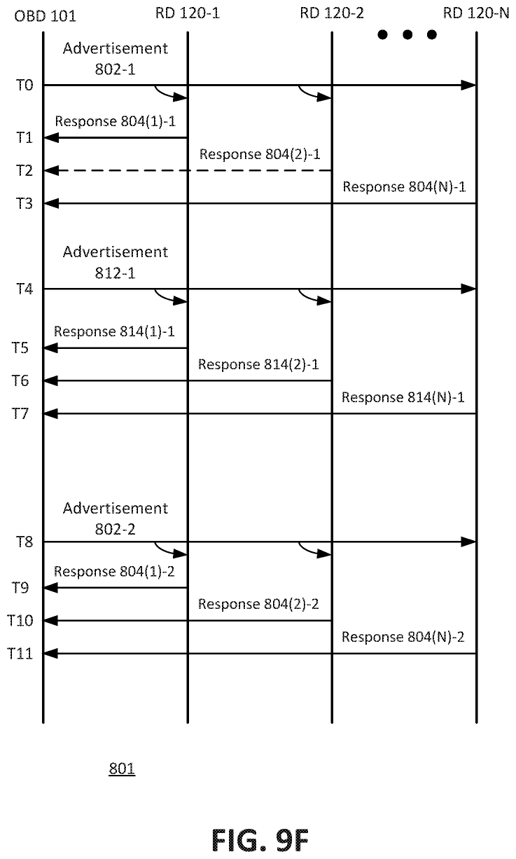

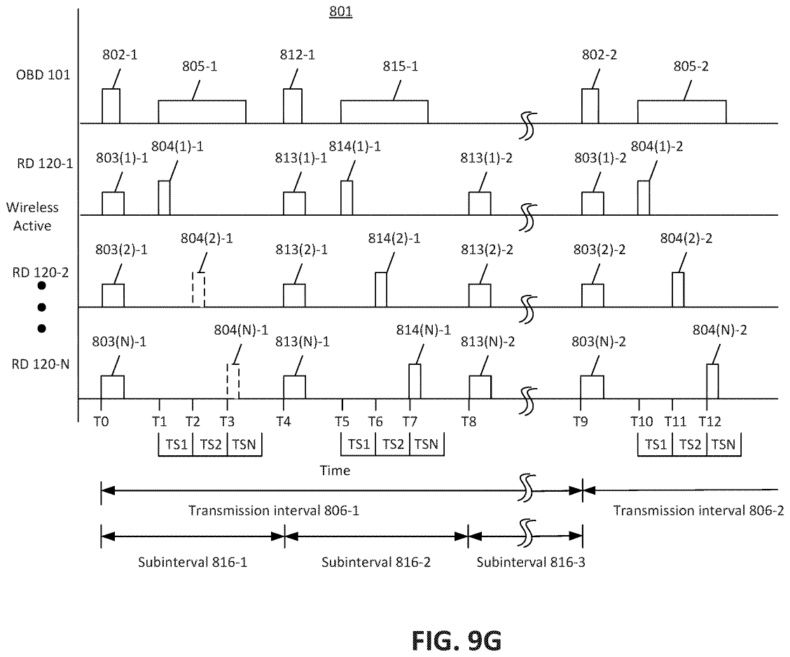

In other embodiments where valid receipt of analyte data has not occurred, OBD 101 can proceed with the sending of a subinterval advertisement. FIG. 9A is another sequence diagram depicting an example embodiment of synchronous communication session 801 in a system 100 having an OBD 101 and two or more receiver devices 120-1 through 120-N communicating with a synchronous advertising-based wireless protocol with at least one subinterval advertisement. FIG. 9B is a timing diagram depicting this embodiment of session 801 with the times at which the wireless communication electronics of OBD 101 and receiver devices 120-1 through 120-N are active (e.g., powered on with the desired power for transmission and/or reception of a wireless signal). The description proceeds here with reference to both FIGS. 9A and 9B. Because OBD 101 and receiver devices 120 operate here in similar fashion to the embodiments described with respect to FIGS. 8A-8F, the same and similar features will not be discussed, with emphasis instead being placed on differences in operation between the embodiments.

Here, OBD 101 has activated its wireless communication electronics and transmits a first advertisement 802-1 having first information indicative of an analyte level of a wearer of OBD 101, e.g., first analyte data, to receiver devices 120-1, 120-2, and 120-N at time T0, and these receiver devices monitor for advertisement 802-1 during the respective receive windows 803(1)-1, 803(2)-1, and 803(N)-1. Receiver devices 120-1, 120-2, and 120-N can validate, decrypt, and read the received advertisement in accordance with the implementation, as discussed in relation to FIGS. 8A-8F.

At time T1, OBD 101's wireless communication electronics is active and is monitoring for responses 804(1)-1, 804(2)-1, and 804(N)-1 from receiver devices 120-1, 120-2, and 120-N, respectively. In this example, OBD 101 receives response 804(1)-1 during TS1 and response 804(N)-1 during TSN, but either does not receive response 804(2)-1 during TS2, receives a response 804(2)-1 that could not be validated, or receives a response 804(2)-1 that is validated but indicates non-acknowledgement (failure) of receiver device 120-2 to receive advertisement 804-1. These possibilities are indicated by response 804(2)-1 being shown in dashed line in FIGS. 9A and 9B. Regardless of the reason, OBD 101 proceeds by transmitting the first information again in a subsequent advertisement 812-1 at time T4.

In the embodiments herein, transmission (primary) interval 806 can be divided into transmission subintervals 816, such that an integer multiple of subintervals 816 constitutes a transmission interval 806. A subinterval advertisement 812 can be transmitted by OBD 101 in each transmission subinterval 816. A subinterval advertisement 812 can be transmitted only in subintervals 816 where the advertisement 812 is required, such as when a receiver device 120 fails to validly receive an advertisement 802 (e.g., indicated either by OBD 101 not receiving the response 804 or receiving a response 804 with a non-acknowledgement). A subinterval advertisement 812 can be followed by a subinterval response 814 indicating whether or not the most recent subinterval advertisement 812 was validly received. System 100 can be configured to permit the transmission of multiple subinterval advertisements 812 and receipt of corresponding subinterval responses 814 in the transmission interval between successive advertisements 802.

In FIGS. 9A and 9B, OBD 101 transmits subinterval advertisement 812-1 at time T4, which is synchronized to the transmission scheme, e.g., after a first transmission subinterval 816 from time T0. Receiver devices 120-1 through 120-N can activate receive windows 813-1 through 813-N, respectively, to monitor for and read the subinterval advertisement 812-1. This can be advantageous as the receiver devices 120 do not yet know whether their last responses 804 were validly received by OBD 101. The subinterval advertisement 812-1 can be addressed (e.g., a directed advertisement) to only the receiver device 120-2 that did not validly indicate receipt of advertisement 802-1 (e.g., an unresponsive or non-responding receiver device), in which case the other receiver devices 120-1 and 120-N receive and read subinterval advertisement 812-1, determine that they are not the addressed recipient, and thus gain confirmation that their prior responses 804 were validly received by OBD 101. No further subinterval response 814 from receiver devices 120-1 and 120-N is made. These non-addressed receiver devices 120-1 and 120-N can then enter a relatively lower power mode until the next advertisement 802.

Receiver device 120-2, as the addressed recipient, can now validly receive and read the analyte data that was not obtained previously. Receiver device 120-2 can then respond with a subinterval response 814(2)-1 containing an acknowledgement that the most recent subinterval advertisement 812-1 was validly received. In this and the other embodiments herein, this subinterval response 814(2)-1 can be transmitted in the same time slot (TS2) assigned to receiver device 120-2 during regular interval responses 804. Alternatively, as shown in FIG. 9B, because the subinterval advertisement 812-1 is addressed to only receiver device 120-2, that receiver device 120-2 can respond in the first response time slot TS1, potentially shortening the time during which OBD 101's wireless communication electronics is activated. The subinterval advertisement 812-1 itself can include an instruction of which time slot receiver device 120-2 is to use when responding, or alternatively the addressed receiver device 120-2 can be programmed to automatically use the first time slot (or another predesignated slot) when it detects that the subinterval advertisement 812-1 was addressed only to itself (receiver device 120-2). The embodiments can be configured such that one or more response time slots are designated as unassigned within the communication protocol. For example, a subinterval response 814 sent by a receiver device 120 can be transmitted in the unassigned time slot which may be a time slot greater than the number of devices 120 (e.g., the seventh or eighth time slot in a system having a maximum of six receiver devices 120 and six corresponding time slots). In this way the allocation of time slots can be efficiently managed to save power and ensure no collisions occur.

Upon valid receipt of response 814(2)-1, OBD 101 can deactivate its wireless communication electronics or otherwise enter the lower power mode and wait until the expiration of the transmission interval. Receiver device 120-2 can activate another receive window 813(2)-2 at time T8, which is the time of the next transmission subinterval, to detect if OBD 101 has sent another subinterval advertisement 812 addressed to device 120-2. (Receiver devices 120-1 and 120-N need not reactivate a receive window as the last subinterval advertisement 812-1 was not addressed to them, indicating that communications with those receiver devices 120-1 and 120-N have ceased for the remainder of this interval 806-1. In this case, because OBD 101 validly received response 814(2)-1, no further subinterval advertisement 812 is made and receiver device 120-2, detecting no further advertisement 812, deactivates its wireless communication electronics or otherwise enters the lower power mode and wait until the next transmission interval. The next transmission interval occurs at T9, at which point the next advertisement 802-2 containing the next analyte data can be transmitted to receiver devices 120-1 through 120-N, which in turn transmit responses 804 back to OBD 101 in their assigned time slots in the manner already discussed.

Advertisements 802 and 812 and responses 804 and 814 can be in a packet format. The format can include a header and a payload. The packet can also include one or more error correction bits, such as cyclic redundancy check (CRC) bits. All or a portion (e.g., payload) of the packet can be encrypted.

The header can include information designating the type of packet, the length of the payload, and/or addressing information, such as an address for the communication session and/or an address for a receiver device 120 or OBD 101 that is the intended recipient of the packet.

The payload of advertisements can include the data indicative of the level of the analyte (for advertisements), information indicating whether or not a response was previously received from each receiver device 120, information about mitigation effort in the case of an unresponsive receiver device 120 (e.g., instructing the unresponsive receiver device 120 to attempt a different frequency or response time slot), setup information for continuing session 801 (e.g., such as a new frequency channel, new response slot assignments, new interval lengths, new subinterval lengths, new response slot delays, and the like), and/or any of the other information types described herein.

The payload of responses can include information indicating whether or not an advertisement was previously received from OBD 101, information about recommended mitigation effort in the case of an inability to receive transmissions from OBD 101 (e.g., requesting the OBD 101 to attempt a different frequency or physical format), all types of receiver response information (as described further herein), and/or recommended or requested setup changes to continue session 801 (e.g., such as a new frequency channel, new response slot assignment, new interval length, new subinterval length, new response slot delay, and the like).

Advertisements 802 and 812 and responses 804 and 814 can be formatted according to any protocol used to perform session 801 such as any of those capable protocols set forth in the “Statement of Wireless Communication Protocols for the Present Embodiments” section herein. In some embodiments session 801 is performed according to a Bluetooth or Bluetooth Low Energy protocol (Bluetooth Low Energy itself is a Bluetooth protocol) and the advertisements 802 and 812 and responses 804 and 814 can be configured as packets in accordance with those protocols. Bluetooth Low Energy packets can be configured with a first part having a preamble and access address, a second part that is a protocol data unit (PDU), and a third part with error check bits. The PDU can include both a header and a payload.

In the embodiments herein, a receiver device 120 addressed by a subinterval advertisement 812 can respond in multiple response time slots (e.g., two slots, three slots, etc., or all slots) where the slots can be adjacent or non-adjacent, in order to increase the likelihood that OBD 101 receives the response 814. This can be beneficial in noisy environments. The response time slots can be assigned by the OBD 101 in the subinterval advertisement 812-1, or can be preassigned such that the receiver device 120-2 automatically knows which slots to respond in without having to be instructed in subinterval advertisement 812-1. FIG. 9C is a timing diagram depicting an example embodiment of session 801 similar to that of FIG. 9B, but where the addressed receiver device 120-2 transmits response 814(2)-1 in its normal response time slot TS2 as well as in a backup slot TSN+1, which is one slot interval later than the slot interval of the Nth receiver device (120-N). FIG. 9D is a timing diagram depicting an example embodiment of session 801 similar to that of FIG. 9B, but where the addressed receiver device 120-2 transmits response 814(2)-1 in all of the response slots TS1 through TSN, optionally including one or more backup slots (not shown). In this and all embodiments herein, OBD 101 can be configured to adjust the length of receive windows 805 and/or 815 to remain active until all response time slots scheduled to contain a response have passed. OBD 101 can be programmed to recognize the conditions where additional time slots will be used, such as in the embodiments of FIGS. 9C and 9D and otherwise and can be programmed to adjust the length of receive windows 805 and/or 815 accordingly.

If multiple receiver devices 120 did not confirm valid receipt of advertisement 802-1, then the OBD 101 can address subinterval advertisement 812-1 to each of those multiple receiver devices 120, in which case those addressed receiver devices 120 can respond in their regular assigned response slot or in response slots reassigned in the subinterval advertisement 812-1. Because OBD 101 knows when to expect subinterval responses 814-1, OBD 101 can shorten the receive window 815-1 and activate it only during those one or more time slots during which subinterval responses 814-1 are expected, thus conserving power by reducing, or even minimizing, the time during which OBD 101's receiver is active. The reassignment of response slots can last only for the present subinterval, or can constitute a relatively more permanent reassignment of response slot in which case other receiver devices 120 may also have their response slots reassigned accordingly in the next advertisement 802-2. FIG. 9E is a timing diagram depicting an example embodiment of session 801 similar to that of FIG. 9B, but where receiver device 120-N also failed to validly confirm receipt of advertisement 802-1. OBD 101 transmits subinterval advertisement 812-1 addressed to both receiver devices 120-2 and 120-N, where that subinterval advertisement 812-1 repeats the analyte data and also includes an instruction for receiver device 120-N to alternatively use time slot TS1 in response and (optionally) for receiver device 120-2 to use response slot TS2 as originally assigned since that time slot is immediately adjacent to time slot TS1 and thus does not need reassignment to minimize OBD 101's receiver window duration. Receiver device 120-N then transmits response 814(N)-1 in time slot TS1 and receiver device 120-2 transmits response 814(2)-1 in time slot TS2.

System 100 can also be configured such that the subinterval advertisement 812-1 is not addressed to a particular receiver device 120 (e.g., a non-directed advertisement). FIGS. 9F and 9G are a sequence diagram and timing diagram, respectively, depicting an example embodiment of session 801 performed with system 100 where receiver device 120-2 is the only receiver device to fail to validly confirm receipt of advertisement 802-1. Here, all receiver devices 120-1 through 120-N monitor for subinterval advertisement 812-1 during their respective receive windows 813-1. Receiver devices 120-1 through 120-N read the analyte data contained within subinterval advertisement 812-1 and determine if that analyte data has already been received. If it has, as would be the case for receiver devices 120-1 and 120-N, then that analyte data can be ignored. If the analyte data has not yet been received, as would be the case for receiver device 120-2, then receiver device 120-2 can store that analyte data in memory and/or display it in accordance with the implementation. Because the receiver devices 120 do not yet have confirmation if their prior responses 804 were validly received by OBD 101, then each receiver device 120-1 through 120-N, including receiver device 120-2, can again respond with an acknowledgement of valid receipt in subinterval responses 814-1 sent in the same time slots allocated for regular responses 804. Each receiver device 120-1 through 120-N then monitors for a subsequent subinterval advertisement 812 and, receiving none from OBD 101, enters a lower power mode until the next transmission interval.

System 100 can experience short disruptions in communication between a particular receiver device 120 and OBD 101 such as the receiver device 120 being temporarily moved out of range of OBD 101, or another transmitter transmitting at the same time as a receiver device 120 resulting in collisions during the allocated time slots for response. In these and other such situations, OBD 101 and a receiver device 120 may need to make multiple attempts to transmit the analyte data of an advertisement and validly confirm receipt.

The embodiments and variations thereto described with respect to FIGS. 9A-9G can be implemented along with all of the clock accuracy embodiments described herein, including each one of those embodiments described with respect to FIGS. 5A, 5B, 5C, 5D, 6A, 6B, 6C, 7A, 7B, 7C, and 7D. For example, the clock accuracy parameter of receiver devices 120-1 through 120-N can be supplied to OBD 101 by the sending of a response 804 or can be supplied to OBD 101 as part of a preceding connection establishment negotiation. OBD 101 can elect to take a power management action in response to the clock accuracy parameter, such as determining whether or not to communicate with an iteration of receiver device 120 using the synchronous advertising-based wireless protocol described with respect to FIGS. 9A-9G, determining the duration of receive windows 805 based on the clock accuracy parameter, or determining a time slot assignment for a particular receiver device 120 based on its clock accuracy parameter (e.g., where a receiver device with a relatively lower clock accuracy is assigned an earlier slot to minimize time until transmission, or a later time slot to permit longer receive windows that do not interfere with subsequent transmissions in later time slots), and/or another one of the power management actions described herein.

FIGS. 10A and 10B are a sequence diagram and timing diagram, respectively, depicting an example embodiment of synchronous communication session 801 where multiple attempts are made. The description proceeds here with reference to both FIGS. 10A and 10B. Because OBD 101 and receiver devices 120 operate here in similar fashion to the embodiments described with respect to FIGS. 9A-9B, the same and similar features will not be discussed, with emphasis instead being placed on differences in operation between the embodiments. In this embodiment OBD 101 optionally keeps the wireless communication electronics 312 in an active state between sending advertisements 812 and starting receive windows 815. In some embodiments maintaining electronics 312 in an active state can consume less power as it eliminates a current inrush event provided the electronics 312 would otherwise only be briefly deactivated.

As in the embodiment of FIGS. 9A and 9B, the embodiment of FIGS. 10A and 10B depict progression of communication where an advertisement 802-1 has been transmitted to receiver devices 120-1, 120-2, and 120-N, and receiver devices 120-1 and 120-N have transmitted acknowledgements of valid receipt in responses 804(1)-1 and 804(N)-1, respectively, while receiver device 120-2 has not acknowledged valid receipt in response 804(2)-1, either because advertisement 802-1 was corrupted when received and/or read by receiver device 120-2 and receiver device 120-2 responded with a non-acknowledgement in response 804(2)-1, or because no response 804(2)-1 was sent by receiver device 120-2, and/or because no response 804(2)-1 was received by OBD 101. In any event, OBD 101 attempts to transmit the analyte data again in a subinterval advertisement 812-1, along with an instruction to respond in slot TS1 similar to the embodiment of FIGS. 9A and 9B.

However, as shown in FIGS. 10A and 10B, receiver device 120-2 and OBD 101 again fail to confirm valid receipt, as receiver device 120-2 either did not receive subinterval advertisement 812-1 and responded with subinterval response 814-1 indicating the same at T2, or did receive subinterval advertisement 812-1 but found that it was corrupted and responded with subinterval response 814-1 indicating invalid receipt (or a generic non-acknowledgment), or receiver device 120-2 did validly receive subinterval advertisement 812-1 and transmitted a subinterval response 814(2)-1 that was not validly received by OBD 101, or receiver device 120-2 did not transmit a subinterval response 814(2)-1.

If receiver device 120-2 receives subinterval advertisement 812-1 and sends response 814(2)-1 during reassigned time slot TS1, and OBD 101 receives response 814(2)-1 but cannot validate it, then OBD 101 can deactivate receive window 815-1 after receipt in slot TS1. But if receiver device 120-2 does not receive subinterval advertisement 812-1 or does receive it but cannot validate it due to, e.g., corruption, then receiver device 120-2 may be unaware of the time slot reassignment to TS1 and may transmit a non-acknowledgement response 814(2)-1 during TS2. OBD 101, having not received response 814(2)-1 during TS1, can extend receive window 815-1 to continue monitoring for a response during the previously assigned response slot TS2 (or other as the case may be). This potential extension to receive window 815-1 is indicated here with dashed line. OBD 101 can be configured to monitor for a response 814(2)-1 during the entire set of response time slots TS1 through TSN or beyond if desired, to increase, or even maximize, the probability of receipt if synchronization with receiver device 120-2 has degraded or been lost.

OBD 101 responds to the lack of acknowledgement of valid receipt by transmitting a second subinterval advertisement 812-2 at time T7 and activating receive window 815-2 to monitor for a response. In this example, for example due to a noisy environment, OBD 101 and receiver device 120-2 again fail to validly confirm receipt as indicated by response 814(2)-2 in dashed line. OBD 101 extends receive window 815-2 to monitor in time slots TS1 and TS2, and optionally beyond, without success. OBD 101 again responds to the lack of acknowledgement of valid receipt by transmitting a third subinterval advertisement 812-3 at time T10 and activating receive window 815-3 to monitor for a response. Receiver device 120-2 validly receives subinterval advertisement 812-3 and responds with subinterval response 814(2)-3 at T11 in time slot TS1, which is received by OBD 101 and receive window 815-3 is ended after the transmission 814(2)-3 is fully received by OBD 101. Receiver device 120-2 has now received the analyte data that was originally missed and has confirmed the same. Device 120-2 activates receive window 813(2)-4 to monitor for another subinterval advertisement 812 from OBD 101 in the fourth subinterval window, and receiving nothing, can assume OBD 101 received the prior response 814(2)-3 and has ceased to transmit advertisements for that transmission interval.

The embodiments and variations thereto described with respect to FIGS. 10A-10B can be implemented along with all of the clock accuracy embodiments described herein, including each one of those embodiments described with respect to FIGS. 5A, 5B, 5C, 5D, 6A, 6B, 6C, 7A, 7B, 7C, and 7D. For example, the clock accuracy parameter of receiver devices 120-1 through 120-N can be supplied to OBD 101 by the sending of a response 804 or can be supplied to OBD 101 as part of a preceding connection establishment negotiation. OBD 101 can elect to take a power management action in response to the clock accuracy parameter, such as determining whether or not to communicate with an iteration of receiver device 120 using the synchronous advertising-based wireless protocol described with respect to FIGS. 10A-10B, determining the duration of receive windows 805 based on the clock accuracy parameter, or determining a time slot assignment or reassignment for a particular receiver device 120 based on its clock accuracy parameter (e.g., where a receiver device with a relatively lower clock accuracy is assigned an earlier slot to minimize time until transmission, or a later time slot to permit longer receive windows that do not interfere with subsequent transmissions in later time slots), and/or another one of the power management actions described herein.

In this example receipt of the analyte data was successfully confirmed to OBD 101 after the fourth attempt in the third subinterval. In other examples more (or less) attempts may be need for successful confirmation. System 100 can be configured to limit the number of attempts at confirmation without success so as to conserve bandwidth and power. Attempts for successful confirmation (where the sending of one advertisement to a given receiver device and the failure to receive a response, or receipt of an invalid response, or receipt of a valid non-acknowledgement to that one advertisement from that given receiver device is considered one attempt) can be limited based on a threshold number of attempts, a threshold duration of time (time limit), and/or a combination of the two.

The following examples can be applied to all embodiments of synchronous communication in system 100 described herein, including all of those described with respect to FIGS. 8A through 10B and FIGS. 11-16B. OBD 101 can be configured to make a threshold number of attempts (e.g., two or more) to an unresponsive receiver device 120, including advertisements and/or subinterval advertisements, before taking one or more subsequent actions (described in more detail below). OBD 101 can be configured to make at least two attempts to an unresponsive receiver device 120, including both at least one advertisement and at least one subinterval advertisement, until a threshold duration of time is reached, at which point OBD 101 can take one or more subsequent actions. The threshold duration of time can be less than or more than one transmission interval.

By way of further examples, OBD 101 can be configured to attempt in every subinterval after a first advertisement until the next advertisement is reached, then take one or more subsequent actions. OBD 101 can be configured to attempt a given number of one or more subintervals after a first advertisement where the given number is less than all subintervals of the transmission threshold before the next advertisement, then take one or more subsequent actions. OBD 101 can be configured to attempt in one or more subintervals, up to and including all subintervals, after a first advertisement, and to attempt again in the second advertisement and in zero, one or more subinterval advertisements, up to and including all subintervals, after the second advertisement, and optionally continue in this fashion with a third advertisement and so forth, until either an attempt threshold limit (based on all attempts or only advertisement attempts) or time limit is reached, at which point OBD 101 can then take one or more subsequent actions.

System 100 can be configured to implement the synchronous advertising-based wireless communication protocol using any protocol set forth in the “Statement of Wireless Communication Protocols for the Present Embodiments” section that permits synchronous communication in the manner described herein, including the non-proprietary and non-standardized protocols, and the wireless communication electronics 312 of OBD 101 and the wireless communication electronics 358 of receiver devices 120 can have the hardware and/or software to perform wireless communications in accordance with the selected compatible protocol. In all of the embodiments herein, wireless communication electronics 312 of OBD 101 and wireless communication electronics 358 of receiver devices 120 can be configured to communicate according to a Bluetooth protocol, such as a Bluetooth Low Energy protocol.

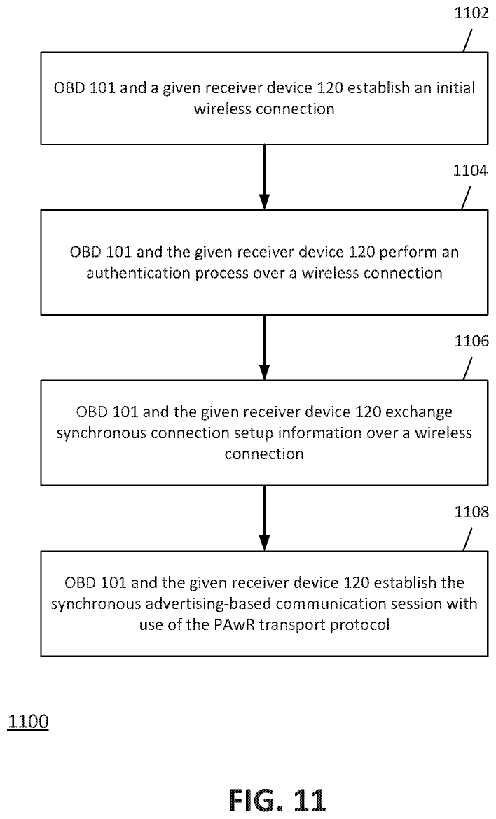

FIG. 11 is a flow chart depicting an example embodiment of a method 1100 performed by system 100 configured to implement the synchronous advertising-based wireless communication embodiments described herein with a Bluetooth Low Energy protocol, such as an extended advertising protocol. One example of a suitable advertising protocol is periodic advertising with response (PAwR), first introduced in the Bluetooth Core Specification Version 5.4 released in February 2023. Advertisements 802, 812 and responses 804, 814 sent using PAwR can be sent on secondary channels (e.g., channels 0-36) as opposed to the primary legacy advertisement channels (e.g., channels 37-39) of Bluetooth and BLE.

At 1102, OBD 101 and a given receiver device 120 establish an initial wireless connection. The initial connection can be a single transmission or can be formulation of a continuous or ongoing connection, such as communication using BLE asynchronous connection-oriented logical transport (LE ACL) in the form of a 1:1 pairing or a BLE Advertising Broadcast (ADVB) logical transport. One of the devices, preferably OBD 101 but alternatively the given receiver device 120, can transmit a connectable and/or scannable advertisement and the other device, preferably the given receiver device 120 but alternatively OBD 101, monitors for the advertisement, receives it, and responds, e.g., with a connection request or scan request. The advertisement can include the advertising address of the advertising device, and optionally the device name, service universally unique identifier (UUID), and/or manufacturer-specific data. The advertisement can be a legacy advertisement sent on a primary channel (e.g., any of channels 37, 38, 39 of the Bluetooth standard) or optionally be an extended advertisement sent on a primary channel (e.g., any of channels 37, 38, 39) that comprises a pointer that directs the receiving device to a secondary channel (e.g., any of channels 0-36) on which further extended advertisement transmissions are made. In other embodiments the initial connection can occur by the given receiver device 120 performing a wireless transmit and receive close range RF scan of OBD 101 (e.g., with NFC where the devices are within six inches or 15 cm). In still other embodiments, the initial connection can be performed through an intermediary such as a remote server accessible via an internet connection, e.g., the cloud.

At 1104, OBD 101 and the given receiver device 120 perform an authentication process over a wireless connection, such as in one of the manners described herein, in order to verify that OBD 101 and the given receiver device 120 are authentic and optionally exchange encryption keys. Step 1104 can be performed using a communication session established in step 1102 if desired. Step 1104 is optional and may be omitted in some embodiments.

Synchronous connection setup information can be exchanged (or transferred) at 1106 over a wireless connection, such as in one of the manners described herein (e.g., using an LE ACL connection or using ADVB (legacy or extended)). This step can be performed independently after authentication, or some of the setup information can be exchanged prior to or during authentication.

The setup information includes information that enables OBD 101 and the given receiver device 120 to communicate in synchronous communication session 801 using the PAwR transport protocol. The PAwR session 801 can be a new session being initially established for communication between OBD 101 and the given receiver device 120. The PAwR session 801 can alternatively be an existing session 801 already in use between OBD 101 and one or more other receiver devices 120, to which the given receiver device 120 is seeking to join.

With respect to step 1106, the setup information can include the length of the transmission interval 806. Also or alternatively, the setup information can include the length of a transmission subinterval 816 (e.g., time from the start of a subevent to the start of the next subevent). Also or alternatively, the setup information can include the number of subintervals per transmission interval (e.g., the number of subevents per PAwR event). The length of the transmission interval 806 can be indicated by a time duration, or by multiplying the length of the transmission subinterval 816 by the number of subintervals. Also or alternatively, the setup information can include the time from the start of transmission interval 806 (or the start of the first subevent 816) to the time of the first response slot TS1, also referred to herein as the response slot delay. Also or alternatively, the setup information can include the time duration of each response slot, e.g., the time from the start of one response slot to the beginning of the next response slot. Also or alternatively, the setup information can include the access address for the PAwR session 801. Also or alternatively, the setup information can include the channel(s) for transmissions and responses in the PAwR session 801. Also or alternatively, the setup information can include format requirements and/or allotments for protocol data units (PDUs) in the PAwR session 801.

As part of the exchange of setup information 1106, OBD 101 and the given receiver device 120 can negotiate any further desired parameters and/or timing for OBD 101 and the given receiver device 120 to be in communication in the PAwR session 801. This can include communication of a clock accuracy parameter of the given receiver device 120 (which can occur in any of steps 1102, 1104, or 1106). The clock accuracy parameter can be used by OBD 101, whether exchanged with setup information or at another time or location, as described in the embodiments herein to evaluate whether the given receiver device 120 has sufficient clock accuracy for synchronized communication with OBD 101. Embodiments implementing the synchronous advertising-based protocol for wireless transmissions can incorporate a minimum clock accuracy threshold that is required for a receiver device 120 to join synchronous advertising-based communication with an OBD 101. Such a threshold will vary depending upon the capabilities and operating parameters of the components of system 100 as implemented. In some embodiments, the minimum clock accuracy can be 500 ppm or less, 400 ppm or less, 300 ppm or less, 200 ppm or less, 100 ppm or less, 50 ppm or less, or 25 ppm or less, to name a few non-exhaustive examples.

If a different communication session has already been established to exchange setup information, e.g., a 1:1 pairing using LE ACL, communication using ADVB, or close-range induced communication scan or scans using NFC or RFID, then that different communication session can be terminated in favor of continuing communication using the PAwR session 801.

In addition to using various transport protocols of Bluetooth or BLE for steps 1102, 1104, and 1106, other protocols can be used as stated. One, two, or all of steps 1102, 1104, and 1106 can be performed using NFC communication between the given receiver device 120 and OBD 101. Similarly, one, two, or all of steps 1102, 1104, and 1106 can be performed using an intermediary between OBD 101 and receiver device 120, such as a remote server in the cloud (e.g., by a WiFi connection (IEEE 802.11), or a mobile telephony connection (e.g., 5G or LTE)), or other wireless protocol such as those described in the Statement of Wireless Communication Protocols for the Present Embodiments. If less than all of steps 1102, 1104, and 1106 are performed with one particular protocol, then the remaining one or two steps can be performed with one or two other (different) protocols.

In this embodiment OBD 101 is configured to act as the PAwR advertiser, in which case the given receiver device 120 act as an observer, alongside any other receiver devices 120. OBD 101 can alternatively be configured as an observer in other embodiments where a receiver device 120 acts as an advertiser, in which case OBD 101 can transmit analyte data in response to receiver device 120 querying for the same in advertisements.

At 1108, the synchronous communication session 801 is now established with use of the PAwR transport protocol. OBD 101 sends an advertisement 802 (e.g., a AUX_SYNC_SUBEVENT_IND PDU or an AUX_CONNECT_REQ protocol data unit (PDU)) to the given receiver device 120 and any other receiver devices 120 already in the PAwR session 801, to which all receiver devices 120 respond (e.g., an AUX_SYNC_SUBEVENT_RSP PDU) in their assigned time slots as described with respect to the various embodiments herein, e.g., FIGS. 8A-16B. At this stage, OBD 101 is able to send analyte data at the specified transmission intervals, keeping each receiver device 120 informed of the most current analyte data, and receiving responses from receiver devices 120 either confirming valid receipt or not, where OBD 101 retransmits as desired and OBD 101 then takes one or more subsequent actions as desired.

For the embodiments described herein, the subsequent action can include sending a instruction to the unresponsive receiver device 120 (or optionally all receiver devices 120) to reconnect to the OBD 101, such as by sending a connectable Bluetooth packet to that unresponsive receiver device 120 to establish a conventional 1:1 pairing (e.g., LE ACL). This connectable Bluetooth packet can be sent in a subsequent advertisement 802 or 812. The subsequent action can also or alternatively include making one or more additional attempts on another frequency (or frequency channel) and/or instructing the unresponsive receiver device 120 to make one or more additional attempts on another frequency (or frequency channel). The subsequent action can also or alternatively include making one or more additional attempts with a different physical format, such as a different data rate and/or modulation index (e.g., 1M, 2M, and Coded PHY for Bluetooth Low Energy) and/or instructing the unresponsive receiver device 120 to make one or more additional attempts with the different physical format. The receiver devices 120 and OBD 101 can be configured to transition to a predetermined or prearranged frequency (or channel) or physical format (e.g., programmed in software installed during manufacturing and/or manufacturing testing, or negotiated or instructed during establishment of the communication session between OBD 101 and the unresponsive receiver device 120) after prolonged lack of transmission from OBD 101. The subsequent action can also or alternatively include ceasing making attempts for the given analyte data to that unresponsive receiver device 120 altogether but continue to attempt to send new analyte data in one or more subsequent advertisements. The subsequent action can also or alternatively include transmitting a final addressed advertisement to the unresponsive receiver device 120 comprising a notification that no further attempts to communicate with the unresponsive receiver device 120 will be made, and optionally that the unresponsive receiver device 120 needs to repeat the connection process and be assigned a new response slot to regain access to session 801. The notification itself can be a flag that the unresponsive receiver device 120 is programmed to interpret as such. The subsequent action can also or alternatively include ceasing transmissions to the unresponsive receiver device 120 altogether (including new analyte data advertisements).

In the embodiments described herein, OBD 101 can instruct receiver devices 120 to adjust the amount of time (an increase or decrease) in each response time slot to generate a new timing pattern that may not collide with transmissions coming from other devices in the area (e.g., in a noisy environment such as a retail location or sporting event). OBD 101 can instruct the receiver devices 120 to adjust the amount of time if one or more failed attempts have occurred, and thus this action can qualify as one of the subsequent actions just described. Also or alternatively, in the embodiments described herein, OBD 101 can reassign time slots to receiver devices 120, time slots that are either consecutive or with time gaps (e.g., one or more unassigned time slots intermixed with assigned time slots) before the first time slot, or before one or more of the subsequent time slots (e.g., alternating between assigned and unassigned time slots) to generate the new timing pattern. OBD 101 can reassign time slots to the receiver devices 120 if one or more failed attempts have occurred, and thus this action can qualify as one of the subsequent actions just described. The OBD 101 can be configured to take these time adjustment or reassignment actions, optionally, when a failed attempt has occurred but after successful communication has been reestablished, to mitigate the possibility that one or more receiver devices 120 do not receive the updated instruction.

System 100 can be configured such that a first receiver device 120 that joins communication with OBD 101 over the synchronous advertising-based wireless protocol described in the embodiments herein is allocated the first response slot TS1, i.e., the response slot that is closest in time to the advertisement 802 transmitted by OBD 101. In embodiments where OBD 101 maintains its wireless communication electronics 312 in an activated state between transmission of advertisement 802 and receiving window 805, the allocation of the first response slot TS1 to the receiver device 120 allows OBD 101 to minimize the amount of time the wireless communication electronics is kept active, as it can be deactivated after receipt of response 804 in window 805. Accordingly, when a second receiver device 120 joins the synchronous communication session 801, that receiver device can be allocated the second response slot TS2, which is the response slot closest in time after response slot TS1. This can continue with each subsequently joining receiver device 120 given the next sequential response slot (TSM+1) closest in time to the immediately preceding response slot (TSM) in order to limit the amount of time the wireless electronics 312 of OBD 101 are active.

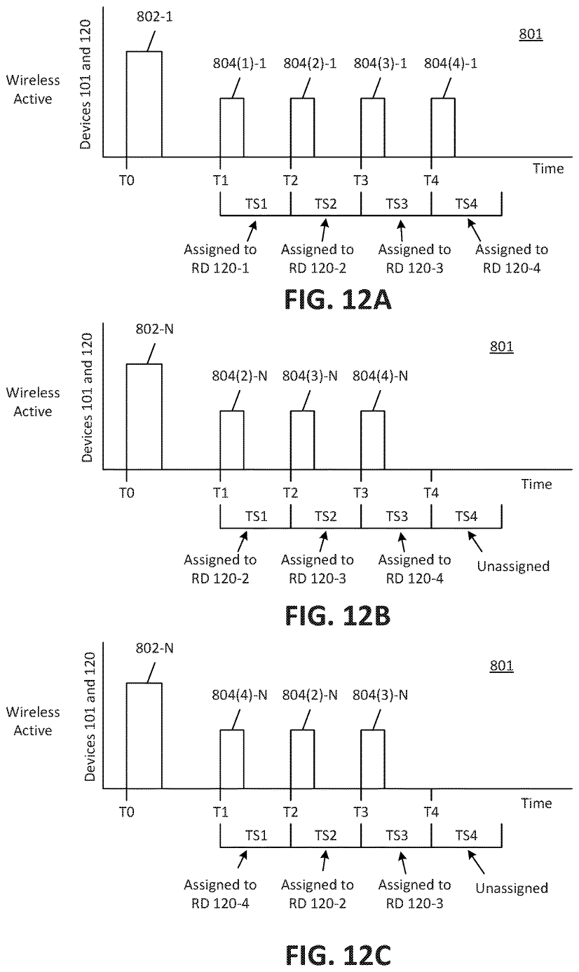

FIGS. 12A, 12B, and 12C are timing diagrams depicting an advertisement 802 and responses 804 thereto in example embodiments of a session 801. If a receiver device 120 that is part of session 801 leaves the session 801, either by action of the user or failure of the receiver 120 or other reason, then system 100 can be configured to either leave the response slot assignments as currently allocated, or to reassign the response slots to eliminate any gaps where a response slot does not have an assigned receiver device 120. For example, if four receiver devices (120-1, 120-2, 120-3, and 120-4) are in a session 801 with OBD 101 with response slots assigned as shown in the timing diagram of FIG. 12A, and receiver device 120-1 leaves the session, then OBD 101 can reassign the response slots to the allocation shown in FIG. 12B, where the remaining receiver devices have each been shifted forward in time by one response slot, and have a reduced delay from the last communication from OBD 101. Alternatively, OBD 101 can reassign receiver device 120-4 to the response slot TS1 to obtain similar effect as depicted in FIG. 12C.

OBD 101 can be configured to assign response slots in reverse order of clock accuracy of each receiver device 120. The accuracy can be for a sleep clock of the receiver device or an active mode clock depending on the application and state of receiver devices 120 prior to receiving and making response transmissions. For example, OBD 101 can assign the first response slot TS1 to the receiver device 120 having the relative lowest (worst) clock accuracy (highest ppm accuracy bounds), the second response slot TS2 to the receiver device 120 having the relative next-to-lowest clock accuracy, and so forth, with the last response slot assigned to the receiver device with the relative highest (best) clock accuracy (lowest ppm accuracy bounds). Thus the receiver devices 120 relatively most susceptible to drift wait the relatively shortest amount of time between receipt and transmission.

Memory 308 of OBD 101 can store instructions that cause processing electronics 302 to assign or allocate response slots in the manners described. OBD 101 can transmit the response slot reassignments to the receiver devices 120 as part of the next advertisement 802 and can proceed with reassignment once all receiver devices have confirmed receipt of the respective assignments, such as by transmission of a valid response 804 that is received by OBD 101.

The embodiments and variations thereto described with respect to FIGS. 12A-12C can be implemented along with all of the clock accuracy embodiments described herein, including each one of those embodiments described with respect to FIGS. 5A, 5B, 5C, 5D, 6A, 6B, 6C, 7A, 7B, 7C, and 7D. For example, the clock accuracy parameter of receiver devices 120-1 through 120-N can be supplied to OBD 101 by the sending of a response 804 or can be supplied to OBD 101 as part of a preceding connection establishment negotiation. OBD 101 can elect to take a power management action in response to the clock accuracy parameter, such as determining whether or not to communicate with an iteration of receiver device 120 using the synchronous advertising-based wireless protocol described with respect to FIGS. 12A-12C, determining the duration of receive windows 805 based on the clock accuracy parameter, or determining a time slot assignment or reassignment for a particular receiver device 120 based on its clock accuracy parameter relative to other receiver devices (e.g., where a receiver device with a relatively lower clock accuracy is assigned an earlier slot to minimize time until transmission, or a later time slot to permit longer receive windows that do not interfere with subsequent transmissions in later time slots), and/or another one of the power management actions described herein.

System 100 is adaptable to many manners of use by a user potentially involving communication between OBD 101 and many different receiver devices 120, in environments that may be noisy requiring multiple retransmissions of analyte data. As such it can be difficult to predict the amount of power or energy that will be required from power source 320 of OBD 101, and that available power or energy may need to be monitored by OBD 101 in order to determine whether OBD 101 will meet the target wear duration. For these and/or other reasons, such as improving efficiency of communication and/or improving robustness of communications even in a case where ample power or energy exists in source 320, it is desirable to configure system 100 to make adjustments to one or more operating aspects of communication session 801.

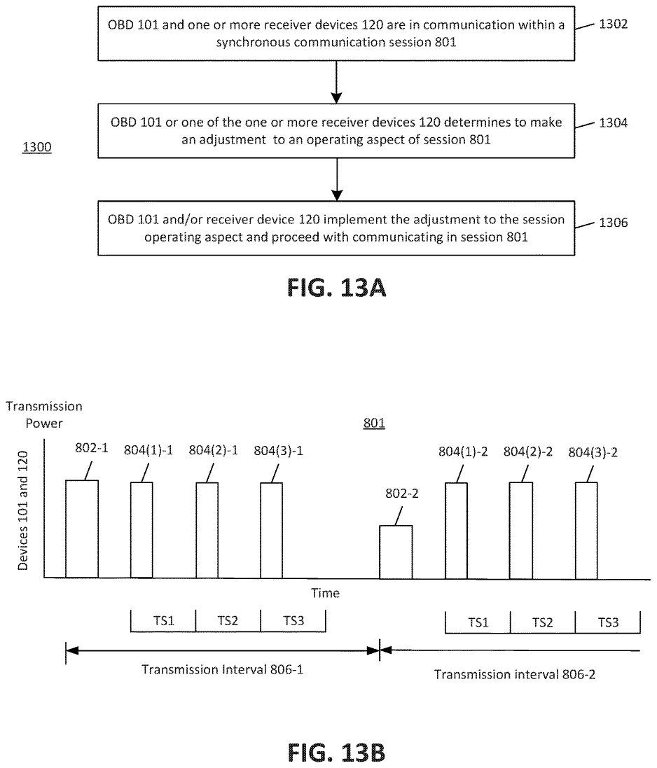

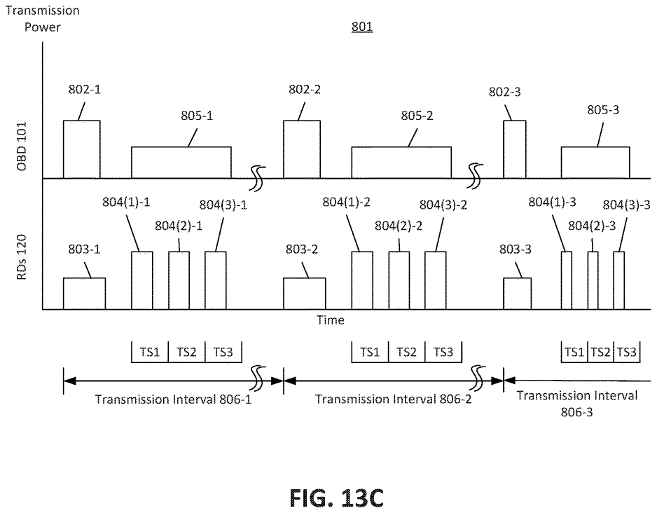

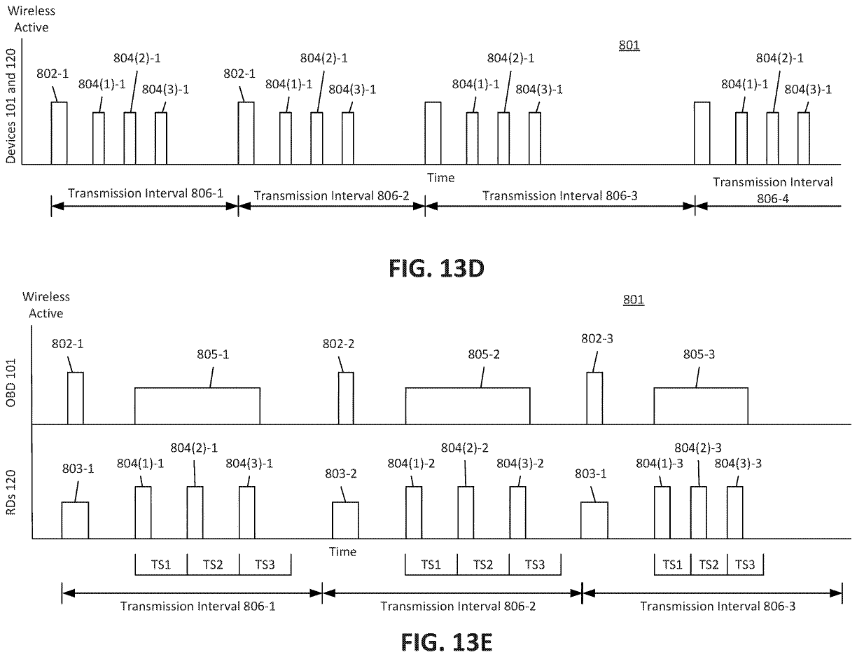

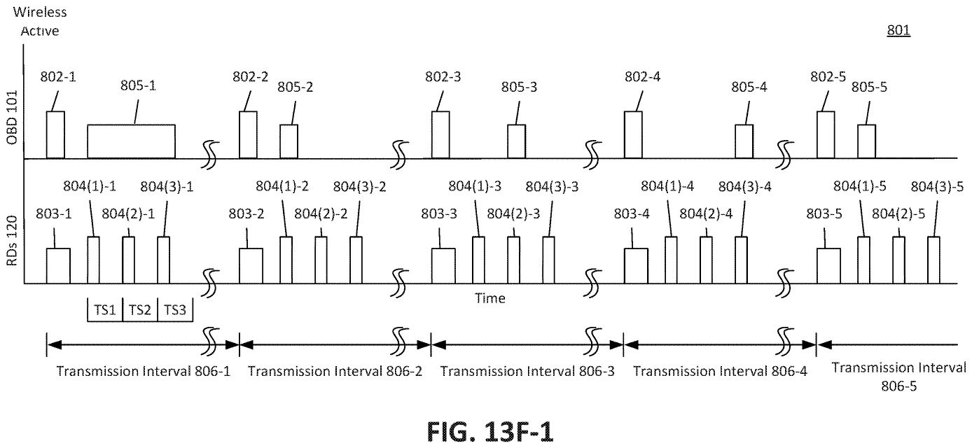

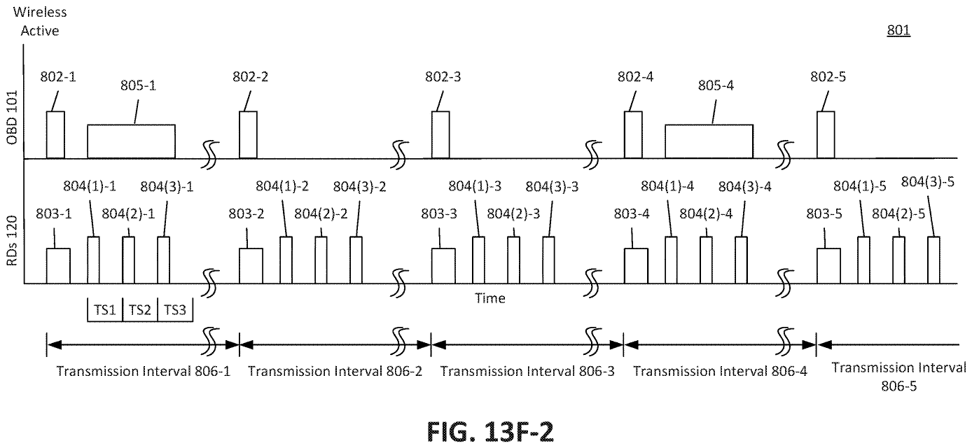

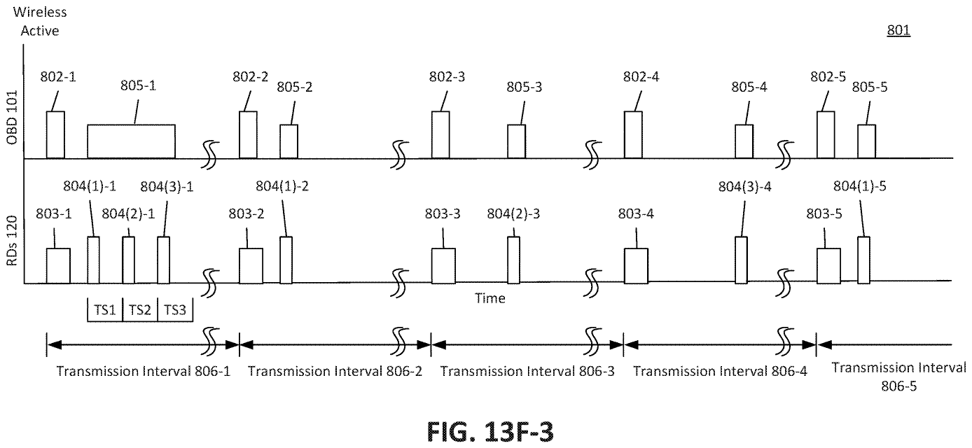

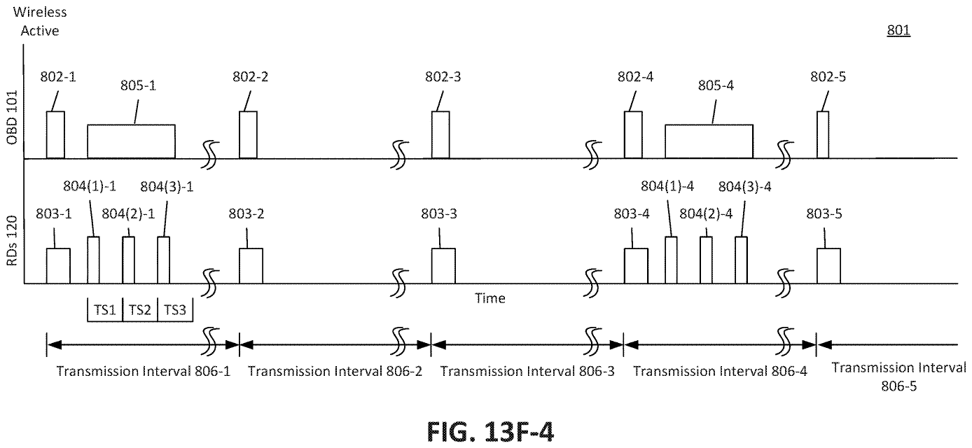

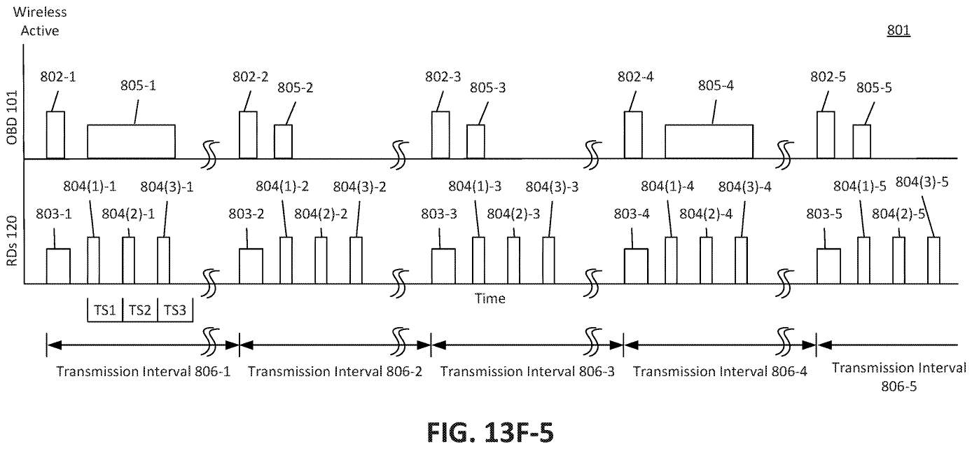

FIG. 13A is a flow diagram depicting an example embodiment of a method 1300 of adjusting an operating aspect of a synchronous communication session 801. At 1302, OBD 101 and one or more receiver devices 120 are in communication within a synchronous communication session 801, which can be a session 801 performed in accordance with any of the embodiments described herein, such as those described with respect to FIGS. 8A-16B.

At 1304, either OBD 101 or one of the one or more receiver devices 120 determines to make an adjustment (e.g., an increase, decrease, or other change) to an operating aspect of session 801. OBD 101 can determine to make the adjustment and in what form the adjustment will take and can communicate the adjustment (optionally, if desired) to the one or more receiver devices 120. Alternatively, OBD 101 can solicit that an adjustment be made (optionally, if desired) to the one or more receiver devices 120, which in turn determines whether to make the adjustment and/or in what form. One of the receiver devices 120 can determine to make the adjustment and in what form the adjustment will take, and can communicate the adjustment (optionally, if desired) to OBD 101, which in turn, if desired, informs the one or more receiver devices in session 801 of the adjustment. Alternatively, one of the receiver devices 120 can solicit that an adjustment be made (optionally, if desired) to OBD 101, which in turn determines whether to make the adjustment and/or in what form and, if desired, informs the one or more receiver devices in session 801 of the adjustment. Communication of the adjustment from one device to another (e.g., from OBD 101 to the one or more receiver devices 120) can be performed as part of session 801, such as by communicating the adjustment in advertisement 802. Alternatively, the adjustment can be communicated as part of a transmission separate and distinct from session 801, such as through a different LE ACL or ADVB transport session, or through NFC communication, or a cloud-based intermediary server.

The determination to make the adjustment can be based on one or more conditions detected by either or both of devices 101 and/or 120. For example, OBD 101 can be configured to monitor the power or energy level of OBD source 320 (e.g., state of charge) and, based on the level of power or energy, e.g., if the level of power or energy crosses a low power or energy threshold, determine to make an adjustment that conserves power or energy of source 320. The receiver devices 120 can perform this action as well in relation to the level of power or energy in receiver source 364.

By way of another example, in addition to or as an alternative to monitoring power, OBD 101 can be configured to monitor for an analyte monitoring condition state, and based on the occurrence of this state, determine to make an adjustment. OBD 101 can detect the analyte monitoring condition state directly or can be notified of its occurrence by one or more of the receiver devices 120 in session 801. The analyte monitoring condition state can be the event where the OBD wearer is detected as going to sleep, or the time at which the user is scheduled for sleep occurs or is detected, in which case the adjustment can be made with less impact on the user's real-time demand for analyte level measurements. The analyte monitoring condition state can be the event where the OBD wearer is detected as having a relatively stable analyte level that is outside of a margin for low analyte level and/or high analyte level alarms (e.g., at an acceptable analyte level not within 10%, 20%, or 30%, etc. of the low or high alarm threshold), in which case the adjustment can be made with less impact on the user's real-time demand for analyte level measurements. The analyte monitoring condition state can be the event where the OBD 101 is in a state where analyte level measurements are masked (e.g., not being displayed to the user and/or OBD wearer). Such a masked condition may occur during a clinical trial, or during certain treatment regimens for diabetes and/or other conditions. In these cases the adjustment can be made without impact on the user's real-time demand for analyte level measurements. The analyte monitoring condition state can be the event where a user (e.g., the OBD wearer) sets manually placed OBD 101 in a low activity state (e.g., to conserve power), such as by selecting or engaging the state through the user interface of one of the receiver devices 120. Alternatively the user can enable a schedule where the low activity state will occur automatically on a periodic or other basis (e.g., daily, weekly, monthly, or custom). Thus the analyte monitoring condition state can be detected by direct monitoring by the OBD 101 and/or one or more receiver devices 120, by the occurrence of a software event (e.g., an interrupt) such as where a scheduled time is reached, by the occurrence of an event based on the measured analyte level of the OBD wearer, or otherwise.

As described in more detail herein, the session operating aspect being adjusted can include, but is not limited to, the transmission interval, the transmission subinterval, the length of response slots, the allocation of one or more response slots, a transmission power, a transmission format, the length and/or time of occurrence of a state of power consumption (e.g., a sleep state, an awake state), the enabling of a receive window, the elimination or addition of a subinterval, and/or the termination (or temporary suspension) of the session 801 altogether.

The devices 101 and 120 of system 100 can be configured (e.g., can have the hardware electronics and/or software) to adjust any combination of two, three, four, five, six, seven, eight, nine, and/or all of the aforementioned operating aspects such that they are in effect at the same time or at different times (e.g., the combined adjustments are initiated at different times but operate concurrently, the combined adjustments are initiated at different times and do not operate concurrently, or a combination of both). At 1306, the devices 101 and/or 120 (both or either one, as desired) implement the adjustment to the session operating aspect and proceed with communicating with each other in session 801, which can be a continuation of the previous session 801 or can be a new session 801 altogether.

As mentioned, the session operating aspect that is adjusted at 1306 can be a transmission power. FIG. 13B is a timing diagram depicting an example embodiment of an OBD 101 and three receiver devices 120-1, 120-2, and 120-3 communicating in a synchronous communication session 801, according to any of the embodiments described herein. In first transmission interval 806-1, OBD 101 transmits first advertisement 802-1 at a first power level, and receiver devices 120-1 through 120-3 receive advertisement 802-1, and transmit responses 804(1)-1, 804(2)-1, and 804(3)-1 at the same or a similar transmit power. Devices 101 and 120 are thus operating in a session 801 in accordance with step 1302. Prior to the start of the next transmission interval 806-2, OBD 101 determines to adjust its transmit power to save power, in accordance with step 1304. In this embodiment, OBD 101 need not notify receiver devices 120 of the adjustment. At the next transmission interval 806-2, OBD 101 transmits advertisement 802-2 at a second transmit power lower than the first transmit power, in accordance with step 1306. Receiver devices 120-1, 120-2, and 120-3 are within range and receive advertisement 802-2 and respond with responses 804(1)-2, 804(2)-2, and 804(3)-2. OBD 101 can continue with operation at this lower transmission power in the subsequent transmission intervals 806. In other embodiments, OBD 101 can notify receiver devices 120 of the adjustment to transmission power, and receiver devices 120 can likewise lower their transmit powers, or alternatively increase their transmit powers to facilitate the receipt of responses 804 by OBD 101.

The session operating aspect that is adjusted at 1306 can also, or alternatively, be a physical format, such as a data rate, modulation index, modulation scheme, and/or coding scheme (e.g., the application of error correction). FIG. 13C is a timing diagram depicting an example embodiment of an OBD 101 and three receiver devices 120-1, 120-2, and 120-3 communicating in a synchronous communication session 801, according to any of the embodiments described herein. In first transmission interval 806-1, OBD 101 transmits first advertisement 802-1 according to a first physical format having a first data rate, and receiver devices 120-1 through 120-3 receive advertisement 802-1, and transmit responses 804(1)-1, 804(2)-1, and 804(3)-1 at the same or a similar data rate. Devices 101 and 120 are thus operating in a session 801 in accordance with step 1302. Prior to the start of the next transmission interval 806-2, OBD 101 determines to adjust its physical format in accordance with step 1304.

The adjustment to physical format can impact power consumption, susceptibility to noise, latency, rate of communication, and/or range of transmission. For example, a physical format with a higher data rate will require more power during transmission as compared to a lower data rate but will result in the transmission circuitry being on for a shorter period of time, and thus can either result in net power savings or cost. The higher data rate can increase rate of communication because data is transferred more quickly but can also make the transmission more susceptible to noise, and the opposite applies for a lower data rate. The higher data rate can similarly decrease latency because less time is required to transmit and receive responses and permits more frequent data exchanges between devices 101 and 120. A physical format with a higher modulation index will require more power during transmission as compared to a lower modulation index but will make the transmission less susceptible to noise, and the opposite applies for a lower modulation index. A physical format that is a modulation scheme (e.g., Binary Phase Shift Keying, Quadrature Phase Shift Keying, Quadrature Amplitude Modulation, Gaussian Frequency Shift Keying) can have different characteristics that impact power consumption, data rates, range, and/or susceptibility to noise. System 100 can be configured to change modulation schemes to gain benefit of one characteristic (such as power consumption or data rate) in favor of another (such as range or susceptibility to noise). A physical format with a more robust coding scheme, such as one that introduces additional bits for error correction, will require more power during transmission as compared to a coding scheme that is less robust (e.g., does not apply error correction) but the more robust scheme will make the transmission less susceptible to noise and can increase its range, particularly if combined with a higher transmit power. The opposite applies for a less robust coding scheme. The embodiments herein can adjust the physical format to achieve results in terms of power consumption, susceptibility to noise, rate of communication, latency, and/or range as desired for each application of system 100.

At the next transmission interval 806-2, OBD 101 transmits advertisement 802-2 again according to the first physical format, but includes a notification in advertisement 802-2 that OBD 101 will switch to a second physical format. The second physical format may either shorten or lengthen the time required to transmit, and thus the notification can also include instructions to similarly adjust (e.g., shorten or lengthen) the transmission interval 806 and/or the transmission subinterval 816, adjust (e.g., increase or decrease) the number of subintervals per transmission interval, adjust (e.g., shorten or lengthen) the time duration of each response slot, and/or adjust (e.g., increase or decrease) the number of response slots per transmission interval 806 or transmission subinterval 816.

Receiver devices 120-1, 120-2, and 120-3 are within range and receive advertisement 802-2 and respond with acknowledging responses 804(1)-2, 804(2)-2, and 804(3)-2. At the next transmission interval 806-3, OBD 101 transmits advertisement 802-3 to the second physical format in accordance with step 1306. Here, the second physical format has a higher data rate and lower modulation index and thus permits the transmission to be completed more quickly. Receiver devices 120-1, 120-2, and 120-3 receive advertisement 802-3 and respond with acknowledging responses 804(1)-3, 804(2)-3, and 804(3)-3. To save additional power, OBD 101 has instructed receiver devices 120 that the response time slots TS1, TS2, TS3 have a new duration that is shorter than the previous response time slot duration, and this allows receive window 805-3 to be shortened in corresponding fashion (to the sum of the durations of the occupied response time slots, or optionally to the terminus of receipt of the last response which may be before the end of the last time slot). Receiver device receive windows 803-3 can also be shortened (one receive window 803 is shown for convenience, although each receiver device 120 would operate its own receive window 803). The power consumption parameters of systems 100 will vary by implementation and application, but in this example the transition to the higher data rate and lower modulation index results in a net power saving due to the shorter duration of time OBD 101's wireless circuitry is active. OBD 101 can continue with operation at this higher data rate and lower modulation index in the subsequent transmission intervals 806.

Standardized and non-standardized wireless communication protocols can have defined physical formats from which communicating devices can communicate. For example, physical formats available in Bluetooth Low Energy include LE Coded Phy, 1 Megabit (1M) Phy, and 2 Megabit (2M) Phy, each of which can be used as the adjusted operating aspect in the embodiments herein.

The session operating aspect that is adjusted at 1306 can also, or alternatively, be the length of transmission interval 806 and/or transmission subinterval 816. FIG. 13D is a timing diagram depicting an example embodiment of an OBD 101 and three receiver devices 120-1, 120-2, and 120-3 communicating in a synchronous communication session 801, according to any of the embodiments described herein. In first transmission interval 806-1 having a first length (duration), OBD 101 transmits first advertisement 802-1 and receiver devices 120-1 through 120-3 receive advertisement 802-1, and transmit responses 804(1)-1, 804(2)-1, and 804(3)-1. Devices 101 and 120 are thus operating in a session 801 in accordance with step 1302. Prior to the start of the next transmission interval 806-2, OBD 101 determines to adjust the length of transmission interval 806 in accordance with step 1304.

The adjustment to the length of transmission interval 806 can impact power consumption and latency. For example, lengthening the transmission interval 806 will reduce the number of transmissions and responses per unit time, thus requiring less power to be used by devices 101 and 120 (e.g., a net power savings). The longer interval will increase latency as OBD 101 will report analyte data to receiver devices 120 less frequently. Conversely, shortening the transmission interval 806 will increase the number of transmissions and responses per unit time, thus requiring more power to be used by devices 101 and 120 (e.g., a net power cost). The shorter interval will decrease latency as OBD 101 will report analyte data to receiver devices 120 more frequently. The embodiments herein can adjust transmission interval 806 to achieve results in terms of power consumption and latency as desired for each application of system 100.

At the next transmission interval 806-2, OBD 101 transmits advertisement 802-2 again but includes a notification in advertisement 802-2 that OBD 101 will switch to a longer transmission interval 806 in the next iteration to conserve power. In other examples, the advertisement 802-2 can either shorten or lengthen interval 806, and thus the notification can also include instructions to adjust other aspects to accommodate the change to the interval 806 or otherwise as desired, such as to adjust (e.g., shorten or lengthen) the transmission subinterval 816, adjust (e.g., increase or decrease) the number of subintervals per transmission interval, adjust (e.g., shorten or lengthen) the time duration of each response slot, and/or adjust (e.g., increase or decrease) the number of response slots per transmission interval 806 or transmission subinterval 816.

Receiver devices 120-1, 120-2, and 120-3 receive advertisement 802-2 and respond with acknowledging responses 804(1)-2, 804(2)-2, and 804(3)-2. At the end of transmission interval 806-2, and the start of the next transmission interval 806-3, OBD 101 transmits advertisement 802-3 in accordance with step 1306. Receiver devices 120-1, 120-2, and 120-3 receive advertisement 802-3 and respond with acknowledging responses 804(1)-3, 804(2)-3, and 804(3)-3. Interval 806-3 now lasts the longer duration. OBD 101 can continue with operation at this lengthier interval 806 in the subsequent transmission intervals 806-4 and beyond.

The session operating aspect that is adjusted at 1306 can also, or alternatively, be the length of response slots. FIG. 13E is a timing diagram depicting an example embodiment of an OBD 101 and three receiver devices 120-1, 120-2, and 120-3 communicating in a synchronous communication session 801, according to any of the embodiments described herein. In first transmission interval 806-1, OBD 101 transmits first advertisement 802-1 and receiver devices 120-1 through 120-3 receive advertisement 802-1, and transmit responses 804(1)-1, 804(2)-1, and 804(3)-1 in response slots TS1, TS2, and TS3 respectively. Devices 101 and 120 are thus operating in a session 801 in accordance with step 1302. Prior to the start of the next transmission interval 806-2, OBD 101 determines to adjust the length of the response slots in accordance with step 1304.

The adjustment to the length of response slots can impact power consumption and latency. For example, shortening the response slots will reduce the number of responses per unit time, thus requiring less power to be used by devices 101 and 120 (e.g., a net power savings). The shorter time slots will decrease latency as OBD 101 will have confirmation of valid receipt (or not) from receiver devices 120 more quickly. Shortening response slots may permit the transmission subinterval 816 to be shortened as well, further decreasing latency. Conversely, lengthening the response slot length will decrease the number of responses per unit time, thus requiring more power to be used by devices 101 and 120 (e.g., a net power cost). The longer interval will increase latency as OBD 101 will receive responses at a slower rate. Lengthening response slots may require the transmission subinterval 816 to be lengthened as well, further increasing latency. Shorter response slot durations may increase susceptibility to noise, and thus may require more advertisements 802 and responses 804 in subsequent subintervals 816 to confirm transmission, which can require more power. Conversely, lengthening the response slots could reduce noise susceptibility and increase robustness, thus actually saving power. Determining which adjustment provides the appropriate benefit will vary based on the implementation and application of system 100. The embodiments herein can adjust transmission interval 806 to achieve results in terms of power consumption and latency as desired for each embodiment of system 100.

At the next transmission interval 806-2, OBD 101 transmits advertisement 802-2 again but includes a notification in advertisement 802-2 that OBD 101 will switch to a shorter response slots in the next iteration to conserve power. In other examples, the advertisement 802-2 can either shorten or lengthen the response slots, and thus the notification can also include instructions to adjust other aspects to accommodate the change to the slot lengths or otherwise as desired, such as to adjust (e.g., shorten or lengthen) the transmission interval 806, adjust (e.g., shorten or lengthen) the transmission subinterval 816, adjust (e.g., increase or decrease) the number of subintervals per transmission interval 806, and/or adjust (e.g., increase or decrease) the number of response slots per transmission interval 806 or transmission subinterval 816.

Receiver devices 120-1, 120-2, and 120-3 receive advertisement 802-2 and respond with acknowledging responses 804(1)-2, 804(2)-2, and 804(3)-2. At the end of transmission interval 806-2, and the start of the next transmission interval 806-3, OBD 101 transmits advertisement 802-3 in accordance with step 1306. Receiver devices 120-1, 120-2, and 120-3 receive advertisement 802-3 and respond with acknowledging responses 804(1)-3, 804(2)-3, and 804(3)-3 using the shorter response slots. OBD 101 can continue with operation at these shorter response slots in the subsequent transmission intervals 806-4 and beyond.

The session operating aspect that is adjusted at 1306 can also, or alternatively, be a number and identity of response slots that OBD 101 monitors, the number being less than all available response slots. By selectively monitoring a subset of response slots, OBD 101 can reduce the amount of time wireless communication electronics 312 (not shown) are active. FIG. 13F-1 is a timing diagram depicting an example embodiment of an OBD 101 and three receiver devices 120-1, 120-2, and 120-3 communicating in a synchronous communication session 801, according to any of the embodiments described herein. In first transmission interval 806-1, OBD 101 transmits first advertisement 802-1 and receiver devices 120-1 through 120-3 receive advertisement 802-1, and transmit responses 804(1)-1, 804(2)-1, and 804(3)-1 in response slots TS1, TS2, and TS3 respectively, which OBD 101 monitors via receive window 805-1. Devices 101 and 120 are thus operating in a session 801 in accordance with step 1302.

Prior to the start of the next transmission interval 806-2, OBD 101 determines to monitor a reduced number of response slots in accordance with step 1304. Monitoring a reduced number of response slots can impact power consumption and latency. For example, monitoring less response slots will require less power to be used by OBD 101 as the wireless electronics 312 are active for a shorter amount of time as compared to monitoring all response slots that receiver devices 120 are using. In monitoring less response slots, the information that would otherwise be carried by the responses is received less frequently, thereby increasing latency. However this may be a worthwhile tradeoff for the power savings, depending on the implementation and application of system 100. The embodiments herein can adjust transmission interval 806 to achieve results in terms of power consumption and latency as desired for each embodiment of system 100.

At the next transmission interval 806-2, OBD 101 transmits advertisement 802-2. Receiver devices 120-1, 120-2, and 120-3 receive advertisement 802-2 and respond with acknowledging responses 804(1)-2, 804(2)-2, and 804(3)-2. OBD 101 activates its electronics 312 to only monitor response slot TS1 in accordance with step 1306. At the next transmission interval 806-3, OBD 101 transmits advertisement 802-3 and receiver devices 120-1, 120-2, and 120-3 receive advertisement 802-3 and respond with acknowledging responses 804(1)-3, 804(2)-3, and 804(3)-3. OBD 101 activates its electronics 312 to only monitor response slot TS2. At the next transmission interval 806-4, OBD 101 transmits advertisement 802-4 and receiver devices 120-1, 120-2, and 120-3 receive advertisement 802-4 and respond with acknowledging responses 804(1)-4, 804(2)-4, and 804(3)-4. OBD 101 activates its electronics 312 to only monitor response slot TS3. At the next transmission interval 806-5, the process can repeat with OBD 101 reverting to monitoring of response slot TS1, and then in interval 806-6 monitoring response slot TS2 only, and so forth continually repeating. As such, OBD 101 shortens the length of receive window 805 by approximately 66% each interval 806, and thus is able to conserve power.

System 100 can be configured such that for those receiver devices 120 where OBD 101 did not monitor for the response 804 in a particular interval 806 (e.g., devices 120-2 and 120-3 in interval 806-2), those receiver devices 120 do not continue to retransmit responses 804 until OBD 101 acknowledges their receipt. OBD 101 can be configured to inform the receiver devices 120 that OBD 101 is entering into a selective response monitoring state (such as in advertisement 802-2 in the embodiment of FIG. 13F-1), and to limit the number of attempts to seek confirmation from OBD 101. OBD 101 can also, or alternatively, be configured to inform those receiver devices 120 where OBD 101 did not monitor for the response 804 in a particular interval 806 that OBD 101 received the responses in the subsequent advertisement (which may be an advertisement of the next subinterval 816). If a receiver device 120 did not receive an advertisement 802 and needs the analyte data to be retransmitted, then the receiver device 120 can continue to transmit a requisition for that missed analyte data in each subsequent response 804 until OBD 101 transmits the missed data in a subsequent advertisement 802 after monitoring for and receiving that response.

FIG. 13F-2 is a timing diagram depicting an example embodiment of an OBD 101 and three receiver devices 120-1, 120-2, and 120-3 communicating in a synchronous communication session 801, according to any of the embodiments described herein. In first transmission interval 806-1, OBD 101 transmits first advertisement 802-1 and receiver devices 120-1 through 120-3 receive advertisement 802-1, and transmit responses 804(1)-1, 804(2)-1, and 804(3)-1 in response slots TS1, TS2, and TS3 respectively, which OBD 101 monitors via receive window 805-1. Devices 101 and 120 are thus operating in a session 801 in accordance with step 1302. At the next transmission interval 806-2, OBD 101 transmits advertisement 802-2. Receiver devices 120-1, 120-2, and 120-3 receive advertisement 802-2 and respond with acknowledging responses 804(1)-2, 804(2)-2, and 804(3)-2. OBD 101 does not activate its electronics 312 to monitor response slots during this interval 806-2. OBD 101 and receiver devices 120 repeat these same actions in the subsequent interval 806-3. At the next transmission interval 806-4, OBD 101 transmits advertisement 802-4 and receiver devices 120-1, 120-2, and 120-3 receive advertisement 802-4 and respond with acknowledging responses 804(1)-4, 804(2)-4, and 804(3)-4, at which time OBD 101 has activated its electronics 312 to monitor all response slots TS1, TS2, and TS3 in receive window 805-4. At the next transmission interval 806-5, the process can repeat with OBD 101 reverting to monitoring the response slots during only every third interval 806, and so forth continually repeating. As such, OBD 101 reduces the number of times receive window 805 is activated by approximately 66% and thus is able to conserve power.

System 100 can be configured such that for those receiver devices 120 where OBD 101 did not monitor for the response 804 in a particular interval 806 (e.g., devices 120-2 and 120-3 in interval 806-2 of FIG. 13F-1, or all three receivers 120-1, 120-2, 120-3 during interval 806-2 of FIG. 13F-2), those receiver devices 120 do not continue to retransmit responses 804 until OBD 101 acknowledges their receipt. OBD 101 can be configured to inform the receiver devices 120 that OBD 101 is entering into a selective response monitoring state (such as in advertisement 802-2 in the embodiment of FIG. 13F-1), and to limit the number of attempts to seek confirmation from OBD 101. OBD 101 can also, or alternatively, be configured to inform those receiver devices 120 where OBD 101 did not monitor for the response 804 in a particular interval 806 that OBD 101 received the responses 804 in the subsequent advertisement (which may be an advertisement of the next subinterval 816). If a receiver device 120 did not receive an advertisement 802 and needs the analyte data to be retransmitted, then the receiver device 120 can continue to transmit a requisition for that missed analyte data in each subsequent response 804 until OBD 101 transmits the missed data in a subsequent advertisement 802 after monitoring for and receiving that response.

System 100 can be configured such that the receiver devices 120 and OBD 101 enter into a response scheme where the receiver devices 120 know which response slots OBD 101 will actively monitor for a response from each receiver device. FIG. 13F-3 is a timing diagram depicting an example embodiment of an OBD 101 and three receiver devices 120-1, 120-2, and 120-3 communicating in a synchronous communication session 801, according to any of the embodiments described herein. In first transmission interval 806-1, OBD 101 transmits first advertisement 802-1 and receiver devices 120-1 through 120-3 receive advertisement 802-1, and transmit responses 804(1)-1, 804(2)-1, and 804(3)-1 in response slots TS1, TS2, and TS3 respectively, which OBD 101 monitors via receive window 805-1. Devices 101 and 120 are thus operating in a session 801 in accordance with step 1302.

Prior to the start of the next transmission interval 806-2, OBD 101 determines to monitor a reduced number of response slots in accordance with step 1304. At the next transmission interval 806-2, OBD 101 transmits advertisement 802-2 notifying receiver devices 120 which response slots will be monitored. Alternatively, OBD can notify receiver devices 120 that OBD 101 will be entering into a predetermined response monitoring scheme, which can be referenced by a flag or code transmitted as part of advertisement 802-2. Receiver devices 120-1, 120-2, and 120-3 receive the flag or code and thus are informed through correlating the flag or code to prior programming to determine what the response monitoring scheme is. In this embodiment, the response monitoring scheme is the same as that described with respect to FIG. 13F-1, and thus in those response slots where OBD 101 is not monitoring for responses, the affected receiver devices 120 abstain from transmitting responses 804 as shown in FIG. 13F-3. As such, OBD 101 shortens the length of receive window 805 by approximately 66% each interval 806, and thus is able to conserve power, as are the receiver devices 120.

FIG. 13F-4 is a timing diagram depicting an example embodiment of an OBD 101 and three receiver devices 120-1, 120-2, and 120-3 communicating in a synchronous communication session 801, according to any of the embodiments described herein. In this embodiment, OBD 101's response monitoring scheme is the same as that described with respect to FIG. 13F-2, where responses are monitored only in every third interval 806. Like the embodiment of FIG. 13F-3, OBD 101 can inform the receiver devices 120 that OBD 101 will be entering into a selective response monitoring scheme, and the receiver devices can abstain (or refrain) from transmitting responses 804 during those times OBD 101 will not be monitoring.

When selectively monitoring response slots, system 100 can be configured such that responses are monitored according to a known fixed scheme as described with respect to FIGS. 13F-1 through 13F-4. Alternatively, system 100 can be configured to monitor responses on a random or pseudorandom basis, e.g., where OBD 101 determines which slots to monitor in real time not according to a known pattern or scheme. Still alternatively, system 100 can be configured to monitor responses a number (N) instances per unit time per device, e.g., OBD 101 can monitor one response slot every five minutes for each receiver device 120, where the monitored response slot can be fixed (or known ahead of time) or can be randomly selected.

System 100 can be configured to more frequently monitor response slots of a particular receiver device 120 which accords a benefit to the system. For example, OBD 101 can always (or more frequently) monitor the response slots for a receiver device 120 designated as a CPOC as described herein, but not monitor (or less frequently monitor) the response slots from the other non-CPOC receiver devices 120. The other, non-CPOC receiver devices 120 can receive advertisements 802, 812 from OBD 101 and direct responses to the CPOC receiver device 120, which in turn can notify OBD 101 if any receiver device 120 (CPOC and non-CPOC) failed to receive the advertisement 802, 812, in which case OBD 101 can retransmit. The CPOC receiver device 120 can notify the OBD 101 in a response 804, 814 to an advertisement 802, 812 in the same interval or subinterval. For example, in a group of N receiver devices 120 with one CPOC receiver device 120 and N−1 non-CPOC receiver devices 120, the non-CPOC receiver devices 120 can respond to the CPOC receiver device 120 in time slots TS1 through TSN−1, and the CPOC receiver device 120 can receive those responses 804, 814, process them, and respond to OBD 101 in time slot TSN with a response indicating if any retransmission by OBD 101 is required in accordance with the embodiments described herein. Also, or alternatively, the CPOC receiver device 120 can receive advertisements 802, 812 from OBD 101 and relay the content of those advertisements 802, 812 to the other, non-CPOC receiver devices 120 directly, such that the other, non-CPOC receiver devices 120 do not receive directly from OBD 101. The other, non-CPOC receiver devices 120 can then transmit responses directly to CPOC receiver device 120 (e.g., as shown in second state 712 of FIG. 7B). In such embodiments, the synchronous advertising-based protocol communication session can be established between the CPOC receiver device 120 and the other, non-CPOC receiver devices 120, with OBD 101 communicating with the CPOC receiver device according to a separate synchronous advertising-based protocol or other non-synchronous or non-advertising-based protocol. As such, all of the embodiments of synchronous advertising-based protocols described herein, including those based on PAwR, can likewise be applied to a group of N receiver devices 120 by themselves within system 100 without inclusion of OBD 101 in that same communication session.

Similarly, one or more receiver devices 120 in communication with OBD 101 can be designated as a primary receiver device 120 with which the system 100 and/or user wishes to maintain communication at all times, or at relatively more times, as compared to the other (secondary) receiver devices 120 which can then be subjected to a selected response monitoring scheme for power savings. FIG. 13F-5 is a timing diagram depicting an example embodiment of an OBD 101 and three receiver devices 120-1, 120-2, and 120-3 communicating in a synchronous communication session 801, according to any of the embodiments described herein. In this embodiment, OBD 101's response monitoring scheme is the same as that described with respect to FIG. 13F-2, where responses are monitored only in every third interval 806. However in this embodiment receiver device 120-1 is designated as a primary receiver device and OBD 101 monitors for a response from that receiver device 120-1 during every interval 806. Receiver devices 120-2 and 120-3 are designated as secondary receiver devices and are subjected to the selective response monitoring scheme for power savings. A similar approach to that shown in FIG. 13F-5 can be taken in all of the other selective monitoring embodiments described herein, including each of those described with respect to FIGS. 13F-1, 13F-3, and 13F-4, where the primary receiver device 120 transmits a response 804 every interval (or at least more often that it would otherwise respond) for those embodiments of FIGS. 13F-3 and 13F-4.

The receiver device 120 designated as the primary receiver device 120 is one that is excepted from the selective response monitoring scheme, e.g., such that responses from those certain receiver devices 120 are always monitored, or at least monitored more often than the secondary receiver devices 120. The primary receiver device 120 can be one where communication integrity is a priority amongst the various receiver devices 120 in communication with OBD 101. For example, a receiver device 120 that is a drug delivery device 126 may be designated as a primary as communication failure with the drug delivery device 126 might result in lack of needed drug delivery action taking place or the presentation of alarms and/or alerts to the wearer or other user. Several (of many) other examples of receiver devices 120 that can be designated as a primary receiver device include a smartphone 122, which may be the user's main electronic device that is most often carried with the user, or a smartwatch 122 that the user often wears, or a designated receiver device 124. A device of system 100, e.g., OBD 101, can be programmed to treat certain receiver devices 120 as primary receiver devices as soon as such receiver devices 120 join in communication with OBD 101 and report their type to OBD 101 such that OBD 101 recognizes the receiver device type as being one that should be designated as a primary, e.g., as a drug delivery device 126 or smartwatch 122. Alternatively, each receiver device 120 can notify OBD 101 directly when joining in communication whether that receiver device is to be treated as a primary, such as by designating one or more bits or a flag in a packet of a response to an advertisement 802 from OBD 101. Alternatively, a user can designate a particular receiver device as a primary through a user interface, such as an app or other software running on that particular receiver device 120 or another receiver device 120.

The embodiments and variations thereto described with respect to FIGS. 13A through 13F-5 can be implemented along with all of the clock accuracy embodiments described herein, including each one of those embodiments described with respect to FIGS. 5A, 5B, 5C, 5D, 6A, 6B, 6C, 7A, 7B, 7C, and 7D. For example, the clock accuracy parameter of receiver devices 120-1 through 120-N can be supplied to OBD 101 by the sending of a response 804 or can be supplied to OBD 101 as part of a preceding connection establishment negotiation. OBD 101 can elect to take a power management action in response to the clock accuracy parameter, such as determining whether or not to communicate with an iteration of receiver device 120 using the synchronous advertising-based wireless protocol described with respect to FIGS. 13A through 13F-5, determining the duration of receive windows 805 based on the clock accuracy parameter, determining a time slot assignment or reassignment for a particular receiver device 120 based on its clock accuracy parameter (e.g., where a receiver device with a relatively lower clock accuracy is assigned an earlier slot to minimize time until transmission, or a later time slot to permit longer receive windows that do not interfere with subsequent transmissions in later time slots), determining to adjust a transmit power, physical format, transmission interval, and/or response time slot length based on a received clock accuracy parameter (e.g., such that the adjustment conserves power in light of the extra power that may be required to maintain communication with the receiver device having the relatively lower clock accuracy parameter), and/or another one of the power management actions described herein.

System 100 can make higher level adjustments for purposes of power savings, susceptibility to noise, data rate, latency, and/or range. For example, system 100 can be configured to adjust the transport protocol between OBD 101 and receiver devices 120. The devices 101 and/or 120 can determine whether or not to utilize a synchronous communication session 801 in accordance with the embodiments described herein or to utilize a different transport protocol. For example, devices 101 and/or 120 can switch from a synchronous communication session 801 to a non-synchronous communication session, such as according to a BLE ACL or BLE ADVB protocol. Conversely, devices 101 and/or 120 can switch from a non-synchronous communication session to a synchronous communication session 801.

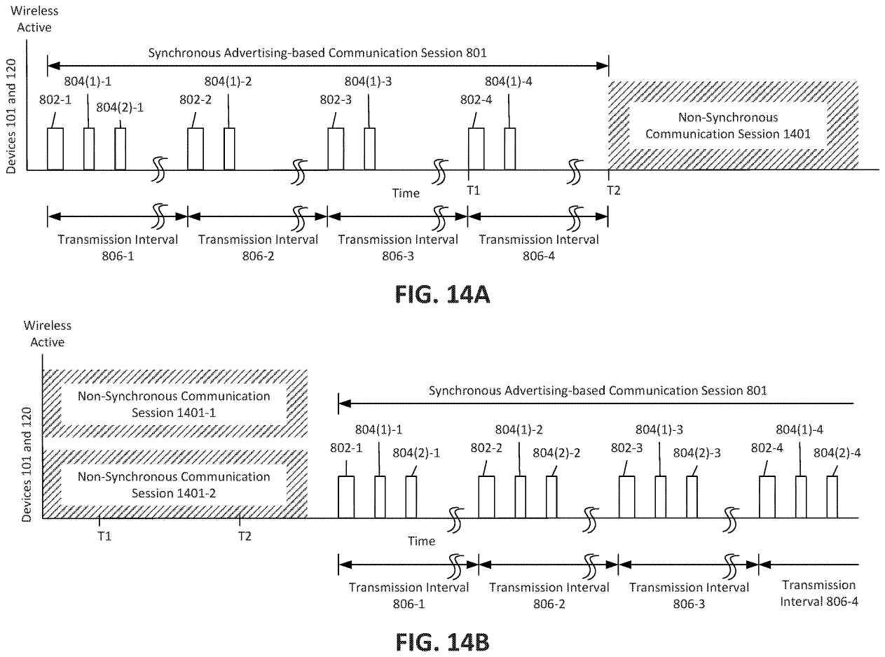

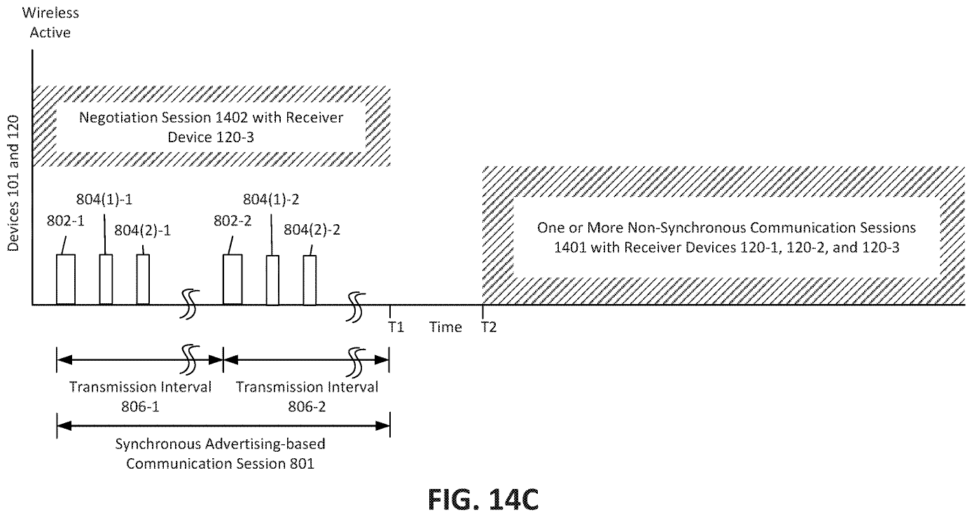

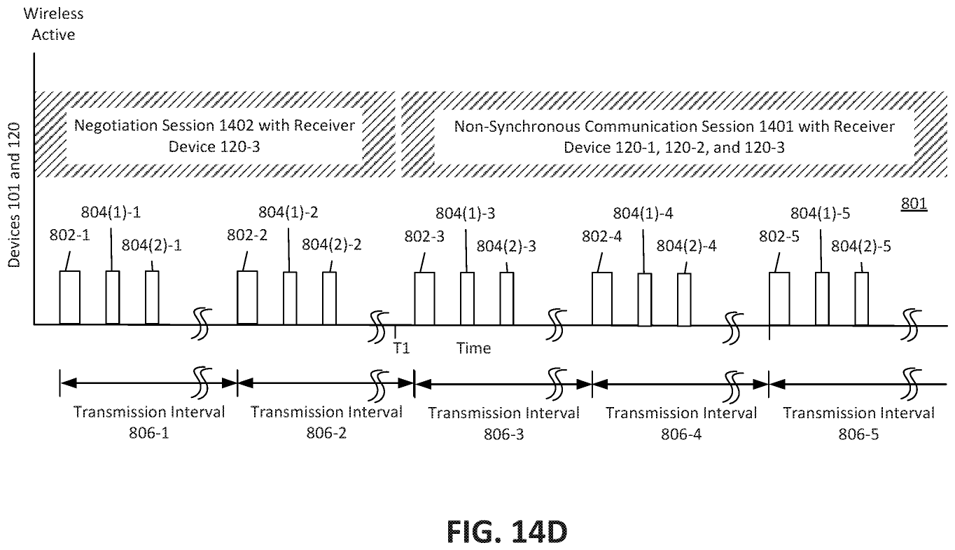

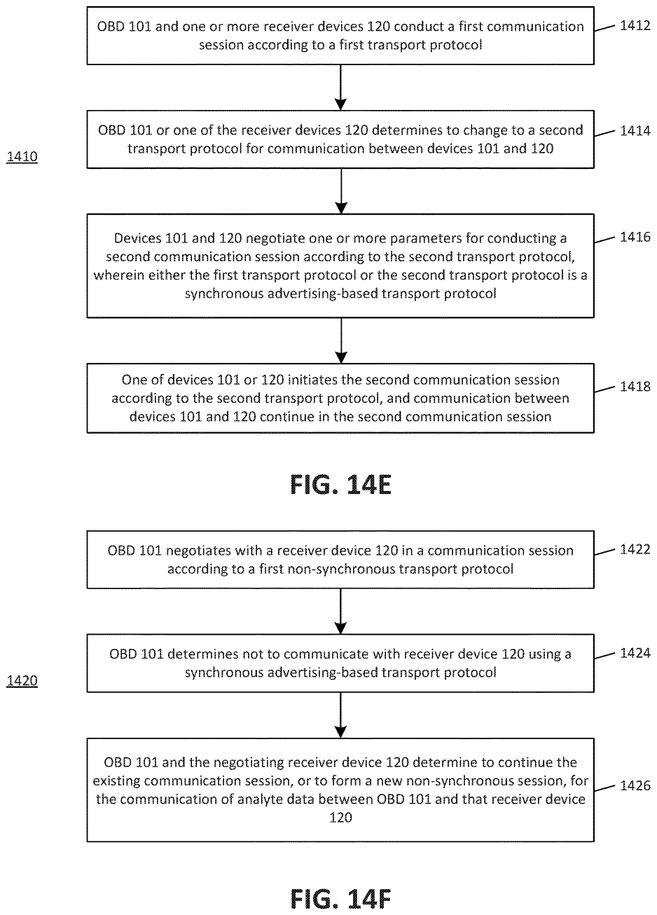

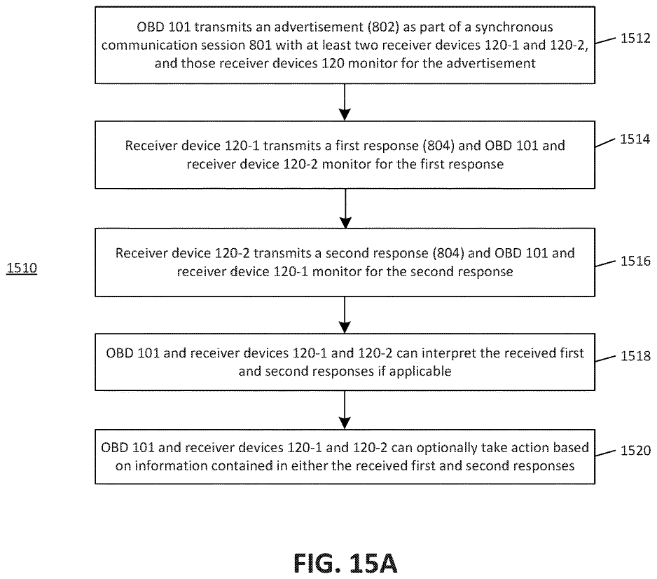

FIGS. 14A, 14B, 14C, and 14D are timing diagrams depicting examples of communication sessions conducted by devices in system 100 where OBD 101 and one or more receiver devices 120 switch between transport protocols. In the embodiment of FIG. 14A, OBD 101 is conducting a synchronous communication session 801 with receiver devices 120-1 and 120-2. After interval 806-1, receiver device 120-2 no longer transmits responses 804 to advertisements 802-2 and 802-3. At time T1 OBD 101 determines that receiver device 120-2 is no longer available for communication. This can occur, for example, by OBD 101 repeatedly attempting to contact receiver device 120-2 during multiple subintervals 816 of interval 806-2 and during multiple subintervals 816 of interval 806-3 without response and subsequently determining to cease communication with receiver device 120-2, the process of which is described elsewhere herein and not shown for ease of illustration. OBD 101 determines to terminate synchronous session 801 in favor of a non-synchronous session 1401, such as a 1:1 pairing according to LE ACL or an advertising and scan response transport protocol like ADVB. OBD 101 transmits advertisement 802-A to receiver device 120-1 negotiating the transition of sessions, and device 120-1 issues response 804-A confirming the transition. In some embodiments multiple advertisements 802 and responses 804 may be necessary to fully negotiate the transition. At time T2, OBD 101 and receiver device 120-1 terminate session 801 and communicate according to non-synchronous session 1401.

In the embodiment of FIG. 14B, OBD 101 is conducting two non-synchronous communication sessions 1401-1 and 1401-2 concurrently with receiver devices 120-1 and 120-2, respectively. Non-synchronous communication sessions 1401-1 and 1401-2 can be a 1:1 pairing according to LE ACL or an advertising and scan response transport protocol like ADVB, or other non-synchronous transport protocol. At time T1, OBD 101 determines to cease sessions 1401-1 and 1402-2 and to consolidate communication with receiver devices 120-1 and 120-2 in a single synchronous communication session 801. Between times T1 and T2 OBD negotiates the synchronous communication session setup information (described earlier herein) with receiver devices 120-1 and 120-2. At time T3, OBD 101 terminates sessions 1401-1 and 1401-2 and initiates session 801 by transmitting advertisement 802-1 at an agreed upon time beginning interval 806-1. Receiver devices 120-1 and 120-2 receive advertisement 802-1 and respond with responses 804(1)-1 and 804(2)-1, which are validly received by OBD 101. Communication can thus proceed between devices 101 and 120 in session 801 in accordance with any of the embodiments described herein.

In the embodiment of FIG. 14C, OBD 101 is conducting a synchronous communication session 801 with receiver devices 120-1 and 120-2, respectively. Concurrently, OBD 101 is conducting a negotiation session 1402 with a third receiver device 120-3 seeking to join session 801. The negotiation session 1402 is using a different transport protocol (e.g., non-synchronous communication session such as a one-to-one (1:1) paired transport protocol session such as LE ACL, a 1:1 or one-to-many (1:M) non-synchronous advertising-based protocol session such as LE ADVB). At time T1, OBD 101 determines that a characteristic of receiver device 120-3 renders it unable or not preferable to join session 801 (e.g., an insufficient clock accuracy as demonstrated by a clock accuracy parameter exchanged during the negotiation), and OBD 101 determines to terminate session 801 and conduct one or more non-synchronous sessions 1401 with receiver devices 120-1, 120-2, and 120-2, such as three 1:1 paired transport protocol sessions (one with each device 120-1, 120-2, and 120-3) or a 1:M non-synchronous advertising-based protocol session with all three receiver devices 120-1, 120-2, and 120-3. OBD 101 can negotiate the transfer to the new session(s) with each receiver device 120-1 and 120-2 (such as by using advertisement 802 and response 804) and with receiver device 120-3 using the existing negotiation session 1402. OBD 101 can then terminate the session 801 and transition communication to the one or more non-synchronous sessions 1401 with receiver devices 120-1 and 120-2, which receiver device 120-3 can also join, as depicted in FIG. 14C. New session 1401 can be independent 1:1 pairings with each receiver device 120-1, 120-2, and 120-3, which results in three sessions 1401, and the session with device 120-3 may be a continuation of session 1402 used to negotiate.

The example embodiment of FIG. 14D is similar to that of FIG. 14C, except that instead of terminating session 801, OBD 101 determines to continue session 801 with receiver devices 120-1 and 120-2, and to operate a second session 1401 with receiver device 120-3 concurrently (e.g., a 1:1 pairing or a 1:1 advertising-based session). The second session 1401 can be a continuation of negotiation session 1402 or can be a new session. In this and other embodiments herein, OBD 101 is configured to operate the second session 1401 at times when the wireless circuitry of OBD 101 is not occupied with transmission and receive windows supporting session 801. If transmission intervals 806 are sufficiently long, there may be ample time to conduct a concurrent session 1401 after the first subinterval 816, or first few subintervals 816. OBD 101 can also, or alternatively, be configured to temporarily pause session 801 (and communicate this to receiver devices 120 via advertisement 902) while negotiating with another receiver device 120 to join session 801.

FIG. 14E is a flow diagram depicting an example embodiment of a method 1410 for switching between communication sessions having different transport protocols. At 1412, OBD 101 and one or more receiver devices 120 conduct a first communication session according to a first transport protocol, such as a one-to-one (1:1) paired transport protocol such as LE ACL, a 1:1 or one-to-many (1:M) non-synchronous advertising based protocol such as LE ADVB, or a synchronous advertising-based protocol such as the embodiments described herein, including PAwR. At 1414, OBD 101 or one of the receiver devices 120 determines to change to a second transport protocol for communication between devices 101 and 120. The second transport protocol is different from the first transport protocol, such as a different one of the 1:1 paired transport protocol, the non-synchronous advertising-based protocol, or the synchronous advertising-based protocol. The basis for changing protocols can be power consumption benefit, efficiency benefit, clock accuracy, or otherwise, including switching between a session 801 having one receiver device 120 to a 1:1 session as described with respect to FIG. 14A, and switching from multiple 1:1 sessions to a single session 801 as described with respect to FIG. 14B, switching between a non-synchronous advertising-based session (either 1:1 or 1:M) to a session 801 (or vice-versa), or switching between a session 801 with multiple receiver devices 120 and one or more non-synchronous sessions 1401 with those devices 120 as described with respect to FIG. 14C. At 1416, devices 101 and 120 negotiate one or more parameters for conducting a second communication session according to the second transport protocol. The negotiation can occur within the first communication session. At 1418, one of devices 101 or 120 initiates the second communication session according to the second transport protocol, and communications between devices 101 and 120 continue in the second communication session. The first communication session can be terminated if desired.

FIG. 14F is a flow diagram depicting an example embodiment of a method 1420 for evaluating whether to add a receiver device 120 to a synchronous communication session 801 (or whether to initiate a new session 801 having a first receiver device 120). At 1422, OBD 101 negotiates with a receiver device 120 in a communication session according to a first non-synchronous transport protocol (e.g., 1:1 pairing or non-synchronous advertising-based protocol). At 1424, OBD 101 determines not to communicate with receiver device 120 using a synchronous advertising-based protocol such as the embodiments described herein. This can be because, for example, receiver device 120 does not have a sufficient clock accuracy, or otherwise. A synchronous session 801 may already be operating concurrently with other receiver devices 120, as shown in FIG. 14D, or no session 801 may yet have been established. At 1426, OBD 101 and the negotiating receiver device 120 determine to continue the existing communication session, or to form a new non-synchronous session, for the communication of analyte data between OBD 101 and that receiver device 120, e.g., as described with respect to FIG. 14D. If a session 801 is already in existence with other receiver devices 120, then the two sessions can operate concurrently for the transfer of analyte data to all receiver devices.

The embodiments and variations thereto described with respect to FIGS. 14A through 14F can be implemented along with all of the clock accuracy embodiments described herein, including each one of those embodiments described with respect to FIGS. 5A, 5B, 5C, 5D, 6A, 6B, 6C, 7A, 7B, 7C, and 7D. For example, the clock accuracy parameter of receiver devices 120-1 through 120-N can be supplied to OBD 101 by the sending of a response 804 or can be supplied to OBD 101 as part of a preceding connection establishment negotiation. OBD 101 can elect to take a power management action in response to the clock accuracy parameter, such as determining whether or not to communicate with an iteration of receiver device 120 using the synchronous advertising-based wireless protocol described with respect to FIGS. 14A through 14F, determining the duration of receive windows 805 based on the clock accuracy parameter, determining a time slot assignment or reassignment for a particular receiver device 120 based on its clock accuracy parameter (e.g., where a receiver device with a relatively lower clock accuracy is assigned an earlier slot to minimize time until transmission, or a later time slot to permit longer receive windows that do not interfere with subsequent transmissions in later time slots), determining to adjust a transmit power, physical format, transmission interval, and/or response time slot length based on a received clock accuracy parameter (e.g., such that the adjustment conserves power in light of the extra power that may be required to maintain communication with the receiver device having the relatively lower clock accuracy parameter), and/or another one of the power management actions described herein.

System 100 can be configured such that receiver devices 120 monitor the responses of other receiver devices 120 during a synchronous session 801. In this manner receiver devices 120 can share information without having to first use OBD 101 (or another device) as an intermediary. This is advantageous because it can reduce delays in communicating information through system 100, as an intermediary will be tied to the synchronous schedule of session 801. Receiver response information types that can be communicated in a receiver response 804 include, but are not limited to: alarm activation and/or deactivation states (e.g., deactivation of an alarm by a user); alarm threshold settings (e.g., high analyte level threshold, low analyte level threshold, etc.); alarm enablement settings (e.g., rate-of-change alerts, predictive alerts); alarm notification format settings (e.g., audible, vibratory); alarm mode settings (e.g., do not disturb mode enabled and/or disabled and/or schedule for the same, user asleep mode enabled and/or disabled and/or schedule for the same); analyte data (e.g., most recent analyte data or historical analyte data); user-specific settings such as personal profile settings (e.g., age, weight, height, time zone, unit of measurement (mg/dl) or mmol/L)); OBD battery level (e.g., low or high); strength of the signal received from the OBD at the receiver device (e.g., low or moderate indicating range; also indication of out of range); calibration information (e.g., fingerstick blood glucose entries); and/or insulin (or other drug therapy) information, such as if a user is also using a pump or insulin pen (e.g., insulin delivery rate(s), correction factor(s), carb ratio(s), therapy schedule(s)).

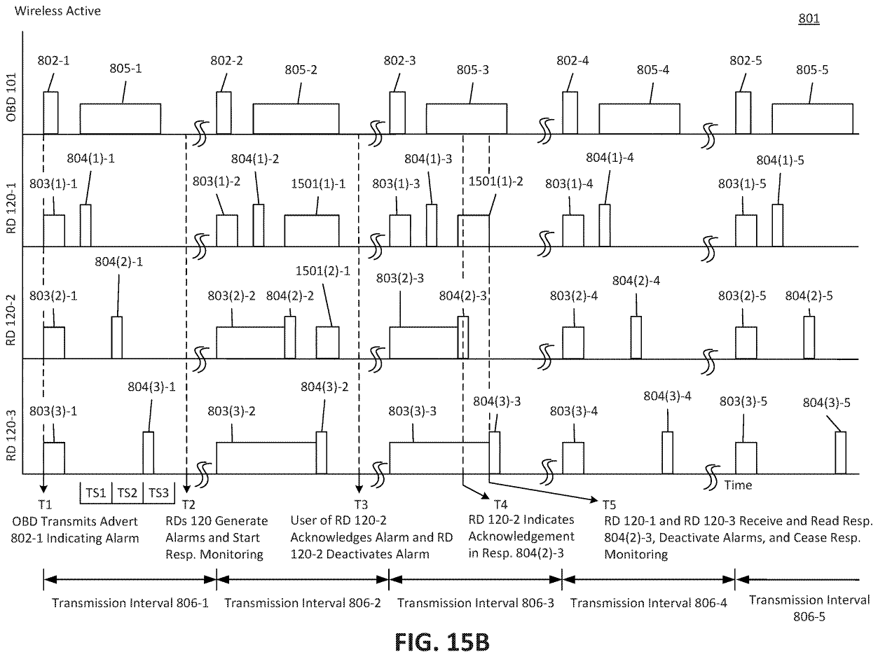

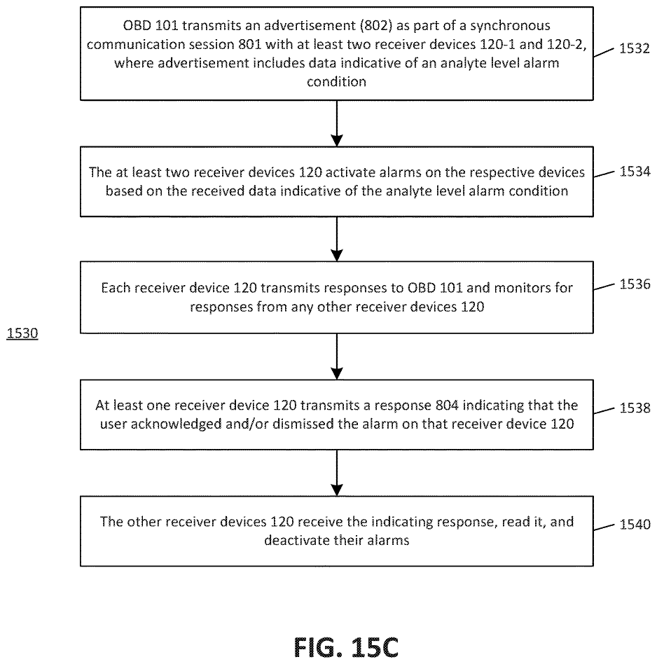

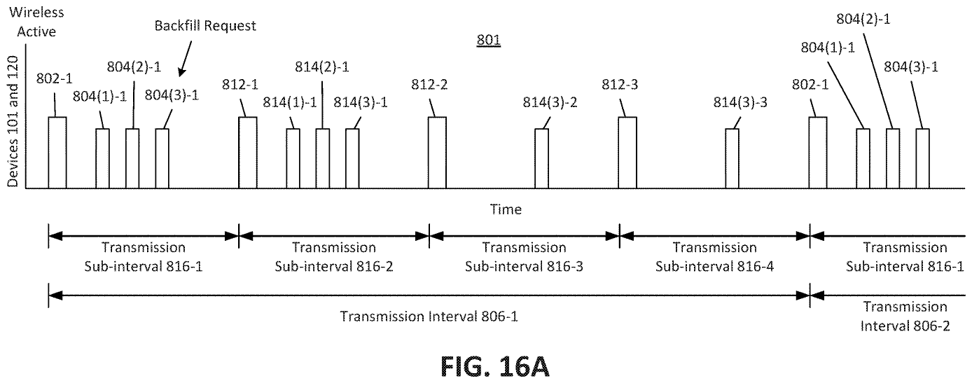

FIG. 15A is a flow diagram depicting an example method 1510 of monitoring responses 804 by system 100. At 1512, OBD 101 transmits an advertisement (802) as part of a synchronous communication session 801 with at least two receiver devices 120-1 and 120-2, and those receiver devices 120 monitor for the advertisement. If conditions are favorable the receiver devices 120 can also validly receive the advertisement. At 1514, receiver device 120-1 transmits a first response (804) and OBD 101 and receiver device 120-2 monitor for the first response with active receive windows. If conditions are favorable OBD 101 and receiver device 120-2 can also validly receive the first response. At 1516, receiver device 120-2 transmits a second response (804) and OBD 101 and receiver device 120-1 monitor for the second response with active receive windows. If conditions are favorable OBD 101 and receiver device 120-1 can also validly receive the second response. At 1518, OBD 101 and receiver devices 120-1 and 120-2 can interpret the received first and second responses (as applicable, and if not filtered first by front end filter logic identifying a lack of need to interpret or process). At 1520, OBD 101 and receiver devices 120-1 and 120-2 can optionally take action based on information contained in either the received first and second responses (as applicable).

The action can be a corollary to the type of information exchanged in the responses. For example, the types of receiver-to-receiver information described herein can be conveyed between receiver devices 120 so that the user does not have to update each receiver device 120 separately, or alternatively does not have to rely on an intermediary to transfer setting information (such as a cloud-based server). Updates (e.g., changes) to settings, calibration information, insulin information, and/or alarm state can be communicated across receiver devices 120 more rapidly. Actions that can be taken by a receiver device 120 receiving new information in a response 804 (step 1520) include updating or first storing information about settings, analyte data, calibration, insulin, and alarms where that information was not previously available to or stored in the response monitoring receiving device 120.

FIG. 15B is a timing diagram depicting transmit and receive operations for OBD 101 and three receiver devices 120-1, 120-2, and 120-3 communicating in a synchronous communication session 801 according to any of the embodiments herein. At T1, interval 806-1 begins and OBD 101 transmits an advertisement 802-1 containing data indicative of an analyte level of the wearer. The data is such that an analyte alarm (e.g., high, low, rate of change) is warranted based on an alarm threshold. The data can indicate the analyte level and the receiver devices 120 can initiate the alarms after making the alarm threshold comparison, and/or the data can indicate the alarm state directly to the receiver devices 120. Receiver devices 120-1, 120-2, and 120-3 activate receive windows 803(1)-1, 803(2)-1, and 803(3)-1, respectively, to monitor for and receive advertisement 802-1. Receiver devices 120-1, 120-2, and 120-3 transmit responses 804(1)-1, 804(2)-1, and 804(3)-1 acknowledging receipt in time slots TS1, TS2, and TS3, respectively. At time T2, receiver devices 120-1, 120-2, and 120-3 activate alarms on their respective display and input interface electronics 368, where the alarms require user acknowledgement to be deactivated. OBD 101 receives responses 804(1)-1, 804(2)-1, and 804(3)-1 in receive window 805-1.

OBD 101 transmits the next advertisement 802-2 at the beginning of interval 806-2 and receiver devices 120-1, 120-2, and 120-3 activate receive windows 803(1)-2, 803(2)-2, and 803(3)-2, respectively, to monitor for and receive advertisement 802-2. Receiver device 120-1 transmits response 804(1)-2 in time slot TS1 indicating receipt. In this embodiment receiver devices 120 are configured to monitor responses 804 of other receiver devices when an alarm is active. Receiver devices 120-2 and 120-3 maintain receive windows 803(2)-2 and 803(3)-2, respectively, in their active states during time slot TS1 to monitor for and receive response 804(1)-2 from receiver device 120-1. Receiver devices 120-2 and 120-3 can read response 804(1)-2 to determine if the user has acknowledged the alarm on receiver device 120-1 (e.g., by touching an acknowledgement or dismissal field on a touchscreen of receiver device 120-1). In this example, the user does not acknowledge the alarm until time T3 on receiver device 102-2, so there is no indication of alarm acknowledgement in response 804(1)-2.

Receiver device 120-2 transmits response 804(2)-2 in time slot TS2 indicating receipt and receiver devices 120-1 and 120-3 monitor for that response in receive windows 1501(1)-1 and 803(3)-2 respectively. Receiver devices 120-1 and 120-3 can read response 804(2)-2 and determine that the user has not yet acknowledged the alarm on receiver device 120-2. Similarly, receiver device 120-3 transmits response 804(3)-2 in time slot TS3 indicating receipt and receiver devices 120-1 and 120-2 monitor for that response in receive windows 1501(1)-1 and 1501(2)-1 respectively. Receiver devices 120-1 and 120-2 can read response 804(3)-2 and determine that the user has not yet acknowledged the alarm on receiver device 120-3. OBD 101 receives responses 804(1)-2, 804(2)-2, and 804(3)-2 in receive window 805-2.

At time T3, the user of receiver device 120-2 acknowledges the alarm on the input interface of that receiver device 120-2. OBD 101 transmits the next advertisement 802-3 at the beginning of interval 806-3 and receiver devices 120-1, 120-2, and 120-3 activate receive windows 803(1)-3, 803(2)-3, and 803(3)-3, respectively, to monitor for and receive advertisement 802-3. Receiver device 120-1 transmits response 804(1)-2 in time slot TS1 indicating receipt and receiver devices 120-2 and 120-3 maintain receive windows 803(2)-3 and 803(3)-3, respectively, in their active states during time slot TS1 to monitor for and receive response 804(1)-3 from receiver device 120-1. Receiver devices 120-2 and 120-3 can read response 804(1)-3 and determine that the user has not yet acknowledged the alarm on receiver device 120-1.

Receiver device 120-2 transmits response 804(2)-3 in time slot TS2 indicating receipt and also indicating that the user has acknowledged the alarm. Receiver devices 120-1 and 120-3 monitor for that response in receive windows 1501(1)-2 and 803(3)-3, respectively. Receiver devices 120-1 and 120-3 can read response 804(2)-3 and determine that the user has acknowledged the alarm on receiver device 120-2. Receiver devices 120-1 and 120-3 can then deactivate the alarms on those respective devices. As such, system 100 is able to quickly convey deactivation of an alarm to other receiver devices communicating concurrently with OBD 101 and the user is not required to acknowledge and/or dismiss the alarm on each receiver device 120 independently. Receiver device 120-3 transmits response 804(3)-3 in time slot TS3 indicating receipt, but receiver devices 120-1 and 120-2 no longer monitor for that response as the alarm condition is no longer present.

In this embodiment, receiver devices 120 monitored responses only when an alarm was active. In other embodiments, receiver devices 120 can be configured to monitor all responses allowing information to be conveyed more rapidly between receiver devices 120. Receiver devices 120 need not monitor for responses but can still include the aforementioned receiver response information (all types) in their responses 804. OBD 101 can receive the information and relay it to the remaining receiver devices 120 in the next or a subsequent advertisement 802, eliminating the need for receiver devices 120 to keep receive windows active for response monitoring at all times.

FIG. 15C is a flow diagram depicting an example method 1530 of monitoring responses 804 related to an alarm within system 100. At 1532, OBD 101 transmits an advertisement (802) as part of a synchronous communication session 801 with at least two receiver devices 120-1 and 120-2, and those receiver devices 120 monitor for and, here, receive the advertisement. The advertisement contains data indicative of an analyte level alarm condition. At 1534, the at least two receiver devices 120 activate alarms on the respective devices based on the received data indicative of the analyte level alarm condition. At 1536, each receiver device 120 transmits responses to OBD 101 and monitors for responses from any other receiver devices 120. The receiver devices 120 read the responses from other receiver devices and monitor for an indication that a user has acknowledged and/or dismissed the alarm on a given receiver device 120. Step 1536 can occur for the transmission of at least one response 804 and can continue through multiple intervals 806 of advertisements 802 and responses 804 until an indication that the user has acknowledged and/or dismissed the alarm is received. At 1538, at least one receiver device 120 transmits a response 804 indicating that the user acknowledged and/or dismissed the alarm on that receiver device 120. At 1540, the other receiver devices 120 receive the indicating response, read it, and deactivate their alarms.

The embodiments and variations thereto described with respect to FIGS. 15A through 15C can be implemented along with all of the clock accuracy embodiments described herein, including each one of those embodiments described with respect to FIGS. 5A, 5B, 5C, 5D, 6A, 6B, 6C, 7A, 7B, 7C, and 7D. For example, the clock accuracy parameter of receiver devices 120-1 through 120-N can be supplied to OBD 101 by the sending of a response 804 or can be supplied to OBD 101 as part of a preceding connection establishment negotiation. OBD 101 can elect to take a power management action in response to the clock accuracy parameter, such as determining whether or not to communicate with an iteration of receiver device 120 using the synchronous advertising-based wireless protocol described with respect to FIGS. 15A through 15C (such as designating a receiver device having a relatively higher clock accuracy parameter as being the device that other receivers monitor), determining the duration of receive windows 805 based on the clock accuracy parameter, determining a time slot assignment or reassignment for a particular receiver device 120 based on its clock accuracy parameter (e.g., where a receiver device with a relatively lower clock accuracy is assigned an earlier slot to minimize time until transmission, or a later time slot to permit longer receive windows that do not interfere with subsequent transmissions in later time slots), determining to adjust a transmit power, physical format, transmission interval, and/or response time slot length based on a received clock accuracy parameter (e.g., such that the adjustment conserves power in light of the extra power that may be required to maintain communication with the receiver device having the relatively lower clock accuracy parameter), and/or another one of the power management actions described herein.

System 100 can be configured such that receiver devices 120 can ask for analyte data that was previously not received by issuing a supplementary information request. The supplementary information requested can be related to any functional aspect of system 100, including, but not limited to: alarm activation or deactivation state (e.g., deactivation of an alarm by a user); alarm threshold settings (e.g., high analyte level threshold, low analyte level threshold, etc.); alarm enablement settings (e.g., rate-of-change alerts, predictive alerts); alarm notification format settings (e.g., audible, vibratory); alarm mode settings (e.g., do not disturb mode enabled or disabled or schedule for the same, user asleep mode enabled or disabled or schedule for the same); analyte data (e.g., most recent analyte data, historical analyte data, and/or analyte data that was missed); user-specific settings such as personal profile settings (e.g., age, weight, height, time zone, unit of measurement (mg/dl) or mmol/L)); calibration information (e.g., fingerstick blood glucose entries); and/or insulin (or other drug therapy) information, such as if a user is also using a pump or insulin pen (e.g., insulin delivery rate(s), correction factor(s), carb ratio(s), therapy schedule(s)).

For ease of discussion, the following embodiments will describe the use of supplementary information requests as they pertain to missing analyte data. A receiver device 120 that did not receive analyte data for a particular time period is not able to display that data to a user, and thus at times where that analyte data is missing any graph and/or textual output to the user may be blank or otherwise have an indication that the analyte data is missing. By requesting the missing data, the receiver device 120 can retroactively fill missing data gaps and thus display a more comprehensive analyte data set to the user. The missing data may be a few minutes old or older, including multiple hours or days old, with an age as old as the wear duration of OBD 101, provided that OBD 101 has sufficient memory to store that entire data set.

FIG. 16A is a timing diagram depicting an example embodiment of communicating missing analyte data from an OBD 101 to a receiver device 120 using synchronous communication session 801 according to any of the applicable embodiments herein. In this embodiment OBD 101 is in communication with three receiver devices 120-1, 120-2, and 120-3 in session 801. In a first transmission subinterval 816-1 of a transmission interval 806-1, OBD 101 transmits advertisement 802-1 including a most recent measurement or set of measurements of analyte data for the wearer collected at a most recent time or over a most recent time period. OBD 101 receives responses 804(1)-1, 804(2)-1, and 804(3)-1 from receiver devices 120-1, 120-2, and 120-3 acknowledging receipt of advertisement 802-1. In this example, receiver device 120-3 is missing analyte data from one or more times or time periods prior to the most recent analyte data contained in advertisement 802-1.

Receiver device 120-3 includes a supplementary information request for the missing analyte data in response 804(3)-1, which may also be referred to as a backfill request, and specifies what analyte data is missing with particularity sufficient for OBD 101 to locate the analyte data. The analyte data can be identified by time stamp, such as corresponding to a time of collection. The analyte data can also or alternatively be identified by an index, code, or sequence number that uniquely identifies the analyte data with respect to other analyte data from the same OBD 101. Thus a first analyte data can be time-correlated, indexed, or sequence-correlated to a different second analyte data.

OBD 101 transmits subinterval advertisement 812-1 in the next subinterval 816-2. Subinterval advertisement 812-1 is or includes an acknowledgement to receiver devices 120-1, 120-2, and 120-3 that OBD 101 received responses 804(1)-1, 804(2)-1, and 804(3)-1, respectively. Subinterval advertisement 812-1 also includes at least a portion of the missing analyte data requested by receiver device 120-3. Receiver devices 120-1, 120-2, and 120-3 respond with responses 814(1)-1, 814(2)-1, and 814(3)-1 acknowledging receipt. If a significant amount of missing analyte data is requested, as in this example, then one subinterval advertisement 812 may not have enough data capacity to transmit all of the missing analyte data, and multiple subinterval advertisements 812 may be necessary. OBD 101 transmits subinterval advertisement 812-2 in the next subinterval 816-3. Subinterval advertisement 812-2 includes at least another different portion of the missing analyte data requested by receiver device 120-3. Receiver device 120-3 responds with response 814(3)-2 acknowledging receipt. Receiver devices 120-1 and 120-2, having received all data and confirming the same, do not need to monitor for further advertisements nor respond until the next transmission interval 806-2. OBD 101 transmits subinterval advertisement 812-3 in the next subinterval 816-4. Subinterval advertisement 812-3 includes at least another different portion of the missing analyte data requested by receiver device 120-3, which in this embodiment is the final group of missing analyte data. Receiver device 120-3 responds with response 814(3)-3 acknowledging receipt, and OBD 101 can optionally confirm receipt of response 814(3)-3 in a subinterval advertisement 812-4 in subinterval 816-5 (not shown). No further transmissions and responses need be made by the devices until OBD 101 next transmits advertisement 802-2 at interval 806-2.

FIG. 16B is a flow diagram depicting an example embodiment of a method 1600 of communicating missing analyte data using a synchronous communication session 801, similar to that described with respect to FIG. 16A. At 1602, OBD 101 transmits an advertisement, to which a receiver device 120 responds with a response including a request for missing analyte data. At 1604, in a subsequent advertisement, which can be a subinterval advertisement, OBD 101 transmits at least a portion of the missing analyte data, to which receiver device 120 responds with a response acknowledging receipt. If the entirety of the requested missing analyte data was included in the advertisement, then OBD 101 can optionally send an additional advertisement, which may be a subinterval advertisement, confirming receipt of the response. Otherwise, step 1604 is repeated until all requested missing analyte data has been transmitted to receiver device 120 at 1606. After all of the requested missing analyte data has been transmitted to receiver device 120, the devices 101 and 120 can end the backfilling process and communicate as normal.

FIG. 16C is a timing diagram depicting an example embodiment of communicating missing analyte data from an OBD 101 to a receiver device 120 using extended advertising according to a Bluetooth Low Energy protocol. OBD 101 sends advertisements 1612-1, 1613-1, and 1614-1 over primary channels 37, 38, and 39, respectively. Advertisements 1612-1, 1613-1, and 1614-1 are scannable advertisements including most recent analyte data measured by OBD 101. A receiver device 120 responds with a scan request 1616 on one of the three primary channels requesting missing analyte data from a time or time period prior to the analyte data transmitted in advertisements 1612-1, 1613-1, and 1614-1. In this embodiment, receiver device 120 responds to advertisement 1614-1 on primary channel 39. OBD 101 responds with a second advertisement 1614-2. The second advertisement 1614-2 can include some of the requested missing data but in this example additional advertisements are needed to transfer all of the requested missing analyte data. The second advertisement 1614-2 includes a pointer (e.g., AuxPtr) designating a secondary channel (e.g., one of channels 0 to 36) and a time offset. The second advertisement 1614-2 can be an ADV_EXT_IND protocol data unit (PDU) and can optionally be a directed advertisement addressed to the receiver device 120 requesting the missing analyte data.

Receiver device 120 monitors the designated secondary channel after the designated time offset and receives an extended advertisement 1618-1 including the requested missing analyte data. The extended advertisement 1618-1 can be in the form of an AUX_ADV_IND PDU. This advertisement 1618-1 can in some cases include the remainder of the missing analyte data, depending on the amount of missing analyte data. If one or more additional extended advertisements are required to complete the missing analyte data transfer, then extended advertisement 1618-1 can include another pointer to another (or the same) secondary channel with another time offset. Receiver device 120 monitors the next secondary channel and receives second extended advertisement 1618-2. Second extended advertisement 1618-2 can be in the form of an AUX_CHAIN_IND PDU. Second extended advertisement 1618-2 can in some cases include the remainder of the missing analyte data, depending on the amount of missing analyte data. If one or more additional extended advertisements are required to complete the missing analyte data transfer, then extended advertisement 1618-2 can include another pointer to another (or the same) secondary channel with another time offset, where receiver device 120 can monitor to receive a third extended advertisement 1618-3 with additional missing analyte data. Third extended advertisement 1618-3 can also be in the form of an AUX_CHAIN_IND PDU. This process can repeat until all of the missing analyte data has been transferred from OBD 101 to receiver device 120.

FIG. 16D is flow diagram depicting an example embodiment of a method 1630 of receiving missing an analyte data on a secondary channel, similar to the embodiment described with respect to FIG. 16C. At 1632, OBD 101 transmits scannable advertisements 1612, 1613, and 1614 on primary channels 37, 38, and 39. The scannable advertisements can include recent or current analyte data measured by OBD 101. At 1634, receiver device 120 responds to one of the advertisements with a scan request 1616, where the scan request requests missing analyte data that the receiver device 120 does not have. At 1636, OBD 101 transmits a second advertisement, which can be a directed advertisement on the same channel on which receiver device 120 responded, that optionally includes some of the missing analyte data. The second advertisement also includes a pointer designating a secondary channel and a time offset. At 1638, receiver device 120 monitors the secondary channel after the designated time offset and receives an extended advertisement including additional missing analyte data. If this is the remainder of the missing analyte data, then the transfer is complete. If more analyte data is missing and needs to be transferred, then the extended advertisement can include a pointer designating a secondary channel (which can be the same or different) and a time offset, similar to the advertisement in step 1636. Receiver device 120 can then monitor the newly designated secondary channel after the designated time offset, and receive yet another extended advertisement containing missing analyte data similar to step 1638. This process of steps 1636 and 1638 can repeat until all missing analyte data is transferred from OBD 101 to receiver device 120.

The extended advertising technique can be used to provide setup information for a receiver device 120 seeking to join the synchronous communication session 801 with OBD 101. Advertisements (e.g., a PDU of type ADV_EXT_IND) sent on the primary channels can direct a receiver device to an extended advertisement on a secondary channel (e.g., a PDU of type AUX_ADV_IND) by supplying the secondary channel and time offset, which in turn can direct the receiver device to an advertisement 802 in session 801 (e.g., a PDU of type AUX_SYNC_IND).

The embodiments described with respect to FIGS. 16A-16D are set forth in the context of providing missing or supplemental data, e.g., as part of a backfill request. These embodiments can likewise be applied to the regular transmission and receipt of any data including data being sent for the first time that is not missing nor part of a backfill request. These embodiments can be applied to any lengthy data transmission to divide that transmission into smaller segments separated by a time gap where no transmission occurs. Such division can provide the benefit of avoiding a lengthy time period of continuous transmission, which can apply strain to a power source of a device, such as power source 320 of OBD 101. Relatively small and low voltage power supplies can, at times, be strained by a continuous current demand for a lengthy transmission, which can be avoided if the continuous current demand is divided into multiple relatively shorter duration current draws.

The embodiments and variations thereto described with respect to FIGS. 16A through 16D can be implemented along with all of the clock accuracy embodiments described herein, including each one of those embodiments described with respect to FIGS. 5A, 5B, 5C, 5D, 6A, 6B, 6C, 7A, 7B, 7C, and 7D. For example, the clock accuracy parameter of receiver devices 120-1 through 120-N can be supplied to OBD 101 by the sending of a response 804 or can be supplied to OBD 101 as part of a preceding connection establishment negotiation. OBD 101 can elect to adjust the backfilling technique based on the received clock accuracy parameter (e.g., such as by using extended advertising instead of a synchronous advertising based approach if the clock accuracy parameter is relatively low or below a threshold).

In many embodiments herein, wireless communication electronics are shown and described with respect to active states. The active state can be a state where the entirety of the communication electronics are fully powered and operating, or it can be a state where only part of the wireless communication electronics are fully powered and another part is operating in a relatively lower power, e.g., dormant or sleep, state. The specific needs of a particular implementation will often determine what level of active state the wireless communication electronics should be in, and whether that active state should continue between transmissions, between transmission and a receive window, and between receive windows. While in many cases the wireless communication electronics are described and/or depicted, such as with respect to FIGS. 8B, 8F, 9B-9E, 9G, 10B, 12A-12C, 13B-13E, 13F-1 through 13F-5, 14A-14D, 15B, and 16A, as deactivating between adjacent transmissions and/or receive windows, the present description fully encompasses embodiments where the wireless communication electronics remain active between adjacent transmissions, remain active between an adjacent transmission and receive window, remain active between adjacent receive windows, and/or any combination thereof.

The embodiments described herein can be utilized to extend the designed wear duration of on-body devices 101. The resulting extended designed wear durations will depend on the actual implementation of OBD 101 and the sensor 160 it operates in conjunction with. For OBDs 101 (and systems 100), the designed wear durations can in various embodiments be (or can be up to) 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23, days, 24 days, 25 days, 26 days, 27 days, 28 days, 29 days, 30 days, 31 days, 45 days, 60 days, 90 days, and other amounts therebetween those listed or longer. The designed wear durations can be in ranges such as 1 to 7 days, 8 to 14 days, 15 to 30 days, 30 to 45 days, and 45 days or greater.

Formulas (1) and (2) are presented herein as examples of just a few of the various ways the subject matter described herein can be beneficially utilized. Implementation of formula (1) and/or (2) is not required nor essential for the practicing of any embodiment, nor are formulas (1) and (2) required to be practiced by any claim absent explicit recitation therein.

All of the embodiments described herein with respect to wireless communications (e.g., pertaining to clock accuracy and/or synchronous advertising-based protocols) are adapted for use specifically with analyte monitoring systems 100 with small form devices, and are particularly adapted for use with wireless transmissions to and from an on-body device 101. As such, these embodiments can be differentiated from rechargeable general use systems such as those where larger form rechargeable devices (e.g., a smartphone, a vehicle, a head set, a speaker, and the like) are wirelessly communicating with another larger form rechargeable device.

The embodiments described herein are reiterated and/or supplemented in the following paragraphs which do so without explicit reference to the figures. These paragraphs reference clauses which provide explicit written support for claims of like character.
Clause A1. An on-body glucose monitoring system, comprising: an on-body device configured to be held on the body of a subject, the on-body device comprising a housing configured to hold first electronics, wherein the first electronics are configured to: interface with a sensor configured to measure a glucose level of the subject; receive a wireless communication from a receiver device having a clock, wherein the wireless communication comprises data indicative of a clock accuracy of the clock; assess the data indicative of the clock accuracy of the clock; and progress at least one power management action based on the assessment of the data indicative of the clock accuracy.
Clause A2. The system of clause A1, wherein the first electronics are configured to produce at least one of a first and a second output from the assessment of the data indicative of the clock accuracy, wherein the first output is that the clock accuracy is acceptable and the second output is that the clock accuracy is not acceptable.
Clause A3. The system of any of clauses A1 through A2, wherein the data indicative of the clock accuracy comprises an indication of a type of the receiver device.
Clause A4. The system of clause A3, wherein the indication of the type of the receiver device is an indication of the manufacturer and/or the model of the receiver device.
Clause A5. The system of any of clauses A1 through A4, wherein the data indicative of the clock accuracy of the clock comprises a clock accuracy parameter.
Clause A6. The system of clause A5, wherein the first electronics are configured to, if the clock accuracy is not acceptable, indicate a degree by which the clock accuracy differs from an acceptable value for the clock accuracy.
Clause A7. The system of any of clauses A5-A6, wherein the first electronics are configured to assess the clock accuracy parameter by a comparison of the clock accuracy parameter to a threshold accuracy value.
Clause A8. The system of any of clauses A5 through A7, wherein the first electronics are configured to assess the clock accuracy parameter by identification of the received clock parameter in a data structure.
Clause A9. The system of clause A8, wherein the data structure comprises at least one of a list, array, table, or look up table, and wherein the data structure contains values of potential clock accuracy parameters and associated indications of whether or not those clock accuracy parameters are acceptable.
Clause A10. The system of any of clauses A5 through A9, wherein the first electronics are configured to assess the clock accuracy parameter by reference to a single clock accuracy threshold for acceptability assessment during a wear duration of the on-body device.
Clause A11. The system of any of clauses A5 through A10, wherein the first electronics are configured to assess the clock accuracy parameter by reference to a plurality of clock accuracy thresholds for acceptability assessment during a wear duration of the on-body device.
Clause A12. The system of clause A11, wherein each one of the plurality of clock accuracy thresholds corresponds to a different amount of time left in a target wear duration for the on-body device.
Clause A13. The system of any of clauses A5 through A12, wherein the first electronics are configured to determine a threshold with which to assess the clock accuracy parameter.
Clause A14. The system of clause A13, wherein the first electronics are configured to determine the threshold by processing a plurality of conditions according to an algorithm, formula, or set of rules, wherein the plurality of conditions comprise a time condition and a remaining power or energy condition.
Clause A15. The system of any of clauses A5 through A14, wherein the first electronics are configured to assess the clock accuracy parameter with software executed by the first electronics.
Clause A16. The system of any of clauses A5 through A15, wherein the first electronics are configured to assess the clock accuracy parameter with hardware of the first electronics.
Clause A17. The system of any of clauses A1 through A16, wherein the first electronics are configured to progress the at least one power management action by initiation of the power management action or, if the power management action is already in effect, then by furtherance of the power management action.
Clause A18. The system of any of clauses A1 through A17, wherein the at least one power management action comprises a determination of one or more power or energy consequences to the on-body device.
Clause A19. The system of any of clauses A1 through A18, wherein the at least one power management action comprises an adjustment to an operating aspect of the on-body device.
Clause A20. The system of any of clauses A1 through A19, wherein the at least one power management action comprises a determination of a target wear duration consequence to the on-body device.
Clause A21. The system of any of clauses A1 through A20, wherein the at least one power management action comprises an issuance of a notification to a user.
Clause A22. The system of any of clauses A1 through A21, wherein the at least one power management action comprises determination of one or more power or energy consequences, optionally followed by either or both of an adjustment to an operating aspect of the on-body device and/or issuance of a notification to a user.
Clause A23. The system of any of clauses A1 through A22, wherein the at least one power management action comprises adjustment to an operating aspect of the on-body device, optionally followed by issuance of a notification to a user.
Clause A24. The system of any of clauses A1 through A23, wherein the at least one power management action comprises determination of a target wear duration consequence of the on-body device, optionally followed by either or both of adjustment to an operating aspect of the on-body device and/or issuance of a notification to a user.
Clause A25. The system of any of clauses A1 through A24, wherein the at least one power management action comprises issuance of a notification to a user, optionally followed by one, two, or all of a determination of one or more power or energy consequences, an adjustment to an operating aspect of the on-body device, and/or determination of a target wear duration consequence of the on-body device.
Clause A26. The system of any of clauses A1 through A25, wherein the at least one power management action comprises a determination of one or more power or energy consequences of the on-body device that comprises at least one of the following: a determination of a new rate of power or energy consumption, a determination of a power or energy budget, and/or a determination of an adjustment to rate of power or energy consumption.
Clause A27. The system of clause A26, wherein determination of one or more power or energy consequences of the on-body device comprises a determination of a power budget by comparison of a new cumulative amount of power required by the on-body device with a remaining power available in a power source of the on-body device.
Clause A28. The system of clause A26, wherein determination of one or more power or energy consequences of the on-body device comprises a determination of a power budget, wherein the first electronics are further configured to: adjust a new rate of power consumption for the on-body device to maintain operation of the on-body device through the end of a target wear duration; lower the target wear duration to accommodate greater use of power; or a combination of both.
Clause A29. The system of any of clauses A1 through A27, wherein the at least one power management action comprises a determination of a target wear duration consequence of the on-body device that comprises determination of a new target wear duration for the on-body device and/or determination of an adjustment to a present target wear duration of the on-body device.
Clause A30. The system of clause A29, wherein the first electronics are further configured to notify a user of the target wear duration consequence.
Clause A31. The system of any of clauses A1 through A28, wherein the at least one power management action comprises an adjustment to an operating aspect of the on-body device that comprises implementation of a new power or energy management scheme in the on-body device to conserve power or energy.
Clause A32. The system of clause A31, wherein adjustment to an operating aspect of the on-body device comprises implementation of a new power or energy management scheme in the on-body device to conserve power or energy and maintain operation of the on-body device through an entirety of a target wear duration.
Clause A33. The system of clause A31 or A32, wherein adjustment to an operating aspect of the on-body device comprises alteration of a schedule of operation to increase an amount of time that the on-body device is in a first lower power state as compared to a second higher power state.
Clause A34. The system of any of clauses A31 through A33, wherein adjustment to an operating aspect of the on-body device comprises an alteration of a schedule of operation to increase the amount of time that the on-body device is in a sleep state relative to a partially awake state or a fully awake state, wherein the sleep state, the partially awake state, and the fully awake state are states of operation of processing electronics of the on-body device.
Clause A35. The system of any of clauses A31 through A34, wherein adjustment to an operating aspect of the on-body device comprises an alteration of a schedule of operation to increase the amount of time that the on-body device is in a sleep state relative to a partially awake state or a fully awake state, wherein the sleep state, the partially awake state, and the fully awake state are states of operation of wireless communication electronics of the on-body device.
Clause A36. The system of any of clauses A31 through A35, wherein adjustment to an operating aspect of the on-body device comprises alteration of a schedule of communication with the receiver device.
Clause A37. The system of any of clauses A31 through A36, wherein adjustment to an operating aspect of the on-body device comprises a switch from a first rate of transmission of glucose level measurements to a second rate of transmission of glucose level measurements that is slower than the first rate.
Clause A38. The system of any of clauses A31 through A37, wherein adjustment to an operating aspect of the on-body device comprises alteration of a schedule of communication with the receiver device.
Clause A39. The system of any of clauses A31 through A38, wherein adjustment to an operating aspect of the on-body device comprises transition from a mode where analyte measurements are automatically transmitted from the on-body device to the receiver device to a mode where only alarms are transmitted from the on-body device to the receiver device.
Clause A40. The system of any of clauses A31 through A39, wherein adjustment to an operating aspect of the on-body device comprises an adjustment to a mode of wireless communication between the on-body device and the receiver device.
Clause A41. The system of any of clauses A31 through A40, wherein adjustment to an operating aspect of the on-body device comprises adjustment to a length of a receive window of the on-body device based on the clock accuracy of the receiver device, optionally where the adjustment is to increase a length of the receive window if the clock accuracy of the receiver device fails to satisfy a threshold.
Clause A42. The system of any of clauses A31 through A40, wherein adjustment to an operating aspect of the on-body device comprises a transition from a first mode of communication to a second mode of communication, wherein the first mode of communication has a first power requirement, the second mode of communication has a second power requirement, and the second power requirement is less than the first power requirement.
Clause A43. The system of clause A42, wherein the first mode of communication is a connection-oriented mode of communication and the second mode of communication is a connection-less mode of communication.
Clause A44. The system of clause A42, wherein the first mode of communication is a connection-less mode of communication and the second mode of communication is a connection-oriented mode of communication.
Clause A45. The system of any of clauses A41 through A44, wherein the second power requirement is less than the first power requirement at least in part due to a slower rate of transmission for the second mode as compared to the first mode.
Clause A46. The system of any of clauses A1 through A45, wherein the at least one power management action is an adjustment to an operating aspect of the on-body device that comprises: a modification to a rate of transmissions from the on-body device to the receiver device; and a change to a mode of communication between the on-body device and the receiver device.
Clause A47. The system of any of clauses A31 through A46, wherein the on-body device is configured to adjust an operating aspect during a time period of relative inactivity by a user.
Clause A48. The system of any of clauses A31 through A47, wherein the on-body device is configured to adjust an operating aspect during a time period wherein the on-body device has detected a temperature threshold violation.
Clause A49. The system of clause A48, wherein the on-body device comprises a temperature sensor and is configured to detect a temperature threshold violation with the temperature sensor.
Clause A50. The system of any of clauses A1 through A46, wherein the at least one power management action comprises the issuance of a notification to a user.
Clause A51. The system of any of clauses A1 through A50, wherein the at least one power management action progressed by the on-body device comprises the issuance of a notification to a user on a display of the receiver device.
Clause A52. The system of clause A50 or A51, wherein the first electronics are configured to send an instruction to the receiver device to issue the notification.
Clause A53. The system of clause A51, further comprising the receiver device, wherein the receiver device comprises a display.
Clause A54. The system of any one of clauses A50 through A53, wherein the notification comprises a window, a screen, and/or a banner on the display.
Clause A55. The system of any of clauses A53 or A54, wherein the notification comprises a modification to an icon or indicator on the display.
Clause A56. The system of any of clauses A53 through A55, wherein the receiver device comprises a speaker, and wherein the notification comprises an audible notification.
Clause A57. The system of any of clauses A53 through A56, wherein the receiver device is configured to produce haptic motion, and wherein the notification comprises a vibration.
Clause A58. The system of clause A51, wherein the notification comprises an alarm or alert.
Clause A59. The system of any of clauses A51 through A58, wherein the receiver device comprises a display, and wherein the notification comprises at least one of the following: information to a user of a result of utilization of the receiver device having an unacceptable clock accuracy; a recommendation that a user take an action; a recommendation that a user cease use of the receiver device having an unacceptable clock accuracy; a recommendation that a user switch to a second receiver device having a relatively higher quality clock accuracy; an indication that the receiver device has an insufficient quality level or clock accuracy; a revision to a target wear duration of the on-body device; a notification to a user that a remaining target wear duration of the on-body device has been adjusted; a notification of a change to a state of operation of the on-body device; a notification of a change to a communication schedule of the on-body device; and/or a notification of a change to a glucose measurement transmission rate of the on-body device.
Clause A60. The system of any of clauses A1 through A59, wherein the power management action is the issuance of a notification to the user that comprises a prompt or query to the user.
Clause A61. The system of any of clauses A1 through A60, wherein the power management action is the issuance of a notification to the user that comprises a prompt or query to the user, a response to which indicates whether an additional power management action to be taken.
Clause A62. The system of any of clauses A1 through A61, wherein the at least one power management action progressed by the on-body device comprises the issuance of a notification to a user by a user interface of the on-body device.
Clause A63. The system of any of clauses A1 through A62, wherein the first electronics are configured to send a wireless request to the receiver device for the clock accuracy parameter.
Clause A64. The system of any of clauses A1 through A63, wherein the clock accuracy parameter is an actual value of the clock accuracy, a range or a limit of the clock accuracy, or a code that is representative of the actual value, the range, or the limit of the clock accuracy.
Clause A65. The system of any of clauses A1 through A64, further comprising a receiver device comprising second electronics configured to process data and wirelessly communicate with the on-body device.
Clause A66. The system of clause A65, wherein the second electronics are configured to execute an application on the receiver device.
Clause A67. The system of clause A66, wherein the application is configured to request the data indicative of the clock accuracy as part of a programmed routine of the application, wherein the programmed routine is configured for execution upon installation of the application on the receiver device, or wherein the programmed routine is configured for execution after establishing communication with the on-body device.
Clause A68. The system of clause A67, wherein the application is configured to transmit the data indicative of the clock accuracy automatically without an independent requisition from the on-body device.
Clause A69. The system of any of clauses A1 through A68, further comprising the receiver device configured to progress a power management action.
Clause A70. The system of clause A69, wherein the receiver device is a multi-purpose mobile receiver device, a dedicated receiver device, or drug delivery device.
Clause A71. The system of clause A69 or A70, wherein the receiver device comprises second electronics that comprise processing electronics, wireless communication electronics, wired communication electronics; and the clock in communication with each other.
Clause A72. The system of any of clauses A69 through A71, wherein the receiver device is configured to wirelessly communicate with one or more other receiver devices and a remote server over a network.
Clause A73. The system of clause A72, wherein the remote server is configured to perform a power management action and communicate a result to the receiver device.
Clause A74. The system of any of clauses A1 through A73, wherein the on-body device comprises the sensor configured to measure a glucose level of the subject.
Clause A75. The system of clause A74, wherein the sensor is partially implantable and configured to measure the glucose level in a bodily fluid within a body of the subject.
Clause A76. The system of any of clauses A1 through A75, comprising the sensor configured to measure the glucose level while wholly implanted within a body of the subject.
Clause A77. The system of clause A76, wherein the first electronics are configured to wirelessly communicate with the wholly implantable sensor.
Clause A78. The system of any of clauses A1 through A77, wherein the first electronics of the on-body device comprises sensor interface electronics, processing electronics, wireless communication electronics, and memory on which a plurality of instructions are stored that are executable by the processing electronics.
Clause A79. The system of clause A78, wherein the plurality of instructions, when executed by the processing electronics, are configured to assess the clock accuracy parameter and progress the power management action.
Clause A80. The system of clause A78 or A79, wherein the wireless communication electronics comprise an antenna communicatively coupled with a transceiver.
Clause A81. The system of any of clauses A1 through A80, further comprising an adhesive pad coupled with the housing of the on-body device.
Clause A82. The system of any of clauses A1 through A81, wherein the on-body device is configured to receive the wireless communication from the receiver device according to one of the following protocols: Wi-Fi, Bluetooth, Bluetooth Low Energy, Zigbee, Z-Wave, Near Field Communication (NFC), Ultra-Wide Band (UWB), or a Personal Area Network (PAN) protocol.
Clause A83. The system of any of clauses A1 through A82, wherein the on-body device is configured to receive the wireless communication from the receiver device according to a non-standard protocol.
Clause A84. The system of any of clauses A1 through A83, wherein the on-body device is configured to perform wireless communications with the receiver device according to a Bluetooth Low Energy protocol.
Clause A85. The system of any of clauses A1 through A84, wherein the on-body device is configured to perform wireless communications with the receiver device according to a Near Field Communication (NFC) protocol.
Clause A86. The system of any of clauses A1 through A85, wherein the on-body device is configured to perform wireless communications with the receiver device according to a Near Field Communication (NFC) protocol and a Bluetooth Low Energy protocol.
Clause A87. The system of any of clauses A1 through A86, wherein the on-body device comprises a clock configured to provide a clock signal for timing wireless communications with the receiver device.
Clause A88. The system of clause A87, wherein the clock of the on-body device has a clock accuracy of 40 parts per million or less.
Clause A89. The system of any of clauses A1 through A88, wherein the on-body device is configured to receive the wireless communication from the receiver device according to one of the following protocols: Bluetooth; Bluetooth Low-Energy (“BLE”); Near-Field Communication (“NFC”); ultra-wideband (UWB); mobile protocols (e.g., Fifth Generation (5G) technology, Long Term Evolution (LTE), Global System for Mobile Communications (GSM), Code Division Multiple Access (CDMA)); Zigbee; Z-Wave; a personal area network according to Institute of Electrical and Electronics Engineers (IEEE) 802.15 protocols, Adaptive Network Topology (ANT), or IPv6 over Low-Power Wireless Personal Area Networks (6LoWPAN); IEEE 802.11 protocols (e.g., 802.11a, 802.11b, 802.11g, 802.11n (aka Wi-Fi 4), 802.11ac (aka Wi-Fi 5), 802.11ah (Wi-Fi HaLow), 802.11ax (aka Wi-Fi 6)); infrared communications according to the Infrared Data Association standards (IrDA); Light Fidelity (Li-Fi); low-power wide-area network (LPWAN) protocols such as LoRa (Long Range); and wireless communication protocols that are proprietary, customized, and not subject to standardization by an international, national, nor regional standard setting body.
Clause A90. The system of any of clauses A1 through A89, where the sensor is further configured to measure a second analyte level of the subject.
Clause A91. The system of any of clauses A1 through A90, where the sensor is further configured to measure a ketone level of the subject.
Clause A92. The system of any of clauses A1 through A91, wherein the on-body device has a target wear duration referenceable by the first electronics, wherein the target wear duration is between 15 and 30 days.
Clause A93. The system of any of clauses A1 through A92, wherein the on-body device has a target wear duration referenceable by the first electronics, wherein the target wear duration is between 30 and 45 days.
Clause A94. The system of any of clauses A1 through A93, wherein the on-body device has a target wear duration referenceable by the first electronics, wherein the target wear duration is 45 days or greater.
Clause A95. The system of any of clauses A1 through A94, wherein the on-body device further comprises:
a sensor; and
an adhesive patch adhesively coupled with the housing,
wherein the first electronics comprise: wireless communication electronics; and sensor interface electronics configured to receive a signal from the sensor, the signal indicative of a glucose level of a user;
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the glucose level in the bodily fluid and output the signal to the sensor interface electronics
Clause A96. An on-body analyte monitoring system, comprising: an on-body device configured to be held on the body of a subject, the on-body device comprising a housing configured to hold first electronics, wherein the first electronics are configured to: interface with a sensor configured to measure a level of an analyte of the subject; receive a wireless communication from a receiver device having a clock, wherein the wireless communication comprises data indicative of a clock accuracy of the clock; assess the data indicative of the clock accuracy of the clock; and progress at least one power management action based on the assessment of the data indicative of a clock accuracy of the clock.
Clause A97. The system of clause A96, wherein the analyte is selected from the list consisting of: glucose, one or more ketones, lactate, and oxygen.
Clause A98. The system of clause A96, wherein the analyte is selected from the list consisting of: ammonium, asparagine, aspartate, cholesterol, choline, creatinine, one or more diaphorase inhibitors, ethanol, glucose, glutamate, glycerol, one or more ketones, lactate, lactose, oxygen, phosphate, potassium, pyruvate, and uric acid.
Clause A99. The system of clause A96, wherein the analyte is a first analyte, and the sensor is further configured to measure a level of a second analyte of the subject.
Clause A100. The system of clause A96, wherein the first analyte is glucose and the second analyte is a ketone.
Clause A101. The system of clause A96, wherein the first and second analytes are selected from the list consisting of: ammonium, asparagine, aspartate, cholesterol, choline, creatinine, one or more diaphorase inhibitors, ethanol, glucose, glutamate, glycerol, one or more ketones, lactate, lactose, oxygen, phosphate, potassium, pyruvate, and uric acid.
Clause A102. The system of any of clauses A96 through A101, wherein the on-body device further comprises:
a sensor; and
an adhesive patch adhesively coupled with the housing,
wherein the first electronics comprise: wireless communication electronics; and sensor interface electronics configured to receive a signal from the sensor, the signal indicative of an analyte level of a user;
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the analyte level in the bodily fluid and output the signal to the sensor interface electronics
Clause B1. A method of power management in an on-body glucose monitoring system, comprising: receiving, by an on-body device, a wireless communication from a receiver device having a clock, wherein the wireless communication comprises data indicative of the clock accuracy of the clock; assessing the data indicative of the clock accuracy of the clock; and progressing at least one power management action based on the assessment of the data indicative of the clock accuracy of the clock.
Clause B2. The method of clause B1, further comprising outputting that the clock accuracy is acceptable or not acceptable based on the assessment of the data indicative of the clock accuracy of the clock.
Clause B3. The method of any of clauses B1 through B #, wherein the data indicative of the clock accuracy comprises an indication of a type of the receiver device.
Clause B4. The method of clause B3, wherein the indication of the type of the receiver device is an indication of the manufacturer and/or the model of the receiver device.
Clause B5. The method of any of clauses B1 through B #, wherein the data indicative of the clock accuracy of the clock comprises a clock accuracy parameter.
Clause B6. The method of clause B5, wherein, if the clock accuracy is not acceptable, indicating a degree by which the clock accuracy differs from an acceptable value for the clock accuracy.
Clause B7. The method of any of clauses B5 or B6, further comprising assessing the clock accuracy parameter by comparing the clock accuracy parameter to a threshold accuracy value.
Clause B8. The method of any of clauses B5 through B7, further comprising assessing the clock accuracy parameter by identifying the received clock parameter in a data structure.
Clause B9. The method of clause B8, wherein the data structure is accessible from a memory of the on-body device.
Clause B10. The method of any of clauses B5 through B9, further comprising assessing the clock accuracy parameter by referencing a single clock accuracy threshold for acceptability assessment during a wear duration of the on-body device.
Clause B11. The method of any of clauses B5 through B10, further comprising assessing the clock accuracy parameter by referencing a plurality of clock accuracy thresholds for acceptability assessment during a wear duration of the on-body device.
Clause B12. The method of clause B11, wherein each one of the plurality of clock accuracy thresholds each clock accuracy corresponds to a different amount of time left in a target wear duration of the on-body device.
Clause B13. The method of any of clauses B5 through B12, further comprising determining a threshold with which to assess the clock accuracy parameter.
Clause B14. The method of clause B13, further comprising determining the threshold by processing a plurality of conditions according to an algorithm, formula, or set of rules.
Clause B15. The method of clause B14, wherein the plurality of conditions comprises a time condition and a remaining power or energy condition.
Clause B16. The method of any of clauses B1 through B15, further comprising progressing the at least one power management action by initiating the power management action or, if the power management action is already in effect, then by furthering the power management action.
Clause B17. The method of any of clauses B1 through B16, wherein progressing the at least one power management action comprises determining one or more power or energy consequences to the on-body device.
Clause B18. The method of any of clauses B1 through B17, wherein progressing the at least one power management action comprises adjusting an operating aspect of the on-body device.
Clause B19. The method of any of clauses B1 through B18, wherein progressing the at least one power management action comprises determining a target wear duration consequence to the on-body device.
Clause B20. The method of any of clauses B1 through B19, wherein progressing the at least one power management action comprises issuing a notification to a user.
Clause B21. The method of any of clauses B1 through B20, wherein the at least one power management action comprises determining one or more power or energy consequences, optionally followed by either or both of adjusting an operating aspect of the on-body device and/or issuing of a notification to a user.
Clause B22. The method of any of clauses B1 through B21, wherein the at least one power management action comprises adjusting an operating aspect of the on-body device, optionally followed by issuing a notification to a user.
Clause B23. The method of any of clauses B1 through B22, wherein the at least one power management action comprises determining a target wear duration consequence of the on-body device, optionally followed by either or both of adjusting an operating aspect of the on-body device and/or issuing a notification to a user.
Clause B24. The method of any of clauses B1 through B23, wherein the at least one power management action comprises issuing a notification to a user, optionally followed by one, two, or all of determining one or more power or energy consequences, adjusting an operating aspect of the on-body device, and/or determining a target wear duration consequence of the on-body device.
Clause B25. The method of any of clauses B1 through B24, wherein the at least one power management action comprises determining one or more power or energy consequences of the on-body device that comprises at least one of the following: determining a new rate of power or energy consumption, determining a power or energy budget, and/or determining an adjustment to rate of power or energy consumption.
Clause B26. The method of clause B25, wherein determining one or more power or energy consequences of the on-body device comprises determining a power budget by comparing a new cumulative amount of power required by the on-body device with a remaining power available in a power source of the on-body device.
Clause B27. The method of clause B25, wherein determining one or more power or energy consequences of the on-body device comprises determining a power budget, wherein the method further comprises adjusting a new rate of power consumption for the on-body device to maintain operation of the on-body device through the end of a target wear duration; lowering the target wear duration to accommodate greater use of power; or both.
Clause B28. The method of any of clauses B1 through B27, wherein the at least one power management action comprises determining a target wear duration consequence of the on-body device that comprises determining a new target wear duration for the on-body device and/or determining an adjustment to a present target wear duration of the on-body device.
Clause B29. The method of clause B28, wherein the first electronics are further configured to notify a user of the target wear duration consequence.
Clause B30. The method of any of clauses B1 through B29, wherein the at least one power management action comprises adjusting an operating aspect of the on-body device that comprises implementing a new power or energy management scheme in the on-body device to conserve power or energy.
Clause B31. The method of clause B30, wherein adjusting an operating aspect of the on-body device comprises implementing a new power or energy management scheme in the on-body device to conserve power or energy and maintain operation of the on-body device through an entirety of a target wear duration.
Clause B32. The method of clause B30 or B31, wherein adjusting an operating aspect of the on-body device comprises altering a schedule of operation to increase an amount of time that the on-body device is in a first lower power state as compared to a second higher power state.
Clause B33. The method of any of clauses B30 through B32, wherein adjusting an operating aspect of the on-body device comprises altering a schedule of operation to increase the amount of time that the on-body device is in a sleep state relative to a partially awake state or a fully awake state.
Clause B34. The method of any of clauses B30 through B33, wherein adjusting an operating aspect of the on-body device comprises altering a schedule of communication with the receiver device.
Clause B35. The method of any clauses B30 through B34, wherein adjusting an operating aspect of the on-body device comprises switching from a first rate of transmission of glucose level measurements to a second rate of transmission of glucose level measurements that is slower than the first rate.
Clause B36. The method of any of clauses B30 through B35, wherein adjusting an operating aspect of the on-body device comprises altering a schedule of communication with the receiver device.
Clause B37. The method of any of clauses B30 through B36, wherein adjusting an operating aspect of the on-body device comprises transitioning from a mode where analyte measurements are automatically transmitted from the on-body device to the receiver device to a mode where only alarms are transmitted from the on-body device to the receiver device.
Clause B38. The method of any of clauses B30 through B37, wherein adjusting an operating aspect of the on-body device comprises adjusting a mode of wireless communication between the on-body device and the receiver device.
Clause B39. The method of any of clauses B30 through B38, wherein adjusting an operating aspect of the on-body device comprises transitioning from a first mode of communication having a first power requirement to a second mode of communication having a second power requirement, wherein the second power requirement is less than the first power requirement.
Clause B40. The method of clause B39, wherein the first mode of communication is a connection-oriented mode of communication and the second mode of communication is a connection-less mode of communication.
Clause B41. The method of clause B39, wherein the first mode of communication is a connection-less mode of communication and the second mode of communication is a connection-oriented mode of communication.
Clause B42. The method of any of clauses B39 through B41, wherein the second power requirement is less than the first power requirement at least in part due to a slower rate of transmission for the second mode as compared to the first mode.
Clause B43. The method of any of clauses B1 through B42, wherein the at least one power management action comprises adjusting an operating aspect of the on-body device by modifying a rate of transmissions from the on-body device to the receiver device; and changing a mode of communication between the on-body device and the receiver device.
Clause B44. The method of any of clauses B30 through B43, wherein the at least one power management action comprises adjusting an operating aspect of the on-body device during a first time period of relatively less activity by a user as compared to a second time period.
Clause B45. The method of any of clauses B30 through B44, wherein the at least one power management action comprises adjusting an operating aspect of the on-body device during a time period wherein the on-body device has detected a temperature threshold violation.
Clause B46. The method of clause B45, wherein the on-body device comprises a temperature sensor and is configured to detect a temperature threshold violation with the temperature sensor.
Clause B47. The method of any of clauses B1 through B43, wherein the at least one power management action comprises issuing a notification to a user.
Clause B48. The method of any of clauses B1 through B47, wherein the at least one power management action progressed by the on-body device comprises issuing a notification to a user on a display of the receiver device.
Clause B49. The method of clauses B47 or B48, further comprising wirelessly sending an instruction from the on-body device to the receiver device to issue the notification.
Clause B50. The method of any of clauses B47 through B49, wherein issuing the notification further comprises modifying an icon or indicator on the display.
Clause B51. The method of any of clauses B47 through B50, wherein issuing the notification further comprises issuing an audible notification.
Clause B52. The method of any of clauses B47 through B51, wherein issuing the notification further comprises vibrating the receiver device.
Clause B53. The method of any of clauses B47 through B52, wherein the notification comprises at least one of the following: information to a user of a result of utilization of the receiver device having an unacceptable clock accuracy; a recommendation that a user take an action; a recommendation that a user cease use of the receiver device having an unacceptable clock accuracy; a recommendation that a user switch to a second receiver device having a relatively higher quality clock accuracy; an indication that the receiver device has an insufficient quality level or clock accuracy; a revision to a target wear duration of the on-body device; a notification to a user that a remaining target wear duration of the on-body device has been adjusted; a notification of a change to a state of operation of the on-body device; a notification of a change to a communication schedule of the on-body device; an alarm or alert; and/or a notification of a change to a glucose measurement transmission rate of the on-body device.
Clause B54. The method of any of clauses B1 through 53 wherein the power management action comprises issuing a notification to the user that comprises a prompt or query to the user.
Clause B55. The method of any of clauses B1 through B54, wherein the power management action is issuing a notification to the user that comprises a prompt or query to the user, the method further comprising receiving a response that indicates whether an additional power management action to be taken.
Clause B56. The method of any of clauses B1 through B55, wherein the at least one power management action progressed by the on-body device comprises issuing a notification to a user by a user interface of the on-body device.
Clause B57. The method of any of clauses B1 through B56, further comprising sending a wireless request from the on-body device to the receiver device for the data indicative of the clock accuracy.
Clause B58. The method of any of clauses B1 through B57, wherein the data indicative of the clock accuracy comprises a clock accuracy parameter that is an actual value of the clock accuracy, a range or a limit of the clock accuracy, or a code that is representative of the actual value, the range, or the limit of the clock accuracy.
Clause B59. The method of any of clauses B1 through B58, further comprising executing an application on the receiver device and transmitting the data indicative of the clock accuracy as part of a programmed routine of the application, wherein the programmed routine is executed upon installation of the application on the receiver device, or wherein the programmed routine is executed after establishing communication with the on-body device.
Clause B60. The method of any of clauses B1 through B56, further comprising transmitting the data indicative of the clock accuracy automatically to the on-body device without an independent requisition from the on-body device.
Clause B61. The method of any of clauses B1 through B60, further comprising progressing a power management action by the receiver device.
Clause B62. The method of any of clauses B1 through B61, wherein glucose measurements are wirelessly communicated from the on-body device to the receiver according to one of the following protocols: Wi-Fi, Bluetooth, Bluetooth Low Energy, Zigbee, Z-Wave, Near Field Communication (NFC), Ultra-Wide Band (UWB), or a Personal Area Network (PAN) protocol.
Clause B63. The method of any of clauses B1 through B62, wherein the on-body device is configured to wirelessly communicate with the receiver device according to a Bluetooth Low Energy protocol.
Clause B64. The method of any of clauses B1 through B63, wherein the on-body device is configured to wirelessly communicate with the receiver device according to a Near Field Communication (NFC) protocol.
Clause B65. The method of any of clauses B1 through B64, wherein the on-body device is configured to wirelessly communicate with the receiver device according to a non-standard protocol.
Clause B66. The method of any of clauses B1 through B65, wherein the on-body device is configured to wirelessly communicate with the receiver device according to at least two of the following: a Bluetooth protocol, a Bluetooth Low Energy Protocol, a Near-Field Communication (NFC) protocol, and a Wi-Fi protocol.
Clause B67. The method of any of clauses B1 through B66, wherein the on-body device is configured to perform wireless communications with the receiver device according to a Near Field Communication (NFC) protocol and a Bluetooth Low Energy protocol.
Clause B68. The method of any of clauses B1 through B67, wherein the on-body device is configured to receive the wireless communication from the receiver device according to one of the following protocols: Bluetooth; Bluetooth Low-Energy (“BLE”); Near-Field Communication (“NFC”); ultra-wideband (UWB); mobile protocols (e.g., Fifth Generation (5G) technology, Long Term Evolution (LTE), Global System for Mobile Communications (GSM), Code Division Multiple Access (CDMA)); Zigbee; Z-Wave; a personal area network according to Institute of Electrical and Electronics Engineers (IEEE) 802.15 protocols, Adaptive Network Topology (ANT), or IPv6 over Low-Power Wireless Personal Area Networks (6LoWPAN); IEEE 802.11 protocols (e.g., 802.11a, 802.11b, 802.11g, 802.11n (aka Wi-Fi 4), 802.11ac (aka Wi-Fi 5), 802.11ah (Wi-Fi HaLow), 802.11ax (aka Wi-Fi 6)); infrared communications according to the Infrared Data Association standards (IrDA); Light Fidelity (Li-Fi); low-power wide-area network (LPWAN) protocols such as LoRa (Long Range); and wireless communication protocols that are proprietary and not subject to standardization by an international, national, nor regional standard setting body.
Clause B69. The method of any of clauses B1 through B68, further comprising measuring a glucose level of a subject with a sensor operating in conjunction with the on-body device.
Clause B70. The method of clause B69, further comprising measuring a ketone level of the subject with the sensor.
Clause B71. The system of any of clauses B1 through B70, wherein the on-body device comprises:
a sensor;
wireless communication electronics;
sensor interface electronics configured to receive a signal from the sensor, the signal indicative of a glucose level of a user;
a housing configured to retain the wireless communication electronics and sensor interface electronics; and
an adhesive patch adhesively coupled with the housing,
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the glucose level in the bodily fluid and output the signal to the sensor interface electronics
Clause B72. A method of power management in an on-body analyte monitoring system, comprising: receiving, by an on-body device, a wireless communication from a receiver device having a clock, wherein the wireless communication comprises data indicative of the clock accuracy of the clock; assessing the data indicative of the clock accuracy of the clock; and progressing at least one power management action based on the assessment of the data indicative of the clock accuracy of the clock.
Clause B73. The method of clause B72, further comprising measuring an analyte level by a sensor operating in conjunction with the on-body device, wherein the analyte is selected from the list consisting of: glucose, one or more ketones, lactate, and oxygen.
Clause B74. The method of clause B72, further comprising measuring an analyte level by a sensor operating in conjunction with the on-body device, wherein the analyte is selected from the list consisting of: ammonium, asparagine, aspartate, cholesterol, choline, creatinine, one or more diaphorase inhibitors, ethanol, glucose, glutamate, glycerol, one or more ketones, lactate, lactose, oxygen, phosphate, potassium, pyruvate, and uric acid.
Clause B75. The method of clause B72, further comprising measuring a first analyte level and a second analyte level by a sensor operating in conjunction with the on-body device.
Clause B76. The method of clause B75, wherein the first analyte is glucose and the second analyte is a ketone.
Clause B77. The method of clause B75, wherein the first and second analytes are selected from the list consisting of: ammonium, asparagine, aspartate, cholesterol, choline, creatinine, one or more diaphorase inhibitors, ethanol, glucose, glutamate, glycerol, one or more ketones, lactate, lactose, oxygen, phosphate, potassium, pyruvate, and uric acid.
Clause B78. The method of any of clauses B72 through B77, wherein the on-body device comprises:
a sensor;
wireless communication electronics;
sensor interface electronics configured to receive a signal from the sensor, the signal indicative of an analyte level of a user;
a housing configured to retain the wireless communication electronics and sensor interface electronics; and
an adhesive patch adhesively coupled with the housing,
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the level of the analyte in the bodily fluid and output the signal to the sensor interface electronics.
Clause C1. An on-body glucose monitoring system, comprising: a receiver device comprising a display, processing electronics, a clock having a clock accuracy, memory communicatively coupled with the processor electronics, and a glucose monitoring software application stored in the memory, wherein the glucose monitoring software application is executable by the processor electronics to: read glucose data received from an on-body device on a subject; output a glucose level for presentation on the display; assess the clock accuracy; and progress a power management action based on the assessment of the clock accuracy.
Clause C2. The system of clause C1, wherein the receiver device is configured to operate a protocol stack comprising an application layer and a lower layer, and the application is configured to issue a request from the application layer to the lower layer for a clock accuracy parameter indicative of the clock accuracy, and the lower layer is configured to output the clock accuracy parameter to the application layer.
Clause C3. The system of clause C2, comprising a host-controller interface between the application layer and the lower layer through which the request passes.
Clause C4. The system of any of clauses C1 through C3, wherein the power management action is a determination of one or more power or energy consequences of the on-body device.
Clause C5. The system of clause C4, wherein the receiver device is configured to wirelessly communicate the one or more power or energy consequences to the on-body device.
Clause C6. The system of any of clauses C1 through C5, wherein the power management action is a determination of an adjustment to an operating aspect of the on-body device.
Clause C7. The system of clause C6, wherein the receiver device is configured to instruct the on-body device to implement the adjustment by a wireless communication.
Clause C8. The system of any of clauses C1 through C7, wherein the power management action is a determination of a target wear duration consequence of the on-body device.
Clause C9. The system of clause C8, wherein the receiver device is configured to wirelessly communicate the one or more target wear duration consequences to the on-body device.
Clause C10. The system of any of clauses C1 through C9, wherein the receiver device is configured to determine a notification to be issued to a user and issue the notification to the user.
Clause C11. The system of any of clauses C1 through C10, wherein the receiver device is a smart-phone or smart-wearable device.
Clause C12. The system of any of clauses C1 through C11, further comprising the on-body device.
Clause C13. The system of any of clauses C1 through C12, wherein the on-body device is configured to receive the wireless communication from the receiver device according to one of the following protocols: Wi-Fi, Bluetooth, Bluetooth Low Energy, Zigbee, Z-Wave, Near Field Communication (NFC), Ultra-Wide Band (UWB), or a Personal Area Network (PAN) protocol.
Clause C14. The system of any of clauses C1 through C13, wherein the on-body device is configured to wirelessly communicate with the receiver device according to at least two of the following: a Bluetooth protocol, a Bluetooth Low Energy Protocol, a Near-Field Communication (NFC) protocol, and a Wi-Fi protocol.
Clause C15. The system of any of clauses C1 through C14, wherein the on-body device is configured to receive the wireless communication from the receiver device according to a non-standard protocol.
Clause C16. The system of any of clauses C1 through C15, wherein the on-body device is configured to perform wireless communications with the receiver device according to a Bluetooth Low Energy protocol.
Clause C17. The system of any of clauses C1 through C16, wherein the on-body device is configured to perform wireless communications with the receiver device according to a Near Field Communication (NFC) protocol.
Clause C18. The system of any of clauses C1 through C17, wherein the on-body device is configured to perform wireless communications with the receiver device according to a Near Field Communication (NFC) protocol and a Bluetooth Low Energy protocol.
Clause C19. The system of any of clauses C1 through C18, further comprising the on-body device, wherein the on-body device comprises:
a sensor;
wireless communication electronics;
sensor interface electronics configured to receive a signal from the sensor, the signal indicative of a glucose level of a user;
a housing configured to retain the wireless communication electronics and sensor interface electronics; and
an adhesive patch adhesively coupled with the housing,
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the glucose level in the bodily fluid and output the signal to the sensor interface electronics.
Clause C20. An on-body analyte monitoring system, comprising: a receiver device comprising a display, processing electronics, a clock having a clock accuracy, memory communicatively coupled with the processor electronics, and an analyte monitoring software application stored in the memory, wherein the analyte monitoring software application is executable by the processor electronics to: read analyte data received from an on-body device on a subject; output an analyte level for presentation on the display; assess the clock accuracy; and progress a power management action based on the assessment of the clock accuracy.
Clause C21. The system of clause C20, further comprising the on-body device, wherein the on-body device comprises:
a sensor;
wireless communication electronics;
sensor interface electronics configured to receive a signal from the sensor, the signal indicative of an analyte level of a user;
a housing configured to retain the wireless communication electronics and sensor interface electronics; and
an adhesive patch adhesively coupled with the housing,
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the analyte level in the bodily fluid and output the signal to the sensor interface electronics.
Clause D1. A method of power management in an on-body glucose monitoring system, comprising: assessing a clock accuracy of a receiver device by an application being executed on the receiver device, wherein the application is configured to monitor glucose levels of a subject; and progressing a power management action based on the assessment of the clock accuracy.
Clause D2. The method of clause D1, further comprising issuing a request from an application layer to a lower layer for a clock accuracy parameter indicative of the clock accuracy, and outputting the clock accuracy parameter from the lower layer to the application layer.
Clause D3. The method of any of clauses D1 through D2, wherein progressing the power management action comprises determining one or more power or energy consequences of the on-body device.
Clause D4. The method of clause D3, further comprising wirelessly communicating the one or more power or energy consequences to the on-body device.
Clause D5. The method of any of clauses D1 through D4, wherein progressing the power management action comprises determining an adjustment to an operating aspect of the on-body device.
Clause D6. The method of clause D5, further comprising instructing the on-body device to implement the adjustment by a wireless communication.
Clause D7. The method of any of clauses D1 through D6, wherein progressing the power management action comprises determining a target wear duration consequence of the on-body device.
Clause D8. The method of clause D7, further comprising wirelessly communicating the one or more target wear duration consequences to the on-body device.
Clause D9. The method of any of clauses D1 through D8, further comprising determining a notification to be issued to a user and issuing the notification to the user.
Clause D10. A method of power management in an on-body analyte monitoring system, comprising: assessing a clock accuracy of a receiver device by an application executing on the receiver device, wherein the application is configured to monitor analyte levels of a subject; and progressing a power management action based on the assessment of the clock accuracy.
Clause E1. A method of wireless communication in an on-body glucose monitoring system, comprising: wirelessly receiving, by an on-body device, data indicative of a first clock accuracy of a clock of a first receiver device; assessing the data indicative of the first clock accuracy; and determining to transmit a glucose measurement to a second receiver device on the basis of the assessed data indicative of the first clock accuracy.
Clause E2. The method of clause E1, wherein the data indicative of the first clock accuracy of the clock of the first receiver device is a first clock accuracy parameter of the clock or an indication of a type of the first receiver device.
Clause E3. The method of clause E1 or E2, wherein the first receiver device is a smart phone, smart wearable device, or tablet computer.
Clause E4. The method of any of clauses E1 through E3, wherein the second receiver device is a dedicated receiver device or a drug delivery device.
Clause E5. The method of any of clauses E1 through E3, wherein the second receiver device is a second smart phone, a second smart wearable device, or a second tablet computer, and has a second clock accuracy higher than the first clock accuracy.
Clause E6. The method of any of clauses E1 through E5, wherein the second receiver device is known to the on-body device as having a higher clock accuracy than the first receiver device.
Clause E7. The method of any of clauses E1 through E6, further comprising: wirelessly receiving, by the on-body device, a communication from the second receiver device that indicates that the second receiver device is of a type having a minimum acceptable clock accuracy.
Clause E8. The method of any of clauses E1 through E7, further comprising: prior to determining to transmit the glucose measurement to the second receiver device, wirelessly receiving, by the on-body device, a second clock accuracy parameter from the second receiver device, wherein the second clock accuracy parameter is indicative of a clock accuracy of a clock of the second receiver device.
Clause E9. The method of clause E8, wherein assessing the data indicative of the first clock accuracy comprises assessing the data indicative of the first clock accuracy relative to the second clock accuracy parameter.
Clause E10. The method of any of clauses E8 or E9, wherein the second receiver device is a smart phone, a smart wearable device, or a tablet computer.
Clause E11. The method of any of clauses E1 through E10, further comprising wirelessly transmitting the glucose measurement to the second receiver device.
Clause E12. The method of clause E10 or E11, further comprising communicating the glucose measurement from the second receiver device to the first receiver device.
Clause E13. The method of clause E11 or E12, further comprising communicating the glucose measurement from the second receiver device to a third receiver device.
Clause E14. The method of any of clauses E1 through E11, further comprising: designating, by the on-body device, the second receiver device as a central point of communication for reporting glucose measurement data; wirelessly transmitting a succession of glucose measurements from the on-body device to the second receiver device.
Clause E15. The method of clause E14, further comprising wirelessly transmitting, by the second receiver device, the succession of glucose measurements to the first receiver device.
Clause E16. The method of any of clauses E1 through E15, wherein the first receiver device is authorized to receive a glucose measurement from the on-body device.
Clause E17. The method of any of clauses E1 through E16, further comprising performing authentication processes to authorize the first receiver device and the second receiver device to communicate with the on-body device.
Clause E18. The method of any of clauses E1 through E17, further comprising granting concurrent permission to allow the first and second receiver devices to receive and read glucose measurement data from the on-body device.
Clause E19. The method of any of clauses E1 through E18, wherein the glucose measurement is transmitted only to the second receiver device and not the first receiver device on the basis of the first clock accuracy parameter.
Clause E20. The method of any of clauses E1 through E19, further comprising issuing at least one notification to the user.
Clause E21. The method of clause E20, wherein the at least one notification includes one or more of the following: a notification that the on-body device is transmitting the glucose measurement to the second receiver device; a notification of the result of utilization of the system with a receiver device having an unacceptable clock accuracy; a recommendation that the user take an action; a notification that one of the first and second receiver devices having the higher clock accuracy will be designated as a central point of communication for receipt of glucose measurement data; a prompt for the user's permission to treat one of the first and second receiver devices as a central point of communication; a notification of a change in a target wear duration of the on-body device if the user continues with use of the one of the first and second receiver devices having a lower clock accuracy; and/or a notification that a manner of communication with the one of the first and second receiver devices having a lower clock accuracy is changed.
Clause E22. The method of any of clauses E1 through E21, wherein the on-body device comprises:
a sensor;
wireless communication electronics;
sensor interface electronics configured to receive a signal from the sensor, the signal indicative of a glucose level of a user;
a housing configured to retain the wireless communication electronics and sensor interface electronics; and
an adhesive patch adhesively coupled with the housing,
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the glucose level in the bodily fluid and output the signal to the sensor interface electronics
Clause E23. A method of wireless communication in an on-body analyte monitoring system, comprising: wirelessly receiving, by an on-body device, data indicative of a first clock accuracy of a clock of a first receiver device; assessing the first clock accuracy parameter; and determining to transmit an analyte measurement to a second receiver device on the basis of the first clock accuracy parameter.
Clause E24. The system of clause E23, wherein the on-body device comprises:
a sensor;
wireless communication electronics;
sensor interface electronics configured to receive a signal from the sensor, the signal indicative of an analyte level of a user;
a housing configured to retain the wireless communication electronics and sensor interface electronics; and
an adhesive patch adhesively coupled with the housing,
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the analyte level in the bodily fluid and output the signal to the sensor interface electronics.
Clause F1. An on-body glucose monitoring system, comprising: an on-body device configured to be held on the body of a subject, the on-body device comprising a housing configured to hold first electronics, wherein the first electronics are configured to: interface with a sensor configured to measure a glucose level of the subject; receive a wireless communication from a first receiver device, wherein the wireless communication comprises data indicative of a first clock accuracy of a clock of the first receiver device; assess the first clock accuracy parameter; and determine to transmit a glucose measurement to a second receiver device on the basis of the data indicative of a first clock accuracy of a clock of the first receiver device.
Clause F2. The system of clause F1, wherein the data indicative of the first clock accuracy of the clock of the first receiver device is a first clock accuracy parameter of the clock or an indication of a type of the first receiver device.
Clause F3. The system of clauses F1 or F2, wherein the first receiver device is a smart phone, smart wearable device, or tablet computer.
Clause F4. The system of any of clauses F1 through F3, wherein the second receiver device is a dedicated receiver device or a drug delivery device.
Clause F5. The system of any of clauses F1 through F3, wherein the second receiver device is a second smart phone, a second smart wearable device, or a second tablet computer, and has a second clock accuracy higher than the first clock accuracy.
Clause F6. The system of any of clauses F1 through F5, wherein the second receiver device is known to the first electronics of the on-body device as having a higher clock accuracy than the first receiver device.
Clause F7. The system of any of clauses F1 through F6, wherein the first electronics are further configured to: receive a wireless communication from the second receiver device that indicates that the second receiver device is of a type having a minimum acceptable clock accuracy.
Clause F8. The system of any of clauses F1 through F7, wherein the first electronics are further configured to: wirelessly receive a second clock accuracy parameter from the second receiver device prior to determination to transmit the glucose measurement to the second receiver device, wherein the second clock accuracy parameter is indicative of a clock accuracy of a clock of the second receiver device.
Clause F9. The system of clause F8, wherein the data indicative of the first clock accuracy of the clock of the first receiver device comprises a first clock accuracy parameter, and wherein the first electronics are configured to assess the first clock accuracy parameter relative to the second clock accuracy parameter.
Clause F10. The system of any of clauses F8 or F9, wherein the second receiver device is a smart phone, a smart wearable device, or a tablet computer.
Clause F11. The system of any of clauses F1 through F10, wherein the first electronics are further configured to wirelessly transmit the glucose measurement to the second receiver device.
Clause F12. The system of clause F11, further comprising the second receiver device, wherein the second receiver device comprises second electronics configured to wirelessly communicate a glucose measurement received from the on-body device to the first receiver device.
Clause F13. The system of clause F12, wherein the second electronics are further configured to wirelessly communicate the glucose measurement received from the on-body device to a third receiver device.
Clause F14. The system of any of clauses F1 through F11, wherein the first electronics of the on-body device are further configured to: designate the second receiver device as a central point of communication for reporting glucose measurement data; wirelessly transmit a succession of glucose measurements from the on-body device to the second receiver device.
Clause F15. The system of any of clauses F1 through F14, wherein the first electronics of the on-body device are configured to perform authentication to authorize the first receiver device and the second receiver device to communicate with the on-body device.
Clause F16. The system of any of clauses F1 through F15, wherein the first electronics of the on-body device are further configured to grant concurrent permission to allow the first and second receiver devices to receive and read glucose measurement data from the on-body device.
Clause F17. The system of any of clauses F1 through F16, wherein the first electronics are configured to transmit the glucose measurement only to the second receiver device and not the first receiver device on the basis of the data indicative of the first clock accuracy of the clock of the first receiver device.
Clause F18. The system of any of clauses F1 through F17, wherein the second electronics are configured to issue at least one notification to the user that pertains to transmission of the glucose measurement to the second receiver device on the basis of the data indicative of the first clock accuracy of the clock of the first receiver device.
Clause F19. The system of clause F18, wherein the at least one notification includes one or more of the following: a notification of the on-body device transmission of the glucose measurement to the second receiver device; a notification of the result of utilization of the system with a receiver device having an unacceptable clock accuracy; a recommendation that the user take an action to manage on-body device power consumption; a notification that one of the first and second receiver devices having the higher clock accuracy will be designated as a central point of communication for receipt of glucose measurements; a prompt for the user's permission to treat one of the first and second receiver devices as a central point of communication; a notification of a change in a target wear duration of the on-body device if the user continues with use of the one of the first and second receiver devices having a lower clock accuracy; and/or a notification that a manner of communication with the one of the first and second receiver devices having a lower clock accuracy is changed.
Clause F20. The system of any of clauses F1 through F19, wherein the on-body device is configured to receive the wireless communication from the receiver device according to one of the following protocols: Wi-Fi, Bluetooth, Bluetooth Low Energy, Zigbee, Z-Wave, Near Field Communication (NFC), Ultra-Wide Band (UWB), or a Personal Area Network (PAN) protocol.
Clause F21. The system of any of clauses F1 through F20, wherein the on-body device is configured to wirelessly communicate with the receiver device according to at least two of the following: a Bluetooth protocol, a Bluetooth Low Energy Protocol, a Near-Field Communication (NFC) protocol, and a Wi-Fi protocol.
Clause F22. The system of any of clauses F1 through F21, wherein the on-body device is configured to receive the wireless communication from the receiver device according to a non-standard protocol.
Clause F23. The system of any of clauses F1 through F22, wherein the on-body device is configured to perform wireless communications with the receiver device according to a Bluetooth Low Energy protocol.
Clause F24. The system of any of clauses F1 through F23, wherein the on-body device is configured to perform wireless communications with the receiver device according to a Near Field Communication (NFC) protocol.
Clause F25. The system of any of clauses F1 through F24, wherein the on-body device is configured to perform wireless communications with the receiver device according to a Near Field Communication (NFC) protocol and a Bluetooth Low Energy protocol.
Clause F26. The system of any of clauses F1 through F25, wherein the on-body device further comprises:
a sensor; and
an adhesive patch adhesively coupled with the housing,
wherein the first electronics comprise: wireless communication electronics; and sensor interface electronics configured to receive a signal from the sensor, the signal indicative of a glucose level of a user;
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the glucose level in the bodily fluid and output the signal to the sensor interface electronics.
Clause F27. An on-body analyte monitoring system, comprising: an on-body device configured to be held on the body of a subject, the on-body device comprising a housing configured to hold first electronics, wherein the first electronics are configured to: interface with a sensor configured to measure an analyte level of the subject; receive a wireless communication from a first receiver device, wherein the wireless communication comprises data indicative of a first clock accuracy of a clock of the first receiver device; assess the indicative of the first clock accuracy of the clock of the first receiver device; and determine to transmit an analyte measurement to a second receiver device on the basis of the assessed data indicative of the first clock accuracy of the clock of the first receiver device.
Clause F28. The system of clause F27, wherein the on-body device further comprises:
a sensor; and
an adhesive patch adhesively coupled with the housing,
wherein the first electronics comprise: wireless communication electronics; and sensor interface electronics configured to receive a signal from the sensor, the signal indicative of an analyte level of a user;
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the analyte level in the bodily fluid and output the signal to the sensor interface electronics
Clause G1. A method of wireless communication in an on-body glucose monitoring system, comprising: receiving a first wireless communication, by an on-body device, from a first receiver device, wherein the first wireless communication comprises data indicative of a first clock accuracy of the first receiver device; receiving a second wireless communication, by the on-body device, from a second receiver device, wherein the second wireless communication comprises data indicative of a second clock accuracy of the second receiver device; determining a first manner of wireless communication with the first receiver device on the basis of the data indicative of the first clock accuracy; and determining a second manner of wireless communication with the second receiver device on the basis of the data indicative of the second clock accuracy, wherein the first manner of communication is different than the second manner of communication.
Clause G2. The method of clause G1, wherein the first manner of communication is a connection-oriented communication mode and the second manner of communication is a connection-less communication mode.
Clause G3. The method of clause G1 or G2, wherein the first manner of communication is a directed-connection communication mode and the second manner of communication is an advertising communication mode.
Clause G4. The method of any of clauses G1 through G3, wherein the data indicative of the first clock accuracy and the data indicative of the second clock accuracy indicate that the first receiver device has a more accurate clock than the second receiver device.
Clause G5. The method any of clauses G1 through G4, wherein the data indicative of the first clock accuracy and the data indicative of the second clock accuracy indicate that the second receiver device has a more accurate clock than the first receiver device.
Clause G6. The method of any of clauses G1 through G5, wherein the first manner of communication is one or more of a mode of communication, a schedule of communication, and/or a designation of the first receiver device as central point of communication, CPOC, for the receipt of communications from the on-body device.
Clause G7. The method of any of clauses G1 through G6, wherein the second manner of communication is one or more of a mode of communication, a schedule of communication, and/or a designation of the second receiver device as central point of communication, CPOC, for the receipt of communications from the on-body device.
Clause G8. The method of any of clauses G1 through G7, wherein determining a first manner of wireless communication with the first receiver device on the basis of the data indicative of the first clock accuracy comprises designating the first receiver device as a central point of communication with the on-body device.
Clause G9. The method of clause G8, further comprising: wirelessly transmitting a succession of glucose measurements from the on-body device to the first receiver device; and wirelessly transmitting, by the first receiver device, the succession of glucose measurements to the second receiver device.
Clause G10. The method of any of clauses G8 or G9, further comprising transitioning the on-body glucose monitoring system from a one-to-many communication topology where the on-body device transmits glucose measurements to both of the first and second receiver devices to a partial mesh topology where the on-body device transmits a series of glucose measurements to the first receiver device and the first receiver devices transmits the series of glucose measurements to the second receiver device.
Clause G11. The method of any of clauses G1 through G10, wherein the data indicative of the first clock accuracy of the first receiver device comprises a clock accuracy parameter of the first receiver device or an indication of the first receiver device type.
Clause G12. The method of clause G11, wherein the indication of the first receiver device type is an indication of the manufacturer of the first receiver device and/or the model of the first receiver device.
Clause G13. The method of any of clauses G1 through G12, wherein the data indicative of the second clock accuracy of the second receiver device comprises a clock accuracy parameter of the second receiver device or an indication of the second receiver device type.
Clause G14. The method of any of clauses G1 through G13, wherein the on-body device comprises:
a sensor;
wireless communication electronics;
sensor interface electronics configured to receive a signal from the sensor, the signal indicative of a glucose level of a user;
a housing configured to retain the wireless communication electronics and sensor interface electronics; and
an adhesive patch adhesively coupled with the housing,
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the glucose level in the bodily fluid and output the signal to the sensor interface electronics.
Clause G15. A method of wireless communication in an on-body analyte monitoring system, comprising: receiving a first wireless communication, by an on-body device, from a first receiver device, wherein the first wireless communication comprises data indicative of a first clock accuracy of the first receiver device; receiving a second wireless communication, by the on-body device, from a second receiver device, wherein the second wireless communication comprises data indicative of a second clock accuracy of the second receiver device; determining a first manner of wireless communication with the first receiver device on the basis of the data indicative of the first clock accuracy; and determining a second manner of wireless communication with the second receiver device on the basis of the data indicative of the second clock accuracy, wherein the first manner of communication is different than the second manner of communication.
Clause G16. The system of clause G15, wherein the on-body device comprises:
a sensor;
wireless communication electronics;
sensor interface electronics configured to receive a signal from the sensor, the signal indicative of an analyte level of a user;
a housing configured to retain the wireless communication electronics and sensor interface electronics; and
an adhesive patch adhesively coupled with the housing,
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the analyte level in the bodily fluid and output the signal to the sensor interface electronics
Clause H1. An on-body glucose monitoring system, comprising: an on-body device configured to be held on the body of a subject, the on-body device comprising a housing configured to hold first electronics, wherein the first electronics are configured to: receive a first wireless communication from a first receiver device, wherein the first wireless communication comprises data indicative of a first clock accuracy of the first receiver device; receive a second wireless communication from a second receiver device, wherein the second wireless communication comprises data indicative of a second clock accuracy of the second receiver device; determine a first manner of wireless communication with the first receiver device on the basis of the data indicative of the first clock accuracy; and determine a second manner of wireless communication with the second receiver device on the basis of the data indicative of the second clock accuracy, wherein the first manner of communication is different than the second manner of communication.
Clause H2. The system of clause H1, wherein the first manner of communication is a connection-oriented communication mode and the second manner of communication is a connection-less communication mode.
Clause H3. The system of clause H1, wherein the first manner of communication is a directed-connection communication mode and the second manner of communication is an advertising communication mode.
Clause H4. The system of any of clauses H1 through H3, wherein the data indicative of the first clock accuracy and the data indicative of the second clock accuracy indicate that the first receiver device has a more accurate clock than the second receiver device.
Clause H5. The system of any of clauses H1 through H4, wherein the data indicative of the first clock accuracy and the data indicative of the second clock accuracy indicate that the second receiver device has a more accurate clock than the first receiver device.
Clause H6. The system of any of clauses H1 through H5, wherein the first manner of communication comprises at least one of a mode of communication, a schedule of communication, and a designation of the first receiver device as central point of communication, CPOC, for the receipt of communications from the on-body device.
Clause H7. The system of any of clauses H1 through H6, wherein the second manner of communication comprises at least one of a mode of communication, a schedule of communication, and a designation of the second receiver device as central point of communication, CPOC, for the receipt of communications from the on-body device.
Clause H8. The system of any of clauses H1 through H7, wherein the manner of wireless communication with the first receiver device comprises a designation of the first receiver device as a central point of communication with the on-body device.
Clause H9. The system of clause H8, wherein the first electronics are further configured to wirelessly transmit a succession of glucose measurements from the on-body device to the first receiver device, and wherein the system further comprises the first receiver device, wherein the first receiver device comprises second electronics configured to wirelessly transmit the succession of glucose measurements to the second receiver device.
Clause H10. The system of any of clauses H1 through H9, wherein the data indicative of the first clock accuracy of the first receiver device comprises a clock accuracy parameter of the first receiver device or an indication of the first receiver device type.
Clause H11. The system of clause H10, wherein the indication of the first receiver device type is an indication of the manufacturer of the first receiver device or the model of the first receiver device.
Clause H12. The system of any of clauses H1 through H11, wherein the data indicative of the second clock accuracy of the second receiver device comprises a clock accuracy parameter of the second receiver device or an indication of the second receiver device type.
Clause H13. The system of any of clauses H1 through H12, wherein the on-body device is configured to receive the wireless communication from the receiver device according to one of the following protocols: Wi-Fi, Bluetooth, Bluetooth Low Energy, Zigbee, Z-Wave, Near Field Communication (NFC), Ultra-Wide Band (UWB), or a Personal Area Network (PAN) protocol.
Clause H14. The system of any of clauses H1 through H13, wherein the on-body device is configured to wirelessly communicate with the receiver device according to at least two of the following: a Bluetooth protocol, a Bluetooth Low Energy Protocol, a Near-Field Communication (NFC) protocol, and a Wi-Fi protocol.
Clause H15. The system of any of clauses H1 through H14, wherein the on-body device is configured to receive the wireless communication from the receiver device according to a non-standard protocol.
Clause H16. The system of any of clauses H1 through H15, wherein the on-body device is configured to perform wireless communications with the receiver device according to a Bluetooth Low Energy protocol.
Clause H17. The system of any of clauses H1 through H16, wherein the on-body device is configured to perform wireless communications with the receiver device according to a Near Field Communication (NFC) protocol.
Clause H18. The system of any of clauses H1 through H17, wherein the on-body device is configured to perform wireless communications with the receiver device according to a Near Field Communication (NFC) protocol and a Bluetooth Low Energy protocol.
Clause H19. The system of any of clauses H1 through H18, wherein the on-body device further comprises:
a sensor; and
an adhesive patch adhesively coupled with the housing,
wherein the first electronics comprise: wireless communication electronics; and sensor interface electronics configured to receive a signal from the sensor, the signal indicative of a glucose level of a user;
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the level of the glucose in the bodily fluid and output the signal to the sensor interface electronics.
Clause H20. An on-body analyte monitoring system, comprising: an on-body device configured to be held on the body of a subject, the on-body device comprising a housing configured to hold first electronics, wherein the first electronics are configured to: receive a first wireless communication from a first receiver device, wherein the first wireless communication comprises data indicative of a first clock accuracy of the first receiver device; receive a second wireless communication from a second receiver device, wherein the second wireless communication comprises data indicative of a second clock accuracy of the second receiver device; determine a first manner of wireless communication with the first receiver device on the basis of the data indicative of the first clock accuracy; and determine a second manner of wireless communication with the second receiver device on the basis of the data indicative of the second clock accuracy, wherein the first manner of communication is different than the second manner of communication.
Clause H21. The system of clause H20, wherein the on-body device further comprises:
a sensor; and
an adhesive patch adhesively coupled with the housing,
wherein the first electronics comprise: wireless communication electronics; and sensor interface electronics configured to receive a signal from the sensor, the signal indicative of an analyte level of a user;
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the level of the analyte in the bodily fluid and output the signal to the sensor interface electronics.
Clause I1. A method of wireless communication in an on-body analyte monitoring system, comprising: obtaining, by an on-body device on the body of a subject, first information indicative of a level of an analyte of the subject; and wirelessly transmitting a first advertisement from the on-body device, wherein the first advertisement comprises the first information indicative of the level of the analyte of the subject.
Clause I2. The method of clause I1, wherein the first advertisement is transmitted to a receiver device.
Clause I3. The method of any of clauses I1 or I2, wherein the first advertisement comprises a payload, the payload comprising the first information indicative of the level of the analyte of the subject.
Clause I4. The method of clause I3, wherein the first information indicative of the level of the analyte of the subject is time-correlated, indexed, or sequence-correlated with respect to second information indicative of the level of the analyte of the subject not included in the first advertisement.
Clause I5. The method of any of clauses I1 through I4, further comprising wirelessly transmitting a first response from the receiver to the on-body device.
Clause I6. The method of clause I5, further comprising receiving the first response by the on-body device.
Clause I7. The method of clause I6, further comprising validating and/or interpreting the first response by the on-body device.
Clause I8. The method of clause I7, wherein the on-body device validates the first response by authenticating and/or error checking the response.
Clause I9. The method of any of clauses I6 through I8, wherein the on-body device interprets the first response by reading a payload of the response.
Clause I10. The method of any of clauses I5 through I9, wherein the first response indicates that the receiver device acknowledges receipt of the first advertisement.
Clause I11. The method of any of clauses I5 through I10, wherein the on-body device and the receiver device communicate in synchronous fashion, and the first response is transmitted during an assigned response time slot.
Clause I12. The method of any of clauses I6 through I11, further comprising deactivating wireless communication electronics of the on-body device after receiving the first response and maintaining deactivation of the wireless communication electronics until a time for transmission of a next advertisement.
Clause I13. The method of clause I12, wherein the time for transmission of a next advertisement is the time of a next transmission interval.
Clause I14. The method of clause I12, wherein the time for transmission of a next advertisement is the time of a next event according to a Bluetooth protocol.
Clause I15. The method of any of clauses I1 through I4, further comprising failing to receive, by the on-body device, a first valid acknowledgement response from the receiver device.
Clause I16. The method of clause I15, wherein the on-body device fails to receive the first valid acknowledgment response during a set receive window and wherein the on-body device lengthens the set receive window to attempt to receive the first valid acknowledgement response.
Clause I17. The method of any of clauses I15 or I16, wherein failing to receive, by the on-body device, the first valid acknowledgement response from the receiver device comprises failing to receive, by the on-body device, the first valid acknowledgement response from the receiver device because the receiver device did not transmit the first valid acknowledgement response or did transmit the first valid acknowledgement response but was out of range of the on-body device.
Clause I18. The method of any of clauses I15 through I17, wherein the on-body device does not receive the first valid acknowledgement response from the receiver device during a synchronous response time slot for the receiver device.
Clause I19. The method of clause I15, wherein failing to receive, by the on-body device, the first valid acknowledgement response from the receiver device comprises failing to receive, by the on-body device, the first valid acknowledgement response from the receiver device because a response received by the on-body device was determined to be invalid.
Clause I20. The method of clause I19, wherein the response received by the on-body device was determined to be invalid because of failure to be authenticated and/or failure to pass an error check.
Clause I21. The method of clause I15, wherein failing to receive, by the on-body device, the first valid acknowledgement response from the receiver device comprises failing to receive, by the on-body device, the first valid acknowledgement response from the receiver device because a response received by the on-body device was valid but indicated non-acknowledgement of receipt of the first advertisement.
Clause I22. The method of any of clauses I15 through I21, further comprising wirelessly transmitting a second advertisement from the on-body device, wherein the second advertisement comprises the first information indicative of the level of the analyte of the subject.
Clause I23. The method of clause I22, wherein the second advertisement is a directed advertisement addressed to the receiver device from which the on-body device did not receive a first valid acknowledgement response.
Clause I24. The method of clause I22, wherein the second advertisement is not addressed to the receiver device from which the on-body device did not receive a first valid acknowledgement response.
Clause I25. The method of any of clauses I22 through I24, wherein the on-body device and the receiver device wirelessly communicate according to a synchronous protocol, and wherein the first advertisement is wirelessly transmitted at a time of a first transmission subinterval of the synchronous protocol and the second advertisement is wirelessly transmitted at a time of a second transmission subinterval of the synchronous protocol.
Clause I26. The method of clause I25, wherein the first transmission subinterval is a first subevent of a Bluetooth periodic advertisement with response (PAwR) transport protocol and the second transmission subinterval is a second subevent of the Bluetooth PAwR protocol.
Clause I27. The method of any clauses I22 through I26, wherein the receiver device is one of a plurality of receiver devices, wherein each of the plurality of receiver devices has an assigned time slot within the first transmission subinterval, and wherein each of the plurality of receiver devices transmits a response during its assigned time slot.
Clause I28. The method of any of clauses I22 through I24, wherein the on-body device and the receiver device wirelessly communicate according to a synchronous protocol, and wherein the first advertisement is wirelessly transmitted at a time of a first transmission interval of the synchronous protocol and the second advertisement is wirelessly transmitted at a time of a second transmission interval of the synchronous protocol.
Clause I29. The method of clause I28, wherein the first transmission interval is a first event of a Bluetooth periodic advertisement with response (PAwR) transport protocol and the second transmission interval is a second event of the Bluetooth PAwR protocol.
Clause I30. The method of any of clauses I22 through I29, further comprising monitoring, by the on-body device, for a second valid acknowledgement response to the second advertisement from the receiver device.
Clause I31. The method of any of clauses I22 through I30, further comprising failing to receive, by the on-body device, a second valid acknowledgement response to the second advertisement from the receiver device.
Clause I32. The method of any of clauses I22 through I31, further comprising receiving, by the on-body device, a second valid acknowledgement response to the second advertisement from the receiver device.
Clause I33. The method of any of clauses I15 through I21, further comprising wirelessly transmitting, by the on-body device, a notification to the receiver device that a prior response was not received.
Clause I34. The method of any of clauses I15 through I21, further comprising wirelessly transmitting, by the on-body device, a notification to the receiver device that a prior response was received and was determined to be invalid.
Clause I35. The method of any of clauses I33 or I34, wherein the notification is addressed to the unresponsive receiver device.
Clause I36. The method of any of clauses I15 through I21, wherein the receiver device is assigned a first response time slot, the method further comprising wirelessly transmitting, by the on-body device, an instruction to the receiver device to change to a second response time slot.
Clause I37. The method of any of clauses I15 through I21, wherein the receiver device is assigned a first response time slot, the method further comprising wirelessly transmitting, by the on-body device, an instruction to the receiver device to increase the time between consecutive first response time slots.
Clause I38. The method of any of clauses I36 or I37, wherein the first advertisement is wirelessly transmitted in a first transmission interval of a synchronous protocol, and the instruction is sent in a transmission subinterval of the first transmission interval.
Clause I39. The method of any of clauses I36 through I38, wherein the first advertisement is wirelessly transmitted in a first transmission interval of a synchronous protocol, and the instruction is sent in a second transmission interval after the first event.
Clause I40. The method of any of clauses I15 through I21, wherein the on-body device and receiver device communicate according to a connection-less Bluetooth protocol, the method further comprising wirelessly transmitting, by the on-body device, a connectable advertisement to the receiver device, wherein the connectable advertisement comprises a solicitation to initiate a connection-oriented Bluetooth pairing between the on-body device and the receiver device.
Clause I41. The method of any of clauses I15 through I21 and I40, further comprising establishing a connection-oriented Bluetooth paired session between the on-body device and the receiver device.
Clause I42. The method of any of clauses I15 through I21, further comprising wirelessly transmitting, by the on-body device, an instruction to the unresponsive receiver device to reconnect to the on-body device.
Clause I43. The method of any of clauses I15 through I21 and I42, further comprising wirelessly transmitting, by the on-body device, an additional attempt to connect to the unresponsive receiver device on a different frequency.
Clause I44. The method of any of clauses I15 through I21 and I42 or I43, further comprising wirelessly transmitting, by the on-body device, an additional attempt to connect to the unresponsive receiver device with a different physical format.
Clause I45. The method of any of clauses I15 through I21 and I42 through I44, further comprising ceasing attempts to transmit analyte data to the unresponsive receiver device according to the communication session that was established with the unresponsive receiver device, and advertising for a new communication session with the unresponsive receiver device.
Clause I46. The method of any of clauses I15 through I21 and I42 through I45, further comprising wirelessly transmitting, by the on-body device, a final addressed advertisement to the unresponsive receiver device comprising a notification that no further attempts to communicate with the unresponsive receiver device will be made.
Clause I47. The method of any of clauses I15 through I21 and I42 through I46, further comprising wirelessly transmitting, by the on-body device, an instruction to receiver devices to adjust the amount of time in each response time slot to generate a new timing pattern.
Clause I48. The method of any of clauses I15 through I21 and I42 through I47, further comprising wirelessly transmitting, by the on-body device, an instruction that reassigns at least one time slot
Clause I49. The method of any of clauses I15 through I21, further comprising wirelessly transmitting, by the on-body device, a plurality of subsequent advertisements after the first advertisement.
Clause I50. The method of clause I49, wherein each subsequent advertisement of the plurality of subsequent advertisements comprises the first information indicative of the level of the analyte of the subject.
Clause I51. The method of any of clauses I49 or I50, wherein the on-body device and the receiver device wirelessly communicate according to a synchronous protocol, and wherein the plurality of subsequent advertisements are wirelessly transmitted at successive transmission subintervals of a first transmission interval of the synchronous protocol.
Clause I52. The method of any of clauses I49 or I50, wherein the on-body device and the receiver device wirelessly communicate according to a synchronous protocol, and wherein the plurality of subsequent advertisements are wirelessly transmitted at successive transmission intervals of the synchronous protocol.
Clause I53. The method of any of clauses I49 or I50, wherein the on-body device and the receiver device wirelessly communicate according to a synchronous protocol having a first event and a second event, and wherein the plurality of subsequent advertisements are wirelessly transmitted at at least one subevent of the first event and at least one subevent of the second event.
Clause I54. The method of any of clauses I49 through I53, further comprising failing to receive, by the on-body device, at least one valid acknowledgement response, from the receiver device, in response to the plurality of subsequent advertisements.
Clause I55. The method of any of clauses I49 through I53, further comprising receiving, by the on-body device, at least one response from the receiver device, wherein the at least one response is determined to be invalid.
Clause I56. The method of clause I55, further comprising determining to cease wireless transmission, by the on-body device, of any further advertisement comprising the first information indicative of the level of the analyte of the subject.
Clause I57. The method of clause I56, wherein the determination to cease wireless transmission is based, at least in part, on having reached a threshold number of wireless transmissions of advertisements comprising the first information indicative of the level of the analyte of the subject.
Clause I58. The method of clause I57, wherein the threshold number is two or more.
Clause I59. The method of clause I56, wherein the determination to cease wireless transmission is based, at least in part, on having reached a threshold duration of time since receiving any response from the receiver device.
Clause I60. The method of clause I56, wherein the determination to cease wireless transmission is based, at least in part, on having reached a threshold duration of time since receiving any valid acknowledgment response from the receiver device.
Clause I61. The method of any of clauses I1 through I60, wherein the receiver device is a first receiver device, the method further comprising establishing communication between the on-body device and a second receiver device while maintaining communication with the first receiver device.
Clause I62. The method of clause I61, wherein establishing communication between the on-body device and the second receiver device comprises wirelessly transmitting, by the second receiver device, a solicitation to form a connection-oriented Bluetooth pairing with the on-body device.
Clause I63. The method of any of clauses I61 or I62, wherein establishing communication between the on-body device and the second receiver device comprises establishing a connection-oriented Bluetooth paired session between the on-body device and the second receiver device.
Clause I64. The method of clause I63, further comprising using the connection-oriented Bluetooth paired session to exchange setup information to form a synchronous advertising-based Bluetooth communication session between the on-body device and the second receiver device.
Clause I65. The method of clause I64, wherein the synchronous advertising-based Bluetooth communication session is a periodic advertising with response (PAwR) communication session.
Clause I66. The method of clause I64 or I65, wherein the setup information comprises at least one of: the length of the transmission interval; the length of a transmission subinterval; the number of subintervals per transmission interval; the response slot delay; the time duration of each response slot; the access address; a channel for transmission and response; a format requirement for a protocol data unit (PDU); and/or a response time slot assignment.
Clause I67. The method of any of clauses I64 through I66, wherein the setup information comprises a clock accuracy parameter of the second receiver device.
Clause I68. The method of clause I67, further comprising selecting, by the on-body device, a transport protocol for communication with the second receiver device based, at least in part, on the clock accuracy parameter.
Clause I69. The method of any of clauses I64 through I68, further comprising ending the connection-oriented Bluetooth paired session between the on-body device and the second receiver device and communicating between the on-body device and the second receiver device with the synchronous advertising-based Bluetooth communication session.
Clause I70. The method of any of clauses I64 through I68, further comprising maintaining the synchronous advertising-based Bluetooth communication session between the on-body device and the first receiver device while maintaining connection-oriented Bluetooth paired session between the on-body device and the second receiver device.
Clause I71. The method of any of clauses I69 or I70, wherein the synchronous advertising-based Bluetooth communication session is a periodic advertising with response (PAwR) communication session.
Clause I72. The method of any of clauses I61 through I70, wherein establishing communication between the on-body device and the second receiver device comprises wirelessly transmitting, by the on-body device, a non-synchronous advertisement and receiving, by the on-body device, a response to the non-synchronous advertisement transmitted by the second receiver device.
Clause I73. The method of clause I72, wherein establishing communication between the on-body device and the second receiver device comprises establishing communication between the on-body device and the second receiver device with a non-synchronous advertising session.
Clause I74. The method of clause I73, further comprising using the non-synchronous advertising-based session to exchange setup information to form a synchronous advertising-based session between the on-body device and the second receiver device.
Clause I75. The method of clause I74, wherein the synchronous advertising-based session is a periodic advertising with response (PAwR) communication session.
Clause I76. The method of clause I74 or I75, wherein the setup information comprises a clock accuracy parameter of the second receiver device.
Clause I77. The method of clause I76, further comprising selecting, by the on-body device, a transport protocol for communication with the second receiver device based, at least in part, on the clock accuracy parameter.
Clause I78. The method of any of clauses I72 through I77, further comprising ending the nonsynchronous advertising session between the on-body device and the second receiver device and communicating between the on-body device and the second receiver device with the synchronous advertising-based session.
Clause I79. The method of any of clauses I72 through I77, further comprising maintaining synchronous advertising communication between the on-body device and the first receiver device while maintaining non-synchronous advertising-based communication between the on-body device and the second receiver device.
Clause I80. The method of any of clauses I72 through I80, wherein the synchronous advertising-based session is a periodic advertising with response (PAwR) communication session.
Clause I81. The method of any of clauses I1 through I60, wherein the receiver device is a first receiver device, wherein the on-body device and the first receiver device communicate with a synchronous advertising-based session, and the method further comprises establishing communication between the on-body device and a second receiver device.
Clause I82. The method of clause I81, wherein establishing communication between the on-body device and the second receiver device comprises the second receiver device joining the synchronous advertising-based session.
Clause I83. The method of clause I82, wherein the second receiver device joins the synchronous advertising-based session without first establishing communication with the on-body device according to a Bluetooth transport protocol other than synchronous advertising-based communication.
Clause I84. The method of any of clauses I82 or I83, further comprising communicating synchronous advertising-based session setup information from the on-body device to the second receiver using a non-Bluetooth communication protocol.
Clause I85. The method of clause I84, wherein the non-Bluetooth communication protocol is Near Field Communication (NFC).
Clause I86. The method of any of clauses I84 or I85, wherein the synchronous advertising-based session setup information comprises a clock accuracy parameter of the second receiver device.
Clause I87. The method of clause I86, further comprising selecting, by the on-body device, a transport protocol for communication with the second receiver device based, at least in part, on the clock accuracy parameter.
Clause I88. The method of any of clauses I82 or I83, further comprising monitoring, by the second receiver device, for a synchronous advertisement from the on-body device.
Clause I89. The method of clause I88, further comprising responding, by the second receiver device, to the synchronous advertisement in a predetermined response time slot.
Clause I90. The method of clause I89, wherein the predetermined response time slot is reserved for a new receiver device seeking to join communication with the on-body device.
Clause I91. The method of any of clauses I61 through I90, further comprising communicating a second receiver response time slot to the second receiver device.
Clause I92. The method of clause I91, wherein the second receiver response time slot is immediately adjacent a response time slot for the first receiver device.
Clause I93. The method of any of clauses I61 through I92, further comprising reassigning a response time slot to at least one receiver device when another receiver device ceases communication with the on-body device.
Clause I94. The method of clause I93, wherein the reassigned response time slot has a reduced delay from the last communication from the on-body device.
Clause I95. The method of any of clauses I1 through I94, wherein the first receiver device is one of a plurality of receiver devices, the method further comprising conducting a synchronous advertising-based communication session with the plurality of receiver devices, wherein the on-body device transmits advertisements periodically during the synchronous advertising-based communication session, wherein transmitted advertisements comprise information indicative of an analyte level of the subject and is configured for receipt by all of the plurality of receiver devices.
Clause I96. The method of clause I95, wherein the on-body device dos not monitor for all responses from the plurality of receiver devices.
Clause I97. The method of any of clauses I95 or I96, wherein the on-body device does not monitor for a response from every receiver device after every advertisement.
Clause I98. The method of any of clauses I95 through I97, wherein the on-body device selectively monitors for responses from the plurality of receiver devices such that the on-body device does not monitor for selective responses transmitted by the plurality of receiver devices.
Clause I99. The method of any of clauses I95 through I98, wherein the on-body device monitors for responses from a subset of one or more of the receiver devices in the plurality of receiver devices, but not all receiver devices in the plurality of receiver devices, after transmission of an advertisement to all of the receiver devices in the plurality of receiver devices.
Clause I100. The method of clauses I95 through I99, wherein the on-body device does not monitor for a response from any of the receiver devices in the plurality of receiver devices after transmission of an advertisement to all of the receiver devices in the plurality of receiver devices.
Clause I101. The method of any of clauses I95 through I100, wherein at least a first receiver device of the plurality of receiver devices is a primary receiver device and at least a second receiver device of the plurality of receiver devices is a secondary receiver device, wherein the on-body device monitors for responses from the primary receiver device more frequently than from the secondary receiver device after transmitting advertisements to both the primary and secondary receiver devices.
Clause I102. The method of clause I95, further comprising making a modification to the synchronous advertising-based communication session.
Clause I103. The method of clause I102, wherein the modification to the synchronous advertising-based communication session comprises a modification to a transmission interval.
Clause I104. The method of clause I102, wherein the on-body device transmits advertisements periodically according to a first transmission interval, and wherein making a modification to the synchronous advertising-based communication session comprises: informing the plurality of receiver devices of a second transmission interval that is greater than the first transmission interval; and conducting the synchronous advertising-based communication session by transmitting advertisements periodically according to the second transmission interval.
Clause I105. The method of clause I104, further comprising reverting to the first transmission interval.
Clause I106. The method of clause I102, wherein the modification to the synchronous advertising-based communication session comprises a modification to a transmission subinterval.
Clause I107. The method of clause I106, wherein the on-body device transmits advertisements periodically according to a first transmission interval comprising a plurality of first transmission subintervals each having a first duration, and wherein making a modification to the synchronous advertising-based communication session comprises: informing the plurality of receiver devices of a change that modifies the first duration of the first transmission subintervals to a second duration that is different from the first duration; and conducting the synchronous advertising-based communication session according to the second duration of the first plurality of transmission subintervals.
Clause I108. The method of clause I107, further comprising reverting to communication according to the first duration.
Clause I109. The method of clause I102, wherein the modification to the synchronous advertising-based communication session comprises a modification to a duration of a response time slot.
Clause I110. The method of clause I109, wherein the plurality of receiver devices respond in response time slots each having a first duration, and wherein making a modification to the synchronous advertising-based communication session comprises: informing the plurality of receiver devices of a second duration for response time slots that is less than the first duration; and conducting the synchronous advertising-based communication session by transmitting responses in response time slots having the second duration.
Clause I111. The method of clause I110, further comprising reverting to the first duration.
Clause I112. The method of clause I102, wherein the modification to the synchronous advertising-based communication session comprises a modification to a first operating aspect of monitoring for responses.
Clause I113. The method of clause I112, wherein the on-body device monitors for responses from the plurality of receiver devices according to the first operating aspect, and wherein making a modification to the synchronous advertising-based communication session comprises: conducting the synchronous advertising-based communication session by monitoring for responses according to a second operating aspect, wherein the second operating aspect corresponds to monitoring less frequently than the first operating aspect.
Clause I114. The method of clause I113, wherein the first operating aspect corresponds to monitoring for responses at least once in every transmission interval.
Clause I115. The method of clause I113, wherein the first operating aspect corresponds to monitoring for responses at least twice in every transmission interval.
Clause I116. The method of clause I113, wherein the first operating aspect corresponds to monitoring for responses at least once in a predetermined duration of time.
Clause I117. The method of clause I113, further comprising reverting to the first operating aspect.
Clause I118. The method of any of clauses I113 through I117, wherein in the second operating aspect of monitoring for responses the on-body device does not monitor for at least one response transmitted by the plurality of receiver devices.
Clause I119. The method of any of clauses I113 through I118, wherein modification to the first operating aspect of monitoring for responses is made by the on-body device and the plurality of receiver devices continue to respond without modification.
Clause I120. The method of any of clauses I113 through I119, wherein at least a first receiver device of the plurality of receiver devices is a primary receiver device and at least a second receiver device of the plurality of receiver devices is a secondary receiver device, wherein in the second operating aspect of monitoring for responses the on-body device monitors for responses from the primary receiver device more frequently than from the secondary receiver device.
Clause I121. The method of clause I120, further comprising inputting into at least one device of the analyte monitoring system, by a user, a designation of which of the plurality of receiver devices is the primary receiver device.
Clause I122. The method of clause I102, wherein the modification to the synchronous advertising-based communication session comprises a modification to a power consumption.
Clause I123. The method of clause I102, wherein making a modification to the synchronous advertising-based communication session comprises deactivating wireless communication electronics of the on-body device at least once during every transmission interval.
Clause I124. The method of clause I102, wherein making a modification to the synchronous advertising-based communication session comprises increasing a time of deactivation of wireless communication electronics of the on-body device during every transmission interval.
Clause I125. The method of clause I102, wherein making a modification to the synchronous advertising-based communication session comprises transmitting advertisements by the on-body device with a relatively lower transmit power than prior to the modification.
Clause I126. The method of clause I102, wherein making a modification to the synchronous advertising-based communication session comprises changing a physical format of communication.
Clause I127. The method of any of clauses I102 through I126, further comprising, prior to making a modification to the synchronous advertising-based communication session, determining to make the modification to the synchronous advertising-based communication session based, at least in part, on a state of power of the on-body device.
Clause I128. The method of any of clauses I102 through I126, further comprising, prior to making a modification to the synchronous advertising-based communication session, determining to make the modification to the synchronous advertising-based communication session based, at least in part, on a schedule.
Clause I129. The method of any of clauses I102 through I126, further comprising, prior to making a modification to the synchronous advertising-based communication session, determining to make the modification to the synchronous advertising-based communication session based, at least in part, on detected analyte monitoring condition state.
Clause I130. The method of any of clauses I102 through I126, further comprising, prior to making a modification to the synchronous advertising-based communication session, determining to make the modification to the synchronous advertising-based communication session based, at least in part, on stability of analyte levels of the subject.
Clause I131. The method of any of clauses I102 through I126, further comprising, prior to making a modification to the synchronous advertising-based communication session, determining to make the modification to the synchronous advertising-based communication session based, at least in part, on a mask condition of the on-body device.
Clause I132. The method of any of clauses I102 through I126, further comprising, prior to making a modification to the synchronous advertising-based communication session, determining to make the modification to the synchronous advertising-based communication session based, at least in part, on a change in the number of receiver devices communicating with the on-body device.
Clause I133. The method of any of clauses I102 through I126, further comprising, prior to making a modification to the synchronous advertising-based communication session, determining to make the modification to the synchronous advertising-based communication session based, at least in part, on a clock accuracy parameter of at least one receiver device of the plurality of receiver devices.
Clause I134. The method of any of clauses I1 through I133, further comprising receiving, by the on-body device, a supplementary information request, wherein the supplementary information request requests second information indicative of the level of the analyte of the subject that was not transmitted in the first advertisement.
Clause I135. The method of clause I134, further comprising transmitting, by the on-body device, the second information indicative of the level of the analyte of the subject.
Clause I136. The method of clause I135, wherein the second information indicative of the level of the analyte of the subject is transmitted by the on-body device on a secondary communication channel of a Bluetooth protocol.
Clause I137. The method of any of clauses I135 or I136, wherein the supplementary information request is received during a first transmission interval of a synchronous advertising-based communication session, and the second information indicative of the level of the analyte of the subject is transmitted during the first transmission interval.
Clause I138. The method of any of clauses I135 or I136, wherein the supplementary information request is received during a first transmission subinterval of a first transmission interval of a synchronous advertising-based communication session, and the second information indicative of the level of the analyte of the subject is transmitted during a subsequent transmission subinterval of the first transmission interval.
Clause I139. The method of any of clauses I135 or I136, wherein the supplementary information request is received during a first transmission interval of a synchronous advertising-based communication session, and the second information indicative of the level of the analyte of the subject is transmitted during a second transmission interval.
Clause I140. The method of any of clauses I135 through I139, further comprising transmitting a pointer by the on-body device, wherein the pointer identifies a secondary channel on which the second information indicative of the level of the analyte of the subject will be transmitted.
Clause I141. The method of clause I140, wherein the pointer further identifies a time offset at which the second information indicative of the level of the analyte of the subject will be transmitted.
Clause I142. The method of any of clauses I135 through I141, wherein the second information indicative of the level of the analyte of the subject is transmitted as a directed communication to the receiver device that issued the supplementary information request.
Clause I143. The method of any of clauses I135 through I141, further comprising wirelessly transmitting, by the on-body device, the second information indicative of the level of the subject in a plurality of chained advertisements on one or more secondary communication channels of the Bluetooth protocol.
Clause I144. The method of any of clauses I1 through I143, further comprising: wirelessly transmitting, by a first receiver device, a response to the first advertisement; and wirelessly receiving, by a second receiver device, the response to the first advertisement.
Clause I145. The method of clause I144, wherein the response comprises an alarm activation or deactivation state.
Clause I146. The method of any of clauses I144 or I145, wherein the response comprises an alarm threshold setting.
Clause I147. The method of any of clauses I144 through I146, wherein the response comprises an alarm enablement setting.
Clause I148. The method of any of clauses I144 through I147, wherein the response comprises an alarm notification format setting.
Clause I149. The method of any of clauses I144-I148, wherein the response comprises an alarm mode setting.
Clause I150. The method of any of clauses I144 through I149, wherein the response comprises analyte data.
Clause I151. The method of any of clauses I144 through I150, wherein the response comprises a user-specific setting.
Clause I152. The method of any of clauses I144 through I151, wherein the response comprises a personal profile setting.
Clause I153. The method of any of clauses I144 through I152, wherein the response comprises calibration information.
Clause I154. The method of any of clauses I144 through I153, wherein the response comprises drug therapy and/or insulin information.
Clause I155. The method of any of clauses I144 through I154, further comprising interpreting, by the second receiver device, the response to the first advertisement.
Clause I156. The method of clause I155, further comprising taking an action, by the second receiver device, based on information contained in the response to the first advertisement.
Clause I157. The method of clause I156, wherein taking an action comprises updating or first storing information in the second receiver device.
Clause I158. The method of any of clauses I1 through I143, further comprising: wirelessly receiving, by a first receiver device and a second receiver device, the first advertisement, wherein the advertisement comprises data indicative of an analyte level alarm condition; and activating alarms, by the first receiver device and second receiver device, based on the received data indicative of the level of the analyte alarm condition.
Clause I159. The method of clause I158, further comprising wirelessly transmitting by the first receiver device, a response to the first advertisement indicating that a user acknowledged and/or dismissed the alarm on the first receiver device.
Clause I160. The method of clause I159, further comprising wirelessly receiving, by the second receiver device, the response to the first advertisement indicating that a user acknowledged and/or dismissed the alarm on the first receiver device.
Clause I161. The method of clause I160, further comprising deactivating, by the second receiver device, the alarm on the second receiver device based on the received response to the first advertisement indicating that a user acknowledged and/or dismissed the alarm on the first receiver device.
Clause I162. The method of any of clauses I1 through I161, wherein the analyte is selected from the list consisting of: glucose, one or more ketones, lactate, and oxygen.
Clause I163. The method of any of clauses I1 through I161, wherein the analyte is selected from the list consisting of: ammonium, asparagine, aspartate, cholesterol, choline, creatinine, one or more diaphorase inhibitors, ethanol, glucose, glutamate, glycerol, one or more ketones, lactate, lactose, oxygen, phosphate, potassium, pyruvate, and uric acid.
Clause I164. The method of any of clauses I1 through I161, wherein the analyte is a first analyte, and the on-body device is further configured to obtain second information indicative of a second analyte level of the subject.
Clause I165. The method of clause I164, wherein the first analyte is glucose and the second analyte is a ketone.
Clause I166. The method of clause I164, wherein the first and second analytes are selected from the list consisting of: ammonium, asparagine, aspartate, cholesterol, choline, creatinine, one or more diaphorase inhibitors, ethanol, glucose, glutamate, glycerol, one or more ketones, lactate, lactose, oxygen, phosphate, potassium, pyruvate, and uric acid.
Clause I167. The method of any of clauses I1 through I166, wherein the on-body device comprises a partially implantable sensor configured to measure the level of the analyte of the subject.
Clause I168. The method of any of clauses I1 through I166, wherein the on-body device is configured to wirelessly communicate with a wholly implantable sensor configured to measure the level of the analyte of the subject.
Clause I169. The method of any of clauses I1 through I166, wherein the on-body device comprises a wholly ex vivo sensor configured to measure the level of the analyte of the subject.
Clause I170. The method of any of clauses I2 through I169 to the extent dependent on clause I2, wherein the receiver device is a multi-purpose mobile receiver device.
Clause I171. The method of clause I170, wherein the receiver device is smart phone.
Clause I172. The method of clause I170, wherein the receiver device is a smart watch.
Clause I173. The method of any of clauses I2 through I169 to the extent dependent on clause I2, wherein the receiver device is a dedicated receiver device.
Clause I174. The method of any of clauses I2 through I169 to the extent dependent on clause I2, wherein the receiver device is drug delivery device.
Clause I175. The method of any of clauses I2 through I169 to the extent dependent on clause I2, wherein the receiver device is a wireless computing device.
Clause I176. The method of any of clauses I1 through I175, wherein the on-body device receives a clock accuracy parameter of a receiver device.
Clause I177. The method of any of clauses I1 through I176 in combination with the features of the method of any of clauses B1 through B70 or B72 through B77.
Clause I178. The method of any of clauses I1 through I176 in combination with the features of the method of any of clauses D1 through D9 or D10.
Clause I179. The method of any of clauses I1 through I176 in combination with the features of the method of any of clauses E1 through E21 or E23.
Clause I180. The method of any of clauses I1 through I176 in combination with the features of the method of any of clauses G1 through G13 or G15.
Clause I181. The method of any of clauses I1 through I176 in combination with the features of the method of any of clauses AE1 through AE22.
Clause I182. The method of any of clauses I1 through I181, wherein the on-body device comprises:
a sensor;
wireless communication electronics;
sensor interface electronics configured to receive a signal from the sensor, the signal indicative of an analyte level of a user;
a housing configured to retain the wireless communication electronics and sensor interface electronics; and
an adhesive patch adhesively coupled with the housing,
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the analyte level in the bodily fluid and output the signal to the sensor interface electronics.
Clause J1. An on-body analyte monitoring system, comprising: an on-body device configured to be held on the body of a subject, the on-body device comprising a housing configured to hold first electronics, wherein the first electronics are configured to: obtain first information indicative of a level of an analyte of the subject; and wirelessly transmit a first advertisement comprising the first information indicative of the level of the analyte of the subject.
Clause J2. The system of clause J1, wherein the first electronics are configured to transmit the first advertisement to a receiver device.
Clause J3. The system of any of clauses J1 or J2, wherein the first advertisement comprises a payload, the payload comprising the first information indicative of the level of the analyte of the subject.
Clause J4. The system of clause J3, wherein the first information indicative of the level of the analyte of the subject is time-correlated, indexed, or sequence-correlated with respect to second information indicative of the level of the analyte of the subject not included in the first advertisement.
Clause J5. The system of any of clauses J1 through J4, wherein the first electronics are configured to wirelessly receive a first response from a receiver device.
Clause J6. The system of clause J5, wherein the first electronics are configured to validate and/or interpret the first response.
Clause J7. The system of clause J6, wherein the first electronics are configured to validate the first response with authentication and/or an error check.
Clause J8. The system of any of clauses J5 through J7, wherein the first electronics are configured to interpret the first response by reading a payload of the response.
Clause J9. The system of any of clauses J5 through J8, wherein the first response indicates that the receiver device acknowledges receipt of the first advertisement.
Clause J10. The system of any of clauses J5 through J9, wherein the on-body device is configured to communicate with a receiver device in synchronous fashion, and wherein the first electronics are configured to receive the first response during an assigned response time slot.
Clause J11. The system of any of clauses J5 through J10, wherein the first electronics are configured to deactivate wireless communication electronics of the on-body device after receipt of the first response and maintain deactivation of the wireless communication electronics until a time for transmission of a next advertisement.
Clause J12. The system of clause J11, wherein the time for transmission of a next advertisement is the time of a next transmission interval.
Clause J13. The system of clause J11, wherein the time for transmission of a next advertisement is the time of a next event according to a Bluetooth protocol.
Clause J14. The system of any of clauses J1 through J3, wherein the first electronics are configured to recognize a failure to receive a first valid acknowledgement response from the receiver device.
Clause J15. The system of clause J14, wherein the on-body device is configured to lengthen the duration of a set receive window to attempt to receive the first valid acknowledgement response.
Clause J16. The system of clause J14, wherein the first electronics are configured to determine that a received response is invalid.
Clause J17. The system of clause J16, wherein the first electronics are configured to determine that the received response is invalid because of failure to be authenticated and/or failure to pass an error check.
Clause J18. The system of clause J14, wherein the first electronics are configured to determine that the received response is valid but indicated non-acknowledgement of receipt of the first advertisement.
Clause J19. The system of any of clauses J14 through J18, wherein the first electronics are configured to wirelessly transmit a second advertisement comprising the first information indicative of the level of the analyte of the subject.
Clause J20. The system of clause J19, wherein the second advertisement is a directed advertisement addressed to the receiver device from which the on-body device did not receive a first valid acknowledgement response.
Clause J21. The system of any of clauses J19 or J20, wherein the first electronics are configured to wirelessly communicate according to a synchronous protocol and transmit the first advertisement at a time of a first transmission subinterval of the synchronous protocol and the second advertisement at a time of a second transmission subinterval of the synchronous protocol.
Clause J22. The system of clause J21, wherein the first transmission subinterval is a first subevent of a Bluetooth periodic advertisement with response (PAwR) transport protocol and the second transmission subinterval is a second subevent of the Bluetooth PAwR protocol.
Clause J23. The system of any clauses J21 or J22, wherein the receiver device is one of a plurality of receiver devices, wherein each of the plurality of receiver devices has an assigned time slot within the first transmission subinterval, and wherein each of the plurality of receiver devices transmits a response during its assigned time slot.
Clause J24. The system of any of clauses J19 or J20, wherein the first electronics are configured to wirelessly communicate according to a synchronous protocol and transmit the first advertisement at a time of a first transmission interval of the synchronous protocol and the second advertisement at a time of a second transmission subinterval of the synchronous protocol.
Clause J25. The system of clause J24, wherein the first transmission interval is a first event of a Bluetooth periodic advertisement with response (PAwR) transport protocol and the second transmission interval is a second event of the Bluetooth PAwR protocol.
Clause J26. The system of any of clauses J19 through J25, wherein the first electronics are configured to monitor for a second valid acknowledgement response to the second advertisement from the receiver device.
Clause J27. The system of any of clauses J19 through J26, wherein the first electronics are configured to recognize a failure to receive a second valid acknowledgement response to the second advertisement from the receiver device.
Clause J28. The system of any of clauses J19 through J27, wherein the first electronics are configured to receive a second valid acknowledgement response to the second advertisement from the receiver device.
Clause J29. The system of any of clauses J14 through J18, wherein the first electronics are configured to wirelessly transmit a notification to the receiver device that a prior response was not received.
Clause J30. The system of any of clauses J14 through J18, wherein the first electronics are configured to wirelessly transmit a notification to the receiver device that a prior response was received and was determined to be invalid.
Clause J31. The system of any of clauses J29 or J30, wherein the notification is addressed to the unresponsive receiver device.
Clause J32. The system of any of clauses J14 through J18, wherein the receiver device is assigned a first response time slot, and wherein the first electronics are configured to wirelessly transmit an instruction to the receiver device to change to a second response time slot.
Clause J33. The system of any of clauses J14 through J18, wherein the receiver device is assigned a first response time slot, and wherein the first electronics are configured to wirelessly transmit an instruction to the receiver device to increase the time between consecutive first response time slots.
Clause J34. The system of any of clauses J32 or J33, wherein the first electronics are configured to wirelessly transmit the first advertisement in a first transmission interval of a synchronous protocol, and wirelessly transmit the instruction in a transmission subinterval of the first transmission interval.
Clause J35. The system of any of clauses J32-J33, wherein the first electronics are configured to wirelessly transmit the first advertisement in a first transmission interval of a synchronous protocol, and wirelessly transmit the instruction in a second transmission interval of the synchronous protocol.
Clause J36. The system of any of clauses J14 through J18, wherein the first electronics are configured to communicate with the receiver device according to a connection-less Bluetooth protocol, and configured to wirelessly transmit a connectable advertisement to the receiver device, wherein the connectable advertisement comprises a solicitation to initiate a connection-oriented Bluetooth pairing between the on-body device and the receiver device.
Clause J37. The system of any of clauses J14 through J18 and J36, wherein the first electronics are configured to establish a connection-oriented Bluetooth paired session between the on-body device and the receiver device.
Clause J38. The system of any of clauses J14 through J18, further comprising wirelessly transmitting, by the on-body device, an additional attempt to connect to the unresponsive receiver device on a different frequency.
Clause J39. The method of any of clauses J14 through J18 and J38, further comprising wirelessly transmitting, by the on-body device, an additional attempt to connect to the unresponsive receiver device with a different physical format.
Clause J40. The method of any of clauses J14 through J18 and J38 or J39, further comprising ceasing attempts to transmit analyte data to the unresponsive receiver device according to the communication session that was established with the unresponsive receiver device, and advertising for a new communication session with the unresponsive receiver device.
Clause J41. The method of any of clauses J14 through J18 and J38 through J40, further comprising wirelessly transmitting, by the on-body device, a final addressed advertisement to the unresponsive receiver device comprising a notification that no further attempts to communicate with the unresponsive receiver device will be made.
Clause J42. The method of any of clauses J14 through J18 and J38 through J41, further comprising wirelessly transmitting, by the on-body device, an instruction to receiver devices to adjust the amount of time in each response time slot to generate a new timing pattern.
Clause J43. The method of any of clauses J14 through J18 and J38 through J42, further comprising wirelessly transmitting, by the on-body device, an instruction that reassigns at least one time slot.
Clause J44. The system of any of clauses J14 through J18, wherein the first electronics are configured to wirelessly transmit a plurality of subsequent advertisements after the first advertisement.
Clause J45. The system of clause J44, wherein each subsequent advertisement of the plurality of subsequent advertisements comprises the first information indicative of the level of the analyte of the subject.
Clause J46. The system of any of clauses J44 or J45, wherein the first electronics are configured to wirelessly communicate with the receiver device according to a synchronous protocol, and wherein the first electronics are configured to wirelessly transmit the plurality of subsequent advertisements at successive transmission subintervals of a first transmission interval of the synchronous protocol.
Clause J47. The system of any of clauses J44 or J45, wherein the first electronics are configured to wirelessly communicate with the receiver device according to a synchronous protocol, and wherein the first electronics are configured to wirelessly transmit the plurality of subsequent advertisements at successive transmission intervals of the synchronous protocol.
Clause J48. The system of any of clauses J44 or J45, wherein the first electronics are configured to wirelessly communicate with the receiver device according to a synchronous protocol having a first event and a second event, and wherein the first electronics are configured to wirelessly transmit the plurality of subsequent advertisements at at least one subevent of the first event and at least one subevent of the second event.
Clause J49. The system of any of clauses J44 through J48, wherein the first electronics are configured to receive at least one response from the receiver device and determine whether the at least one response is valid or invalid.
Clause J50. The system of clause J49, wherein the first electronics are configured to determine to cease wireless transmission of any further advertisement comprising the first information indicative of the level of the analyte of the subject, based, at least in part, on a threshold number of wireless transmissions of advertisements comprising the first information indicative of the level of the analyte of the subject.
Clause J51. The system of clause J50, wherein the threshold number is two or more.
Clause J52. The system of any of clauses J49 through J51, wherein the first electronics are configured to determine to cease wireless transmission of any further advertisement comprising the first information indicative of the level of the analyte of the subject, based, at least in part, on a threshold duration of time since receiving any response from the receiver device.
Clause J53. The system of clause J52, wherein the first electronics are configured to determine to cease wireless transmission of any further advertisement comprising the first information indicative of the level of the analyte of the subject, based, at least in part, on a threshold duration of time since receiving any valid acknowledgment response from the receiver device.
Clause J54. The system of any of clauses J2 through J53, wherein the receiver device is a first receiver device, wherein the first electronics are configured to establish communication with a second receiver device while maintaining communication with the first receiver device.
Clause J55. The system of clause J54, wherein the receiver device is a first receiver device, wherein the first electronics are configured to establish communication by wirelessly receiving, from the second receiver device, a solicitation to form a connection-oriented Bluetooth pairing with the on-body device.
Clause J56. The system of any of clauses J54 or J55, wherein the first electronics are configured to establish a connection-oriented Bluetooth paired session with the second receiver device.
Clause J57. The system of clause J56, wherein the first electronics are configured to use the connection-oriented Bluetooth paired session to exchange setup information to form a synchronous advertising-based Bluetooth communication session with the second receiver device.
Clause J58. The system of clause J57, wherein the synchronous advertising-based Bluetooth communication session is a periodic advertising with response (PAwR) communication session.
Clause J59. The system of any of clauses J57 or J58, wherein the setup information comprises the length of the transmission interval.
Clause J60. The system of any of clauses J57 through J59, wherein the setup information comprises the length of a transmission subinterval.
Clause J61. The system of any of clauses J57 through J60, wherein the setup information comprises the number of subintervals per transmission interval.
Clause J62. The system of any of clauses J57 through J61, wherein the setup information comprises the response slot delay.
Clause J63. The system of any of clauses J57 through J62, wherein the setup information comprises the time duration of each response slot.
Clause J64. The system of any of clauses J57 through J63, wherein the setup information comprises the access address.
Clause J65. The system of any of clauses J57 through J64, wherein the setup information comprises a channel for transmission and response.
Clause J66. The system of any of clauses J57 through J65, wherein the setup information comprises a format requirement for a protocol data unit (PDU).
Clause J67. The system of any of clauses J57 through J66, wherein the setup information comprises a response time slot assignment.
Clause J68. The system of any of clauses J57 through J67, wherein the setup information comprises a clock accuracy parameter of the second receiver device.
Clause J69. The system of clause J68, wherein the first electronics are configured to select a transport protocol for communication with the second receiver device based, at least in part, on the clock accuracy parameter.
Clause J70. The system of any of clauses J56 through J69, wherein the first electronics are configured to end the connection-oriented Bluetooth paired session between the on-body device and the second receiver device and communicate with the second receiver device with the synchronous advertising-based Bluetooth communication session.
Clause J71. The system of any of clauses J56 through J69, wherein the first electronics are configured to maintain the synchronous advertising-based Bluetooth communication session with the first receiver device and the connection-oriented Bluetooth paired session with the second receiver device.
Clause J72. The system of any of clauses J70 or J71, wherein the synchronous advertising-based Bluetooth communication session is a periodic advertising with response (PAwR) communication session.
Clause J73. The system of clause J54, wherein the first electronics are configured to establish communication with the second receiver device by wireless transmission of a non-synchronous advertisement, and receipt of a response to the non-synchronous advertisement from the second receiver device.
Clause J74. The system of clause J73, wherein the first electronics are configured to establish communication with the second receiver device with a non-synchronous advertising session.
Clause J75. The system of clause J74, wherein the first electronics are configured to use the non-synchronous advertising-based session to exchange setup information to form a synchronous advertising-based session with the second receiver device.
Clause J76. The system of clause J75, wherein the synchronous advertising-based session is a periodic advertising with response (PAwR) communication session.
Clause J77. The system of clause J75, wherein the setup information comprises a clock accuracy parameter of the second receiver device.
Clause J78. The system of clause J75, wherein the first electronics are configured to select a transport protocol for communication with the second receiver device based, at least in part, on the clock accuracy parameter.
Clause J79. The system of any of clauses J74 through J78, further comprising ending the non-synchronous advertising session between the on-body device and the second receiver device and communicating between the on-body device and the second receiver device with the synchronous advertising-based session.
Clause J80. The system of any of clauses J73 through J79, wherein the first electronics are configured to maintain synchronous advertising communication with the first receiver device and maintain non-synchronous advertising-based communication with the second receiver device.
Clause J81. The system of any of clauses J70 through J80, wherein the synchronous advertising-based session is a periodic advertising with response (PAwR) communication session.
Clause J82. The system of any of clauses J2 through J53, wherein the receiver device is a first receiver device, and wherein the first electronics are configured to communicate with a second receiver device with a synchronous advertising-based session while maintaining communication with the first receiver device.
Clause J83. The system of clause J82, wherein the first electronics are configured to join the second receiver device directly into the synchronous advertising-based session.
Clause J84. The system of any of clauses J82 or J83, wherein the first electronics are configured to communicate synchronous advertising-based session setup information to the second receiver using a non-Bluetooth communication protocol.
Clause J85. The system of clause J84, wherein the non-Bluetooth communication protocol is Near Field Communication (NFC).
Clause J86. The system of any of clauses J83 through J85, wherein the synchronous advertising-based session setup information comprises a clock accuracy parameter of the second receiver device.
Clause J87. The system of clause J86, wherein the first electronics are configured to select a transport protocol for communication with the second receiver device based, at least in part, on the clock accuracy parameter.
Clause J88. The system of any of clauses J82 or J83, wherein the first electronics are configured to receive a response to a synchronous advertisement in a predetermined response time slot.
Clause J89. The system of clause J88, wherein the predetermined response time slot is reserved for a new receiver device seeking to join communication with the on-body device.
Clause J90. The system of any of clauses J54 through J89, wherein the first electronics are configured to communicate a second receiver response time slot to the second receiver device.
Clause J91. The system of clause J90, wherein the second receiver response time slot is immediately adjacent a response time slot for the first receiver device.
Clause J92. The system of any of clauses J54 through J91, wherein the first electronics are configured to reassign a response time slot to at least one receiver device when another receiver device ceases communication with the on-body device.
Clause J93. The system of clause J92, wherein the reassigned response time slot has a reduced delay from the last communication from the on-body device.
Clause J94. The system of any of clauses J1 through J93, wherein the first receiver device is one of a plurality of receiver devices, wherein the first electronics are configured to conduct a synchronous advertising-based communication session with the plurality of receiver devices, wherein the first electronics are configured to transmit advertisements periodically during the synchronous advertising-based communication session, wherein each transmitted advertisement comprises information indicative of an analyte level of the subject and is configured for receipt by all of the plurality of receiver devices.
Clause J95. The system of clause J94, wherein the on-body device dos not monitor for all responses from the plurality of receiver devices.
Clause J96. The method of any of clauses J94 or J95, wherein the on-body device does not monitor for a response from every receiver device after every advertisement.
Clause J97. The method of any of clauses J94 through J96, wherein the on-body device selectively monitors for responses from the plurality of receiver devices such that the on-body device does not monitor for selective responses transmitted by the plurality of receiver devices.
Clause J98. The method of any of clauses J94 through J97, wherein the on-body device monitors for responses from a subset of one or more of the receiver devices in the plurality of receiver devices, but not all receiver devices in the plurality of receiver devices, after transmission of an advertisement to all of the receiver devices in the plurality of receiver devices.
Clause J99. The method of clauses J94 through J98, wherein the on-body device does not monitor for a response from any of the receiver devices in the plurality of receiver devices after transmission of an advertisement to all of the receiver devices in the plurality of receiver devices.
Clause J100. The method of any of clauses J94 through J99, wherein at least a first receiver device of the plurality of receiver devices is a primary receiver device and at least a second receiver device of the plurality of receiver devices is a secondary receiver device, wherein the on-body device monitors for responses from the primary receiver device more frequently than from the secondary receiver device after transmitting advertisements to both the primary and secondary receiver devices.
Clause J101. The system of clause J94, wherein the first electronics are configured to make a modification to the synchronous advertising-based communication session.
Clause J102. The system of clause J101, wherein the modification to the synchronous advertising-based communication session comprises a modification to a transmission interval.
Clause J103. The system of clause J94, wherein the first electronics are configured to transmit advertisements periodically according to a first transmission interval, and wherein the first electronics are configured to make a modification to the synchronous advertising-based communication session by informing the plurality of receiver devices of a second transmission interval that is greater than the first transmission interval and conducting the synchronous advertising-based communication session by transmitting advertisements periodically according to the second transmission interval. J103
Clause J104. The system of clause 288, wherein the first electronics are configured to revert to the first transmission interval.
Clause J105. The system of clause J101, wherein the modification to the synchronous advertising-based communication session comprises a modification to a transmission subinterval.
Clause J106. The system of clause J105, wherein the first electronics are configured to transmit advertisements periodically according to a first transmission interval comprising a plurality of first transmission subintervals each having a first duration, and wherein the first electronics are configured to make a modification to the synchronous advertising-based communication session by: the provision of notification to the plurality of receiver devices of a change that modifies the first duration of the first transmission subintervals to a second duration that is different from the first duration; and by performance of the synchronous advertising-based communication session according to the second duration of the first plurality of transmission subintervals.
Clause J107. The system of clause J106, wherein the first electronics are configured to revert to communication according to the first duration.
Clause J108. The system of clause J101, wherein the first electronics are configured to modify a duration of a response time slot.
Clause J109. The system of clause J108, wherein the plurality of receiver devices respond in response time slots each having a first duration, and wherein the first electronics are configured to make a modification to the synchronous advertising-based communication session by informing the plurality of receiver devices of a second duration for response time slots that is less than the first duration and conducting the synchronous advertising-based communication session by transmitting responses in response time slots having the second duration.
Clause J110. The system of clause J109, wherein the first electronics are configured to revert to the first duration.
Clause J111. The system of clause J101, wherein the first electronics are configured to modify a first operating aspect of monitoring for responses.
Clause J112. The system of clause J111, wherein the first electronics are configured to monitor for responses from the plurality of receiver devices according to the first operating aspect, and wherein the first electronics are configured to make a modification to the synchronous advertising-based communication session by conducting the synchronous advertising-based communication session by monitoring for responses according to a second operating aspect, wherein the second operating aspect corresponds to monitoring less frequently than the first operating aspect.
Clause J113. The system of clause J112, wherein the first operating aspect corresponds to monitoring for responses at least once in every transmission interval.
Clause J114. The system of clause J112, wherein the first operating aspect corresponds to monitoring for responses at least twice in every transmission interval.
Clause J115. The system of clause J112, wherein the first operating aspect corresponds to monitoring for responses at least once in a predetermined duration of time.
Clause J116. The system of any of clauses J112 through J115, wherein the first electronics are configured to not monitor for at least one response transmitted by the plurality of receiver devices when in operation according to the second operating aspect.
Clause J117. The system of any of clauses J112 through J116, wherein at least a first receiver device of the plurality of receiver devices is a primary receiver device and at least a second receiver device of the plurality of receiver devices is a secondary receiver device, wherein the on-body device is configured to monitor for responses from the primary receiver device more frequently than from the secondary receiver device when in operation according to the second operating aspect.
Clause J118. The system of clause J117, wherein at least one receiver device of the plurality of receiver devices is configured to accept an input by a user that designates which of the plurality of receiver devices is the primary receiver device.
Clause J119. The system of any of clauses J112 through J118, wherein the first electronics are configured to modify a power consumption of the synchronous advertising-based communication session.
Clause J120. The system of clause J101, wherein the first electronics are configured to make a modification to the synchronous advertising-based communication session by deactivating wireless communication electronics of the on-body device at least once during every transmission interval.
Clause J121. The system of clause J101, wherein the first electronics are configured to make a modification to the synchronous advertising-based communication session by increasing a time of deactivation of wireless communication electronics of the on-body device during every transmission interval.
Clause J122. The system of clause J101, wherein the first electronics are configured to make a modification to the synchronous advertising-based communication session by transmitting advertisements by the on-body device with a relatively lower transmit power than prior to the modification.
Clause J123. The system of clause J101, wherein the first electronics are configured to make a modification to the synchronous advertising-based communication session by changing a physical format of communication.
Clause J124. The system of any of clauses J101 through J123, wherein the first electronics are configured to determine to make the modification to the synchronous advertising-based communication session based, at least in part, on a state of power of the on-body device.
Clause J125. The system of any of clauses J101 through J124, wherein the first electronics are configured to determine to make the modification to the synchronous advertising-based communication session based, at least in part, on a schedule.
Clause J126. The system of any of clauses J101 through J125, wherein the first electronics are configured to determine to make the modification to the synchronous advertising-based communication session based, at least in part, on detected analyte monitoring condition state.
Clause J127. The system of any of clauses J101 through J126, wherein the first electronics are configured to determine to make the modification to the synchronous advertising-based communication session based, at least in part, on stability of analyte levels of the subject.
Clause J128. The system of any of clauses J101 through J127, wherein the first electronics are configured to determine to make the modification to the synchronous advertising-based communication session based, at least in part, on a mask condition of the on-body device.
Clause J129. The system of any of clauses J101 through J128, wherein the first electronics are configured to determine to make the modification to the synchronous advertising-based communication session based, at least in part, on a change in the number of receiver devices communicating with the on-body device.
Clause J130. The system of any of clauses J101 through J129, wherein the first electronics are configured to determine to make the modification to the synchronous advertising-based communication session based, at least in part, on a clock accuracy parameter of at least one receiver device of the plurality of receiver devices.
Clause J131. The system of any of clauses J1 through J130, wherein the first electronics are configured to receive a supplementary information request that requests second information indicative of the level of the analyte of the subject that was not transmitted in the first advertisement.
Clause J132. The system of clause J131, wherein the first electronics are configured to transmit the second information indicative of the level of the analyte of the subject in response to the request.
Clause J133. The system of clause J132, wherein the first electronics are configured to transmit the second information indicative of the level of the analyte of the subject on a secondary communication channel of a Bluetooth protocol.
Clause J134. The system of any of clauses J132 or J133, wherein the first electronics are configured to receive the supplementary information request during a first transmission interval of a synchronous advertising-based communication session and configured to transmit the second information indicative of the level of the analyte of the subject during the first transmission interval.
Clause J135. The system of any of clauses J132 or J133, wherein the first electronics are configured to receive the supplementary information request during a first transmission subinterval of a first transmission interval of a synchronous advertising-based communication session and configured to transmit the second information indicative of the level of the analyte of the subject during a subsequent transmission subinterval of the first transmission interval.
Clause J136. The system of any of clauses J132 or J133, wherein the first electronics are configured to receive the supplementary information request during a first transmission interval of a synchronous advertising-based communication session and configured to transmit the second information indicative of the level of the analyte of the subject during a second transmission interval.
Clause J137. The system of any of clauses J133 through J136, wherein the first electronics are configured to transmit a pointer that identifies a secondary channel on which the second information indicative of the level of the analyte of the subject will be transmitted.
Clause J138. The system of clause J137, wherein the pointer further identifies a time offset at which the second information indicative of the level of the analyte of the subject will be transmitted.
Clause J139. The system of any of clauses J132 through J138, wherein the second information indicative of the level of the analyte of the subject is transmitted as a directed or addressed communication to the receiver device that issued the supplementary information request.
Clause J140. The system of any of clauses J132 through J138, wherein the first electronics are configured to transmit the second information indicative of the level of the subject in a plurality of chained advertisements on one or more secondary communication channels of the Bluetooth protocol.
Clause J141. The system of any of clauses J2 through J140, further comprising the first receiver device and a second receiver device, wherein the first receiver device is configured to wirelessly transmit a response to the first advertisement and the second receiver device is configured to wirelessly receive the response to the first advertisement.
Clause J142. The system of clause J141, wherein the response comprises at least one of: an alarm activation or deactivation state; an alarm threshold setting; an alarm enablement setting; an alarm notification format setting; an alarm mode setting; analyte data; a user-specific setting; a personal profile setting; calibration information; and/or drug therapy and/or insulin information.
Clause J143. The system of any of clauses J141 or J142, wherein the second receiver device is configured to interpret the response to the first advertisement.
Clause J144. The system of clause J143, wherein the second receiver device is configured to take an action based on information contained in the response to the first advertisement.
Clause J145. The system of clause J144, wherein the second receiver device is configured to take an action by updating or first storing information in the second receiver device.
Clause J146. The system of any of clauses J1 through J145, wherein the first electronics are configured to transmit a second advertisement comprising data indicative of an analyte level alarm condition and wherein the first receiver device and second receiver device are configured to activate alarms based on the received data indicative of the level of the analyte alarm condition.
Clause J147. The system of clause J146, wherein the first receiver device is configured to wirelessly transmit a response to the first advertisement indicating that a user acknowledged and/or dismissed the alarm on the first receiver device.
Clause J148. The system of clause J147, wherein the second receiver device is configured to wirelessly receive the response to the first advertisement indicating that a user acknowledged and/or dismissed the alarm on the first receiver device.
Clause J149. The system of clause J148, wherein the second receiver device is configured to deactivate the alarm on the second receiver device based on the received response to the first advertisement indicating that a user acknowledged and/or dismissed the alarm on the first receiver device.
Clause J150. The system of any of clauses J1 through J149, wherein the analyte is selected from the list consisting of: glucose, one or more ketones, lactate, and oxygen.
Clause J151. The system of any of clauses J1 through J150, wherein the analyte is selected from the list consisting of: ammonium, asparagine, aspartate, cholesterol, choline, creatinine, one or more diaphorase inhibitors, ethanol, glucose, glutamate, glycerol, one or more ketones, lactate, lactose, oxygen, phosphate, potassium, pyruvate, and uric acid.
Clause J152. The system of any of clauses J1 through J151, wherein the analyte is a first analyte, and the on-body device is further configured to obtain second information indicative of a level of a second analyte of the subject.
Clause J153. The system of clause J152, wherein the first analyte is glucose and the second analyte is a ketone.
Clause J154. The system of clause J152, wherein the first and second analytes are selected from the list consisting of: ammonium, asparagine, aspartate, cholesterol, choline, creatinine, one or more diaphorase inhibitors, ethanol, glucose, glutamate, glycerol, one or more ketones, lactate, lactose, oxygen, phosphate, potassium, pyruvate, and uric acid.
Clause J155. The system of any of clauses J1 through J154, wherein the on-body device comprises a partially implantable sensor configured to measure the level of the analyte of the subject.
Clause J156. The system of any of clauses J1 through J154, wherein the on-body device is configured to wirelessly communicate with a wholly implantable sensor configured to measure the level of the analyte of the subject.
Clause J157. The system of clause J156, further comprising the wholly implantable sensor.
Clause J158. The system of any of clauses J1 through J154, wherein the on-body device comprises a wholly ex vivo sensor configured to measure the level of the analyte of the subject.
Clause J159. The system of any of clauses J2 through J154 to the extent dependent on clause J2, wherein the receiver device is a multi-purpose mobile receiver device.
Clause J160. The system of clause J159, wherein the receiver device is smart phone.
Clause J161. The system of clause J159, wherein the receiver device is a smart watch.
Clause J162. The system of any of clauses J2 through J154 to the extent dependent on clause J2, further comprising the receiver device.
Clause J163. The system of any of clauses J2 through J154 to the extent dependent on clause J2, wherein the receiver device is a first receiver device, the system further comprising a second receiver device.
Clause J164. The system of clause J163, wherein the first receiver device is a smart phone and the second receiver device is a smart watch.
Clause J165. The system of any of clauses J2 through J154 and J162 or J163 to the extent dependent on clause 187, wherein the receiver device is a dedicated receiver device.
Clause J166. The system of any of clauses J2 through J154 and J162 or J163 to the extent dependent on clause 187, wherein the receiver device is drug delivery device.
Clause J167. The system of any of clauses J2 through J154 and J162 or J163 to the extent dependent on clause 187, wherein the receiver device is a wireless computing device.
Clause J168. The system of any of clauses J1 through J167, wherein the on-body device further comprises a housing coupled with an adhesive pad.
Clause J169. The system of any of clauses J1 through J168, further comprising an applicator or inserter for applying the on-body device to a subject's body.
Clause J170. The system of any of clauses J1 through J169 in combination with the features of the system of any of clauses A1 through A94 or A96 through A101.
Clause J171. The system of any of clauses J1 through J169 in combination with the features of the system of any of clauses C1 through C18 or C20.
Clause J172. The system of any of clauses J1 through J169 in combination with the features of the system of any of clauses F1 through F25 or F27.
Clause J172. The system of any of clauses J1 through J169 in combination with the features of the system of any of clauses H1 through H18 or H20.
Clause J173. The system of any of clauses J1 through J169 in combination with the features of the system of any of clauses AF1 through AF22.
Clause J174. The system of any of clauses J1 through J173, wherein the on-body device further comprises:
a sensor; and
an adhesive patch adhesively coupled with the housing,
wherein the first electronics comprise: wireless communication electronics; and sensor interface electronics configured to receive a signal from the sensor, the signal indicative of an analyte level of a user;
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the level of the analyte in the bodily fluid and output the signal to the sensor interface electronics.
Clause K1. A method of wireless communication in an on-body analyte monitoring system, comprising: wirelessly transmitting, by an on-body device on the body of a subject, a first advertisement comprising first information indicative of a level of an analyte of the subject; wirelessly receiving, by the on-body device, a first response from a first receiver device, wherein the first response confirms whether or not the first advertisement was received by the first receiver device; and wirelessly receiving, by the on-body device, a second response from a second receiver device, wherein the second response confirms whether or not the first advertisement was received by the second receiver device, wherein the first and second responses are transmitted synchronously in first and second time slots, respectively, at times known by the on-body device.
Clause K2. The method of clause K1 in combination with the features of the method of any of clauses B1 through B70 or B72 through B77.
Clause K3. The method of clause K1 in combination with the features of the method of any of clauses D1 through D9 or D10.
Clause K4. The method of clause K1 in combination with the features of the method of any of clauses E1 through E21 or E23.
Clause K5. The method of clause K1 in combination with the features of the method of any of clauses G1 through G13 or G15.
Clause K6. The method of clause K1 in combination with the features of the method of any of clauses AE1 through AE22.
Clause K7. The method of any of clauses K1 through K6, wherein the on-body device comprises:
a sensor;
wireless communication electronics;
sensor interface electronics configured to receive a signal from the sensor, the signal indicative of an analyte level of a user;
a housing configured to retain the wireless communication electronics and sensor interface electronics; and
an adhesive patch adhesively coupled with the housing,
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the analyte level in the bodily fluid and output the signal to the sensor interface electronics.
Clause L1. An on-body analyte monitoring system, comprising: an on-body device configured to be held on the body of a subject, the on-body device comprising a housing configured to hold first electronics, wherein the first electronics are configured to: wirelessly transmit a first advertisement comprising first information indicative of the level of the analyte of the subject; wirelessly receive a first response from a first receiver device, wherein the first response confirms whether or not the first advertisement was received by the first receiver device; and wirelessly receive a second response from a second receiver device, wherein the second response confirms whether or not the first advertisement was received by the second receiver device, wherein the first electronics are configured to receive the first and second responses synchronously in first and second time slots, respectively, at times known to the first electronics.
Clause L2. The system of clause L2, further comprising the first and second receiver devices.
Clause L3. The system of any of clauses L1 or L2 in combination with the features of the system of any of clauses A1 through A94 or A96 through A101.
Clause L4. The system of any of clauses L1 or L2 in combination with the features of the system of any of clauses C1 through C18 or C20.
Clause L5. The system of any of clauses L1 or L2 in combination with the features of the system of any of clauses F1 through F25 or F27.
Clause L6. The system of any of clauses L1 or L2 in combination with the features of the system of any of clauses H1 through H18 or H20.
Clause L7. The system of any of clauses L1 or L2 in combination with the features of the system of any of clauses AF1 through AF22.
Clause L8. The system of any of clauses L1 through L7, wherein the on-body device further comprises:
a sensor; and
an adhesive patch adhesively coupled with the housing,
wherein the first electronics comprise: wireless communication electronics; and sensor interface electronics configured to receive a signal from the sensor, the signal indicative of an analyte level of a user;
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the level of the analyte in the bodily fluid and output the signal to the sensor interface electronics.
Clause M1. A method of wireless communication in an on-body analyte monitoring system, comprising: establishing an initial wireless connection between an on-body device on a body of a subject and a first receiver device; performing an authentication process for at least one of the on-body device and the first receiver device; exchanging synchronous connection setup information; and establishing a wireless synchronous advertising-based communication session using the synchronous connection setup information.
Clause M2. The method of clause M1, wherein the initial wireless connection is operated according to a non-synchronous transport protocol.
Clause M3. The method of clause M1, wherein the initial wireless connection is operated according to a Near-Field Communication (NFC) protocol.
Clause M4. The method of any of clauses M1 through M3, wherein the authentication process is performed using the initial wireless connection.
Clause M5. The method of any of clauses M1 through M3, wherein the synchronous connection setup information is exchanged using the initial wireless connection.
Clause M6. The method of any of clauses M1 through M5, wherein the synchronous advertising-based communication session is operated according to a Bluetooth protocol.
Clause M7. The method of any of clauses M1 through M6 in combination with the features of the method of any of clauses B1 through B70 or B72 through B77.
Clause M8. The method of any of clauses M1 through M6 in combination with the features of the method of any of clauses D1 through D9 or D10.
Clause M9. The method of any of clauses M1 through M6 in combination with the features of the method of any of clauses E1 through E21 or E23.
Clause M10. The method of any of clauses M1 through M6 in combination with the features of the method of any of clauses G1 through G13 or G15.
Clause M11. The method of any of clauses M1 through M6 in combination with the features of the method of any of clauses AE1 through AE22.
Clause M12. The method of any of clauses M1 through M11, wherein the on-body device comprises:
a sensor;
wireless communication electronics;
sensor interface electronics configured to receive a signal from the sensor, the signal indicative of an analyte level of a user;
a housing configured to retain the wireless communication electronics and sensor interface electronics; and
an adhesive patch adhesively coupled with the housing,
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the analyte level in the bodily fluid and output the signal to the sensor interface electronics.
Clause N1. An on-body analyte monitoring system, comprising: an on-body device configured to be held on the body of a subject, the on-body device comprising a housing configured to hold first electronics, wherein the first electronics are configured to: establish an initial wireless connection with a receiver device; perform an authentication process for the receiver device; exchange synchronous connection setup information with the receiver device; and establish a wireless synchronous advertising-based communication session with the receiver device according to the synchronous connection setup information.
Clause N2. The system of clause N1 in combination with the features of the system of any of clauses A1 through A94 or A96 through A101.
Clause N3. The system of clause N1 in combination with the features of the system of any of clauses C1 through C18 or C20.
Clause N4. The system of clause N1 in combination with the features of the system of any of clauses F1 through F25 or F27.
Clause N5. The system of clause N1 in combination with the features of the system of any of clauses H1 through H18 or H20.
Clause N6. The system of clause N1 in combination with the features of the system of any of clauses AF1 through AF22.
Clause N7. The system of any of clauses N1 through N6, wherein the on-body device further comprises:
a sensor; and
an adhesive patch adhesively coupled with the housing,
wherein the first electronics comprise: wireless communication electronics; and sensor interface electronics configured to receive a signal from the sensor, the signal indicative of an analyte level of a user;
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the level of the analyte in the bodily fluid and output the signal to the sensor interface electronics.
Clause O1. An on-body analyte monitoring system, comprising: a receiver device comprising a housing configured to hold receiver electronics, wherein the receiver electronics are configured to: establish an initial wireless connection with an on-body device; perform an authentication process for the on-body device; exchange synchronous connection setup information with the on-body device; and establish a wireless synchronous advertising-based communication session with the on-body device according to the synchronous connection setup information.
Clause O2. The system of clause O1 in combination with the features of the system of any of clauses A1 through A94 or A96 through A101.
Clause O3. The system of clause O1 in combination with the features of the system of any of clauses C1 through C18 or C20.
Clause O4. The system of clause O1 in combination with the features of the system of any of clauses F1 through F25 or F27.
Clause O5. The system of clause O1 in combination with the features of the system of any of clauses H1 through H18 or H20.
Clause O6. The system of clause O1 in combination with the features of the system of any of clauses AF1 through AF22.
Clause O7. The system of any of clauses O1 through O6, further comprising the on-body device, wherein the on-body device comprises:
a sensor;
wireless communication electronics;
sensor interface electronics configured to receive a signal from the sensor, the signal indicative of an analyte level of a user;
a housing configured to retain the wireless communication electronics and sensor interface electronics; and
an adhesive patch adhesively coupled with the housing,
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the level of the analyte in the bodily fluid and output the signal to the sensor interface electronics.
Clause P1. A method of wireless communication in an on-body analyte monitoring system, comprising: conducting a first communication session according to a first transport protocol between an on-body device and a receiver device; determining, by one of the on-body device and receiver device, to change to a second transport protocol; negotiating, by the on-body device and the receiver device, one or more parameters for conducting a second communication session according to the second transport protocol, wherein either the first transport protocol or the second transport protocol is a synchronous advertising-based transport protocol; and initiating, by one of the on-body device and receiver device, the second communication session according to the second transport protocol and communicating between the on-body device and the receiver device in the second communication session.
Clause P2. The method of clause P1, wherein the first transport protocol is the synchronous advertising-based transport protocol.
Clause P3. The method of clause P1, wherein the second transport protocol is the synchronous advertising-based transport protocol.
Clause P4. The method of any of clauses P1 through P3, wherein either the first transport protocol or the second transport protocol is the synchronous advertising-based transport protocol and the other transport protocol is a non-synchronous transport protocol.
Clause P5. The method of any of clauses P1 through P4 in combination with the features of the method of any of clauses B1 through B70 or B72 through B77.
Clause P6. The method of any of clauses P1 through P4 in combination with the features of the method of any of clauses D1 through D9 or D10.
Clause P7. The method of any of clauses P1 through P4 in combination with the features of the method of any of clauses E1 through E21 or E23.
Clause P8. The method of any of clauses P1 through P4 in combination with the features of the method of any of clauses G1 through G13 or G15.
Clause P9. The method of any of clauses P1 through P4 in combination with the features of the method of any of clauses AE1 through AE22.
Clause P10. The method of any of clauses P1 through P9, wherein the on-body device comprises:
a sensor;
wireless communication electronics;
sensor interface electronics configured to receive a signal from the sensor, the signal indicative of an analyte level of a user;
a housing configured to retain the wireless communication electronics and sensor interface electronics; and
an adhesive patch adhesively coupled with the housing,
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the level of the analyte in the bodily fluid and output the signal to the sensor interface electronics.
Clause Q1. An on-body analyte monitoring system, comprising: an on-body device configured to be held on the body of a subject, the on-body device comprising a housing configured to hold first electronics, wherein the first electronics are configured to: conduct a first communication session according to a first transport protocol with a receiver device; determine to change to a second transport protocol; negotiate one or more parameters for conducting a second communication session according to the second transport protocol, wherein either the first transport protocol or the second transport protocol is a synchronous advertising-based transport protocol; and initiate the second communication session according to the second transport protocol and communicate with the receiver device in the second communication session.
Clause Q2. The system of clause Q1 in combination with the features of the system of any of clauses A1 through A94 or A96 through A101.
Clause Q3. The system of clause Q1 in combination with the features of the system of any of clauses C1 through C18 or C20.
Clause Q4. The system of clause Q1 in combination with the features of the system of any of clauses F1 through F25 or F27.
Clause Q5. The system of clause Q1 in combination with the features of the system of any of clauses H1 through H18 or H20.
Clause Q6. The system of clause Q1 in combination with the features of the system of any of clauses AF1 through AF22.
Clause Q7. The system of any of clauses Q1 through Q6, wherein the on-body device further comprises:
a sensor; and
an adhesive patch adhesively coupled with the housing,
wherein the first electronics comprise: wireless communication electronics; and sensor interface electronics configured to receive a signal from the sensor, the signal indicative of an analyte level of a user;
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the level of the analyte in the bodily fluid and output the signal to the sensor interface electronics.
Clause R1. An on-body analyte monitoring system, comprising: a receiver device comprising a housing configured to hold receiver electronics, wherein the receiver electronics are configured to: conduct a first communication session according to a first transport protocol with an on-body device; determine to change to a second transport protocol; negotiate one or more parameters for conducting a second communication session according to the second transport protocol, wherein either the first transport protocol or the second transport protocol is a synchronous advertising-based transport protocol; and initiate the second communication session according to the second transport protocol and communicate with the on-body device in the second communication session.
Clause R2. The system of clause RI in combination with the features of the system of any of clauses A1 through A94 or A96 through A101.
Clause R3. The system of clause RI in combination with the features of the system of any of clauses C1 through C18 or C20.
Clause R4. The system of clause RI in combination with the features of the system of any of clauses F1 through F25 or F27.
Clause R5. The system of clause RI in combination with the features of the system of any of clauses H1 through H18 or H20.
Clause R6. The system of clause RI in combination with the features of the system of any of clauses AF1 through AF22.
Clause R7. The system of any of clauses RI through R6, further comprising the on-body device, wherein the on-body device comprises:
a sensor;
wireless communication electronics;
sensor interface electronics configured to receive a signal from the sensor, the signal indicative of an analyte level of a user;
a housing configured to retain the wireless communication electronics and sensor interface electronics; and
an adhesive patch adhesively coupled with the housing,
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the level of the analyte in the bodily fluid and output the signal to the sensor interface electronics.
Clause S1. A method of wireless communication in an on-body analyte monitoring system, comprising: negotiating, by an on-body device with a receiver device, in a communication session according to a first non-synchronous transport protocol; determining not to communicate with the receiver device using a synchronous advertising-based transport protocol; determining to continue the communication session, or to form a new non-synchronous session, for communication of analyte data between the on-body device and the receiver device.
Clause S2. The method of clause S1 in combination with the features of the method of any of clauses B1 through B70 or B72 through B77.
Clause S3. The method of clause S1 in combination with the features of the method of any of clauses D1 through D9 or D10.
Clause S4. The method of clause S1 in combination with the features of the method of any of clauses E1 through E21 or E23.
Clause S5. The method of clause S1 in combination with the features of the method of any of clauses G1 through G13 or G15.
Clause S6. The method of clause S1 in combination with the features of the method of any of clauses AE1 through AE22.
Clause S7. The method of any of clauses S1 through S6, wherein the on-body device comprises:
a sensor;
wireless communication electronics;
sensor interface electronics configured to receive a signal from the sensor, the signal indicative of an analyte level of a user;
a housing configured to retain the wireless communication electronics and sensor interface electronics; and
an adhesive patch adhesively coupled with the housing,
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the level of the analyte in the bodily fluid and output the signal to the sensor interface electronics.
Clause T1. An on-body analyte monitoring system, comprising: an on-body device configured to be held on the body of a subject, the on-body device comprising a housing configured to hold first electronics, wherein the first electronics are configured to: negotiate with a receiver device in a communication session according to a first non-synchronous transport protocol; determine not to communicate with the receiver device according to a synchronous advertising-based transport protocol; determine to continue the communication session, or to form a new non-synchronous session, for communication of analyte data between the on-body device and the receiver device.
Clause T2. The system of clause T1 in combination with the features of the system of any of clauses A1 through A94 or A96 through A101.
Clause T3. The system of clause T1 in combination with the features of the system of any of clauses C1 through C18 or C20.
Clause T4. The system of clause T1 in combination with the features of the system of any of clauses F1 through F25 or F27.
Clause T5. The system of clause T1 in combination with the features of the system of any of clauses H1 through H18 or H20.
Clause T6. The system of clause T1 in combination with the features of the system of any of clauses AF1 through AF22.
Clause T7. The system of any of clauses T1 through T6, wherein the on-body device further comprises:
a sensor; and
an adhesive patch adhesively coupled with the housing,
wherein the first electronics comprise: wireless communication electronics; and sensor interface electronics configured to receive a signal from the sensor, the signal indicative of an analyte level of a user;
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the level of the analyte in the bodily fluid and output the signal to the sensor interface electronics.
Clause U1. A method of wireless communication in an on-body analyte monitoring system, comprising: transmitting, as part of a synchronous communication session, an advertisement by an on-body device to a first receiver device and a second receiver device; transmitting, by the first receiver device, a first response; receiving, by the on-body device and the second receiver device, the first response; and interpreting the received first response by the on-body device and the second receiver device.
Clause U2. The method of clause U1 in combination with the features of the method of any of clauses B1 through B70 or B72 through B77.
Clause U3. The method of clause U1 in combination with the features of the method of any of clauses D1 through D9 or D10.
Clause U4. The method of clause U1 in combination with the features of the method of any of clauses E1 through E21 or E23.
Clause U5. The method of clause U1 in combination with the features of the method of any of clauses G1 through G13 or G15.
Clause U6. The method of clause U1 in combination with the features of the method of any of clauses AE1 through AE22.
Clause U7. The method of any of clauses U1 through U6, wherein the on-body device comprises:
a sensor;
wireless communication electronics;
sensor interface electronics configured to receive a signal from the sensor, the signal indicative of an analyte level of a user;
a housing configured to retain the wireless communication electronics and sensor interface electronics; and
an adhesive patch adhesively coupled with the housing,
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the level of the analyte in the bodily fluid and output the signal to the sensor interface electronics.
Clause V1. An on-body analyte monitoring system, comprising: a first receiver device comprising a housing configured to hold receiver electronics, wherein the receiver electronics are configured to: receive, as part of a synchronous communication session, an advertisement from an on-body device; receive, from a second receiver device, a first response to the advertisement; and interpret the received first response.
Clause V2. The system of clause V1, wherein the receiver electronics are configured to take action based on the interpreted first response.
Clause V3. The system of any of clauses V1 or V2 in combination with the features of the system of any of clauses A1 through A94 or A96 through A101.
Clause V4. The system of any of clauses V1 or V2 in combination with the features of the system of any of clauses C1 through C18 or C20.
Clause V5. The system of any of clauses V1 or V2 in combination with the features of the system of any of clauses F1 through F25 or F27.
Clause V6. The system of any of clauses V1 or V2 in combination with the features of the system of any of clauses H1 through H18 or H20.
Clause V7. The system of any of clauses V1 or V2 in combination with the features of the system of any of clauses AF1 through AF22.
Clause V8. The system of any of clauses V1 through V7, further comprising the on-body device, wherein the on-body device further comprises:
a sensor;
wireless communication electronics;
sensor interface electronics configured to receive a signal from the sensor, the signal indicative of an analyte level of a user;
a housing configured to retain the wireless communication electronics and sensor interface electronics; and
an adhesive patch adhesively coupled with the housing,
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the level of the analyte in the bodily fluid and output the signal to the sensor interface electronics.
Clause W1. A method of wireless communication in an on-body analyte monitoring system, comprising: transmitting, as part of a synchronous communication session, a first advertisement by an on-body device to a first receiver device and a second receiver device, wherein the advertisement includes data indicative of an analyte level alarm condition; receiving the first advertisement and activating first and second alarms by the first receiver device and the second receiver device, respectively; receiving, by the first receiver device, a user acknowledgement of the first alarm and deactivating the first alarm; receiving, by the second receiver device, a response from the first receiver device to a second advertisement, wherein the response indicates that the user acknowledged the first alarm on the first receiver device; and deactivating the second alarm by the second receiver device based on the received response.
Clause W2. The method of clause W1, wherein the second receiver device deactivated the second alarm without first receiving user acknowledgement of the second alarm at the second receiver device.
Clause W3. The method of any of clauses W1 or W2 in combination with the features of the method of any of clauses B1 through B70 or B72 through B77.
Clause W4. The method of any of clauses W1 or W2 in combination with the features of the method of any of clauses D1 through D9 or D10.
Clause W5. The method of any of clauses W1 or W2 in combination with the features of the method of any of clauses E1 through E21 or E23.
Clause W6. The method of any of clauses W1 or W2 in combination with the features of the method of any of clauses G1 through G13 or G15.
Clause W7. The method of any of clauses W1 or W2 in combination with the features of the method of any of clauses AE1 through AE22.
Clause W8. The method of any of clauses W1 through W7, wherein the on-body device comprises:
a sensor;
wireless communication electronics;
sensor interface electronics configured to receive a signal from the sensor, the signal indicative of an analyte level of a user;
a housing configured to retain the wireless communication electronics and sensor interface electronics; and
an adhesive patch adhesively coupled with the housing,
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the level of the analyte in the bodily fluid and output the signal to the sensor interface electronics.
Clause X1. An on-body analyte monitoring system, comprising: a first receiver device comprising a housing configured to hold receiver electronics, wherein the receiver electronics are configured to: receive a first advertisement from an on-body device including data indicative of an analyte level alarm condition; activate a first alarm based on the received first advertisement; receive a response from a second receiver device to a second advertisement, wherein the response indicates that the user acknowledged a corresponding alarm on the second receiver device; and deactivate the first alarm based on the received response.
Clause X2. The system of clause X1 in combination with the features of the system of any of clauses A1 through A94 or A96 through A101.
Clause X3. The system of clause X1 in combination with the features of the system of any of clauses C1 through C18 or C20.
Clause X4. The system of clause X1 in combination with the features of the system of any of clauses F1 through F25 or F27.
Clause X5. The system of clause X1 in combination with the features of the system of any of clauses H1 through H18 or H20.
Clause X6. The system of clause X1 in combination with the features of the system of any of clauses AF1 through AF22.
Clause Y1. A method of wireless communication in an on-body analyte monitoring system, comprising: transmitting a first advertisement, by an on-body device in a synchronous advertising-based communication session, to which a receiver device responds with a first response including a supplementary information request; transmitting a second advertisement, by the on-body device in the synchronous advertising-based communication session, at least a portion of the missing analyte data, to which the receiver device responds with a second response acknowledging receipt.
Clause Y2. The method of clause Y1 in combination with the features of the method of any of clauses B1 through B70 or B72 through B77.
Clause Y3. The method of clause Y1 in combination with the features of the method of any of clauses D1 through D9 or D10.
Clause Y4. The method of clause Y1 in combination with the features of the method of any of clauses E1 through E21 or E23.
Clause Y5. The method of clause Y1 in combination with the features of the method of any of clauses G1 through G13 or G15.
Clause Y6. The method of clause Y1 in combination with the features of the method of any of clauses AE1 through AE22.
Clause Y7. The method of clause Y1 in combination with the features of the method of any of clauses I1 through I185.
Clause Y8. The method of clause Y1 in combination with the features of the method of clause K1.
Clause Y9. The method of clause Y1 in combination with the features of the method of any of clauses M1 through M6.
Clause Y11. The method of clause Y1 in combination with the features of the method of any of clauses P1 through P4.
Clause Y13. The method of clause Y1 in combination with the features of the method of clause S1.
Clause Y14. The method of clause Y1 in combination with the features of the method of clause U1.
Clause Y15. The method of clause Y1 in combination with the features of the method of any of clauses W1 or W2.
Clause Y16. The method of any of clauses Y1 through Y15, wherein the on-body device comprises:
a sensor;
wireless communication electronics;
sensor interface electronics configured to receive a signal from the sensor, the signal indicative of an analyte level of a user;
a housing configured to retain the wireless communication electronics and sensor interface electronics; and
an adhesive patch adhesively coupled with the housing,
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the level of the analyte in the bodily fluid and output the signal to the sensor interface electronics.
Clause Z1. An on-body analyte monitoring system, comprising: an on-body device configured to be held on the body of a subject, the on-body device comprising a housing configured to hold first electronics, wherein the first electronics are configured to: transmit a first advertisement in a synchronous advertising-based communication session; receive a first response acknowledging receipt of the first advertisement and including a supplementary information request; transmit a second advertisement in the synchronous advertising-based communication session with at least a portion of the supplementary information requested by the supplementary information request; and receive a second response acknowledging receipt of the second advertisement.
Clause Z2. The system of clause Z1, wherein the supplementary information requested by the supplementary information request is missing analyte data.
Clause Z3. The system of any of clauses Z1 or Z2 in combination with the features of the system of any of clauses A1 through A94 or A96 through A101.
Clause Z4. The system of any of clauses Z1 or Z2 in combination with the features of the system of any of clauses C1 through C18 or C20.
Clause Z5. The system of any of clauses Z1 or Z2 in combination with the features of the system of any of clauses F1 through F25 or F27.
Clause Z6. The system of any of clauses Z1 or Z2 in combination with the features of the system of any of clauses H1 through H18 or H20.
Clause Z7. The system of any of clauses Z1 or Z2 in combination with the features of the system of any of clauses AF1 through AF22.
Clause Z8. The system of any of clauses Z1 or Z2 in combination with the features of the system of any of clauses J1 through J169.
Clause Z9. The system of any of clauses Z1 or Z2 in combination with the features of the system of any of clauses L1 or L2.
Clause Z10. The system of any of clauses Z1 or Z2 in combination with the features of the system of clause N1.
Clause Z11. The system of any of clauses Z1 or Z2 in combination with the features of the system of clause O1.
Clause Z12. The system of any of clauses Z1 or Z2 in combination with the features of the system of clause Q1.
Clause Z13. The system of any of clauses Z1 or Z2 in combination with the features of the system of clause R1.
Clause Z14. The system of any of clauses Z1 or Z2 in combination with the features of the system of clause T1.
Clause Z15. The system of any of clauses Z1 or Z2 in combination with the features of the system of any of clauses V1 or V2.
Clause Z16. The system of any of clauses Z1 or Z2 in combination with the features of the system of clause X1.
Clause Z17. The system of any of clauses Z1 through Z16, wherein the on-body device further comprises:
a sensor; and
an adhesive patch adhesively coupled with the housing,
wherein the first electronics comprise: wireless communication electronics; and sensor interface electronics configured to receive a signal from the sensor, the signal indicative of an analyte level of a user;
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the level of the analyte in the bodily fluid and output the signal to the sensor interface electronics.
Clause AA1. An on-body analyte monitoring system, comprising: a receiver device comprising a housing configured to hold receiver electronics, wherein the receiver electronics are configured to: receive a first advertisement in a synchronous advertising-based communication session; transmit a first response acknowledging receipt of the first advertisement and including a supplementary information request; receive a second advertisement in the synchronous advertising-based communication session with at least a portion of supplementary information requested by the supplementary information request; and transmit a second response acknowledging receipt of the second advertisement.
Clause AA2. The system of clause AA1, wherein the supplementary information requested by the supplementary information request is missing analyte data.
Clause AA3. The system of any of clauses AA1 or AA2 in combination with the features of the system of any of clauses A1 through A94 or A96 through A101.
Clause AA4. The system of any of clauses AA1 or AA2 in combination with the features of the system of any of clauses C1 through C18 or C20.
Clause AA5. The system of any of clauses AA1 or AA2 in combination with the features of the system of any of clauses F1 through F25 or F27.
Clause AA6. The system of any of clauses AA1 or AA2 in combination with the features of the system of any of clauses H1 through H18 or H20.
Clause AA7. The system of any of clauses AA1 or AA2 in combination with the features of the system of any of clauses AF1 through AF22.
Clause AA8. The system of any of clauses AA1 or AA2 in combination with the features of the system of any of clauses J1 through J169.
Clause AA9. The system of any of clauses AA1 or AA2 in combination with the features of the system of any of clauses L1 or L2.
Clause AA10. The system of any of clauses AA1 or AA2 in combination with the features of the system of clause N1.
Clause AA11. The system of any of clauses AA1 or AA2 in combination with the features of the system of clause O1.
Clause AA12. The system of any of clauses AA1 or AA2 in combination with the features of the system of clause Q1.
Clause AA13. The system of any of clauses AA1 or AA2 in combination with the features of the system of clause R1.
Clause AA14. The system of any of clauses AA1 or AA2 in combination with the features of the system of clause T1.
Clause AA15. The system of any of clauses AA1 or AA2 in combination with the features of the system of any of clauses V1 or V2.
Clause AA16. The system of any of clauses AA1 or AA2 in combination with the features of the system of clause X1.
Clause AB1. A method of wireless communication in an on-body analyte monitoring system, comprising: transmitting a first advertisement, by an on-body device, on each of a plurality of primary channels of a Bluetooth protocol; receiving a response from a receiver device on one of the plurality of primary channels that includes a supplementary information request; transmitting a second advertisement, by the on-body device, on the one of the plurality of primary channels, the second advertisement comprising a pointer designating a secondary channel and a time offset; and transmitting a third advertisement, by the on-body device, on the designated secondary channel after the time offset, wherein the third advertisement comprises at least a portion of supplementary information requested by the supplementary information request.
Clause AB2. The method of clause AB1 in combination with the features of the method of any of clauses B1 through B70 or B72 through B77.
Clause AB3. The method of clause AB1 in combination with the features of the method of any of clauses D1 through D9 or D10.
Clause AB4. The method of clause AB1 in combination with the features of the method of any of clauses E1 through E21 or E23.
Clause AB5. The method of clause AB1 in combination with the features of the method of any of clauses G1 through G13 or G15.
Clause AB6. The method of clause AB1 in combination with the features of the method of any of clauses AE1 through AE22.
Clause AB7. The method of clause AB1 in combination with the features of the method of any of clauses I1 through I185.
Clause AB8. The method of clause AB1 in combination with the features of the method of clause K1.
Clause AB9. The method of clause AB1 in combination with the features of the method of any of clauses M1 through M6.
Clause AB11. The method of clause AB1 in combination with the features of the method of any of clauses P1 through P4.
Clause AB13. The method of clause AB1 in combination with the features of the method of clause S1.
Clause AB14. The method of clause AB1 in combination with the features of the method of clause U1.
Clause AB15. The method of clause AB1 in combination with the features of the method of any of clauses W1 or W2.
Clause AB16. The method of any of clauses AB1 through AB15, wherein the on-body device further comprises:
a sensor;
wireless communication electronics;
sensor interface electronics configured to receive a signal from the sensor, the signal indicative of an analyte level of a user;
a housing configured to retain the wireless communication electronics and sensor interface electronics; and
an adhesive patch adhesively coupled with the housing,
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the level of the analyte in the bodily fluid and output the signal to the sensor interface electronics.
Clause AC1. An on-body analyte monitoring system, comprising: an on-body device configured to be held on the body of a subject, the on-body device comprising a housing configured to hold first electronics, wherein the first electronics are configured to: transmit a first advertisement on each of a plurality of primary channels of a Bluetooth protocol; receive a response from a receiver device on one of the plurality of primary channels that includes a supplementary information request; transmit a second advertisement on the one of the plurality of primary channels, the second advertisement comprising a pointer designating a secondary channel and a time offset; and transmit a third advertisement on the designated secondary channel after the time offset, wherein the third advertisement comprises at least a portion of supplementary information requested by the supplementary information request.
Clause AC2. The system of clause AC1, wherein the supplementary information requested by the supplementary information request is missing analyte data.
Clause AC3. The system of any of clauses AC1 or AC2 in combination with the features of the system of any of clauses A1 through A94 or A96 through A101.
Clause AC4. The system of any of clauses AC1 or AC2 in combination with the features of the system of any of clauses C1 through C18 or C20.
Clause AC5. The system of any of clauses AC1 or AC2 in combination with the features of the system of any of clauses F1 through F25 or F27.
Clause AC6. The system of any of clauses AC1 or AC2 in combination with the features of the system of any of clauses H1 through H18 or H20.
Clause AC7. The system of any of clauses AC1 or AC2 in combination with the features of the system of any of clauses AF1 through AF22.
Clause AC8. The system of any of clauses AC1 or AC2 in combination with the features of the system of any of clauses J1 through J169.
Clause AC9. The system of any of clauses AC1 or AC2 in combination with the features of the system of any of clauses L1 or L2.
Clause AC10. The system of any of clauses AC1 or AC2 in combination with the features of the system of clause N1.
Clause AC11. The system of any of clauses AC1 or AC2 in combination with the features of the system of clause O1.
Clause AC12. The system of any of clauses AC1 or AC2 in combination with the features of the system of clause Q1.
Clause AC13. The system of any of clauses AC1 or AC2 in combination with the features of the system of clause R1.
Clause AC14. The system of any of clauses AC1 or AC2 in combination with the features of the system of clause T1.
Clause AC15. The system of any of clauses AC1 or AC2 in combination with the features of the system of any of clauses V1 or V2.
Clause AC16. The system of any of clauses AC1 or AC2 in combination with the features of the system of clause X1.
Clause AC17. The method of any of clauses AC1 through AC16, wherein the on-body device further comprises:
a sensor; and
an adhesive patch adhesively coupled with the housing,
wherein the first electronics comprise: wireless communication electronics; and sensor interface electronics configured to receive a signal from the sensor, the signal indicative of an analyte level of a user;
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the level of the analyte in the bodily fluid and output the signal to the sensor interface electronics.
Clause AD1. An on-body analyte monitoring system, comprising: a receiver device comprising a housing configured to hold receiver electronics, wherein the receiver electronics are configured to: receive a first advertisement on a primary channel of a Bluetooth protocol; transmit a response on the primary channel that includes a supplementary information request for missing analyte data; receive a second advertisement on the primary channel, the second advertisement comprising a pointer designating a secondary channel and a time offset; and receive a third advertisement on the designated secondary channel after the time offset, wherein the third advertisement comprises at least a portion of the supplementary information requested by the supplementary information request.
Clause AD2. The system of clause AD1 in combination with the features of the system of any of clauses A1 through A94 or A96 through A101.
Clause AD3. The system of clause AD1 in combination with the features of the system of any of clauses C1 through C18 or C20.
Clause AD4. The system of clause AD1 in combination with the features of the system of any of clauses F1 through F25 or F27.
Clause AD5. The system of clause AD1 in combination with the features of the system of any of clauses H1 through H18 or H20.
Clause AD6. The system of clause AD1 in combination with the features of the system of any of clauses AF1 through AF22.
Clause AD7. The system of clause AD1 in combination with the features of the system of any of clauses J1 through J169.
Clause AD8. The system of clause AD1 in combination with the features of the system of any of clauses L1 or L2.
Clause AD9. The system of clause AD1 in combination with the features of the system of clause N1.
Clause AD10. The system of clause AD1 in combination with the features of the system of clause O1.
Clause AD11. The system of clause AD1 in combination with the features of the system of clause Q1.
Clause AD12. The system of clause AD1 in combination with the features of the system of clause R1.
Clause AD13. The system of clause AD1 in combination with the features of the system of clause T1.
Clause AD14. The system of clause AD1 in combination with the features of the system of any of clauses V1 or V2.
Clause AD15. The system of clause AD1 in combination with the features of the system of clause X1.
Clause AE1. A method of using an analyte monitoring system, comprising: receiving a first clock accuracy parameter of a first device; substituting a second clock accuracy parameter for the first clock accuracy parameter of the first device if a substitution condition is satisfied; and if the substitution condition is satisfied, utilizing wireless communication electronics of a second device at a time determined, at least in part, based on the second clock accuracy parameter.
Clause AE2. The method of clause AE1, further comprising substituting the first clock accuracy parameter with the second clock accuracy parameter of the first device if the first clock accuracy parameter satisfies a substitution condition.
Clause AE3. The method of clause AE2, wherein the substitution condition is a threshold.
Clause AE4. The method of clause AE3, further comprising substituting the second clock accuracy parameter for the first clock accuracy parameter of the first device if the first clock accuracy parameter is representative of a clock accuracy less accurate than the threshold.
Clause AE5. The method of clause AE2, wherein the substitution condition is a presence of the first clock accuracy parameter in a data structure.
Clause AE6. The method of clause AE1, further comprising substituting the second clock accuracy parameter for the first clock accuracy parameter if the first clock accuracy parameter is present in the data structure.
Clause AE7. The method of any of clauses AE5 or AE6, wherein the data structure further comprises a substitution clock accuracy corresponding to the first clock accuracy parameter, wherein the second clock accuracy parameter is the substitution clock accuracy.
Clause AE8. The method of any of clauses AE1 through AE7, further comprising receiving a wireless transmission at the second device, wherein the wireless transmission comprises the first clock accuracy parameter of the first device, prior to substituting the second clock accuracy parameter for the first clock accuracy parameter.
Clause AE9. The method of any of clauses AE1 through AE8, wherein the second clock accuracy parameter is stored in memory of the on-body device.
Clause AE10. The method of any of clauses AE1 through AE9, further comprising identifying or determining the value of the second clock accuracy parameter prior to substituting the second clock accuracy parameter for the first clock accuracy parameter.
Clause AE11. The method of clause AE10, wherein identifying or determining the second clock accuracy parameter comprises selecting the second clock accuracy parameter from a plurality of second clock accuracy parameters stored in the on-body device.
Clause AE12. The method of any of clauses AE1 through AE11, wherein the second clock accuracy parameter is substituted for the first clock accuracy parameter in at least one algorithm configured to determine timing for activating the wireless communication electronics of the second device.
Clause AE13. The method of clause AE12, wherein the at least one algorithm is configured to determine the time for activating the wireless communication electronics of the second device.
Clause AE14. The method of any of clauses AE1 through AE13, wherein substituting the second clock accuracy parameter for the first clock accuracy parameter comprises overwriting the first clock accuracy parameter with the second clock accuracy parameter in memory of the second device.
Clause AE15. The method of any of clauses AE1 through AE14, wherein the first device is a receiver device and the second device is an on-body device.
Clause AE16. The method of any of clauses AE1 through AE15, further comprising substituting a third clock accuracy parameter for the second clock accuracy parameter.
Clause AE17. The method of clause AE16, the method further comprising activating the wireless communication electronics of the second device at a second time determined, at least in part, based on the third clock accuracy parameter.
Clause AE18. The method of any of clauses AE1 through AE15, wherein the time is a first time, the method further comprising: detecting a failure to receive an incoming transmission from the first device after activating the wireless communication electronics at the first time determined, at least in part, based on the second clock accuracy parameter.
Clause AE19. The method of clause AE18, further comprising: substituting a third clock accuracy parameter for the second clock accuracy parameter; and activating the wireless communication electronics of the second device at a second time determined, at least in part, based on the third clock accuracy parameter.
Clause AE20. The method of any of clauses AE16 through AE19, wherein the third clock accuracy parameter is between the first clock parameter and the second clock accuracy parameter.
Clause AE21. The method of any of clauses AE1 through AE15, further comprising iteratively adjusting the second clock accuracy parameter to different values based on detected failures to receive transmissions from the first device by the second device.
Clause AE22. The method of clause AE21, wherein iterative adjustments continue until a plurality of incoming transmissions are successively received over a predetermined time period or a predetermined number of wireless communication electronics activation instances.
Clause AE23. The method of any of clauses AE1 through AE22, wherein the on-body device further comprises:
a sensor;
wireless communication electronics;
sensor interface electronics configured to receive a signal from the sensor, the signal indicative of an analyte level of a user;
a housing configured to retain the wireless communication electronics and sensor interface electronics; and
an adhesive patch adhesively coupled with the housing,
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the level of the analyte in the bodily fluid and output the signal to the sensor interface electronics.
Clause AF1. An analyte monitoring system, comprising: an on-body device comprising wireless communication electronics, wherein the on-body device is configured to receive a first clock accuracy parameter of a receiver device, substitute a second clock accuracy parameter for the first clock accuracy parameter of the receiver device if a substitution condition is satisfied, and utilize the wireless communication electronics at a time determined, at least in part, with the second clock accuracy parameter if substituted.
Clause AF2. The system of clause AF1, wherein the on-body device is further configured to substitute the second clock accuracy parameter for the first clock accuracy parameter of the receiver device if the first clock accuracy parameter satisfies the substitution condition.
Clause AF3. The system of any of clauses AF1 or AF2, wherein the substitution condition is a threshold.
Clause AF4. The system of clause AF3, wherein the on-body device is further configured to substitute the second clock accuracy parameter for the first clock accuracy parameter if the first clock accuracy parameter is representative of a clock accuracy less accurate than the threshold.
Clause AF5. The system of any of clauses AF1 or AF2, wherein the substitution condition is a presence of the first clock accuracy parameter in a data structure.
Clause AF6. The system of clause AF5, wherein the on-body device is further configured to substitute the second clock accuracy parameter for the first clock accuracy parameter if the first clock accuracy parameter is present in the data structure.
Clause AF7. The system of any of clauses AF5 or AF6, wherein the data structure further comprises a substitution clock accuracy corresponding to the first clock accuracy parameter, wherein the second clock accuracy parameter is the substitution clock accuracy.
Clause AF8. The system of any of clauses AF1 through AF7, wherein the on-body device is further configured to receive a wireless transmission comprising the first clock accuracy parameter of the receiver device.
Clause AF9. The system of any of clauses AF1 through AF8, wherein the second clock accuracy parameter is stored in memory of the on-body device.
Clause AF10. The system of any of clauses AF1 through AF9, wherein the on-body device is further configured to identify or determine the value of the second clock accuracy parameter prior to substituting the second clock accuracy parameter for the first clock accuracy parameter.
Clause AF11. The system of clause AF10, wherein the on-body device is configured to identify or determine the value of the second clock accuracy parameter by selection of the second clock accuracy parameter from a plurality of second clock accuracy parameters stored in the on-body device.
Clause AF12. The system of any of clauses AF1 through AF11, wherein the on-body device is configured to substitute the second clock accuracy parameter for the first clock accuracy parameter in at least one algorithm configured to determine timing for activation of the wireless communication electronics of the on-body device.
Clause AF13. The system of clause AF12, wherein the at least one algorithm is configured to determine the time, and wherein the time is for activation of the wireless communication electronics of the on-body device.
Clause AF14. The system of any of clauses AF1 through AF13, wherein the on-body device is configured to substitute the second clock accuracy parameter for the first clock accuracy parameter by way of an overwrite of the first clock accuracy parameter with the second clock accuracy parameter in memory of the on-body device.
Clause AF15. The system of any of clauses AF1 through AF14, wherein the on-body device is further configured to substitute a third clock accuracy parameter for the second clock accuracy parameter.
Clause AF16. The system of clause AF15, wherein the time is a first time, and wherein the on-body device is further configured to utilize the wireless communication electronics at a second time determined, at least in part, based on the third clock accuracy parameter.
Clause AF17. The system of any of clauses AF1 through AF14, wherein the time is a first time, and wherein the on-body device is further configured to detect a failure to receive an incoming transmission from the receiver device after activation of the wireless communication electronics at the first time determined, at least in part, based on the second clock accuracy parameter.
Clause AF18. The system of clause AF17, wherein the on-body device is further configured to substitute a third clock accuracy parameter for the second clock accuracy parameter, and utilize the wireless communication electronics of the on-body device at a second time determined, at least in part, based on the third clock accuracy parameter.
Clause AF19. The system of any of clauses AF15 through AF18, wherein the third clock accuracy parameter is between the first clock parameter and the second clock accuracy parameter.
Clause AF20. The system of any of clauses AF1 through AF14, wherein the on-body device is further configured to iteratively adjust the second clock accuracy parameter to different values based on detected failures to receive transmissions from the receiver device.
Clause AF21. The system of clause AF20, wherein the on-body device is further configured to iteratively adjust until a plurality of incoming transmissions are successively received over a predetermined time period or a predetermined number of wireless communication electronics activation instances.
Clause AF22. The system of any of clauses AF1 through AF21, further comprising the receiver device.
Clause AF23. The system of any of clauses AF1 through AF22, wherein the an on-body device further comprises: a sensor; sensor interface electronics configured to receive a signal from the sensor, the signal indicative of a level of an analyte of a user; a housing configured to retain the wireless communication electronics and sensor interface electronics; and an adhesive patch adhesively coupled with the housing, wherein the sensor comprises: a proximal portion configured to be positioned above skin of the user and to be electrically coupled with the sensor interface electronics; and a distal portion configured to be transcutaneously positioned through the user's skin and in contact with a bodily fluid of the user, wherein the distal portion of the sensor is further configured to detect an analyte in the bodily fluid and output the signal to the sensor interface electronics.
Clause AG1. A method of using an analyte monitoring system, comprising: receiving a first clock accuracy parameter of a first device and substituting a second clock accuracy parameter for the first clock accuracy parameter of the first device if a substitution condition is satisfied.
Clause AG2. The method of clause AG1, wherein the method further comprises: if the substitution condition is satisfied, utilizing wireless communication electronics of a second device at a time determined, at least in part, based on the second clock accuracy parameter.
Clause AG3. The method of any of clauses AG1 or AG2, wherein the first device is an on-body device that further comprises:
a sensor;
wireless communication electronics;
sensor interface electronics configured to receive a signal from the sensor, the signal indicative of an analyte level of a user;
a housing configured to retain the wireless communication electronics and sensor interface electronics; and
an adhesive patch adhesively coupled with the housing,
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the level of the analyte in the bodily fluid and output the signal to the sensor interface electronics.
Clause AH1. An analyte monitoring system, comprising: an on-body device comprising wireless communication electronics, wherein the on-body device is configured to receive a first clock accuracy parameter of a receiver device and, if a substitution condition is satisfied, substitute a second clock accuracy parameter for the first clock accuracy parameter of the receiver device.
Clause AH2. The system of clause AH1, wherein the on-body device is further configured to: if a substitution condition is satisfied, utilize the wireless communication electronics at a time determined, at least in part, with the second clock accuracy parameter if substituted.
Clause AH3. The system method of any of clauses AH1 or AH2, wherein the on-body device further comprises:
a sensor;
wireless communication electronics;
sensor interface electronics configured to receive a signal from the sensor, the signal indicative of an analyte level of a user;
a housing configured to retain the wireless communication electronics and sensor interface electronics; and
an adhesive patch adhesively coupled with the housing,
wherein the sensor comprises: a proximal portion configured to be positioned above skin of the subject and to be electrically coupled with the sensor interface electronics; and a distal portion configured to be transcutaneously positioned through the subject's skin and in contact with a bodily fluid of the subject, wherein the distal portion of the sensor is further configured to detect the level of the analyte in the bodily fluid and output the signal to the sensor interface electronics.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, and/or that substitute features, elements, components, functions, and steps from one embodiment with those of another, even if the present description does not explicitly state, in a particular instance, that such combinations and/or substitutions are possible. It is explicitly acknowledged that express recitation of every possible combination and substitution is overly burdensome, especially given that the permissibility of each and every such combination and substitution will be readily recognized by those of ordinary skill in the art.

To the extent the embodiments disclosed herein include or operate in association with memory, storage, and/or computer readable media, then that memory, storage, and/or computer readable media are non-transitory. Accordingly, to the extent that memory, storage, and/or computer readable media are covered by one or more claims, then that memory, storage, and/or computer readable media is only non-transitory.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

1-130. (canceled)

131. An analyte monitoring system, comprising:
an on-body device comprising:
a sensor;
on-body device electronics comprising: wireless communication electronics; and
sensor interface electronics configured to receive a signal from the sensor, the signal indicative of a level of an analyte of a user;
a housing configured to retain the on-body device electronics; and
an adhesive patch adhesively coupled with the housing,
wherein the sensor comprises:
a proximal portion configured to be positioned above skin of the user and to be electrically coupled with the sensor interface electronics; and
a distal portion configured to be transcutaneously positioned through the user's skin and in contact with a bodily fluid of the user,
wherein the distal portion of the sensor is further configured to detect an analyte in the bodily fluid and output the signal to the sensor interface electronics;
wherein the on-body device is configured to receive a first clock accuracy parameter of a receiver device, substitute a second clock accuracy parameter for the first clock accuracy parameter of the receiver device if a substitution condition is satisfied, and utilize the wireless communication electronics at a time determined, at least in part, with the second clock accuracy parameter if substituted.

132. The system of claim 131, wherein the on-body device is further configured to substitute the second clock accuracy parameter for the first clock accuracy parameter of the receiver device if the first clock accuracy parameter satisfies the substitution condition.

133. The system of claim 131, wherein the substitution condition is a threshold, and wherein the on-body device is further configured to substitute the second clock accuracy parameter for the first clock accuracy parameter if the first clock accuracy parameter is representative of a clock accuracy less accurate than the threshold.

134. The system of claim 132, wherein the substitution condition is a presence of the first clock accuracy parameter in a data structure.

135. The system of claim 134, wherein the on-body device is further configured to substitute the second clock accuracy parameter for the first clock accuracy parameter if the first clock accuracy parameter is present in the data structure.

136. The system of claim 135, wherein the data structure further comprises a substitution clock accuracy corresponding to the first clock accuracy parameter, wherein the second clock accuracy parameter is the substitution clock accuracy.

137. The system of claim 131, wherein the on-body device is further configured to receive a wireless transmission comprising the first clock accuracy parameter of the receiver device.

138. The system of claim 131, wherein the on-body device is further configured to identify or determine the value of the second clock accuracy parameter prior to substituting the second clock accuracy parameter for the first clock accuracy parameter.

139. The system of claim 131, wherein the on-body device is configured to substitute the second clock accuracy parameter for the first clock accuracy parameter in at least one algorithm configured to determine timing for activation of the wireless communication electronics of the on-body device.

140. The system of claim 139, wherein the at least one algorithm is configured to determine the time, wherein the time is for activation of the wireless communication electronics of the on-body device.

141. The system of claim 131, wherein the on-body device is configured to substitute the second clock accuracy parameter for the first clock accuracy parameter by way of an overwrite of the first clock accuracy parameter with the second clock accuracy parameter in memory of the on-body device.

142. The system of claim 131, wherein the on-body device is further configured to substitute a third clock accuracy parameter for the second clock accuracy parameter.

143. The system of claim 142, wherein the time is a first time, and wherein the on-body device is further configured to utilize the wireless communication electronics at a second time determined, at least in part, based on the third clock accuracy parameter.

144. The system of claim 131, wherein the time is a first time, and wherein the on-body device is further configured to detect a failure to receive an incoming transmission from the receiver device after activation of the wireless communication electronics at the first time determined, at least in part, based on the second clock accuracy parameter.

145. The system of claim 144, wherein the on-body device is further configured to substitute a third clock accuracy parameter for the second clock accuracy parameter, and activate the wireless communication electronics of the on-body device at a second time determined, at least in part, based on the third clock accuracy parameter.

146. A method of using an analyte monitoring system, comprising:
receiving a first clock accuracy parameter of a first device;
substituting a second clock accuracy parameter for the first clock accuracy parameter of the first device if a substitution condition is satisfied; and
utilizing wireless communication electronics of a second device at a time determined, at least in part, based on the second clock accuracy parameter if substituted.

147. The method of claim 146, further comprising substituting the second clock accuracy parameter for the first clock accuracy parameter of the first device if the first clock accuracy parameter satisfies the substitution condition.

148. The method of claim 147, wherein the substitution condition is a presence of the first clock accuracy parameter in a data structure, wherein the method further comprises substituting the second clock accuracy parameter for the first clock accuracy parameter if the first clock accuracy parameter is present in the data structure.

149. The method of claim 146, wherein the second clock accuracy parameter is substituted for the first clock accuracy parameter in at least one algorithm configured to determine timing for activating the wireless communication electronics of the second device.

150. The method of claim 146, wherein substituting the second clock accuracy parameter for the first clock accuracy parameter comprises overwriting the first clock accuracy parameter with the second clock accuracy parameter in memory of the second device.
